(19) United States
(12) Patent Application Publication
(10) Pub. No.: US 2026/0191835 A1
(43) Pub. Date: Jul. 9, 2026

(54) COMBINATIONS COMPRISING A MENIN-MLL INHIBITOR AND AT LEAST ONE OTHER THERAPEUTIC AGENT

(71) Applicant: JANSSEN PHARMACEUTICA NV, Beerse (BE)

(72) Inventors: Nikki DASKALAKIS,, Colts Neck, NJ (US); Diane Christina GUTTKE, Sellersville, PA (US); Min Chul KWON, Mol (BE); Lucille Angela FERRANTE, Winston-Salem, NC (US); Kathryn Elizabeth PACKMAN, Cambridge, MA (US); Eva Christine PIETSCH, Spring House, PA (US); Ulrike PHILIPPAR, Antwerpen (BE); Sumia ALI-AHMED, Beerse (BE); Wei CAI, Shanghai (CN); Johannes Wilhelmus J. THURING, Antwerpen (BE); Fabian HULPIA, Sint-Niklaas (BE); Xuedong DAI, Shanghai (CN); Ming LI, Shanghai (CN); Xiangjun DENG, Shanghai (CN); Chao LIANG, Shanghai (CN); Alicia Tee Fuay NG, Shanghai (CN); Zhen SUN, Shanghai (CN); Zhigao ZHANG, Shanghai (CN); Samuël Dominique DEMIN, Mechelen (BE); Natalia Nikolaevna DYUBANKOVA, Herent (BE); Matthieu Dominique JOUFFROY, Tielen (BE); Susan LEPRI, Turnhout (BE); Nicolas Freddy J DARVILLE, Linkhout (BE); Vineet PANDE, Vosselaar (BE); Wim Bert Griet SCHEPENS, Sint-Katelijne-Waver (BE); James Patrick EDWARDS, Ambler, PA (US); Olivier Alexis Georges QUEROLLE, Saint Vigor (FR)

(21) Appl. No.: 19/132,126

(22) PCT Filed: Nov. 29, 2023
(86) PCT No.: PCT/CN2023/134914

§ 371 Date: May 22, 2025

(30) Foreign Application Priority Data
Nov. 30, 2022 (WO) PCT/CN2022/135444

(51) Int. Cl. A61K 31/4355; A61K 31/437; A61K 31/438; A61K 31/4545; A61K 31/55; A61K 45/06; A61P 35/02

(52) U.S. Cl. CPC A61K 31/4355; A61K 31/437; A61K 31/438; A61K 31/4545; A61K 31/55; A61K 45/06; A61P 35/02

(57) ABSTRACT

Disclosed are combinations comprising a therapeutically effective amount of a menin-MLL inhibitor of Formula (I) or a pharmaceutically acceptable salt or a solvate thereof; and a therapeutically effective amount of at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor. Also disclosed are methods for treating a subject who has been diagnosed with cancer using such combinations. Compounds are represented by Formula (I) as follows:

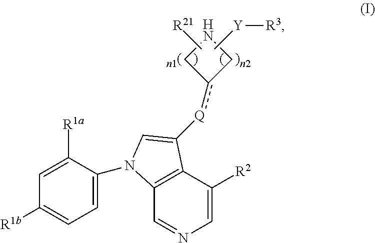

wherein the variables are defined herein.

### FIELD OF THE INVENTION

The present invention relates to novel combinations comprising a therapeutically effective amount of a menin-mixed-lineage leukemia 1 (menin-MLL) inhibitor of Formula (I), or a pharmaceutically acceptable salt or a solvate thereof; and a therapeutically effective amount of at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor; as well as to methods for treating a subject diagnosed with cancer using such combinations.

### BACKGROUND OF THE INVENTION

Cancer is the leading cause of death worldwide. Of the 10 million cancer deaths recorded by GLOBOCAN in 2020, 7.1% are attributed to hematopoietic disorders. Accordingly, new treatment modalities are urgently needed for hematopoietic disorders, including acute myeloid leukemia (AML), myelodysplastic syndrome (MDS) and acute lymphoblastic leukemia (ALL) as further detailed below.

AML is a common hematological malignancy whose incidence rises from 3:100,000 in young adults to greater than 20:100,000 in older adults. For patients<60 years of age, overall survival (OS) is 40 to 50%, but is only 5% for patients>60 years of age. The majority of newly diagnosed patients with AML are over the age of 60. In this patient population, standard induction chemotherapy is often not an option due to increased treatment-related mortality as a result of age and co-morbidities. Standard of care for AML patients unfit for combination chemotherapy is treatment with hypomethylating agents (azacitidine or decitabine) or low dose cytarabine. Relapsed/refractory AML with a FMS-like tyrosine kinase 3 (FLT3) mutation is treated with a FLT3 kinase inhibitor (e.g., gilteritinib, midostaurin). Despite these frontline treatments, median OS is only about 10 months. In all types of AML, disease relapse is common despite an initial therapeutic response and is the most common reason for death. Standard chemotherapy and allogeneic stem cell transplant (when used) often fail to eradicate all tumor-propagating cells and select for chemotherapy-resistant leukemia-propagating subclones. Patients refractory to salvage therapy are treated palliatively, as current treatment options are extremely limited. These patients have a median survival of 2 months. In addition, patients with newly diagnosed intermediate or higher-risk MDS and those who relapse after standard care have a poor prognosis and high risk of progression to AML. Therefore, there is an urgent need for new treatment modalities for relapsed/refractory (R/R) AML and MDS patients, newly diagnosed AML patients ineligible for induction chemotherapy based on age and co-morbidities, and newly diagnosed intermediate/high/very high risk MDS patients.

ALL is a hematologic malignancy propagated by impaired differentiation, proliferation, and accumulation of lymphoid progenitor cells in the bone marrow and/or extramedullary sites. ALL represents 12% of all leukemia cases and is the most common childhood acute leukemia, with a worldwide incidence projected to be 1 to 4.75 per 100,000 people. ALL represents about 20% of adult leukemias. Despite high rates of complete remission (CR) (80% to 90%) with current therapies, the majority of adult patients with ALL relapse. The 5-year overall survival rate is approximately 30 to 40% in adults and elderly patients. Therefore, there is an urgent need for new treatment modalities for the treatment of cancer, in particular relapsed/refractory ALL, more particularly in adult and especially elderly patients.

### SUMMARY OF THE INVENTION

Embodiments of the present invention relate to novel combinations of a menin-MLL inhibitor of Formula (I), or a pharmaceutically acceptable salt or a solvate thereof; and at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

Embodiments of the present invention relate to uses of such combinations for treating a subject who has been diagnosed with a hematopoietic disorder, including but not limited to, blood cancers, using a menin-MLL inhibitor described herein in combination with at least one other therapeutic agent.

Embodiments of the present invention relate to novel methods for treating a subject who has been diagnosed with a hematopoietic disorder using such combinations. Embodiments of the novel methods comprise administering to the subject a therapeutically effective amount of a menin-MLL inhibitor as described herein; and a therapeutically effective amount of at least one other therapeutic agent; wherein the menin-MLL inhibitor is a compound of Formula (I), or a pharmaceutically acceptable salt or a solvate thereof.

Embodiments of the present invention relate to novel methods for treating a subject who has been diagnosed with a hematopoietic disorder using such combinations. Embodiments of the novel methods comprise administering to the subject a therapeutically effective amount of a menin-MLL inhibitor as described herein; and a therapeutically effective amount of at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor; wherein the menin-MLL inhibitor is a compound of Formula (I), or a pharmaceutically acceptable salt or a solvate thereof.

In some embodiments, the present invention is directed to methods for treating a subject who has been diagnosed with a hematopoietic disorder, the methods comprising administering to the subject a therapeutically effective amount of a menin-MLL inhibitor of Formula (I), or a pharmaceutically acceptable salt or a solvate thereof; and a therapeutically effective amount of azacitidine or a pharmaceutically acceptable salt or solvate thereof.

In some embodiments, the present invention is directed to methods for treating a subject who has been diagnosed with a hematopoietic disorder, the methods comprising administering to the subject a therapeutically effective amount of a menin-MLL inhibitor of Formula (I), or a pharmaceutically acceptable salt or a solvate thereof; and a therapeutically effective amount of azacitidine or a pharmaceutically acceptable salt or solvate thereof; wherein the azacitidine, or a pharmaceutically acceptable salt or solvate thereof, is administered to the subject prior to, simultaneous with, or after the administration of the menin-MLL inhibitor.

In embodiments, the menin-MLL inhibitor of Formula (I) is:

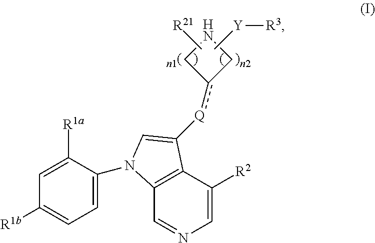

and the tautomers and the stereoisomeric forms thereof, wherein
Q represents —CHR^{y}—, —O—, —C(═O)—, —NR^{q}—, or —CR^{y}═; the dotted line is an optional additional bond to form a double bond in case Q represents —CR^{y}═;
R^{1a} represents hydrogen; cyano; halo; Het; —C(═O)—NR^{xa}R^{xb}; —S(═O)₂—R¹⁸;
—C(═O)—O—C₁₋₄alkyl-NR^{22a}R^{22b}; —C(═O)—O—C₁₋₄alkyl;

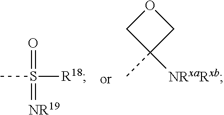

R¹⁸ represents C₁₋₆alkyl or C₃₋₆cycloalkyl;
R¹⁹ represents hydrogen or C₁₋₆alkyl;
or R¹⁸ and R¹⁹ are taken together to form —(CH₂)₃—, —(CH₂)₄— or —(CH₂)₅—;
Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, —C₁₋₄alkyl-OH, halo, CF₃, C₃₋₆cycloalkyl, Het³, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents selected from the group consisting of halo and OR²³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents each independently selected from the group consisting of halo and OR²³;
R²³ represents hydrogen or C₁₋₄alkyl optionally substituted with one, two or three halo;
R^{1b} represents hydrogen, F, Cl, or —O—C₁₋₄alkyl;
R² represents halo, C₃₋₆cycloalkyl, C₁₋₄alkyl, —O—C₁₋₄alkyl, cyano, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or

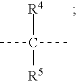

n1 is selected from 1 and 2;
n2 is selected from 1, 2, 3 and 4;
R^{y} represents hydrogen, —OH, C₁₋₄alkyl, —C₁₋₄alkyl-OH, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²; Cy²;
C₁₋₈alkyl; and C₁₋₈alkyl substituted with one, two, three or four substituents each independently selected from the group consisting of —C(═O)—NR^{10a}R^{10b}, —C(═O)—Het^{6a}, —C(═O)—Het^{6b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², Ar¹, and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;
R^{8a} and R^{8b} are each independently selected from the group consisting of hydrogen;
C₁₋₆alkyl; —(C═O)—C₁₋₄alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;
Ar¹ represents phenyl optionally substituted with one, two or three substituents each independently selected from the group consisting of C₁₋₄alkyl, halo, —O—C₁₋₄alkyl, —CF₃, —OH, —S(═O)₂—C₁₋₄alkyl, and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl, 1,2,4-oxadiazolyl, pyridazinyl or triazolyl; which may be optionally substituted on one nitrogen atom with R^{6a};
R⁶ and R^{6a} are each independently selected from the group consisting of
Het³; Het⁴; —C(═O)—NH—Cy¹; —C(═O)—NH—R⁸; —C(═O)—Het^{6a}; —C(═O)—NR^{10d}R^{10e};
—C(═O)—O—C₁₋₄alkyl; —S(═O)₂—C₁₋₄alkyl;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—N(C₁₋₄alkyl)₂, —C(═O)—NH—C₁₋₄alkyl-C₃₋₆cycloalkyl, —C(═O)—OH, —NR^{11a}R^{11b} and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl,
—C(═O)—N(C₁₋₄alkyl)₂, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents hydrogen, —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, —S(═O)₂—C₁₋₄alkyl, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl or —(C═O)—C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and
wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b} and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, —O—C₁₋₄alkyl, cyano, C₁₋₄alkyl C₁₋₄alkyl

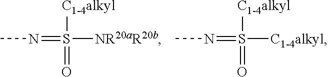

and
C₁₋₄alkyl substituted with one or two substituents each independently selected from the group consisting of Het^{3a}, Het^{6a}, Het^{6b}, and —NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵;
Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, R^{17b}, R^{20a}, R^{20b}, R^{22a} and R^{22b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a}, R^{10b} and R^{10c} are each independently selected from the group consisting of hydrogen, C₁₋₄alkyl, and C₃₋₆cycloalkyl;
R^{10a} and R^{10e} are each independently selected from the group consisting of C₁₋₄alkyl, —O—C₁₋₄alkyl and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het⁷, or Het⁸;
and the pharmaceutically acceptable salts and the solvates thereof.

It should be clear that substituents R²¹ and —Y—R³ in Formula (I) can be attached to any carbon or nitrogen atom of the ring to which they are attached, thereby replacing hydrogens on the same atom or they may replace hydrogen atoms on different atoms in the moiety (including the N-atom). Lines drawn from substituents into ring systems indicate that the bond may be attached to any of the suitable ring atoms.

Additional embodiments, features, and advantages of the invention will be apparent from the following detailed description and through practice of the invention.

### BRIEF DESCRIPTION OF THE DRAWINGS

FIG. 1 is an X-ray powder diffraction (XRPD) pattern of Compound 51 as a crystalline free base Form.

FIG. 2 is an X-ray powder diffraction (XRPD) pattern of Compound 51a as a crystalline HCl salt Form.

FIG. 3 is a Dynamic vapor sorption (DVS) isotherm plot of Compound 51a as a crystalline HCl salt Form.

FIG. 4 is a Dynamic vapor sorption (DVS) change in mass plot of Compound 51a as a crystalline HCl salt Form.

FIG. 5A is a contour plot for maxR which illustrates the effect of Compound 51 in combination with gilteritinib on proliferation of MOLM-13 cells in vitro.

FIG. 5B is a contour plot for maxR which illustrates the effect of Compound 51 in combination with gilteritinib on proliferation of MV4-11 cells in vitro.

FIG. 6A is a contour plot for maxR which illustrates the effect of Compound 51 in combination with midostaurin on proliferation of MOLM-13 cells in vitro.

FIG. 6B is a contour plot for maxR which illustrates the effect of Compound 51 in combination with midostaurin on proliferation of MV4-11 cells in vitro.

FIG. 7A is a contour plot for maxR which illustrates the effect of Compound 51 in combination with idarubicin on proliferation of MOLM-13 cells in vitro.

FIG. 7B is a contour plot for maxR which illustrates the effect of Compound 51 in combination with idarubicin on proliferation of OCI-AML3 cells in vitro.

FIG. 8A is a contour plot for maxR which illustrates the effect of Compound 51 in combination with decitabine on proliferation of MOLM-13 cells in vitro.

FIG. 8B is a contour plot for maxR which illustrates the effect of Compound 51 in combination with decitabine on proliferation of MV4-11 cells in vitro.

FIG. 8C is a contour plot for maxR which illustrates the effect of Compound 51 in combination with decitabine on proliferation of OCI-AML3 cells in vitro.

FIG. 9 depicts a comparison of percent survival as a function of time of mice bearing established MOLM-13 tumors following treatment with vehicle, monotherapy with either azacitidine or Compound 51, or the doublet combination of Compound 51 and azacitidine.

### DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain saturated hydrocarbon radical having from 1 to 4 carbon atoms, such as methyl, ethyl, n-propyl, isopropyl, n-butyl, s-butyl, t-butyl and the like.

Similar, the term ‘C₁₋₆alkyl’ as used herein as a group or part of a group represents a straight or branched chain saturated hydrocarbon radical having from 1 to 6 carbon atoms, such as methyl, ethyl, n-propyl, isopropyl, n-butyl, s-butyl, t-butyl, n-pentyl, n-hexyl and the like.

Similar, the term ‘C₁₋₈alkyl’ as used herein as a group or part of a group represents a straight or branched chain saturated hydrocarbon radical having from 1 to 8 carbon atoms, such as methyl, ethyl, n-propyl, isopropyl, n-butyl, s-butyl, t-butyl, n-pentyl, n-hexyl, n-octyl,

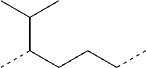

and the like.

The term ‘C₃₋₆cycloalkyl’ as used herein as a group or part of a group defines a saturated, cyclic hydrocarbon radical having from 3 to 6 carbon atoms, such as cyclopropyl, cyclobutyl, cyclopentyl and cyclohexyl.

The term ‘C₃₋₇cycloalkyl’ as used herein as a group or part of a group defines a saturated, cyclic hydrocarbon radical having from 3 to 7 carbon atoms, such as cyclopropyl, cyclobutyl, cyclopentyl, cyclohexyl and cycloheptyl.

It will be clear for the skilled person that S(═O)₂ or SO₂ represents a sulfonyl moiety.

It will be clear for the skilled person that CO or C(═O) represents a carbonyl moiety.

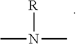

It will be clear for the skilled person that a group such as —NR— represents N. An example of such a group is —NR^{q}—.

Non-limiting examples of ‘monocyclic 5- or 6-membered aromatic rings containing one, two or three nitrogen atoms and optionally a carbonyl moiety’, include, but are not limited to pyrazolyl, imidazolyl, pyridinyl, pyridazinyl, pyrimidinyl, pyrazinyl, triazinyl or 1,2-dihydro-2-oxo-4-pyridinyl.

The skilled person will understand that a monocyclic 5- or 6-membered aromatic ring containing one, two or three nitrogen atoms and a carbonyl moiety includes, but is not limited to

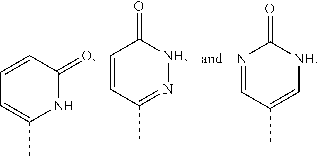

The term ‘monocyclic N-linked 4- to 7-membered fully or partially saturated heterocyclyl containing one N-atom and optionally one or two additional heteroatoms each independently selected from O, S, and N′, defines a fully or partially saturated, cyclic hydrocarbon radical having from 4 to 7 ring members and containing at least 1 nitrogen atom and optionally one or two additional heteroatoms each independently selected from O, S, and N, which is attached to the remainder of the molecule of formula (I) via a nitrogen atom. Examples are N-linked azetidinyl, N-linked pyrrolidinyl, N-linked morpholinyl, N-linked thiomorpholinyl, N-linked piperazinyl, N-linked 1,4-diazepanyl, N-linked piperidinyl, and N-linked 1,2,3,6-tetrahydro-pyridinyl. Two R groups taken together to form together with the N-atom to which they are attached a 4- to 7-membered monocyclic fully or partially saturated heterocyclyl containing one N-atom and optionally one additional heteroatom selected from O, S, and N, are defined similar.

The term ‘monocyclic C-linked 4- to 7-membered fully or partially saturated heterocyclyl containing one, two or three heteroatoms each independently selected from O, S, and N′, defines a fully or partially saturated, cyclic hydrocarbon radical having from 4 to 7 ring members and containing one, two or three heteroatoms each independently selected from O, S, and N, such as for example C-linked azetidinyl, C-linked pyrrolidinyl, C-linked morpholinyl, C-linked tetrahydrofuranyl, C-linked thiolanyl, C-linked oxetanyl, C-linked thietanyl, C-linked tetrahydropyranyl, C-linked tetrahydrothiopyranyl, C-linked piperidinyl, C-linked azepanyl, C-linked 1,3-dioxolanyl, and C-linked 1,2,3,6-tetrahydro-pyridinyl.

For clarity, the 4- to 7-membered fully or partially saturated heterocyclyls have from 4 to 7 ring members including the heteroatoms.

Non-limiting examples of ‘monocyclic C-linked 5- or 6-membered aromatic rings containing one, two or three heteroatoms each independently selected from O, S, and N′, include, but are not limited to C-linked pyrazolyl, C-linked imidazolyl, C-linked pyridinyl, C-linked triazolyl, C-linked pyridazinyl, C-linked pyrimidinyl, C-linked oxazolyl, C-linked furanyl, C-linked isothiazolyl, C-linked thiazolyl, C-linked thiadiazolyl, C-linked oxadiazolyl, or C-linked pyrazinyl.

Within the context of this invention, bicyclic 6- to 11-membered fully or partially saturated heterocyclyl groups, include fused, spiro and bridged bicycles.

Fused bicyclic groups are two cycles that share two atoms and the bond between these atoms.

Spiro bicyclic groups are two cycles that are joined at a single atom.

Bridged bicyclic groups are two cycles that share more than two atoms.

Examples of bicyclic C-linked 6- to 11-membered fully or partially saturated heterocyclyl containing one, two or three heteroatoms each independently selected from O, S, and N, include, but are not limited to

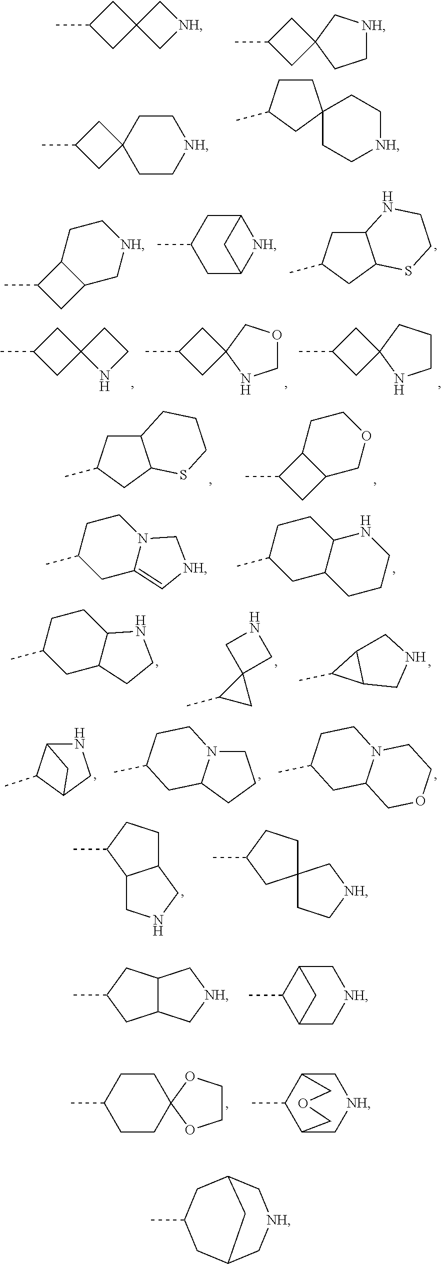

and the like.

Examples of bicyclic N-linked 6- to 11-membered fully or partially saturated heterocyclyl containing one N-atom and optionally one or two additional heteroatoms each independently selected from O, S, and N, include, but are not limited to

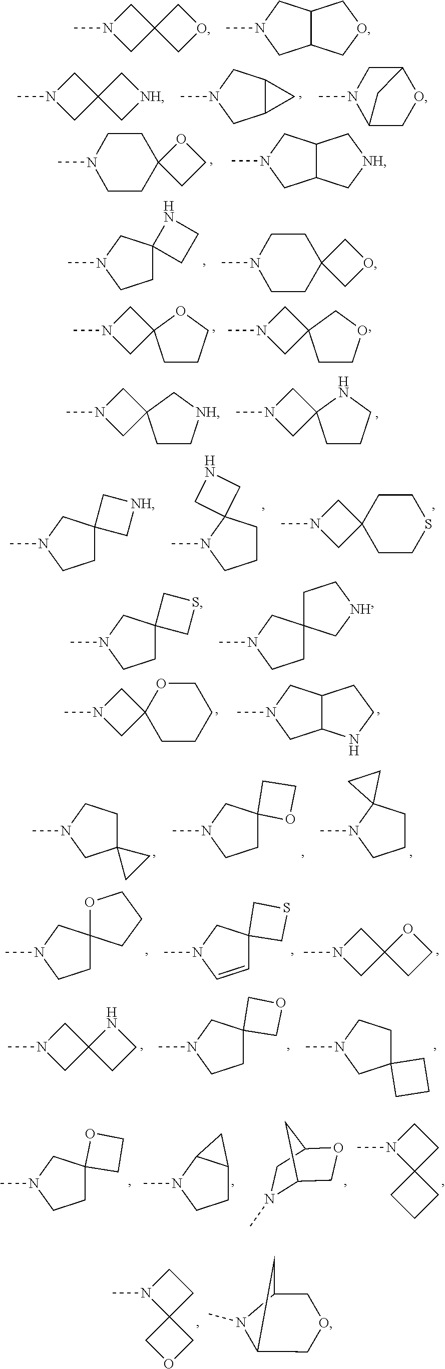

and the like.

Two R groups taken together to form together with the N-atom to which they are attached a 6- to 11-membered bicyclic fully or partially saturated heterocyclyl containing one N-atom and optionally one additional heteroatom selected from O, S, and N, are defined similar.

Examples of fused bicyclic C-linked 9- or 10-membered aromatic ring containing one, two, three or four heteroatoms each independently selected from O, S, and N, include but are not limited to

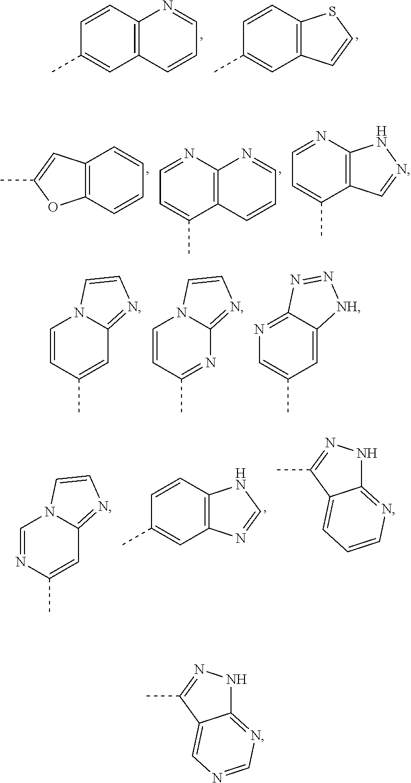

and the like.

As used herein ‘5- to 12-membered saturated carbobicyclic’ systems define saturated fused, spiro and bridged bicyclic hydrocarbon systems having from 5 to 12 carbon atoms. Examples of 5- to 12-membered saturated carbobicyclic’ systems include, but are not limited to

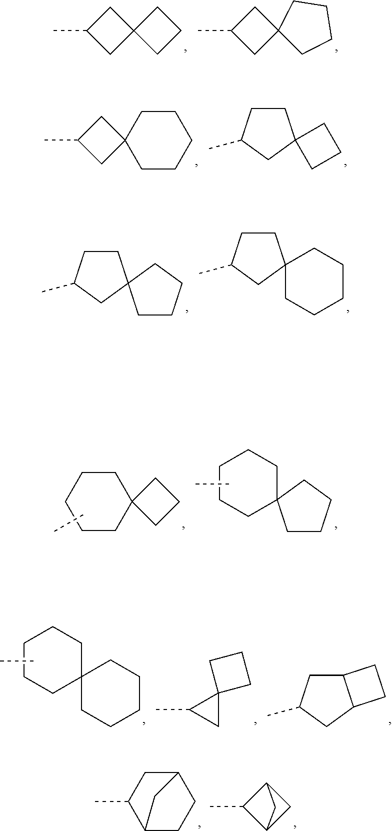

and the like.

Whenever substituents are represented by chemical structure, such as for example

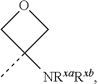

‘ [symbol] ’ represents the bond of attachment to the remainder of the molecule of Formula (I).

When any variable occurs more than one time in any constituent, each definition is independent.

When any variable occurs more than one time in any formula (e.g. Formula (I)), each definition is independent.

It will be clear for a skilled person that when a moiety (for example a heterocyclyl or monocyclic 5- or 6-membered aromatic ring) is substituted with two or more substituents (for example one, two or three substituents) selected from a group, each substituent can be selected independently from said group, even if not explicitly mentioned.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture (isolation after a reaction e.g. purification by silica gel chromatography). In a particular embodiment, when the number of substituents is not explicitly specified, the number of substituents is one.

Combinations of substituents and/or variables are permissible only if such combinations result in chemically stable compounds. ‘Stable compound’ is in this context meant to indicate a compound that is sufficiently robust to survive isolation to a useful degree of purity from a reaction mixture (isolation after a reaction e.g. purification by silica gel chromatography).

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

Within the context of this invention ‘saturated’ means ‘fully saturated’, if not otherwise specified.

Unless otherwise specified or clear from the context, aromatic rings and heterocyclyl groups, can be attached to the remainder of the molecule of Formula (I) through any available ring carbon atom (C-linked) or nitrogen atom (N-linked).

Unless otherwise specified or clear from the context, aromatic rings and heterocyclyl groups, may optionally be substituted, where possible, on carbon and/or nitrogen atoms according to the embodiments. A skilled person will understand that in such a case hydrogens on the carbon and/or nitrogen atoms are replaced by such substituents.

Unless otherwise specified or clear from the context, variable R²¹ and —Y—R³ can be attached to any carbon or nitrogen atom of the ring to which they are attached, provided that when R²¹ represents —Y^{a}—R^{3a}, one of —Y^{a}—R^{3a} and —Y—R³ is attached to the nitrogen atom of the ring.

For example in case R²¹ represents hydrogen, and —Y—R³ is attached to the nitrogen atom of the ring in Formula (I), a compound of subformula (I-x) is obtained:

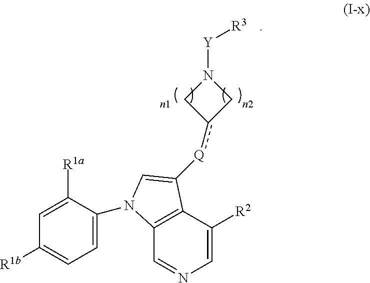

In case Y represents a covalent bond in Formula (I), a compound of subformula (I-y) is obtained:

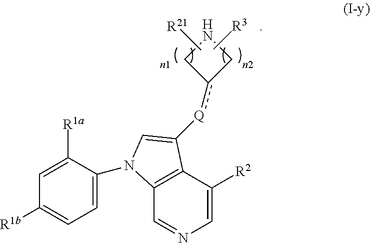

In case Y represents

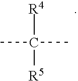

in Formula (I), a compound of subformula (I-z) is obtained:

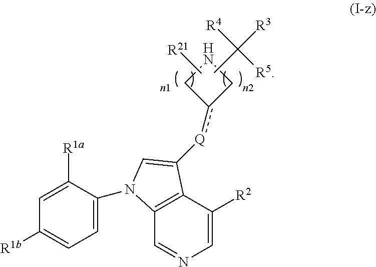

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

Compounds of Formula (I) are Menin-MLL inhibitors of Formula (I).

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

Some of the compounds according to Formula (I) may also exist in their tautomeric form. Such forms in so far as they may exist, although not explicitly indicated in the above Formula (I) are intended to be included within the scope of the present invention. It follows that a single compound may exist in both stereoisomeric and tautomeric form.

Pharmaceutically acceptable salts include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I) and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term “prodrug” includes any compound that, following oral or parenteral administration, in particular oral administration, is metabolised in vivo to a (more) active form in an experimentally-detectable amount, and within a predetermined time (e.g. within a dosing interval of between 0.5 and 24 hours, or e.g. within a dosing interval of between 6 and 24 hours (i.e. once to four times daily)). For the avoidance of doubt, the term “parenteral” administration includes all forms of administration other than oral administration, in particular intravenous (IV), intramuscular (IM), and subcutaneous (SC) injection.

Prodrugs may be prepared by modifying functional groups present on a compound in such a way that the modifications are cleaved in vivo when such prodrug is administered to a mammalian subject. The modifications typically are achieved by synthesising the parent compound with a prodrug substituent. In general, prodrugs include compounds wherein a hydroxyl, amino, sulfhydryl, carboxy or carbonyl group is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elsevier, New York-Oxford (1985).

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹⁵N, ¹⁵O, ¹⁷O, ¹⁸O, ³²P, ³³P, ³⁵S, ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C, ¹³C and ¹⁸F. Preferably, the isotope is selected from the group of ²H, ³H, ¹¹C and ¹⁸F. More preferably, the isotope is ²H, ³H or ¹³C. More preferably, the isotope is ²H or ¹³C. More preferably, the isotope is ²H. In particular, deuterated compounds and ¹³C-enriched compounds are intended to be included within the scope of the present invention. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (3H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy. Additionally, target-specific PET radiotracers may be used as biomarkers to examine and evaluate pathology, by for example, measuring target expression and treatment response.

The present invention also relates to a pharmaceutical composition comprising a therapeutically effective amount of a compound of Formula (I), a pharmaceutically acceptable salt, or a solvate thereof, at least one other therapeutic agent as an active ingredient, and a pharmaceutically acceptable carrier or excipient.

By way of further example, pharmaceutical compositions containing a compound of Formula (I), or a pharmaceutically acceptable salt or a solvate thereof, and at least one other therapeutic agent as an active ingredient can be prepared by mixing the compound(s) with a pharmaceutically acceptable carrier, a pharmaceutically acceptable diluent, and/or a pharmaceutically acceptable excipient.

As used herein, the term “menin-MLL inhibitor” refers to an inhibitor of the protein-protein interaction between menin and mixed-lineage leukemia 1 (MLL1) (also known as histone-lysine N-methyltransferase 2A (KMT2A) protein in the scientific field (UniProt Accession #Q03164)) which inhibits or reduces menin-MLL 1 activity. Menin-MLL inhibitors described herein are disclosed in PCT/CN2022/095901, which is incorporated by reference herein in its entirety, and which also discloses corresponding synthetic schemes and analytical characterizations.

As used herein, the term “therapeutic agent” refers to any agent that treats cancer. In the context of this application, in some embodiments, therapeutic agents are limited to the therapeutic agents explicitly listed herein.

As used herein, the term “hypomethylating agent” refers to an agent that inhibits or reduces DNA methylation.

As used herein, the term “cytidine deaminase inhibitor” refers to an agent that inhibits or reduces cytidine deaminase activity.

As used herein, the term “kinase inhibitor” refers to an agent that inhibits or reduce the activity of at least one kinase (e.g., tyrosine and/or serine kinases such as fins-like receptor tyrosine kinase-3 (FLT3), Bruton tyrosine kinase (BTK), an Abelson tyrosine kinase 1 (ABL), an Aurora serine/tyrosine kinase).

As used herein, the term “FLT-3 inhibitor” refers to tyrosine kinase inhibitors (TKI) classified into first and next generation inhibitors based on their potency and specificity for fms-like receptor tyrosine kinase-3 (FLT3) and their associated downstream targets.

As used herein, the term “CD20 inhibitor” refers to any agent that reduces activity of CD20.

As used herein, the term “isocitrate dehydrogenase (IDH) inhibitor” refers to any agent that interferes with the conversion of isocitrate to α-ketoglutarate (α-KG) in the tricarboxylic acid (TCA) cycle.

As used herein, the term “immunomodulatory agent” refers to any agent that stimulates or suppresses the immune system (e.g., via cytokine modulation, co-stimulation of T cells, down-regulation of co-inhibitory molecules, enhancing natural killer cell activity, inhibition of regulatory T cells, and repairing perturbed synapse formation on T cells). In particular embodiments, immunomodulatory agents, such as monoclonal antibodies, cytokines, and vaccines, affect specific parts of the immune system. In other embodiments, immunomodulatory agents, such as Bacillus Calmette-Guérin (BCG) and levamisole, affect the immune system in a general way.

As used herein, the term “programmed cell death protein 1 (PD-1) inhibitor” refers to any agent that inhibits or reduces PD-1 activity.

As used herein, the term “dihydroorotate dehydrogenase (DHODH) inhibitor” refers to any agent that inhibits or reduces dihydroorotate dehydrogenase activity.

As used herein, unless otherwise noted, the term “affect” or “affected” (when referring to a disease, disorder, or medical condition that is affected by the inhibition or alteration of menin-MLL activity) includes a reduction in the frequency and/or severity of one or more symptoms or manifestations of said hematopoietic disorder; and/or includes the prevention of the development of one or more symptoms or manifestations of said hematopoietic disorder or the development of the hematopoietic disorder.

As used herein, the term “hematopoietic disorder” refers to any disorder associated with the production of the cellular components of blood and blood plasma, including but not limited to blood cancers.

According to an embodiment, the invention provides combinations as described herein.

According to an embodiment, the invention provides combinations as described herein for use as a medicament.

According to an embodiment, the invention provides combinations as described herein for the manufacture of a medicament.

According to an embodiment, the invention provides combinations as described herein for the manufacture of a medicament for the treatment or prevention of any one of the disease conditions mentioned herein.

According to an embodiment, the invention provides combinations as described herein for use in the prevention or treatment, in particular treatment, of diseases as described herein.

According to an embodiment, the invention provides combinations as described herein for use in the prevention or treatment, in particular treatment, of cancer.

According to an embodiment, the invention provides combinations as described herein for use in the prevention or treatment, in particular treatment, of a cancer, including but not limited to solid tumors, sarcomas and hematopoietic disorders.

According to an embodiment, the cancer is selected from, but not limited to, breast cancer, colorectal carcinoma, gastric cancer, glioma, head & neck cancer, hepatocellular carcinoma, lung cancer, multiple myeloma, neuroblastoma, ovarian cancer, pancreatic cancer, prostate cancer, renal cell carcinoma and sarcoma.

According to an embodiment, the sarcoma is selected from, but not limited to, sarcoma of the soft tissue, glioma, osteosarcoma, malignant fibrous histiocytoma, lymphosarcoma, and rhabdomyosarcoma.

According to an embodiment, the invention provides combinations as described herein for use in the prevention or treatment, in particular treatment, of a hematopoietic disorder, including but not limited to blood cancers, including but not limited to lymphomas, myelomas and leukemias.

According to an embodiment, the invention provides combinations as described herein for use in the prevention or treatment, in particular treatment, of a hematopoietic disorder.

According to an embodiment, the hematopoietic disorder is selected from, but not limited to, lymphomas, myelomas, myelodysplasia and leukemias.

According to an embodiment, the hematopoietic disorder is a lymphoma selected from Hodgkin's disease lymphomas and Non-Hodgkin's lymphomas.

According to an embodiment, the lymphoma is a Non-Hodgkin's disease that is Burkitt's lymphoma, anaplastic large cell lymphoma, splenic marginal zone lymphoma, hepatosplenic T-cell lymphoma or angioimmunoblastic T-cell lymphoma (AILT).

According to an embodiment, the hematopoietic disorder is a myeloma. According to an embodiment, the hematopoietic disorder is a multiple myeloma, Waldenström macroglobulinemia or plasmacytoma.

According to an embodiment the hematopoietic disorder is a myelodysplasia including, but not limited to, myelodysplastic syndrome (MDS).

According to an embodiment, the hematopoietic disorder is a leukemia.

According to an embodiment, the hematopoietic disorder is a leukemia selected from acute leukemias and chronic leukemias. According to an embodiment, the leukemia is an acute leukemia. According to an embodiment, the leukemia is chronic leukemia.

According to an embodiment, the hematopoietic disorder is a myeloid leukemia, myelogeneous leukemia, lymphoblastic leukemia, or lymphocytic leukemia, According to an embodiment, the hematopoietic disorder is a leukemia selected from, but not limited to, acute lymphocytic leukemia (ALL), chronic lymphocytic leukemia (CLL), small lymphocytic leukemia (SLL), acute myeloid leukemia (AML), chronic idiopathic myelofibrosis (MF), chronic myelogenous leukemia (CML), T-cell prolymphocytic leukemia (T-PLL), B-cell prolymphocytic leukemia (B-PLL), chronic neutrophilic leukemia (CNL), Hairy cell leukemia (HCL), T-cell large granular lymphocyte leukemia (T-LGL) and aggressive NK-cell leukemia. According to an embodiment, the AML is acute megakaryoblastic leukemia (AMKL).

According to an embodiment, the leukemia is MDS, CLL, SLL, ALL or AML. According to an embodiment, the leukemia is CLL, SLL or AML. According to an embodiment, the leukemia is CLL or SLL. In some embodiments, the CLL or SLL is a CD20 expressing cancer. According to an embodiment, the leukemia is ALL or AML. According to an embodiment, the leukemia is ALL. According to an embodiment, the leukemia is AML. According to an embodiment, the hematopoietic disorder is Waldenström macroglobulinemia.

According to an embodiment, the hematopoietic disorder is a MLL-rearranged leukemia, MLL-partial tandem duplication (PTD) leukemia, MLL amplified leukemia, MLL-positive leukemia, or leukemia exhibiting elevated HOX/MEIS1 gene expression signatures.

According to an embodiment, the leukemia is a MLL-rearranged leukemia &/or a nucleophosmin 1 (NPM1)-mutated leukemia.

According to an embodiment, the hematopoietic disorder is a MLL-rearranged leukemia.

According to an embodiment, the hematopoietic disorder is a nucleophosmin 1 (NPM1)-mutated leukemia (e.g., NPM1c).

According to an embodiment, the invention provides methods for treatment of a hematopoietic disorder that is myelodysplastic syndrome (MDS), a myeloproliferative neoplasm (MPN), acute lymphocytic leukemia (ALL), acute myeloid leukemia (AML), a small lymphocytic lymphoma (SLL) or chronic lymphocytic leukemia (CLL), comprising administering to a subject in need thereof a therapeutically effective amount of a compound of Formula (I), or a pharmaceutically acceptable salt or a solvate thereof, and at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

According to an embodiment, the hematopoietic disorder is myelodysplastic syndrome (MDS) or a myeloproliferative neoplasm (MPN).

According to an embodiment, the hematopoietic disorder is acute lymphocytic leukemia (ALL).

According to an embodiment, the hematopoietic disorder is acute myeloid leukemia (AML).

According to an embodiment, the hematopoietic disorder is a small lymphocytic lymphoma (SLL) or chronic lymphocytic leukemia (CLL).

According to an embodiment, the hematopoietic disorder is a SLL or CLL where SLL or CLL is a CD20-expressing cancer.

According to an embodiment, the hematopoietic disorder is myelodysplastic syndrome (MDS).

According to an embodiment, the hematopoietic disorder is a myeloproliferative neoplasm (MPN).

According to an embodiment, the hematopoietic disorder is a NPM1-mutated leukemia with a FLT3 mutation.

According to an embodiment, the hematopoietic disorder is a FLT3-dependent leukemia.

According to an embodiment, the hematopoietic disorder harbours one or more MLL1 (KMT2A) gene rearrangements or alterations (e.g., duplications or amplification) and/or NPM1 mutations.

According to an embodiment, the hematopoietic disorder harbours (i) one or more MLL1 (KMT2A) gene rearrangements or alterations (e.g., duplications or amplification) and/or NPM1 mutations plus (ii) a FLT3 mutation.

According to an embodiment, the hematopoietic disorder is an MLL-rearranged leukemia.

According to an embodiment, the hematopoietic disorder is acute myeloid leukemia (AML).

According to an embodiment, the hematopoietic disorder is a small lymphocytic lymphoma (SLL).

According to an embodiment, the hematopoietic disorder is a chronic lymphocytic leukemia (CLL).

According to an embodiment, the hematopoietic disorder is an acute leukemia, chronic leukemia, myeloid leukemia, myelogeneous leukemia, lymphoblastic leukemia, lymphocytic leukemia, acute myelogeneous leukemia (AML), chronic myelogenous leukemia (CML), acute lymphoblastic leukemia (ALL), chronic lymphocytic leukemia (CLL), T cell prolymphocytic leukemias (T-PLL), large granular lymphocytic leukemia, Hairy cell leukemia (HCL), MLL-rearranged leukemia, MLL-PTD leukemia, MLL amplified leukemia, MLL-positive leukemia, or leukemia exhibiting elevated HOX/MEIS1 gene expression signatures.

According to an embodiment, the hematopoietic disorder is AML, in particular nucleophosmin (NPM1)-mutated AML (i.e., NPM1^{mut} AML), more in particular abstract NPM1-mutated AML.

According to an embodiment, the hematopoietic disorder is a MLL-rearranged leukemia, in particular MLL-rearranged AML or ALL.

According to an embodiment, the hematopoietic disorder includes a MLL gene alteration, in particular the hematopoietic disorder is AML or ALL with MLL gene alteration(s). In certain embodiments, the MLL gene alteration is a duplication. In certain embodiments the MLL gene alteration is an amplification.

According to an embodiment, the hematopoietic disorder includes a NPM1 gene mutation and/or MLL1 (also known as KMT2A) gene mutation.

According to an embodiment, MLL1 gene mutations include, but are not limited to, MLL1 gene rearrangements, duplications or amplification.

According to an embodiment, the hematopoietic disorder is a mixed-lineage leukemia (MLL), MLL-related leukemia, MLL-associated leukemia, MLL-positive leukemia, MLL-induced leukemia, leukemia associated with a MLL, acute leukemia, chronic leukemia, myelodysplastic syndrome (MDS), or myeloproliferative neoplasms (MPN).

All embodiments described herein for methods for treating a disorder, are also applicable for use in treating said disorder.

All embodiments described herein for use in treating a disorder, are also applicable for methods for treating said disorder.

All embodiments described herein for methods for treating a disorder, are also applicable for use in a method for treating said disorder.

All embodiments described herein for use in a method for treating a disorder, are also applicable for methods for treating said disorder.

In an embodiment, the present invention relates to a novel combination comprising a therapeutically effective amount of a menin-MLL inhibitor of Formula (I), or a tautomer or a stereoisomeric form thereof, or a pharmaceutically acceptable salt or a solvate thereof; and a therapeutically effective amount of at least one other therapeutic agent which is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

According to an embodiment, compounds of Formula (I) are as defined herein, and the tautomers and the stereoisomeric forms thereof, wherein
Q represents —CHR^{y}— or —CR^{y}═; the dotted line is an optional additional bond to form a double bond in case Q represents —CR^{y}═;
R^{1a} represents hydrogen; halo; —C(═O)—NR^{xa}R^{xb}; —S(═O)₂—R¹⁸;
—C(═O)—O—C₁₋₄alkyl; or

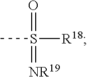

R¹⁸ represents C₁₋₆alkyl;
R¹⁹ represents hydrogen or C₁₋₆alkyl;
or R¹⁸ and R¹⁹ are taken together to form —(CH₂)₃—, —(CH₂)₄— or —(CH₂)₅—;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, —O—C₁₋₄alkyl, and C₁₋₄alkyl substituted with one, two or three OR²³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;
R²³ represents hydrogen or C₁₋₄alkyl;
R^{1b} represents F or —O—C₁₋₄alkyl;
R² represents halo, C₁₋₄alkyl, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or

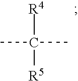

R⁵ represents hydrogen;
n1 is selected from 1 and 2;
n2 is selected from 1, 2 and 3;
R^{y} represents hydrogen;
R³, R^{3a}, and R⁴ are each independently selected from the group consisting of Het¹; C₁₋₈alkyl;
and C₁₋₈alkyl substituted with one, two, three or four substituents each independently selected from the group consisting of —C(═O)—Het^{6a}, —C(═O)—Het^{6b}, —NR¹⁰—C(═O)—C₁₋₄alkyl, —NR^{xc}R^{xd}, —NR^{8a}R^{8b}, —CF₃, halo, —OH, —O—C₁₋₄alkyl, Het¹, Het², Ar¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl, and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
R^{8a} and R^{8b} are each independently selected from the group consisting of hydrogen;
C₁₋₆alkyl; —(C═O)—C₁₋₄alkyl; and C₁₋₆alkyl substituted with one, two or three —O—C₁₋₄alkyl;
Ar¹ represents phenyl optionally substituted with one, two or three substituents each independently selected from the group consisting of C₁₋₄alkyl and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, C₁₋₄alkyl, oxo and —OH;
Het² represents C-linked pyrazolyl, 1,2,4-oxadiazolyl, pyridazinyl or triazolyl;
R⁶ is selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;
C₁₋₆alkyl optionally substituted with one or two substituents each independently selected from the group consisting of Het^{6a}, Het^{6b}, and —OH;
R⁸ represents hydrogen, —O—C₁₋₆alkyl, C₁₋₆alkyl; or C₁₋₆alkyl substituted with one, two or three substituents each independently selected from —OH, —O—C₁₋₄alkyl, cyano, —S(═O)₂—C₁₋₄alkyl, and Het^{3a};
Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen atom with —(C═O)—C₁₋₄alkyl;
Het⁴ represents a monocyclic C-linked 5- or 6-membered aromatic ring containing one, two or three heteroatoms each independently selected from O, S, and N; wherein said aromatic ring is optionally substituted on one or two carbon atoms with in total one or two substituents each independently selected from the group consisting of C₁₋₄alkyl and —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo and —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a —C(═O)—C₁₋₄alkyl;
Cy¹ represents C₃₋₆cycloalkyl optionally substituted with one, two or three —OH;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, and C₁₋₄alkyl;
R^{9a} and R^{9b} are each independently selected from the group consisting of hydrogen;
C₁₋₄alkyl; —C(═O)—C₁₋₄alkyl; —S(═O)₂—C₁₋₄alkyl; and —C(═O)—R¹⁴;
R^{10a}, R^{10b} and R^{10c} are each independently selected from the group consisting of hydrogen and C₁₋₄alkyl;
R^{10d} and R^{10e} are each independently selected from the group consisting of C₁₋₄alkyl and —O—C₁₋₄alkyl;
R¹⁴ represents —O—C₁₋₄alkyl;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—;
R^{1a} represents —C(═O)—NR^{xa}R^{xb}; —S(═O)₂—R¹⁸;
—C(═O)—O—C₁₋₄alkyl; or

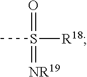

R¹⁸ represents C₁₋₆alkyl;
R¹⁹ represents hydrogen or C₁₋₆alkyl;
or R¹⁸ and R¹⁹ are taken together to form —(CH₂)₃—, —(CH₂)₄— or —(CH₂)₅—;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, —O—C₁₋₄alkyl, and C₁₋₄alkyl substituted with one, two or three OR²³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;
R²³ represents hydrogen or C₁₋₄alkyl;
R^{1b} represents F or —O—C₁₋₄alkyl;
R² represents halo, C₁₋₄alkyl, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} represent a covalent bond;
n1 is selected from 1 and 2;
n2 is selected from 1, 2 and 3;
R^{y} represents hydrogen;
R³ and R^{3a} are each independently selected from the group consisting of Het¹; C₁₋₈alkyl; and
C₁₋₈alkyl substituted with one, two, three or four substituents each independently selected from the group consisting of —C(═O)—Het^{6a}, —C(═O)—Het^{6b}, —NR^{10c}—C(═O)—C₁₋₄alkyl, —NR^{xc}R^{xd}, —NR^{8a}R^{8b}, —CF₃, halo, —OH, —O—C₁₋₄alkyl, Het¹, Het², Ar¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl, and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
R^{8a} and R^{8b} are each independently selected from the group consisting of hydrogen;
C₁₋₆alkyl; —(C═O)—C₁₋₄alkyl; and C₁₋₆alkyl substituted with one, two or three —O—C₁₋₄alkyl;
Ar¹ represents phenyl optionally substituted with one, two or three substituents each independently selected from the group consisting of C₁₋₄alkyl and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, C₁₋₄alkyl, oxo and —OH;
Het² represents C-linked pyrazolyl, 1,2,4-oxadiazolyl, pyridazinyl or triazolyl;
R⁶ is selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;
C₁₋₆alkyl optionally substituted with one or two substituents each independently selected from the group consisting of Het^{6a}, Het^{6b}, and —OH;
R⁸ represents hydrogen, —O—C₁₋₆alkyl, C₁₋₆alkyl; or C₁₋₆alkyl substituted with one, two or three substituents each independently selected from —OH, —O—C₁₋₄alkyl, cyano, —S(═O)₂—C₁₋₄alkyl, and Het^{3a};
Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen atom with —(C═O)—C₁₋₄alkyl;
Het⁴ represents a monocyclic C-linked 5- or 6-membered aromatic ring containing one, two or three heteroatoms each independently selected from O, S, and N; wherein said aromatic ring is optionally substituted on one or two carbon atoms with in total one or two substituents each independently selected from the group consisting of C₁₋₄alkyl and —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo and —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a —C(═O)—C₁₋₄alkyl;
Cy¹ represents C₃₋₆cycloalkyl optionally substituted with one, two or three —OH;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, and C₁₋₄alkyl;
R^{9a} and R^{9b} are each independently selected from the group consisting of hydrogen;
C₁₋₄alkyl; —C(═O)—C₁₋₄alkyl; —S(═O)₂—C₁₋₄alkyl; and —C(═O)—R¹⁴;
R^{10a}, R^{10b} and R^{10c} are each independently selected from the group consisting of hydrogen and C₁₋₄alkyl;
R^{10d} and R^{10e} are each independently selected from the group consisting of C₁₋₄alkyl and —O—C₁₋₄alkyl;
R¹⁴ represents —O—C₁₋₄alkyl;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—;
R^{1a} represents —C(═O)—NR^{xa}R^{xb}; —S(═O)₂—R¹⁸;
—C(═O)—O—C₁₋₄alkyl; or

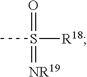

R¹⁸ represents C₁₋₆alkyl;
R¹⁹ represents hydrogen or C₁₋₆alkyl;
or R¹⁸ and R¹⁹ are taken together to form —(CH₂)₃—;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, —O—C₁₋₄alkyl, and C₁₋₄alkyl substituted with one, two or three OR²³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;
R²³ represents hydrogen or C₁₋₄alkyl;
R^{1b} represents F or —O—C₁₋₄alkyl;
R² represents halo, C₁₋₄alkyl, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond;
n1 is selected from 1 and 2;
n2 is selected from 1, 2 and 3;
R^{y} represents hydrogen;
R³ and R^{3a} are each independently selected from the group consisting of Het¹; C₁₋₈alkyl; and
C₁₋₈alkyl substituted with one, two, three or four substituents each independently selected from the group consisting of —C(═O)—Het^{6a}, —C(═O)—Het^{6b}, —NR^{10c}—C(═O)—C₁₋₄alkyl, —NR^{xc}R^{xd}, —NR^{8a}R^{8b}, —CF₃, halo, —OH, —O—C₁₋₄alkyl, Het¹, Het², Ar¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl, and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —(C═O)—C₁₋₄alkyl;
R^{8a} and R^{8b} are each independently selected from the group consisting of hydrogen;
C₁₋₆alkyl; —(C═O)—C₁₋₄alkyl; and C₁₋₆alkyl substituted with one, two or three —O—C₁₋₄alkyl;
Ar¹ represents phenyl optionally substituted with one, two or three —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, C₁₋₄alkyl, oxo and —OH;
Het² represents C-linked pyrazolyl, 1,2,4-oxadiazolyl, or pyridazinyl;
R⁶ is selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;
C₁₋₆alkyl optionally substituted with one or two substituents each independently selected from the group consisting of Het^{6a}, Het^{6b}, and —OH;
R⁸ represents hydrogen, —O—C₁₋₆alkyl, C₁₋₆alkyl; or C₁₋₆alkyl substituted with one, two or three substituents each independently selected from —OH, —O—C₁₋₄alkyl, cyano and Het^{3a};
Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen atom with —(C═O)—C₁₋₄alkyl;
Het⁴ represents a monocyclic C-linked 5- or 6-membered aromatic ring containing one, two or three heteroatoms each independently selected from O, S, and N; wherein said aromatic ring is optionally substituted on one or two carbon atoms with in total one or two —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four halo; and wherein said heterocyclyl is optionally substituted on one nitrogen with a substituent selected from the group consisting of —C(═O)—C₁₋₄alkyl and —S(═O)₂—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a —C(═O)—C₁₋₄alkyl;
Cy¹ represents C₃₋₆cycloalkyl optionally substituted with one, two or three —OH;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, NR^{9a}R^{9b}, —OH, and C₁₋₄alkyl;
R^{9a} and R^{9b} are each independently selected from the group consisting of hydrogen;
C₁₋₄alkyl; —C(═O)—C₁₋₄alkyl; —S(═O)₂—C₁₋₄alkyl; and —C(═O)—R¹⁴;
R^{10a}, R^{10b} and R^{10c} are each independently selected from the group consisting of hydrogen and C₁₋₄alkyl;
R^{10d} and R^{10e} are each independently selected from the group consisting of C₁₋₄alkyl and —O—C₁₋₄alkyl;
R¹⁴ represents —O—C₁₋₄alkyl;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—;
R^{1a} represents —C(═O)—NR^{xa}R^{xb};
R^{xa} represents C₁₋₆alkyl;
R^{xb} represents C₁₋₆alkyl;
R^{1b} represents F;
R² represents C₁₋₄alkyl;
R²¹ represents hydrogen;
Y represents a covalent bond;
n1 is 1;
n2 is selected from 1 and 2;
R^{y} represents hydrogen;
R³ represents Het¹; or C₁₋₈alkyl substituted with one substituent selected from the group consisting of —C(═O)—Het^{6a}, Het¹, Ar¹, and Cy²;
Ar¹ represents phenyl optionally substituted with one —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, C₁₋₄alkyl, oxo and —OH;
R⁶ is selected from the group consisting of —C(═O)—NH—R⁸; —C(═O)—O—C₁₋₄alkyl;
—S(═O)₂—C₁₋₄alkyl; and C₁₋₆alkyl;
R⁸ represents hydrogen, —O—C₁₋₆alkyl, C₁₋₆alkyl; or C₁₋₆alkyl substituted with one substituent selected from —OH, —O—C₁₋₄alkyl, cyano and Het^{3a};
Het^{3a} represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen atom with —(C═O)—C₁₋₄alkyl;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂;
Cy¹ represents C₃₋₆cycloalkyl;
Cy² represents C₃₋₇cycloalkyl; wherein said C₃₋₇cycloalkyl is optionally substituted with one or two substituents each independently selected from the group consisting of R⁶ and —NR^{9a}R^{9b};
R^{9a} and R^{9b} are each independently selected from the group consisting of hydrogen; and —C(═O)—R¹⁴.

R^{10a} and R^{10b} are each independently selected from the group consisting of hydrogen and C₁₋₄alkyl;
R¹⁴ represents —O—C₁₋₄alkyl;
and the pharmaceutically acceptable salts and the solvates thereof.

According to an embodiment, compounds of Formula (I) are as defined herein, and the tautomers and the stereoisomeric forms thereof, wherein
Q represents —CHR^{y}— or —CR^{y}═; the dotted line is an optional additional bond to form a double bond in case Q represents —CR^{y}═;
R^{1a} represents hydrogen; halo; —C(═O)—NR^{xa}R^{xb}; —S(═O)₂—R¹⁸;
—C(═O)—O—C₁₋₄alkyl;

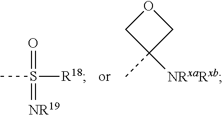

R¹⁸ represents C₁₋₆alkyl;
R¹⁹ represents hydrogen or C₁₋₆alkyl;
or R¹⁸ and R¹⁹ are taken together to form —(CH₂)₃—;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, and C₁₋₄alkyl substituted with one, two or three substituents selected from the group consisting of OR²³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;
R²³ represents hydrogen or C₁₋₄alkyl;
R^{1b} represents F;
R² represents halo, C₁₋₄alkyl, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or

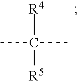

n1 is selected from 1 and 2;
n2 is selected from 1, 2 and 3;
R^{y} represents hydrogen;
R⁵ represents hydrogen;
R³, R^{3a}, and R⁴ are each independently selected from the group consisting of Het¹; Het²; Cy²;
C₁₋₈alkyl; and C₁₋₈alkyl substituted with one, two, three or four substituents each independently selected from the group consisting of —C(═O)—Het^{6a}, —C(═O)—Het^{6b}, NR^{10c}—C(═O)—C₁₋₄alkyl, —NR^{xc}R^{xd}, —NR^{8a}R^{8b}, —CF₃, halo, —OH, —O—C₁₋₄alkyl, Het¹, Het², Ar¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
R^{8a} and R^{8b} are each independently selected from the group consisting of hydrogen;
C₁₋₆alkyl; and C₁₋₆alkyl substituted with one —O—C₁₋₄alkyl;
Ar¹ represents phenyl optionally substituted with one, two or three substituents each independently selected from the group consisting of C₁₋₄alkyl and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, C₁₋₄alkyl, oxo, and —OH;
Het² represents C-linked pyrazolyl, 1,2,4-oxadiazolyl, pyridazinyl or triazolyl;
R⁶ is selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;
C₁₋₆alkyl optionally substituted with one or two —OH substituents; and
C₃₋₆cycloalkyl;
R⁸ represents —O—C₁₋₆alkyl, C₁₋₆alkyl; or C₁₋₆alkyl substituted with one, two or three substituents each independently selected from —OH, —O—C₁₋₄alkyl, cyano, —S(═O)₂—C₁₋₄alkyl, and Het^{3a};
Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one carbon atom with oxo; and
wherein said heterocyclyl is optionally substituted on one nitrogen atom with —(C═O)—C₁₋₄alkyl;
Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two substituents each independently selected from the group consisting of C₁₋₄alkyl and —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo and —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a —C(═O)—C₁₋₄alkyl;
Cy¹ represents C₃₋₆cycloalkyl optionally substituted with one, two or three —OH;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of R⁶, —C(═O)—Het^{6a}, Het^{6a}, Het^{6b}, —NR^{9a}R^{9b}, —OH, and C₁₋₄alkyl;
R^{9a} and R^{9b} are each independently selected from the group consisting of hydrogen;
C₁₋₄alkyl; —C(═O)—C₁₋₄alkyl; —S(═O)₂—C₁₋₄alkyl; and —C(═O)—R¹⁴;
R^{10a}, R^{10b} and R^{10c} are each independently selected from the group consisting of hydrogen and C₁₋₄alkyl;
R^{10d} and R^{10e} are each independently selected from the group consisting of C₁₋₄alkyl and —O—C₁₋₄alkyl;
R¹⁴ represents —O—C₁₋₄alkyl;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—, —O—, —C(═O)—, —NR^{q}—, or —CR^{y}═; the dotted line is an optional additional bond to form a double bond in case Q represents —CR^{y}═;
R^{1a} represents hydrogen; cyano; halo; Het; —C(═O)—NR^{xa}R^{xb}; —S(═O)₂—R¹⁸;

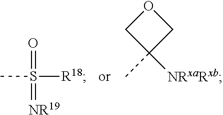

R¹⁸ represents C₁₋₆alkyl or C₃₋₆cycloalkyl;
R¹⁹ represents hydrogen or C₁₋₆alkyl;
Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
R^{1b} represents hydrogen, F or Cl;
R² represents halo, C₃₋₆cycloalkyl, C₁₋₄alkyl, —O—C₁₋₄alkyl, cyano, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or

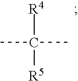

n1 and n2 are each independently selected from 1 and 2;
R^{y} represents hydrogen, —OH, C₁₋₄alkyl, —C₁₋₄alkyl-OH, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²; Cy²;
C₁₋₆alkyl; and C₁₋₆alkyl substituted with one, two, three or four substituents each independently selected from the group consisting of —C(═O)—NR^{10a}R^{10b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;
R^{8a} and R^{8b} are each independently selected from the group consisting of hydrogen;
C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which may be optionally substituted on one nitrogen atom with R^{6a};
R⁶ and R^{6a} are each independently selected from the group consisting of
Het³; Het⁴; —C(═O)—NH—Cy¹; —C(═O)—NH—R⁸; —S(═O)₂—C₁₋₄alkyl;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R¹ b, and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and
wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, (C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b} and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, NR^{9a}R^{9b}, —OH, C₁₋₄alkyl, C₁₋₄alkyl C₁₋₄alkyl

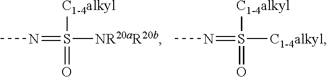

and
C₁₋₄alkyl substituted with one or two substituents each independently selected from the group consisting of Het^{3a}, Het^{6a}, Het^{6b}, and —NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵;
Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, R^{17b}, R^{20a}, and R^{20b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a} and R^{10b} are each independently selected from the group consisting of hydrogen, C₁₋₄alkyl, and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁸, Het⁷, or Het⁸;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—, —O—, —C(═O)—, —NR^{q}—, or —CR^{y}=; the dotted line is an optional additional bond to form a double bond in case Q represents —CR^{y}=;
R^{1a} represents hydrogen; cyano; halo; Het; —C(═O)—NR^{xa}R^{xb}; —S(═O)₂—R¹⁸.

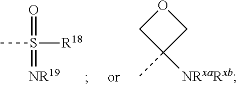

R¹⁸ represents C₁₋₆alkyl or C₃₋₆cycloalkyl;
R¹⁹ represents hydrogen or C₁₋₆alkyl;
Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
R^{1b} represents hydrogen, F or Cl;
R² represents halo, C₃₋₆cycloalkyl, C₁₋₄alkyl, —O—C₁₋₄alkyl, cyano, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or

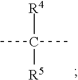

n1 and n2 are each independently selected from 1 and 2;
R^{y} represents hydrogen, —OH, C₁₋₄alkyl, —C₁₋₄alkyl-OH, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²; Cy²;
C₁₋₆alkyl; and C₁₋₆alkyl substituted with one, two, three or four substituents each independently selected from the group consisting of —C(═O)—NR^{10a}R^{10b}, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;
R^{8a} and R^{8b} are each independently selected from the group consisting of hydrogen;
C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which may be optionally substituted on one nitrogen atom with R^{6a};
R⁶ and R^{6a} are each independently selected from the group consisting of
Het³; Het⁴; —C(═O)—NH—Cy¹; —C(═O)—NH—R⁸; —S(═O)₂—C₁₋₄alkyl;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R^{11b} and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and
wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b} and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, C₁₋₄alkyl C₁₋₄alkyl

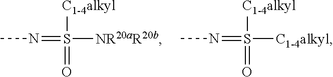

and
C₁₋₄alkyl substituted with one or two substituents each independently selected from the group consisting of Het^{3a}, Het^{6a}, Het^{6b}, and —NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵;
Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, R^{17b}, R^{20a} and R^{20b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a} and R^{10b} are each independently selected from the group consisting of hydrogen, C₁₋₄alkyl, and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het⁷, or Het⁸;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—, —O—, —C(═O)—, —NR^{q}—, or —CR^{y}=; the dotted line is an optional additional bond to form a double bond in case Q represents —CR^{y}=;
R^{1a} represents hydrogen; cyano; halo; Het; —C(═O)—NR^{xa}R^{xb}; —S(═O)₂—R¹⁸;

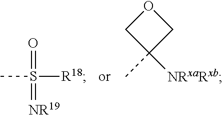

R¹⁸ represents C₁₋₆alkyl or C₃₋₆cycloalkyl;
R¹⁹ represents hydrogen or C₁₋₆alkyl;
Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
R^{1b} represents hydrogen, F or Cl;
R² represents halo, C₃₋₆cycloalkyl, C₁₋₄alkyl, —O—C₁₋₄alkyl, cyano, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or

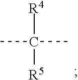

n1 and n2 are each independently selected from 1 and 2;
R^{y} represents hydrogen, —OH, C₁₋₄alkyl, —C₁₋₄alkyl-OH, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²; Cy²;
C₁₋₆alkyl; and C₁₋₆alkyl substituted with one, two, three or four substituents each independently selected from the group consisting of —C(═O)—NR^{10a}R^{10b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;
R^{8a} and R^{8b} are each independently selected from the group consisting of hydrogen;
C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which may be optionally substituted on one nitrogen atom with R^{6a};
R⁶ and R^{6a} are each independently selected from the group consisting of
Het³; Het⁴; —C(═O)—NH—Cy¹; —C(═O)—NH—R⁸; —S(═O)₂—C₁₋₄alkyl;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R^{11b}, and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and
wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b} and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, C₁₋₄alkyl C₁₋₄alkyl

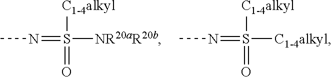

and
C₁₋₄alkyl substituted with one or two substituents each independently selected from the group consisting of Het^{3a}, Het^{6a}, Het^{6b}, and —NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵;
Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, R^{17b}, R^{20a}, and R^{20b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a} and R^{10b} are each independently selected from the group consisting of hydrogen, C₁₋₄alkyl, and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het⁷, or Het⁸;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—, —O—, —C(═O)—, —NR^{q}—, or —CR^{y}=; the dotted line is an optional additional bond to form a double bond in case Q represents —CR^{y}=;
R^{1a} represents hydrogen; cyano; halo; Het; —C(═O)—NR^{xa}R^{xb}; —S(═O)₂—R¹⁸;

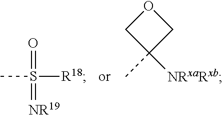

R¹⁸ represents C₁₋₆alkyl or C₃₋₆cycloalkyl;
R¹⁹ represents hydrogen or C₁₋₆alkyl;
Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
R^{1b} represents hydrogen, F or Cl;
R² represents C₁₋₄alkyl; in particular R² represents methyl;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or

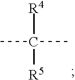

n1 and n2 are each independently selected from 1 and 2;
R^{y} represents hydrogen, —OH, C₁₋₄alkyl, —C₁₋₄alkyl-OH, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²; Cy²;
C₁₋₆alkyl; and C₁₋₆alkyl substituted with one, two, three or four substituents each independently selected from the group consisting of —C(═O)—NR^{10a}R^{10b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;
R^{8a} and R^{8b} are each independently selected from the group consisting of hydrogen;
C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which may be optionally substituted on one nitrogen atom with R^{6a};
R⁶ and R^{6a} are each independently selected from the group consisting of
Het³; Het⁴; —C(═O)—NH—Cy¹; —C(═O)—NH—R⁸; —S(═O)₂—C₁₋₄alkyl;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R^{11b} and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and
wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b} and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl,

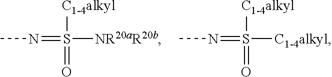

and
C₁₋₄alkyl substituted with one or two substituents each independently selected from the group consisting of Het^{3a}, Het^{6a}, Het^{6b}, and —NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵;
Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{5b}, R^{17a}, R^{17b}, R^{20a} and R^{20b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a} and R^{10b} are each independently selected from the group consisting of hydrogen, C₁₋₄alkyl, and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het⁷, or Het⁸;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—, or —CR^{y}=; the dotted line is an optional additional bond to form a double bond in case Q represents —CR^{y}=;
R^{1a} represents hydrogen; halo; —C(═O)—NR^{xa}R^{xb}; or

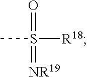

R¹⁸ represents C₁₋₆alkyl or C₃₋₆cycloalkyl;
R¹⁹ represents hydrogen or C₁₋₆alkyl;
R^{xa} and R^{xb} are each independently selected from the group consisting of hydrogen;
Het³; and C₁₋₆alkyl; wherein optionally said C₁₋₆alkyl are substituted with 1, 2 or 3 substituents each independently selected from the group consisting of —OH, and —OC₁₋₄alkyl;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl;
R^{1b} represents F;
R² represents halo, C₁₋₄alkyl, or C₁₋₄alkyl substituted with one, two or three halo substituents;
R²¹ represents hydrogen;
Y represents a covalent bond or

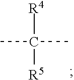

n1 and n2 are each independently selected from 1 and 2;
R^{y} represents hydrogen;
R⁵ represents hydrogen;
R³ and R⁴ are each independently selected from the group consisting of Het¹; Cy²;
C₁₋₆alkyl; and C₁₋₆alkyl substituted with one, two, three or four substituents each independently selected from the group consisting of —NR^{xc}R^{xd}, —NR^{8a}R^{8b}, —CF₃, —OH, Het¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
R^{8a} and R^{8b} are each independently selected from the group consisting of C₁₋₆alkyl; and
C₁₋₆alkyl substituted with one —O—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and
wherein said heterocyclyl is optionally substituted on one or two carbon atoms with in total one or two substituents each independently selected from the group consisting of oxo and —NR^{9a}R^{9b};
R⁶ represents Het⁴; —C(═O)—NH—R⁸; —S(═O)₂—C₁₋₄alkyl; or C₁₋₆alkyl;
R⁸ represents —O—C₁₋₆alkyl, C₁₋₆alkyl; or C₁₋₆alkyl substituted with one, two or three substituents each independently selected from —O—C₁₋₄alkyl, and cyano;
Het³ represents a monocyclic C-linked 4- to 7-membered fully or partially saturated heterocyclyl containing one, two or three heteroatoms each independently selected from O, S, and N;
Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of R⁶, —C(═O)—Het^{6a}, Het^{6a}, Het^{6b}, —NR^{9a}R^{9b},

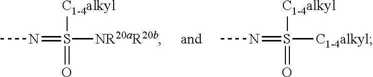

R^{9a} and R^{9b} are each independently selected from the group consisting of hydrogen;
C₁₋₄alkyl; —C(═O)—C₁₋₄alkyl; and —S(═O)₂—C₁₋₄alkyl;
R^{10a} and R^{10b} are each independently selected from the group consisting of hydrogen, C₁₋₄alkyl, and C₃₋₆cycloalkyl;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—, or —CR^{y}=; the dotted line is an optional additional bond to form a double bond in case Q represents —CR^{y}=;
R^{1a} represents hydrogen; halo; or —C(═O)—NR^{xa}R^{xb};
R^{xa} and R^{xb} are each independently selected from the group consisting of hydrogen and C₁₋₆alkyl;
R^{1b} represents F;
R² represents halo, C₁₋₄alkyl, or C₁₋₄alkyl substituted with one, two or three halo substituents;
R²¹ represents hydrogen;
Y represents a covalent bond or

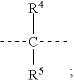

n1 and n2 are each independently selected from 1 and 2;
R^{y} represents hydrogen;
R⁵ represents hydrogen;
R³ and R⁴ are each independently selected from the group consisting of Het¹; Cy²;
C₁₋₆alkyl; and C₁₋₆alkyl substituted with one, two, three or four substituents each independently selected from the group consisting of —NR^{xc}R^{xd}, —NR^{8a}R^{8b}, Het¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
R^{8a} and R^{8b} are each independently selected from the group consisting of C₁₋₆alkyl; and
C₁₋₆alkyl substituted with one —O—C₁₋₄alkyl;
Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with a substituent selected from the group consisting of R⁶ and —C(═O)—R⁸;
R⁶ represents Het⁴; —C(═O)—NH—R⁸; or —S(═O)₂—C₁₋₄alkyl;
R⁸ represents —O—C₁₋₆alkyl, C₁₋₆alkyl; or C₁₋₆alkyl substituted with one, two or three substituents each independently selected from —O—C₁₋₄alkyl, and cyano;
Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one, two, three or four substituents each independently selected from the group consisting of R⁶, Het^{6a}, Het^{6b}, and —NR^{9a}R^{9b};
R^{9a} and R^{9b} are each independently selected from the group consisting of hydrogen;
C₁₋₄alkyl; —C(═O)—C₁₋₄alkyl; and —S(═O)₂—C₁₋₄alkyl;
R^{10a} and R^{10b} are each independently selected from the group consisting of hydrogen and C₁₋₄alkyl;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—;
R^{1a} represents —C(═O)—NR^{xa}R^{xb};
R^{xa} and R^{xb} represent C₁₋₆alkyl;
R^{1b} represents F;
R² represents halo or C₁₋₄alkyl;
R²¹ represents hydrogen;
Y represents a covalent bond or

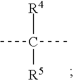

n1 and n2 are each independently selected from 1 and 2;
R^{y} represents hydrogen;
R⁵ represents hydrogen;
R³ is selected from the group consisting of Heti; Cy²; C₁₋₆alkyl; and C₁₋₆alkyl substituted with one, two, three or four substituents each independently selected from the group consisting of —NR^{xc}R^{xd}, Het¹, and Cy²;
R⁴ represents C₁₋₆alkyl; in particular isopropyl;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with a substituent selected from the group consisting of R⁶ and —C(═O)—R⁸;
R⁶ represents Het⁴ or —C(═O)—NH—R⁸;
R⁸ represents C₁₋₆alkyl; or C₁₋₆alkyl substituted with one, two or three substituents each independently selected from —O—C₁₋₄alkyl, and cyano;
Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one, two, three or four substituents each independently selected from the group consisting of R⁶, Het^{6a}, Het^{6b}, and —NR^{9a}R^{9b};
R^{9a} and R^{9b} are each independently selected from the group consisting of hydrogen; and
—S(═O)₂—C₁₋₄alkyl;
R^{10a} and R^{10b} are each independently selected from the group consisting of hydrogen and C₁₋₄alkyl;
and the pharmaceutically acceptable salts and the solvates thereof.

According to an embodiment, compounds of Formula (I) are as defined herein, and the tautomers and the stereoisomeric forms thereof, wherein
Q represents —CHR^{y}—;
R^{1a} represents —C(═O)—NR^{xa}R^{xb};
R^{xa} and R^{xb} represent C₁₋₆alkyl;
R^{1b} represents F;
R² represents C₁₋₄alkyl;
R²¹ represents hydrogen;
Y represents a covalent bond or

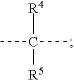

n1 and n2 are each independently selected from 1 and 2;
R^{y} represents hydrogen;
R⁵ represents hydrogen;
R³ is selected from the group consisting of Cy²; and C₁₋₆alkyl substituted with one, two, three or four substituents each independently selected from the group consisting of —NR^{xc}R^{xd}, Het¹, and Cy²;
R⁴ represents C₁₋₆alkyl; in particular isopropyl;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with —C(═O)—R⁸;
R⁶ represents —C(═O)—NH—R⁸;
R⁸ represents C₁₋₆alkyl;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one, two, three or four substituents each independently selected from the group consisting of R⁶ and Het^{6a};
and the pharmaceutically acceptable salts and the solvates thereof.

In an embodiment, the present invention includes compounds of Formula (I) and the pharmaceutically acceptable salts, and the solvates thereof, or any subgroup thereof as mentioned in any of the other embodiments, wherein
Q represents —CHR^{y}—;
R^{1a} represents —C(═O)—NR^{xa}R^{xb};
R^{xa} and R^{xb} are C₁₋₆alkyl optionally substituted with 1, 2 or 3 —OH;
R^{1b} represents F;
R² represents methyl;
R²¹ represents hydrogen or methyl;
Y represents a covalent bond;
n1 is 1;
n2 is selected from 1 and 2;
R^{y} represents hydrogen;
R³ is selected from C₁₋₈alkyl substituted with one, two, three or four substituents each independently selected from the group consisting of —NR^{xc}R^{xd}, Het¹ and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸; and wherein said heterocyclyl is optionally substituted on one carbon atom with oxo;
R⁸ represents C₁₋₆alkyl; or C₁₋₆alkyl substituted with one, two or three substituents each independently selected from —OH, —O—C₁₋₄alkyl and cyano;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one Het^{6a};
and the pharmaceutically acceptable salts and the solvates thereof.

In an embodiment, the present invention includes compounds of Formula (I) and the pharmaceutically acceptable salts, and the solvates thereof, or any subgroup thereof as mentioned in any of the other embodiments, wherein Q represents —CHR^{y}— or —CR^{y}=; the dotted line is an optional additional bond to form a double bond in case Q represents —CR^{y}=.

In an embodiment, the present invention includes compounds of Formula (I) and the pharmaceutically acceptable salts, and the solvates thereof, or any subgroup thereof as mentioned in any of the other embodiments, wherein Q represents —CHR^{y}—.

In an embodiment, the present invention includes compounds of Formula (I) and the pharmaceutically acceptable salts, and the solvates thereof, or any subgroup thereof as mentioned in any of the other embodiments, wherein
R^{1a} represents hydrogen; Het; —C(═O)—NR^{xa}R^{xb}; —S(═O)₂—R¹⁸.

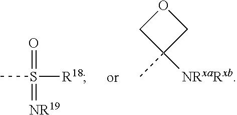

In an embodiment, the present invention includes compounds of Formula (I) and the pharmaceutically acceptable salts, and the solvates thereof, or any subgroup thereof as mentioned in any of the other embodiments, wherein
R^{1a} represents Het; —C(═O)—NR^{xa}R^{xb}; —S(═O)₂—R¹⁸;

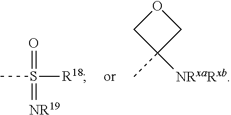

In an embodiment, the present invention includes compounds of Formula (I) and the pharmaceutically acceptable salts, and the solvates thereof, or any subgroup thereof as mentioned in any of the other embodiments, wherein
R^{1a} represents —C(═O)—NR^{xa}R^{xb}; —S(═O)₂—R¹⁸; or

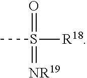

In an embodiment, the present invention includes compounds of Formula (I) and the pharmaceutically acceptable salts, and the solvates thereof, or any subgroup thereof as mentioned in any of the other embodiments, wherein
R^{1a} represents —C(═O)—NR^{xa}R^{xb}; or

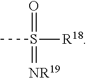

In an embodiment, the present invention includes compounds of Formula (I) and the pharmaceutically acceptable salts, and the solvates thereof, or any subgroup thereof as mentioned in any of the other embodiments, wherein
R^{1a} represents —C(═O)—NR^{xa}R^{xb}.

In an embodiment, the present invention includes compounds of Formula (I) and the pharmaceutically acceptable salts, and the solvates thereof, or any subgroup thereof as mentioned in any of the other embodiments, wherein R¹⁸ represents C₁₋₆alkyl.

In an embodiment, the present invention includes compounds of Formula (I) and the pharmaceutically acceptable salts, and the solvates thereof, or any subgroup thereof as mentioned in any of the other embodiments, wherein R^{xa} and R^{xb} represent hydrogen or C₁₋₆alkyl.

In an embodiment, the present invention includes compounds of Formula (I) and the pharmaceutically acceptable salts, and the solvates thereof, or any subgroup thereof as mentioned in any of the other embodiments, wherein R^{xa} and R^{xb} are each independently selected from the group consisting of hydrogen; Het³; and C₁₋₆alkyl; wherein optionally said C₁₋₆alkyl are substituted with 1, 2 or 3 substituents each independently selected from the group consisting of —OH, and —OC₁₋₄alkyl;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R^{xa} and R^{xb} are each independently selected from the group consisting of hydrogen; Het³; and C₁₋₆alkyl; wherein optionally said C₁₋₆alkyl are substituted with 1, 2 or 3 substituents each independently selected from the group consisting of —OH, and —OC₁₋₄alkyl.

In an embodiment, the present invention includes compounds of Formula (I) and the pharmaceutically acceptable salts, and the solvates thereof, or any subgroup thereof as mentioned in any of the other embodiments, wherein R^{xa} and R^{xb} represent C₁₋₆alkyl.

In an embodiment, the present invention includes compounds of Formula (I) and the pharmaceutically acceptable salts, and the solvates thereof, or any subgroup thereof as mentioned in any of the other embodiments, wherein R^{xa} and R^{xb} are taken together.

In an embodiment, the present invention includes compounds of Formula (I) and the pharmaceutically acceptable salts, and the solvates thereof, or any subgroup thereof as mentioned in any of the other embodiments, wherein R^{xa} and R^{xb} are not taken together.

In an embodiment, the present invention includes compounds of Formula (I) and the pharmaceutically acceptable salts, and the solvates thereof, or any subgroup thereof as mentioned in any of the other embodiments, wherein R^{1b} represents F or Cl.

In an embodiment, the present invention includes compounds of Formula (I) and the pharmaceutically acceptable salts, and the solvates thereof, or any subgroup thereof as mentioned in any of the other embodiments, wherein R^{1b} represents F.

In an embodiment, the present invention includes compounds of Formula (I) and the pharmaceutically acceptable salts, and the solvates thereof, or any subgroup thereof as mentioned in any of the other embodiments, wherein R² represents halo, C₁₋₄alkyl, or C₁₋₄alkyl substituted with one, two or three halo substituents.

In an embodiment, the present invention includes compounds of Formula (I) and the pharmaceutically acceptable salts, and the solvates thereof, or any subgroup thereof as mentioned in any of the other embodiments, wherein R² represents halo or C₁₋₄alkyl.

In an embodiment, the present invention includes compounds of Formula (I) and the pharmaceutically acceptable salts, and the solvates thereof, or any subgroup thereof as mentioned in any of the other embodiments, wherein R² represents C₁₋₄alkyl.

In an embodiment, the present invention includes compounds of Formula (I) and the pharmaceutically acceptable salts, and the solvates thereof, or any subgroup thereof as mentioned in any of the other embodiments, wherein R² represents methyl.

In an embodiment, the present invention includes compounds of Formula (I) and the pharmaceutically acceptable salts, and the solvates thereof, or any subgroup thereof as mentioned in any of the other embodiments, wherein
R² represents methyl; and R^{1a} represents —C(═O)—NR^{xa}R^{xb}.

In an embodiment, the present invention includes compounds of Formula (I) and the pharmaceutically acceptable salts, and the solvates thereof, or any subgroup thereof as mentioned in any of the other embodiments, wherein Y and Y^{a} represent a covalent bond.

In an embodiment, the present invention includes compounds of Formula (I) and the pharmaceutically acceptable salts, and the solvates thereof, or any subgroup thereof as mentioned in any of the other embodiments, wherein R²¹ represents hydrogen.

In an embodiment, the present invention includes compounds of Formula (I) and the pharmaceutically acceptable salts, and the solvates thereof, or any subgroup thereof as mentioned in any of the other embodiments, wherein R²¹ represents hydrogen or methyl.

In an embodiment, the present invention includes compounds of Formula (I) and the pharmaceutically acceptable salts, and the solvates thereof, or any subgroup thereof as mentioned in any of the other embodiments, wherein
R²¹ represents hydrogen; and
Y represents a covalent bond.

In an embodiment, the present invention includes compounds of Formula (I) and the pharmaceutically acceptable salts, and the solvates thereof, or any subgroup thereof as mentioned in any of the other embodiments, wherein
R²¹ represents hydrogen or methyl; and
Y represents a covalent bond.

In an embodiment, the present invention includes compounds of Formula (I) and the pharmaceutically acceptable salts, and the solvates thereof, or any subgroup thereof as mentioned in any of the other embodiments, wherein R²¹ represents —Y^{a}—R^{3a}.

In an embodiment, the present invention includes compounds of Formula (I) and the pharmaceutically acceptable salts, and the solvates thereof, or any subgroup thereof as mentioned in any of the other embodiments, wherein R²¹ represents hydrogen, C₁₋₆alkyl, C₃₋₆cycloalkyl, or C₁₋₆alkyl substituted with 1 substituent selected from the group consisting of halo, —OH, —O—C₁₋₄alkyl, —C(═O)—NR^{10a}R^{10b}, —NR^{10c}—C(═O)—C₁₋₄alkyl, and —S(═O)₂—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R²¹ represents C₁₋₆alkyl, C₃₋₆cycloalkyl, or C₁₋₆alkyl substituted with 1 substituent selected from the group consisting of halo, —OH, —O—C₁₋₄alkyl, —C(═O)—NR^{10a}R^{10b}, —NR^{10c}—C(═O)—C₁₋₄alkyl, and —S(═O)₂—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R^{10c} is selected from the group consisting of hydrogen, C₁₋₄alkyl, and C₃₋₆cycloalkyl.

In an embodiment, the present invention includes compounds of Formula (I) and the pharmaceutically acceptable salts, and the solvates thereof, or any subgroup thereof as mentioned in any of the other embodiments, wherein
R³, R^{3a}, and R⁴ are each independently selected from the group consisting of Het¹; Het²; Cy²;
C₁₋₆alkyl; and C₁₋₆alkyl substituted with one, two, three or four substituents each independently selected from the group consisting of —C(═O)—NR^{10a}R^{10b}, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;
R^{8a} and R^{8b} are each independently selected from the group consisting of hydrogen;
C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, and —C(═O)—NR^{10a}R^{10b}.

In an embodiment, the present invention includes compounds of Formula (I) and the pharmaceutically acceptable salts, and the solvates thereof, or any subgroup thereof as mentioned in any of the other embodiments, wherein
R³, R^{3a}, and R⁴ are not C₁₋₆alkyl substituted with —NR^{10c}—C(═O)—C₁₋₄alkyl;
R^{8a} and R^{8b} are not C₁₋₆alkyl substituted with —NR^{10c}—C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein Y represents a covalent bond.

In an embodiment, the present invention includes compounds of Formula (I) and the pharmaceutically acceptable salts, and the solvates thereof, or any subgroup thereof as mentioned in any of the other embodiments, wherein Y^{a} represents a covalent bond.

In an embodiment, the present invention includes compounds of Formula (I) and the pharmaceutically acceptable salts, and the solvates thereof, or any subgroup thereof as mentioned in any of the other embodiments, wherein Y represents

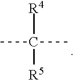

In an embodiment, the present invention includes compounds of Formula (I) and the pharmaceutically acceptable salts, and the solvates thereof, or any subgroup thereof as mentioned in any of the other embodiments, wherein Y^{a} represents

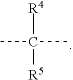

In an embodiment, the present invention includes compounds of Formula (I) and the pharmaceutically acceptable salts, and the solvates thereof, or any subgroup thereof as mentioned in any of the other embodiments, wherein n1 represents 1, and n2 represents 2.

In an embodiment, the present invention includes compounds of Formula (I) and the pharmaceutically acceptable salts, and the solvates thereof, or any subgroup thereof as mentioned in any of the other embodiments, wherein
R⁴ represents C₁₋₆alkyl; oxetanyl; tetrahydropyranyl;

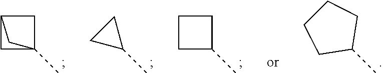

In an embodiment, the present invention includes compounds of Formula (I) and the pharmaceutically acceptable salts, and the solvates thereof, or any subgroup thereof as mentioned in any of the other embodiments, wherein R^{y} represents hydrogen.

In an embodiment, the present invention includes compounds of Formula (I) and the pharmaceutically acceptable salts, and the solvates thereof, or any subgroup thereof as mentioned in any of the other embodiments, wherein
R⁴ represents C₁₋₆alkyl; oxetanyl; tetrahydropyranyl;

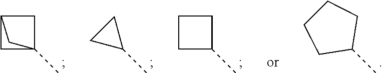

In an embodiment, the present invention includes compounds of Formula (I) and the pharmaceutically acceptable salts, and the solvates thereof, or any subgroup thereof as mentioned in any of the other embodiments, wherein R⁴ represents C₁₋₆alkyl.

In an embodiment, the present invention includes compounds of Formula (I) and the pharmaceutically acceptable salts, and the solvates thereof, or any subgroup thereof as mentioned in any of the other embodiments, wherein R⁴ represents isopropyl.

In an embodiment, the present invention includes compounds of Formula (I) and the pharmaceutically acceptable salts, and the solvates thereof, or any subgroup thereof as mentioned in any of the other embodiments, wherein R⁴ represents C₁₋₈alkyl.

In an embodiment, the present invention includes compounds of Formula (I) and the pharmaceutically acceptable salts, and the solvates thereof, or any subgroup thereof as mentioned in any of the other embodiments, wherein R⁴ represents C₁₋₄alkyl.

In an embodiment, the present invention includes compounds of Formula (I) and the pharmaceutically acceptable salts, and the solvates thereof, or any subgroup thereof as mentioned in any of the other embodiments, wherein R⁵ represents hydrogen.

In an embodiment, the present invention includes compounds of Formula (I) and the pharmaceutically acceptable salts, and the solvates thereof, or any subgroup thereof as mentioned in any of the other embodiments, wherein
R³ and R⁴ are each independently selected from the group consisting of Het¹; Cy²;
C₁₋₆alkyl; and C₁₋₆alkyl substituted with one, two, three or four substituents each independently selected from the group consisting of —NR^{xc}R^{xd}, —NR^{8a}R^{8b}, Het¹, and Cy².

In an embodiment, the present invention includes compounds of Formula (I) and the pharmaceutically acceptable salts, and the solvates thereof, or any subgroup thereof as mentioned in any of the other embodiments, wherein
R³ is selected from the group consisting of Heti; Cy²; C₁₋₆alkyl; and C₁₋₆alkyl substituted with one, two, three or four substituents each independently selected from the group consisting of —NR^{xc}R^{xd}, Het¹, and Cy²; and
R⁴ represents C₁₋₆alkyl; in particular isopropyl.

In an embodiment, the present invention includes compounds of Formula (I) and the pharmaceutically acceptable salts, and the solvates thereof, or any subgroup thereof as mentioned in any of the other embodiments, wherein
R³ is selected from the group consisting of Cy²; and C₁₋₆alkyl substituted with one, two, three or four substituents each independently selected from the group consisting of —NR^{xc}R^{xd}, Het¹, and Cy²; and
R⁴ represents C₁₋₆alkyl; in particular isopropyl.

In an embodiment, the present invention includes compounds of Formula (I) and the pharmaceutically acceptable salts, and the solvates thereof, or any subgroup thereof as mentioned in any of the other embodiments, wherein
R³ is selected from the group consisting of Heti; Cy²; C₁₋₆alkyl; and C₁₋₆alkyl substituted with one, two, three or four substituents each independently selected from the group consisting of —NR^{xc}R^{xd}, Het¹, and Cy².

In an embodiment, the present invention includes compounds of Formula (I) and the pharmaceutically acceptable salts, and the solvates thereof, or any subgroup thereof as mentioned in any of the other embodiments, wherein
R³ is selected from the group consisting of Cy²; and C₁₋₆alkyl substituted with one, two, three or four substituents each independently selected from the group consisting of —NR^{xc}R^{xd}, Het¹, and Cy².

In an embodiment, the present invention includes compounds of Formula (I) and the pharmaceutically acceptable salts, and the solvates thereof, or any subgroup thereof as mentioned in any of the other embodiments, wherein Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —NR^{9a}R^{9b}, and —OH.

In an embodiment, the present invention includes compounds of Formula (I) and the pharmaceutically acceptable salts, and the solvates thereof, or any subgroup thereof as mentioned in any of the other embodiments, wherein
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl.

In an embodiment, the present invention includes compounds of Formula (I) and the pharmaceutically acceptable salts, and the solvates thereof, or any subgroup thereof as mentioned in any of the other embodiments, wherein
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R^{xc} and R^{xd} are taken together.

In an embodiment, the present invention includes compounds of Formula (I) and the pharmaceutically acceptable salts, and the solvates thereof, or any subgroup thereof as mentioned in any of the other embodiments, wherein R^{xc} and R^{xd} are not taken together.

In an embodiment, the present invention includes compounds of Formula (I) and the pharmaceutically acceptable salts, and the solvates thereof, or any subgroup thereof as mentioned in any of the other embodiments, wherein fully or partially saturated heterocyclyl groups are limited to fully saturated heterocyclyl groups.

In an embodiment, the present invention includes compounds of Formula (I) and the pharmaceutically acceptable salts, and the solvates thereof, or any subgroup thereof as mentioned in any of the other embodiments, wherein
Q represents —CHR^{y}—;
R^{1a} represents —C(═O)—NR^{xa}R^{xb};
R^{1b} represents F;
R² represents methyl.

In an embodiment, the present invention includes compounds of Formula (I) and the pharmaceutically acceptable salts, and the solvates thereof, or any subgroup thereof as mentioned in any of the other embodiments, wherein
R^{8a} and R^{8b} are each independently selected from the group consisting of C₁₋₆alkyl; and
C₁₋₆alkyl substituted with one —O—C₁₋₄alkyl.

In an embodiment, the present invention includes compounds of Formula (I) and the pharmaceutically acceptable salts, and the solvates thereof, or any subgroup thereof as mentioned in any of the other embodiments, wherein
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and
wherein said heterocyclyl is optionally substituted on one or two carbon atoms with in total one or two substituents each independently selected from the group consisting of oxo and —NR^{9a}R^{9b}.

In an embodiment, the present invention includes compounds of Formula (I) and the pharmaceutically acceptable salts, and the solvates thereof, or any subgroup thereof as mentioned in any of the other embodiments, wherein R⁶ represents Het⁴ or —C(═O)—NH—R⁸.

In an embodiment, the present invention includes compounds of Formula (I) and the pharmaceutically acceptable salts, and the solvates thereof, or any subgroup thereof as mentioned in any of the other embodiments, wherein R⁸ represents C₁₋₆alkyl; or C₁₋₆alkyl substituted with one, two or three substituents each independently selected from —O—C₁₋₄alkyl, and cyano.

In an embodiment, the present invention includes compounds of Formula (I) and the pharmaceutically acceptable salts, and the solvates thereof, or any subgroup thereof as mentioned in any of the other embodiments, wherein R⁸ represents C₁₋₆alkyl.

In an embodiment, the present invention includes compounds of Formula (I) and the pharmaceutically acceptable salts, and the solvates thereof, or any subgroup thereof as mentioned in any of the other embodiments, wherein R⁸ represents methyl.

In an embodiment, the present invention includes compounds of Formula (I) and the pharmaceutically acceptable salts, and the solvates thereof, or any subgroup thereof as mentioned in any of the other embodiments, wherein
Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b}.

In an embodiment, the present invention includes compounds of Formula (I) and the pharmaceutically acceptable salts, and the solvates thereof, or any subgroup thereof as mentioned in any of the other embodiments, wherein
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of R⁶, —C(═O)—Het^{6a}, Het^{6a}, Het^{6b}, —NR^{9a}R^{9b},

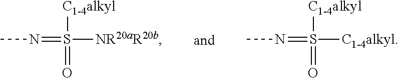

In an embodiment, the present invention includes compounds of Formula (I) and the pharmaceutically acceptable salts, and the solvates thereof, or any subgroup thereof as mentioned in any of the other embodiments, wherein
Cy² represents C₃₋₇cycloalkyl optionally substituted with one, two, three or four substituents each independently selected from the group consisting of R⁶, Het^{6a}, Het^{6b}, NR^{9a}R^{9b}

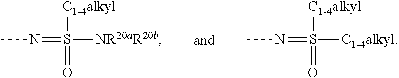

In an embodiment, the present invention includes compounds of Formula (I) and the pharmaceutically acceptable salts, and the solvates thereof, or any subgroup thereof as mentioned in any of the other embodiments, wherein
Cy² represents C₃₋₇cycloalkyl optionally substituted with one, two, three or four substituents each independently selected from the group consisting of R⁶, Het^{6a}, Het^{6b}, and —NR^{9a}R^{9b}.

In an embodiment, the present invention includes compounds of Formula (I) and the pharmaceutically acceptable salts, and the solvates thereof, or any subgroup thereof as mentioned in any of the other embodiments, wherein R^{9a} and R^{9b} are each independently selected from the group consisting of hydrogen; and —S(═O)₂—C₁₋₄alkyl.

In an embodiment, the present invention includes compounds of Formula (I) and the pharmaceutically acceptable salts, and the solvates thereof, or any subgroup thereof as mentioned in any of the other embodiments, wherein R^{10a} and R^{10b} are each independently selected from the group consisting of hydrogen and C₁₋₄alkyl.

In an embodiment, the present invention includes compounds of Formula (I) and the pharmaceutically acceptable salts, and the solvates thereof, or any subgroup thereof as mentioned in any of the other embodiments, wherein when R^{xa} and R^{xb} are taken together to form a monocyclic heterocyclyl they represent 1-pyrrolidinyl or 1-piperidinyl, each optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein when R^{xa} and R^{xb} are taken together to form a bicyclic heterocyclyl they represent

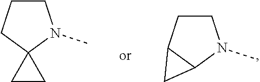

each optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein when R^{xc} and R^{xd} are taken together to form a monocyclic heterocyclyl they represent 1-pyrrolidinyl, 1-piperidinyl, or 1-piperazinyl, each optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein when R^{xc} and R^{xd} are taken together to form a bicyclic heterocyclyl they represent

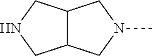

optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein Het¹ represents

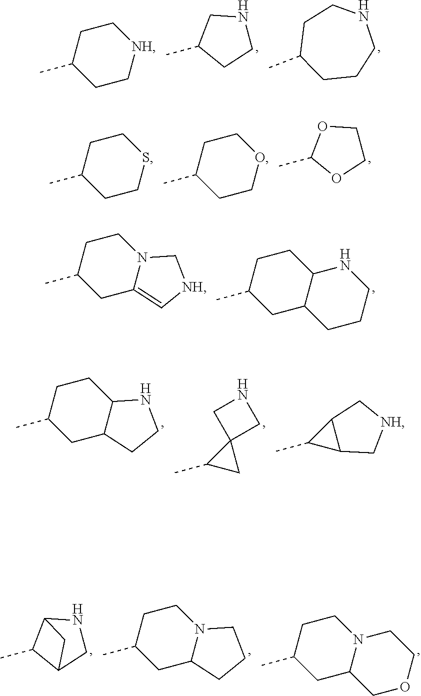

optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein Het¹ represents

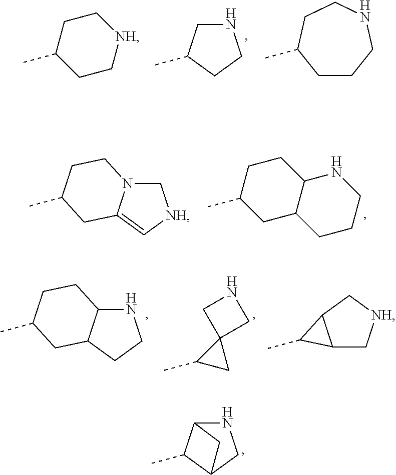

optionally substituted on a nitrogen atom with —C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein Het¹ represents

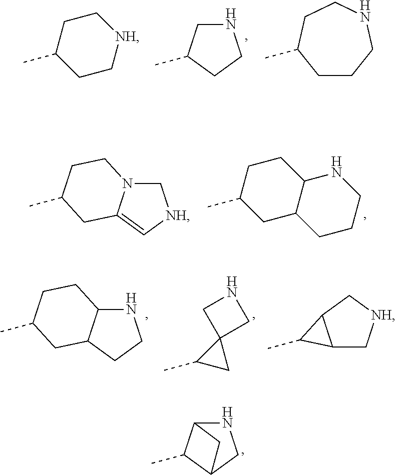

substituted on a nitrogen atom with —C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a fused or spiro bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH.

In an embodiment, the present invention includes compounds of Formula (I) and the pharmaceutically acceptable salts, and the solvates thereof, or any subgroup thereof as mentioned in any of the other embodiments, wherein
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a fused or spiro bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one or two substituents each independently selected from the group consisting of oxo and —NR^{9a}R^{9b}.

In an embodiment, the present invention includes compounds of Formula (I) and the pharmaceutically acceptable salts, and the solvates thereof, or any subgroup thereof as mentioned in any of the other embodiments, wherein
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH.

In an embodiment, the present invention includes compounds of Formula (I) and the pharmaceutically acceptable salts, and the solvates thereof, or any subgroup thereof as mentioned in any of the other embodiments, wherein
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one or two substituents each independently selected from the group consisting of oxo and —NR^{9a}R^{9b}.

In an embodiment, the present invention includes compounds of Formula (I) and the pharmaceutically acceptable salts, and the solvates thereof, or any subgroup thereof as mentioned in any of the other embodiments, wherein Het³ represents

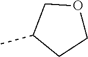

optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein Het⁴ represents C-linked pyrazinyl optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein Het^{6a} represents

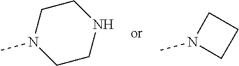

optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein Het^{6a} represents

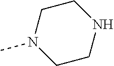

optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein Het^{6a} represents

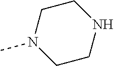

substituted on a nitrogen atom with —C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein Het^{6b} represents

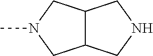

optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein Het^{6b} represents

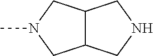

substituted on a nitrogen atom with —C(═O)—C₁₋₄alkyl.

In an embodiment, the present invention includes compounds of Formula (I) and the pharmaceutically acceptable salts, and the solvates thereof, or any subgroup thereof as mentioned in any of the other embodiments, wherein Cy² represents C₃₋₇cycloalkyl,

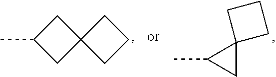

optionally substituted as defined in any of the other embodiments.

In an embodiment, the present invention includes compounds of Formula (I) and the pharmaceutically acceptable salts, and the solvates thereof, or any subgroup thereof as mentioned in any of the other embodiments, wherein C₁₋₈alkyl is limited to C₁₋₆alkyl, in particular wherein C₁₋₈alkyl is limited to C₁₋₄alkyl.

In an embodiment, the present invention includes compounds of Formula (I) and the pharmaceutically acceptable salts, and the solvates thereof, or any subgroup thereof as mentioned in any of the other embodiments, wherein —Y—R³ is attached to the nitrogen atom of the ring.

In an embodiment, the present invention includes compounds of Formula (I) and the pharmaceutically acceptable salts, and the solvates thereof, or any subgroup thereof as mentioned in any of the other embodiments, wherein
R²¹ is hydrogen, and wherein —Y—R³ is attached to the nitrogen atom of the ring.

In an embodiment, the present invention includes compounds of Formula (I) and the pharmaceutically acceptable salts, and the solvates thereof, or any subgroup thereof as mentioned in any of the other embodiments, wherein the compounds of Formula (I) are restricted to compounds of Formula (I-x):

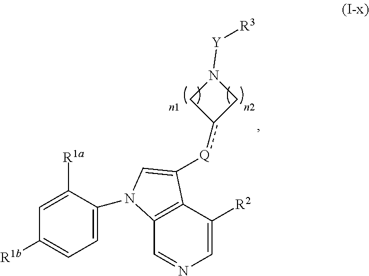

wherein the variables are as defined for the compounds of Formula (I) or any subgroup thereof as mentioned in any of the other embodiments.

In an embodiment, the present invention includes compounds of Formula (I) and the pharmaceutically acceptable salts, and the solvates thereof, or any subgroup thereof as mentioned in any of the other embodiments, wherein the compounds of Formula (I) are restricted to compounds of Formula (I-x1):

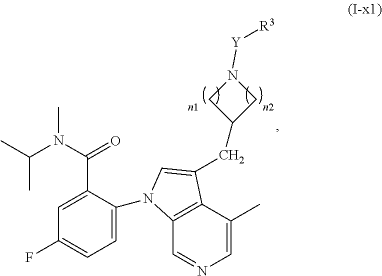

wherein the variables are as defined for the compounds of Formula (I) or any subgroup thereof as mentioned in any of the other embodiments.

In an embodiment, the present invention includes compounds of Formula (I) and the pharmaceutically acceptable salts, and the solvates thereof, or any subgroup thereof as mentioned in any of the other embodiments, wherein the compounds of Formula (I) are restricted to compounds of Formula (I-x2):

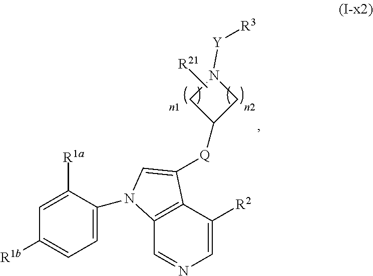

wherein Q represents —CHR^{y}—, —O—, —C(═O)— or —NR^{q}—; and wherein the other variables are as defined for the compounds of Formula (I) or any subgroup thereof as mentioned in any of the other embodiments.

In an embodiment, the present invention in particular includes compounds of Formula (I-x2) as defined herein, and the tautomers and the stereoisomeric forms thereof, wherein
Q represents —CHR^{y}—, —O—, —C(═O)— or —NR^{q}—;
R^{1a} represents hydrogen; cyano; halo; Het; —C(═O)—NR^{xa}R^{xb}; —S(═O)₂—R¹⁸;
—C(═O)—O—C₁₋₄alkyl-NR^{22a}R^{22b}; —C(═O)—O—C₁₋₄alkyl;

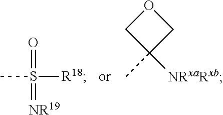

R¹⁸ represents C₁₋₆alkyl or C₃₋₆cycloalkyl;
R¹⁹ represents hydrogen or C₁₋₆alkyl;
or R¹⁸ and R¹⁹ are taken together to form —(CH₂)₃—, —(CH₂)₄— or —(CH₂)₅—;
Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, —C₁₋₄alkyl-OH, halo, CF₃, C₃₋₆cycloalkyl, Het³, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents selected from the group consisting of halo and OR²³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents each independently selected from the group consisting of halo and OR²³;
R²³ represents hydrogen or C₁₋₄alkyl optionally substituted with one, two or three halo;
R^{1b} represents hydrogen, F, Cl, or —O—C₁₋₄alkyl;
R² represents halo, C₃₋₆cycloalkyl, C₁₋₄alkyl, —O—C₁₋₄alkyl, cyano, or C₁₋₄alkyl substituted with one, two or three halo substituents;
R²¹ represents hydrogen or —Y^{a}—R^{3a};
Y and Y^{a} each independently represent a covalent bond or

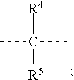

n1 is selected from 1 and 2;
n2 is selected from 1, 2, 3 and 4;
R^{y} represents hydrogen, —OH, C₁₋₄alkyl, —C₁₋₄alkyl-OH, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²; Cy²;
C₁₋₈alkyl; and C₁₋₈alkyl substituted with one, two, three or four substituents each independently selected from the group consisting of —C(═O)—NR^{10a}R^{10b}, —C(═O)—Het^{6a}, —C(═O)—Het^{6b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², Ar¹, and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;
R^{8a} and R^{8b} are each independently selected from the group consisting of hydrogen;
C₁₋₆alkyl; —(C═O)—C₁₋₄alkyl; and C₁₋₆alkyl substituted with one, two or three substituents each independently selected from the group consisting of —OH, cyano, halo, —S(═O)₂—C₁₋₄alkyl, —O—C₁₋₄alkyl,
—C(═O)—NR^{10a}R^{10b}, and —NR^{10c}—C(═O)—C₁₋₄alkyl;
Ar¹ represents phenyl optionally substituted with one, two or three substituents each independently selected from the group consisting of C₁₋₄alkyl, halo, —O—C₁₋₄alkyl, —CF₃, —OH, —S(═O)₂—C₁₋₄alkyl, and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl, 1,2,4-oxadiazolyl, pyridazinyl or triazolyl; which may be optionally substituted on one nitrogen atom with R^{6a};
R⁶ and R^{6a} are each independently selected from the group consisting of
Het³; Het⁴; —C(═O)—NH—Cy¹; —C(═O)—NH—R^{B}; —C(═O)—Het^{6a}; —C(═O)—NR^{10d}R^{10e}; —C(═O)—O—C₁₋₄alkyl; —S(═O)₂—C₁₋₄alkyl;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—N(C₁₋₄alkyl)₂, —C(═O)—NH—C₁₋₄alkyl-C₃₋₆cycloalkyl, —C(═O)—OH, —NR^{11a}R^{11b}, and
—NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —C(═O)—N(C₁₋₄alkyl)₂, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents hydrogen, —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, —S(═O)₂—C₁₋₄alkyl, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl or —(C═O)—C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and
wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b} and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, —O—C₁₋₄alkyl, cyano, C₁₋₄alkyl C₁₋₄alkyl

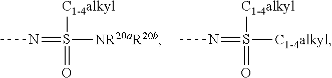

and
C₁₋₄alkyl substituted with one or two substituents each independently selected from the group consisting of Het^{3a}, Het^{6a}, Het^{6b}, and —NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵;
Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, R^{17b}, R^{20a}, R^{20b}, R^{22a}, and R^{22b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a}, R^{10b} and R^{10c} are each independently selected from the group consisting of hydrogen, C₁₋₄alkyl, and C₃₋₆cycloalkyl;
R^{10d} and R^{10e} are each independently selected from the group consisting of C₁₋₄alkyl, —O—C₁₋₄alkyl and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het⁷, or Het⁸;
and the pharmaceutically acceptable salts and the solvates thereof.

In an embodiment, the present invention in particular includes compounds of Formula (I-x2) as defined herein, and the tautomers and the stereoisomeric forms thereof, wherein

Q represents —CHR^{y}—;
R^{1a} represents —C(═O)—NR^{xa}R^{xb};
R^{xa} and R^{xb} are C₁₋₆alkyl optionally substituted with 1, 2 or 3 —OH;
R^{1b} represents F;
R² represents methyl;
R²¹ represents hydrogen or methyl;
Y represents a covalent bond or

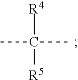

R⁵ represents hydrogen;
n1 is 1;
n2 is selected from 1 and 2;
R^{y} represents hydrogen;
R³ and R⁴ are each independently selected from Het¹, Cy², and C₁₋₈alkyl substituted with one, two, three or four substituents each independently selected from the group consisting of —NR^{xc}R^{xd}, Het¹ and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸; and wherein said heterocyclyl is optionally substituted on one carbon atom with oxo;
R⁸ represents C₁₋₆alkyl; or C₁₋₆alkyl substituted with one, two or three substituents each independently selected from —OH, —O—C₁₋₄alkyl and cyano;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one Het^{6a};
and the pharmaceutically acceptable salts and the solvates thereof.

In an embodiment, the present invention in particular includes compounds of Formula (I-x2) as defined herein, and the tautomers and the stereoisomeric forms thereof, wherein
Q represents —CHR^{y}—;
R^{1a} represents —C(═O)—NR^{xa}R^{xb};
R^{xa} and R^{xb} are C₁₋₆alkyl optionally substituted with 1, 2 or 3 —OH;
R^{1b} represents F;
R² represents methyl;
R²¹ represents hydrogen or methyl;
Y represents a covalent bond or

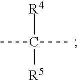

R⁵ represents hydrogen;
n1 is 1;
n2 is selected from 1 and 2;
R^{y} represents hydrogen;
R³ and R⁴ are each independently selected from Het¹, Cy², and C₁₋₆alkyl substituted with one, two, three or four substituents each independently selected from the group consisting of —NR^{xc}R^{xd}, Het¹ and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸; and wherein said heterocyclyl is optionally substituted on one carbon atom with oxo;
R⁸ represents C₁₋₆alkyl; or C₁₋₆alkyl substituted with one, two or three substituents each independently selected from —OH, —O—C₁₋₄alkyl and cyano;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one Het^{6a};
and the pharmaceutically acceptable salts and the solvates thereof.

In an embodiment, the present invention in particular includes compounds of Formula (I-x2) as defined herein, and the tautomers and the stereoisomeric forms thereof, wherein
Q represents —CHR^{y}—;
R^{1a} represents —C(═O)—NR^{xa}R^{xb};
R^{xa} and R^{xb} are C₁₋₆alkyl optionally substituted with 1, 2 or 3 —OH;
R^{1b} represents F;
R² represents methyl;
R²¹ represents hydrogen or methyl;
Y represents a covalent bond;
n1 is 1;
n2 is selected from 1 and 2;
R^{y} represents hydrogen;
R³ is selected from C₁₋₈alkyl substituted with one, two, three or four substituents each independently selected from the group consisting of —NR^{xc}R^{xd}, Het¹ and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸; and wherein said heterocyclyl is optionally substituted on one carbon atom with oxo;
R⁸ represents C₁₋₆alkyl; or C₁₋₆alkyl substituted with one, two or three substituents each independently selected from —OH, —O—C₁₋₄alkyl and cyano;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one Het^{6a};
and the pharmaceutically acceptable salts and the solvates thereof.

In an embodiment, the present invention in particular includes compounds of Formula (I-x2) as defined herein, and the tautomers and the stereoisomeric forms thereof, wherein
Q represents —CHR^{y}—;
R^{1a} represents —C(═O)—NR^{xa}R^{xb};
R^{xa} and R^{xb} are C₁₋₆alkyl;
R^{1b} represents F;
R² represents methyl;
R²¹ represents hydrogen;
Y represents a covalent bond;
n1 is 1;
n2 is selected from 1 and 2;
R^{y} represents hydrogen;
R³ is selected from C₁₋₈alkyl substituted with one substituent selected from the group consisting of —NR^{xc}R^{xd}, Het¹ and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸;
R⁸ represents C₁₋₆alkyl; or C₁₋₆alkyl substituted with one, two or three substituents each independently selected from —OH, —O—C₁₋₄alkyl and cyano;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one Het^{6a};
and the pharmaceutically acceptable salts and the solvates thereof.

In an embodiment, the present invention in particular includes compounds of Formula (I-x2) as defined herein, and the tautomers and the stereoisomeric forms thereof, wherein
Q represents —CHR^{y}—;
R^{1a} represents —C(═O)—NR^{xa}R^{xb};
R^{xa} and R^{xb} are C₁₋₆alkyl;
R^{1b} represents F;
R² represents methyl;
R²¹ represents hydrogen;
Y represents a covalent bond;
n1 is 1;
n2 is selected from 1 and 2;
R^{y} represents hydrogen;
R³ is selected from C₁₋₄alkyl substituted with one Het¹;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸;
R⁸ represents C₁₋₆alkyl; or C₁₋₆alkyl substituted with one, two or three substituents each independently selected from —OH, —O—C₁₋₄alkyl and cyano;
and the pharmaceutically acceptable salts and the solvates thereof.

In an embodiment, the present invention includes compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

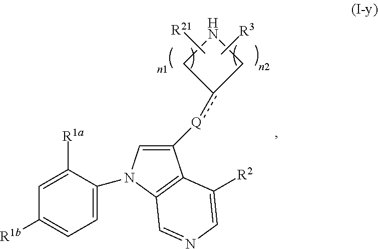

wherein the variables are as defined for the compounds of Formula (I) or any subgroup thereof as mentioned in any of the other embodiments.

In an embodiment, the present invention includes compounds of Formula (I) and the pharmaceutically acceptable salts, and the solvates thereof, or any subgroup thereof as mentioned in any of the other embodiments, wherein the compounds of Formula (I) are restricted to compounds of Formula (I-y1):

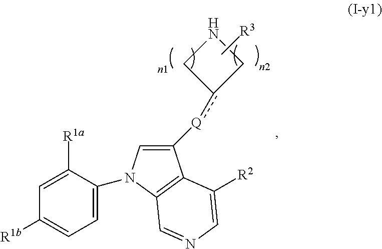

wherein the variables are as defined for the compounds of Formula (I) or any subgroup thereof as mentioned in any of the other embodiments.

In an embodiment, the present invention includes compounds of Formula (I) and the pharmaceutically acceptable salts, and the solvates thereof, or any subgroup thereof as mentioned in any of the other embodiments, wherein the compounds of Formula (I) are restricted to compounds of Formula (I-z):

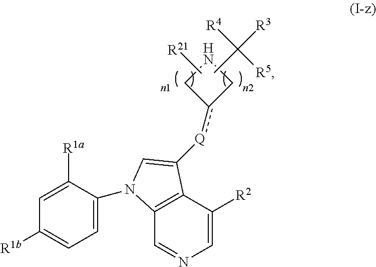

wherein the variables are as defined for the compounds of Formula (I) or any subgroup thereof as mentioned in any of the other embodiments.

In an embodiment, the present invention includes compounds of Formula (I) and the pharmaceutically acceptable salts, and the solvates thereof, or any subgroup thereof as mentioned in any of the other embodiments, wherein the compounds of Formula (I) are restricted to compounds of Formula (I-z1):

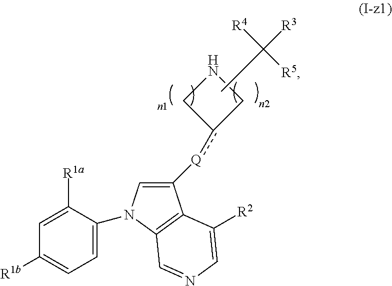

wherein the variables are as defined for the compounds of Formula (I) or any subgroup thereof as mentioned in any of the other embodiments.

In an embodiment, the present invention includes compounds of Formula (I) and the pharmaceutically acceptable salts, and the solvates thereof, or any subgroup thereof as mentioned in any of the other embodiments, wherein the compounds of Formula (I) are restricted to compounds of Formula (I-q):

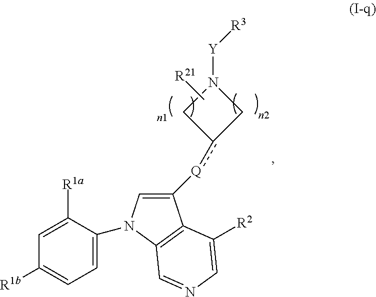

wherein the variables are as defined for the compounds of Formula (I) or any subgroup thereof as mentioned in any of the other embodiments.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof, and the free bases, any pharmaceutically acceptable salts, and the solvates thereof.

In an embodiment the compound of Formula (I) is selected from the group consisting of compounds 43, 51, 51a, 59, 60, 115, 117a, 125, 140, 157, 159, 169a and 207.

In an embodiment the compound of Formula (I) is selected from the group consisting of compounds 43, 51, 51a, 59, 60, 115, 117a, 125, 140, 157, 159, 169a and 207;
tautomers and stereoisomeric forms thereof,
and the free bases, any pharmaceutically acceptable salts, and the solvates thereof.

In a particular embodiment, the solvate is a hydrate. In a particular embodiment, the pharmaceutically acceptable salt is a HCl salt. In a particular embodiment, the compound is a HCl salt hydrate.

In an embodiment the compound of Formula (I) is

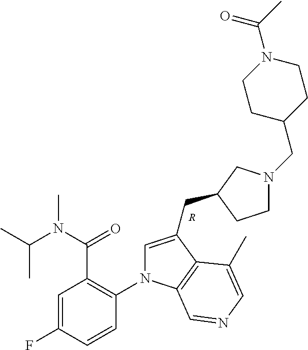

or a pharmaceutically acceptable salt or solvate thereof; in particular a HCl salt, solvate; more in particular a HCl salt, hydrate; more in particular a mono HCl salt, hydrate; even more in particular mono HCl salt, trihydrate.

According to particular embodiments, the menin-MLL inhibitor is selected is selected from the group consisting of compounds 43, 50, 51, 51a, 59, 60, 61, 62, 63, 64, 65, 82, 83, 85, 86, 87, 91, 92, 98, 101, 104, 106, 108, 114, 117a, 120, 121, 125, 126, 135, 156, 169, 169a, 169b, 188a, 188b, 190a, 190b, 191a, 191b, 194, 196, 207, 213a, 213b, 283, 286, 287, 288, 289, 290, 291, 292a, 292b, 293a, 293b, 294a, 294b, 295, 296, 378, 381, 382, 383, 384, 387, 388, 392, 394, 395, 396, 397, 398, 399, 400, 401, 402, 403, 404, 405, 407, 408, 409, 410, 411, 412, 413, 414, 415, 416, 417, 418, 442, 448, 485, 498, 526a, 526b, 531; tautomers and stereoisomeric forms thereof, and the free bases, any pharmaceutically acceptable salts, and the solvates thereof.

According to particular embodiments, the menin-MLL inhibitor is selected is selected from the group consisting of compounds 43, 51, 51a, 59, 60, 117a, 125, 169a, 207; tautomers and stereoisomeric forms thereof, and the free bases, any pharmaceutically acceptable salts, and the solvates thereof.

According to particular embodiments, the menin-MLL inhibitor is Compound 51 or a solvate thereof. According to particular embodiments, the menin-MLL inhibitor is Compound 51 or a hydrate thereof. According to particular embodiments, the menin-MLL inhibitor is Compound 51a.

In some embodiments, provided is a pharmaceutical composition comprising a pharmaceutically acceptable carrier, and as active ingredient a therapeutically effective amount of a combination as described in any of the other embodiments.

In some embodiments, provided is a combination therapy comprising a menin-MLL inhibitor of Formula (I), or a pharmaceutically acceptable salt or a solvate thereof, and at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

According to embodiments, the menin-MLL inhibitor is a compound of Formula (I), or a pharmaceutically acceptable salt or a solvate thereof.

In particular embodiments, the menin-MLL inhibitor may have improved metabolic stability properties.

In particular embodiments, the menin-MLL inhibitor may have extended in vivo half-life (T½).

In particular embodiments, the menin-MLL inhibitor may have improved oral bioavailability.

In particular embodiments, the menin-MLL inhibitor may reduce tumor growth e.g., tumors harbouring MLL (KMT2A) gene rearrangements/alterations and/or NPM1 mutations.

In particular embodiments, the menin-MLL inhibitor may have improved PD properties in vivo during a prolonged period of time, e.g., inhibition of target gene expression such as MEIS1 and upregulation of differentiation marker over a period of at least 16 hours.

In particular embodiments, the menin-MLL inhibitor may have an improved safety profile (e.g., reduced hERG inhibition; improved cardiovascular safety).

In particular embodiments, the menin-MLL inhibitor may be suitable for Q.D. dosing (once daily).

According to embodiments, at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

According to embodiments, the hypomethylating agent includes, but is not limited to, azacitidine, decitabine, or pharmaceutically acceptable salts or solvates thereof.

According to embodiments, the cytidine deaminase inhibitor includes, but is not limited to, cedazuridine or pharmaceutically acceptable salts or solvates thereof.

According to embodiments, the DNA intercalating agent includes, but is not limited to, an anthracycline (e.g., daunorubicin, doxorubicin, idarubicin).

According to embodiments, the DNA intercalating agent is daunorubicin.

According to embodiments, the DNA intercalating agent is doxorubicin.

According to embodiments, the DNA intercalating agent is idarubicin.

According to embodiments, the pyrimidine analog includes, but is not limited to, cytarabine (ARA-C).

According to embodiments, the purine analog is fludarabine.

According to embodiments, the kinase inhibitor is a FLT-3 inhibitor, a BTK inhibitor, an ABL inhibitor, an Aurora inhibitor or a multi-kinase inhibitor of two or more kinase inhibitors thereof.

According to embodiments, the kinase inhibitor is a multi-kinase inhibitor of FLT-3 inhibitor, ABL inhibitor, and Aurora inhibitor. According to embodiments, such multi-kinase inhibitor includes, but is not limited to KW-2449.

According to embodiments, the kinase inhibitor is a tyrosine kinase inhibitor.

According to embodiments, the tyrosine kinase inhibitor is a FLT-3 inhibitor or a BTK inhibitor.

According to embodiments, the FLT3 inhibitor includes, but is not limited to, sorafenib, sunitinib, midostaurin (PKC412), lestaurtinib (CEP-701), tandutinib (MLN518), quizartinib (AC220), gilteritinib (ASP2215), and KW-2449.

According to embodiments, the FLT3 inhibitor is gilteritinib (ASP2215).

According to embodiments, the FLT3 inhibitor is midostaurin (PKC412).

According to embodiments, the BTK inhibitor includes, but is not limited to, ibrutinib.

According to embodiments, the CD20 inhibitor includes, but is not limited to, an anti-CD20 antibody (e.g., obinutuzumab (GA101)).

According to embodiments, the IDH inhibitor includes, but is not limited to, ivosidenib and enasidenib.

According to embodiments, the isocitrate dehydrogenase-1 inhibitor includes, but is not limited to, ivosidenib.

According to embodiments, the isocitrate dehydrogenase-2 inhibitor includes, but is not limited to, enasidenib.

According to embodiments, the immunomodulatory agent includes, but is not limited to, PD-1 inhibitors (e.g., nivolumab, atezolizumab and pembrolizumab), thalidomide, lenalidomide, pomalidomide, Bacillus Calmette-Guérin (BCG) and levamisole.

According to embodiments, the PD-1 inhibitor includes, but is not limited to, nivolumab, atezolizumab and pembrolizumab.

According to embodiments, the DHODH inhibitor includes, but is not limited to, a compound having the structure of Formula (Z):

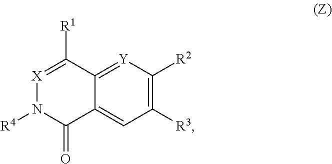

### Wherein

X is CH or N;
Y is CH or N;
R¹ is selected from the group consisting of: C₁₋₆alkyl; C₁₋₆alkyl substituted with OH, or OCH₃;
C₂₋₆alkenyl; C₁₋₆haloalkyl; C₁₋₆haloalkyl substituted with OH, or OCH₃; C₂₋₆haloalkenyl;
N(CH₃)₂; C₃₋₆cycloalkyl; C₃₋₆cycloalkyl substituted with C₁₋₆alkyl; and phenyl;
R² is

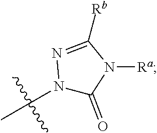

wherein
R^{a} is selected from the group consisting of C₁₋₆alkyl, C₁₋₆haloalkyl, and C₃₋₆cycloalkyl;
R^{b} is C₁₋₆alkyl or C₁₋₆alkyl substituted with a member selected from the group consisting of:
OH, halo, CN, OC₁₋₆alkyl, OC₁₋₆haloalkyl and OC₃₋₆cycloalkyl;
R³ is selected from the group consisting of: H, halo, CH₃ and OCH₃;
R⁴ is selected from the group consisting of:
C₁₋₈alkyl; C₁₋₆alkyl substituted with one or two OCH₃; C₃₋₆cycloalkyl; C₃₋₆cycloalkyl substituted with CH₃, or OCH₃; CH₂—C₃₋₆cycloalkyl; and

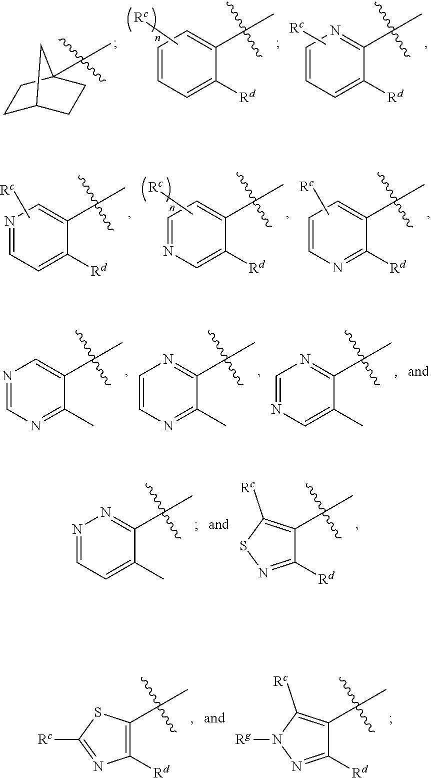

wherein
each R^{c} is independently selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; NO₂; OH; O—CH₂CH₂OH; and OC₁₋₆alkyl;
R^{d} is selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; CN; and OC₁₋₆alkyl;
R^{g} is selected from the group consisting of H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
or a pharmaceutically acceptable salt, isotope, N-oxide, solvate, or stereoisomer thereof;
or a compound selected from

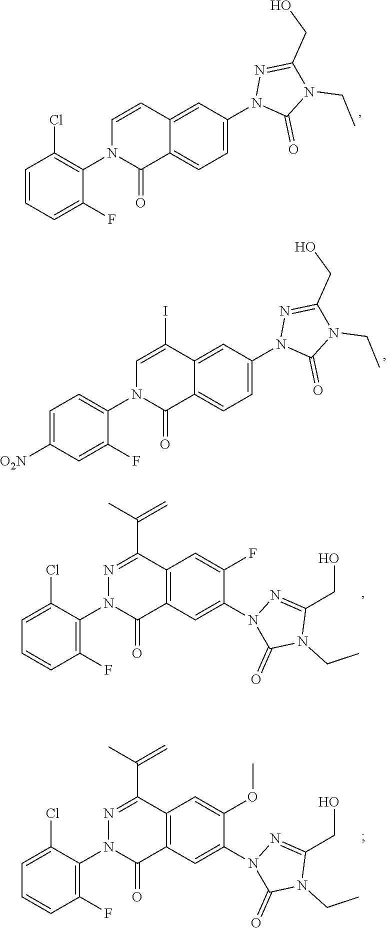 [SMILES: CCn1c(CO)nn(-c2ccc3c(=O)n(-c4c(F)cccc4Cl)ccc3c2)c1=O ; CCn1c(CO)nn(-c2ccc3c(=O)n(-c4ccc([N+](=O)[O-])cc4F)cc(I)c3c2)c1=O ; C=C(C)c1nn(-c2c(F)cccc2Cl)c(=O)c2cc(-n3nc(CO)n(CC)c3=O)c(F)cc12 ; C=C(C)c1nn(-c2c(F)cccc2Cl)c(=O)c2cc(-n3nc(CO)n(CC)c3=O)c(OC)cc12]

or a pharmaceutically acceptable salt, N-oxide, solvate, or stereoisomer thereof.

According to embodiments, the DHODH inhibitor includes, but is not limited to, a compound having the structure of Formula (Z):

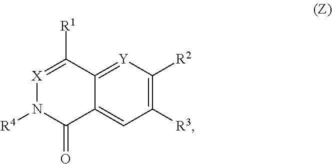

wherein
X is CH or N;
Y is CH or N;
R¹ is selected from the group consisting of: C₁₋₆alkyl; C₁₋₆alkyl substituted with OH, or OCH₃;
C₂₋₆alkenyl; C₁₋₆haloalkyl; C₁₋₆haloalkyl substituted with OH, or OCH₃; C₂₋₆haloalkenyl;
N(CH₃)₂; C₃₋₆cycloalkyl; C₃₋₆cycloalkyl substituted with C₁₋₆alkyl; and phenyl;
R² is

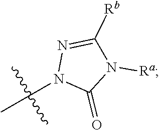

wherein
R^{a} is selected from the group consisting of: C₁₋₆alkyl, C₁₋₆haloalkyl, and C₃₋₆cycloalkyl;
R^{b} is C₁₋₆alkyl or C₁₋₆alkyl substituted with a member selected from the group consisting of:
OH, halo, CN, OC₁₋₆alkyl, OC₁₋₆haloalkyl and OC₃₋₆cycloalkyl;
R³ is selected from the group consisting of: H, halo, CH₃ and OCH₃;
R⁴ is selected from the group consisting of:
C₁₋₆alkyl; C₁₋₆alkyl substituted with one or two OCH₃; C₃₋₆cycloalkyl; C₃₋₆cycloalkyl substituted with CH₃, or OCH₃; CH₂—C₃₋₆cycloalkyl; and

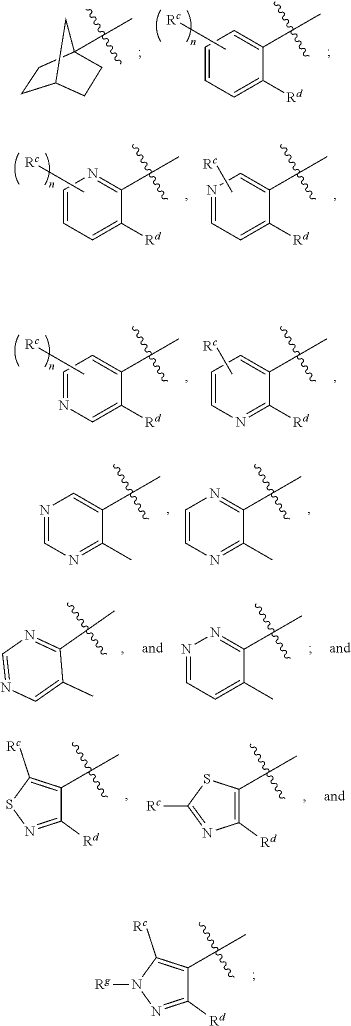

wherein
each R^{c} is independently selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; NO₂; OH; O—CH₂CH₂OH; and OC₁₋₆alkyl;
R^{d} is selected from the group consisting of H; halo; C₁₋₆alkyl; C₁₋₆alkyl substituted with a member selected from the group consisting of: OH, OCH₃, SCH₃, and OCF₃; C₁₋₆haloalkyl;
C₁₋₆haloalkyl substituted with a member selected from the group consisting of: OH, and OCH₃; CN; and OC₁₋₆alkyl;
R^{g} is selected from the group consisting of H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
or a pharmaceutically acceptable salt, isotope, N-oxide, solvate, or stereoisomer thereof.

In the context of Formula (Z), the following definitions apply:

The term “alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl, etc.). The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂₋₆ for straight chain, C₃₋₆ for branched chain).

The term “haloalkyl” refers to a straight- or branched-chain alkyl group having from 1 to 6 carbon atoms in the chain optionally substituting hydrogens with halogens. The term “C₁₋₆ haloalkyl” as used here refers to a straight- or branched-chain alkyl group having from 1 to 6 carbon atoms in the chain, optionally substituting hydrogens with halogens. The term “C₁₋₄ haloalkyl” as used here refers to a straight- or branched-chain alkyl group having from 1 to 4 carbon atoms in the chain, optionally substituting hydrogens with halogens. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “haloalkenyl” includes unsaturated aliphatic groups analogous in length and possible substitution to the alkyls described above, but that contain at least one double bond and having from 1 to 6 carbon atoms in the chain optionally substituting hydrogens with halogens.

The term “aryl” refers to a monocyclic, aromatic carbocycle (ring structure having ring atoms that are all carbon) having 6 atoms per ring. (Carbon atoms in the aryl groups are sp2 hybridized.)

The term “heteroaryl” refers to a monocyclic or fused bicyclic heterocycle (ring structure having ring atoms selected from carbon atoms and up to four heteroatoms selected from nitrogen, oxygen, and sulfur) having from 3 to 9 ring atoms per heterocycle. Illustrative examples of heteroaryl groups include the following entities, in the form of properly bonded moieties:

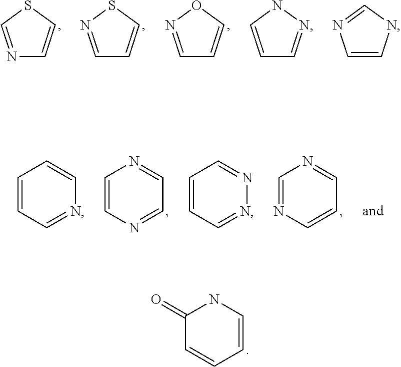

Those skilled in the art will recognize that the species listed or illustrated above are not exhaustive, and that additional species within the scope of these defined terms may also be selected.

The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below.

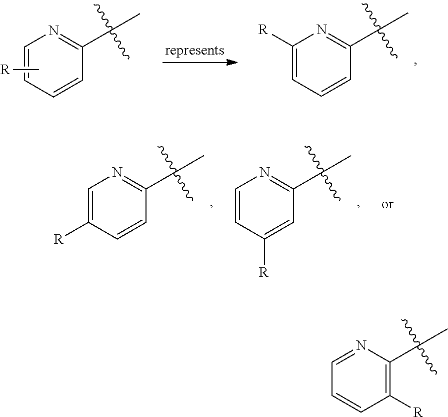

In an embodiment of the invention, the DHODH inhibitor is a compound of Formula (Z) wherein X is CH.

In an embodiment of the invention, the DHODH inhibitor is a compound of Formula (Z) wherein X is N.

In an embodiment of the invention, the DHODH inhibitor is a compound of Formula (Z) wherein Y is CH.

In an embodiment of the invention, the DHODH inhibitor is a compound of Formula (Z) wherein Y is N.

In an embodiment of the invention, the DHODH inhibitor is a compound of Formula (Z) wherein R¹ is C₁₋₄alkyl; C₁₋₄alkyl substituted with OH, or OCH₃; C₂₋₄alkenyl; C₁₋₄haloalkyl; C₁₋₄haloalkyl substituted with OH, or OCH₃; C₂₋₄haloalkenyl; N(CH₃)₂; cyclopropyl; cyclopropyl substituted with C₁₋₄alkyl; or phenyl.

In an embodiment of the invention, the DHODH inhibitor is a compound of Formula (Z) wherein R¹ is CH₃, CH₂CH₃,

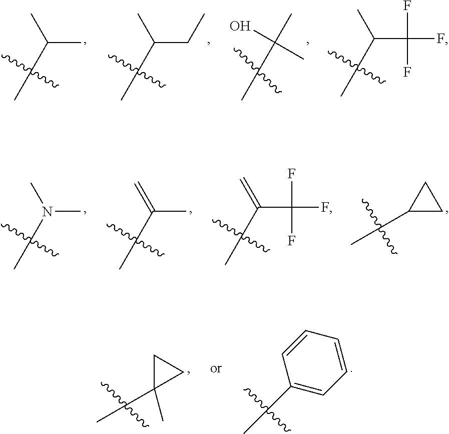

In an embodiment of the invention, the DHODH inhibitor is a compound of Formula (Z) wherein R¹ is

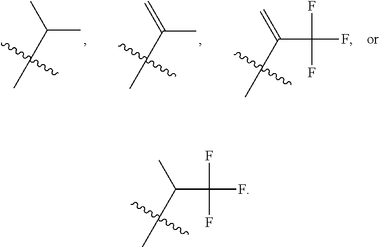

In an embodiment of the invention, the DHODH inhibitor is a compound of Formula (Z) wherein
R² is

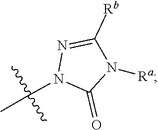

wherein R^{b} is C₁₋₄alkyl substituted with OH, halo, CN, OC₁₋₄alkyl, OC₁₋₄haloalkyl or OC₃₋₆cycloalkyl; and
R^{a} is C₁₋₄alkyl, C₁₋₄haloalkyl, or C₃₋₆cycloalkyl.

In an embodiment of the invention, the DHODH inhibitor is a compound of Formula (Z) wherein R² is

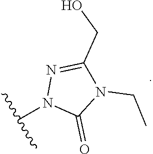 [SMILES: *n1nc(CO)n(CC)c1=O]

In an embodiment of the invention, the DHODH inhibitor is a compound of Formula (Z) wherein R³ is H.

In an embodiment of the invention, the DHODH inhibitor is a compound of Formula (Z) wherein R³ is F.

In an embodiment of the invention, the DHODH inhibitor is a compound of Formula (Z) wherein R³ is CH₃.

In an embodiment of the invention, the DHODH inhibitor is a compound of Formula (Z) wherein R³ is OCH₃.

In an embodiment of the invention, the DHODH inhibitor is a compound of Formula (Z) wherein R⁴ is

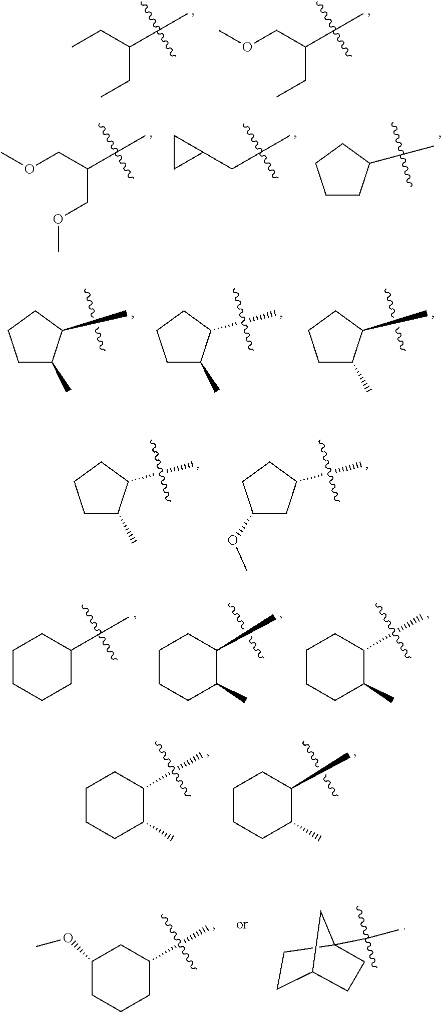

In an embodiment of the invention, the DHODH inhibitor is a compound of Formula (Z) wherein
R⁴ is

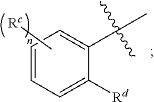

wherein
each R^{c} is independently selected from the group consisting of: H; halo; C₁₋₄alkyl; C₁₋alkyl substituted with a member selected from the group consisting of: OH, OCH₃, SCH₃, and OCF₃;
C₁₋₄haloalkyl; C₁₋₄haloalkyl substituted with a member selected from the group consisting of:
OH, and OCH₃; and NO₂;
R^{d} is selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₄alkyl; CN; and OC₁₋₆alkyl; and
n is 1, or 2.

In an embodiment of the invention, the DHODH inhibitor is a compound of Formula (Z) wherein
R⁴ is

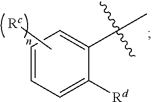

each R^{c} is independently selected from the group consisting of: H, halo, C₁₋₄alkyl, C₁₋₄haloalkyl, NO₂, O—CH₂CH₂OH, and OC₁₋₄alkyl;
R^{d} is selected from the group consisting of: H, halo, C₁₋₄alkyl, CN, and OC₁₋₆alkyl; and
n is 1, or 2.

In an embodiment of the invention, the DHODH inhibitor is a compound of Formula (Z) wherein R⁴ is

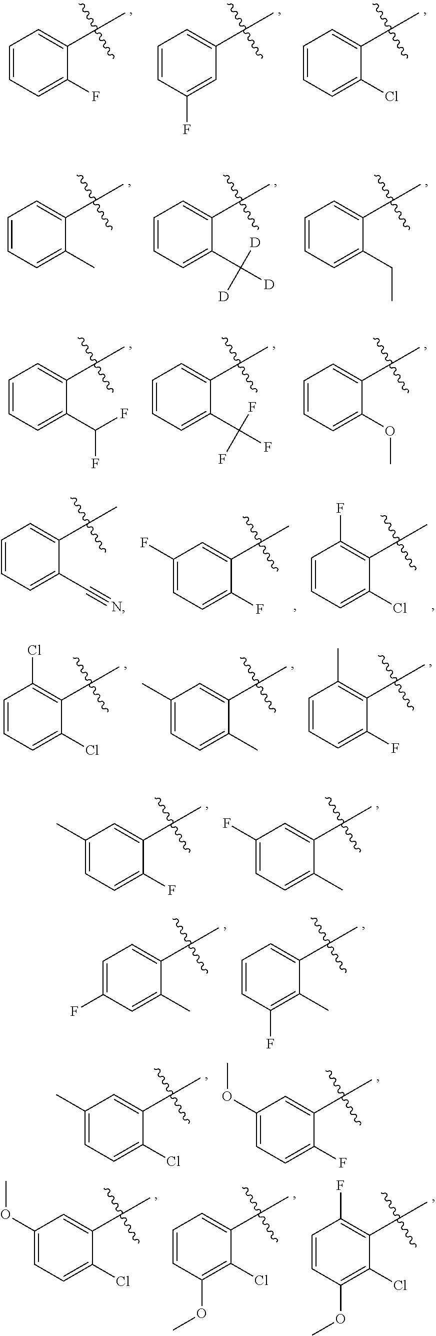

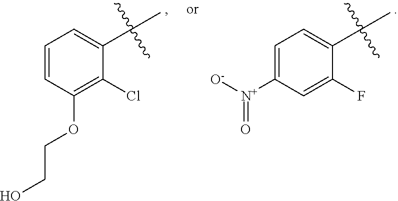 [SMILES: *c1cccc(OCCO)c1Cl ; *c1ccc([N+](=O)[O-])cc1F]

In an embodiment of the invention, the DHODH inhibitor is a compound of Formula (Z) wherein R⁴ is

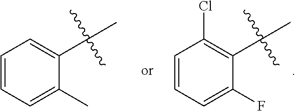

In an embodiment of the invention, the DHODH inhibitor is a compound of Formula (Z) wherein
R⁴ is

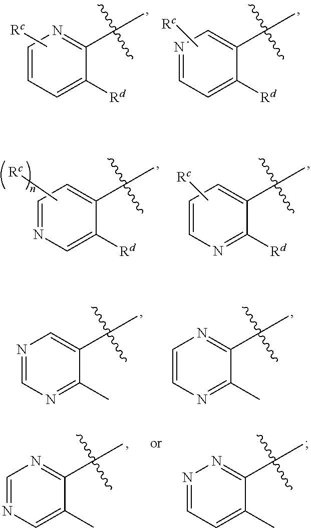

wherein
each R^{c} is independently selected from the group consisting of: H; halo; C₁₋₄alkyl; C₁₋alkyl substituted with a member selected from the group consisting of: OH, OCH₃, SCH₃, and OCF₃; C₁₋₄haloalkyl; C₁₋₄haloalkyl substituted with a member selected from the group consisting of: OH, and OCH₃; and R^{d} is selected from the group consisting of halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₄alkyl; CN; and OC₁₋₆alkyl.

In an embodiment of the invention, the DHODH inhibitor is a compound of Formula (Z) wherein
R⁴ is

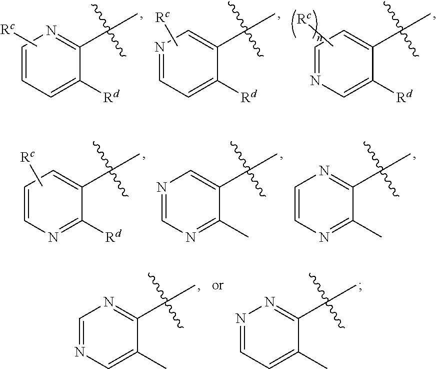

wherein
each R^{c} is independently selected from the group consisting of: H, halo, C₁₋₄alkyl, C₁₋₄haloalkyl, OC₁₋₄alkyl, and OH;
R^{d} is selected from the group consisting of: halo, C₁₋₄alkyl, and OC₁₋₄alkyl; and
n is 1, or 2.

In an embodiment of the invention, the DHODH inhibitor is a compound of Formula (Z) wherein R⁴ is

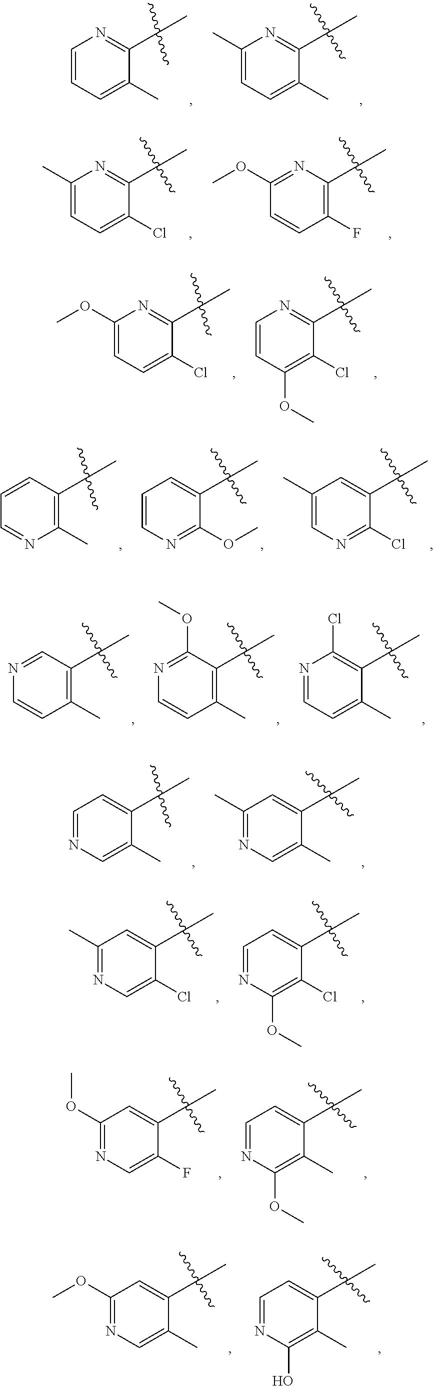

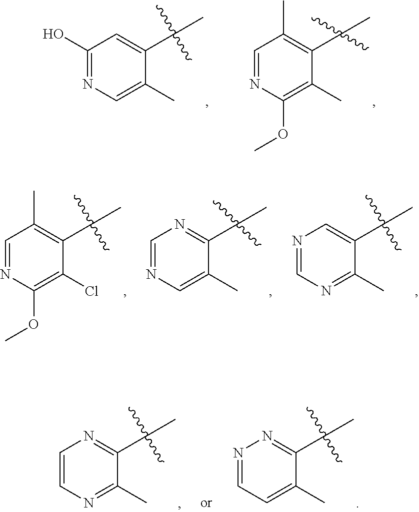

In an embodiment of the invention, the DHODH inhibitor is a compound of Formula (Z) wherein R⁴ is

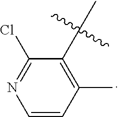 [SMILES: *c1c(C)ccnc1Cl]

In an embodiment of the invention, the DHODH inhibitor is a compound of Formula (Z) wherein
R⁴ is

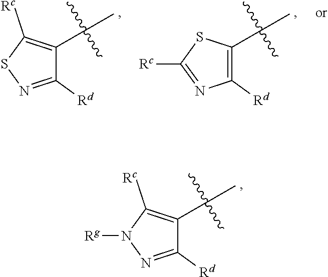

wherein
R^{c} is H; halo; C₁₋₄alkyl; C₁₋₄alkyl substituted with OH, OCH₃, SCH₃, or OCF₃; C₁₋₄haloalkyl;
C₁₋₄haloalkyl substituted with OH, or OCH₃; or OC₁₋₄alkyl;
R^{d} is halo; C₁₋₄alkyl; C₁₋₄alkyl substituted with OH, OCH₃, SCH₃, or OCF₃; C₁₋₄haloalkyl; or
C₁₋₄haloalkyl substituted with OH, or OCH₃; and
R^{g} is H; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃.

In an embodiment of the invention, the DHODH inhibitor is a compound of Formula (Z) wherein
R⁴ is

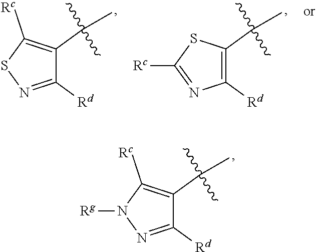

wherein
R^{c} is H or halo;
R^{d} is C₁₋₄alkyl; and
R^{g} is H.

In an embodiment of the invention, the DHODH inhibitor is a compound of Formula (Z) wherein R⁴ is

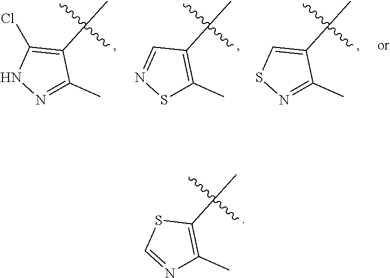

In an embodiment of the invention, the DHODH inhibitor is a compound of Formula (Z) selected from the group consisting of:
6-(4-Ethyl-3-(hydroxymethyl)-5-oxo-4,5-dihydro-1H-1,2,4-triazol-1-yl)-2-(3-fluorophenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-2-(3-fluorophenyl)-4-isopropylisoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-isopropylisoquinolin-1(2H)-one;
6-(4-Ethyl-3-(hydroxymethyl)-5-oxo-4,5-dihydro-1H-1,2,4-triazol-1-yl)-2-(3-fluorophenyl)-4-phenylisoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-(prop-1-en-2-yl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-(3,3,3-trifluoroprop-1-en-2-yl)isoquinolin-1(2H)-one;
2-(2,6-Dichlorophenyl)-6-(4-ethyl-3-(hydroxymethyl)-5-oxo-4,5-dihydro-1H-1,2,4-triazol-1-yl)-4-(1-methylcyclopropyl)isoquinolin-1(2H)-one;
2-(2,6-Dichlorophenyl)-6-(4-ethyl-3-(hydroxymethyl)-5-oxo-4,5-dihydro-1H-1,2,4-triazol-1-yl)-4-(prop-1-en-2-yl)isoquinolin-1(2H)-one;
2-(2-Chloro-6-fluorophenyl)-4-cyclopropyl-6-(4-ethyl-3-(hydroxymethyl)-5-oxo-4,5-dihydro-1H-1,2,4-triazol-1-yl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-4-(prop-1-en-2-yl)-2-(2-(trifluoromethyl)phenyl)isoquinolin-1(2H)-one;
2-(6-(4-Ethyl-3-(hydroxymethyl)-5-oxo-4,5-dihydro-1H-1,2,4-triazol-1-yl)-1-oxo-4-(prop-1-en-2-yl)isoquinolin-2(1H)-yl)benzonitrile;
2-(2-Chlorophenyl)-6-(4-ethyl-3-(hydroxymethyl)-5-oxo-4,5-dihydro-1H-1,2,4-triazol-1-yl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-4-(prop-1-en-2-yl)-2-(o-tolyl)isoquinolin-1(2H)-one;
2-(2-Chlorophenyl)-6-(4-ethyl-3-(hydroxymethyl)-5-oxo-4,5-dihydro-1H-1,2,4-triazol-1-yl)-4-isopropylphthalazin-1(2H)-one;
6-(4-Ethyl-3-(hydroxymethyl)-5-oxo-4,5-dihydro-1H-1,2,4-triazol-1-yl)-2-(3-fluorophenyl)-4-isopropylphthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-isopropylphthalazin-1(2H)-one;
4-Ethyl-6-(4-ethyl-3-(hydroxymethyl)-5-oxo-4,5-dihydro-1H-1,2,4-triazol-1-yl)-2-(3-fluorophenyl)phthalazin-1(2H)-one;
4-Ethyl-6-(4-ethyl-3-(hydroxymethyl)-5-oxo-4,5-dihydro-1H-1,2,4-triazol-1-yl)-2-(o-tolyl)phthalazin-1(2H)-one;
6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one;
2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-(1-methylcyclopropyl)isoquinolin-1(2H)-one;
6-(4-Ethyl-3-(hydroxymethyl)-5-oxo-4,5-dihydro-1H-1,2,4-triazol-1-yl)-2-(3-fluorophenyl)-4-(2-hydroxypropan-2-yl)isoquinolin-1(2H)-one;
4-(Dimethylamino)-6-(4-ethyl-3-(hydroxymethyl)-5-oxo-4,5-dihydro-1H-1,2,4-triazol-1-yl)-2-(o-tolyl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)phthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-methoxy-4-(prop-1-en-2-yl)phthalazin-1(2H)-one;
2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(4-Ethyl-3-(hydroxymethyl)-5-oxo-4,5-dihydro-1H-1,2,4-triazol-1-yl)-3-fluoro-8-(prop-1-en-2-yl)-6-(o-tolyl)-1,6-naphthyridin-5(6H)-one;
2-(4-Ethyl-3-(hydroxymethyl)-5-oxo-4,5-dihydro-1H-1,2,4-triazol-1-yl)-3-fluoro-8-methyl-6-(o-tolyl)pyrido[2,3-d]pyridazin-5(6H)-one;
2-(4-Ethyl-3-(hydroxymethyl)-5-oxo-4,5-dihydro-1H-1,2,4-triazol-1-yl)-3-fluoro-8-isopropyl-6-(o-tolyl)pyrido[2,3-d]pyridazin-5(6H)-one;
6-(2-Chloro-6-fluorophenyl)-2-(4-ethyl-3-(hydroxymethyl)-5-oxo-4,5-dihydro-1H-1,2,4-triazol-1-yl)-3-fluoro-8-(prop-1-en-2-yl)-1,6-naphthyridin-5(6H)-one;
6-(2-Chloro-6-fluorophenyl)-2-(4-ethyl-3-(hydroxymethyl)-5-oxo-4,5-dihydro-1H-1,2,4-triazol-1-yl)-3-fluoro-8-isopropyl-1,6-naphthyridin-5(6H)-one;
(S)-2-(4-Ethyl-3-(hydroxymethyl)-5-oxo-4,5-dihydro-1H-1,2,4-triazol-1-yl)-3-fluoro-6-(o-tolyl)-8-(1,1,1-trifluoropropan-2-yl)-1,6-naphthyridin-5(6H)-one;
(R)-2-(4-Ethyl-3-(hydroxymethyl)-5-oxo-4,5-dihydro-1H-1,2,4-triazol-1-yl)-3-fluoro-6-(o-tolyl)-8-(1,1,1-trifluoropropan-2-yl)-1,6-naphthyridin-5(6H)-one;
6-(4-Ethyl-3-(hydroxymethyl)-5-oxo-4,5-dihydro-1H-1,2,4-triazol-1-yl)-7-fluoro-4-isopropyl-2-(4-methylthiazol-5-yl)isoquinolin-1(2H)-one;
2-(4-Ethyl-3-(hydroxymethyl)-5-oxo-4,5-dihydro-1H-1,2,4-triazol-1-yl)-3-fluoro-8-isopropyl-6-(o-tolyl)-1,6-naphthyridin-5(6H)-one;
6-(4-Ethyl-3-(hydroxymethyl)-5-oxo-4,5-dihydro-1H-1,2,4-triazol-1-yl)-7-fluoro-2-(2-fluoro-5-methylphenyl)-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-5-methylphenyl)-4-(prop-1-en-2-yl)isoquinolin-1(2H)-one;
2-(2-Chloro-5-methylphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-5-methoxyphenyl)-4-(prop-1-en-2-yl)isoquinolin-1(2H)-one;
2-(2-Chlorophenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-4-(prop-1-en-2-yl)-2-(o-tolyl)isoquinolin-1(2H)-one;
2-(2-Chloro-5-methoxyphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
racemic-4-(sec-Butyl)-2-(2-chloro-6-fluorophenyl)-6-(4-ethyl-3-(hydroxymethyl)-5-oxo-4,5-dihydro-1H-1,2,4-triazol-1-yl)-7-fluoroisoquinolin-1(2H)-one;
2-(3-Chloro-6-methoxypyridin-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxy-4-methylpyridin-3-yl)isoquinolin-1(2H)-one;
2-(2-Chloro-6-fluoro-3-methoxyphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(o-tolyl)phthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropylphthalazin-1(2H)-one;
Racemic 6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)phthalazin-1(2H)-one;
(S*)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)phthalazin-1(2H)-one;
(R*)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)phthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-(1-methylcyclopropyl)isoquinolin-1(2H)-one;
6-(4-Ethyl-3-(hydroxymethyl)-5-oxo-4,5-dihydro-1H-1,2,4-triazol-1-yl)-7-fluoro-2-(2-methoxy-4-methylpyridin-3-yl)-4-(prop-1-en-2-yl)isoquinolin-1(2H)-one;
2-(5-Chloro-3-methyl-1H-pyrazol-4-yl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(3-Chloro-2-methoxy-5-methylpyridin-4-yl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-5-methoxyphenyl)-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(4-fluoro-2-methylphenyl)-4-isopropylisoquinolin-1(2H)-one;
2-(2-Chloro-3-(2-hydroxyethoxy)phenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluorophenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(5-fluoro-2-methylphenyl)-4-isopropylisoquinolin-1(2H)-one;
2-(2,5-Difluorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-6-methylphenyl)-4-isopropylisoquinolin-1(2H)-one;
2-(2-Chloro-3-methoxyphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-methoxy-3,5-dimethylpyridin-4-yl)-4-(prop-1-en-2-yl)isoquinolin-1(2H)-one;
2-(2,5-Difluorophenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluoro-6-methylphenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(3-fluoro-2-methylphenyl)-4-isopropylisoquinolin-1(2H)-one;
2-(2,5-Dimethylphenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(4-fluoro-2-methylphenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-fluorophenyl)-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxy-3,5-dimethylpyridin-4-yl)isoquinolin-1(2H)-one;
6-(4-Ethyl-3-(hydroxymethyl)-5-oxo-4,5-dihydro-1H-1,2,4-triazol-1-yl)-7-methyl-4-(prop-1-en-2-yl)-2-(o-tolyl)isoquinolin-1(2H)-one;
2-(2-Chloro-6-fluoro-3-methoxyphenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-2-(o-tolyl)-4-(3,3,3-trifluoroprop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(5-fluoro-2-methylphenyl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-methoxyphenyl)-4-(prop-1-en-2-yl)isoquinolin-1(2H)-one;
2-(2-Chloro-5-methylpyridin-3-yl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(5-fluoro-2-methoxypyridin-4-yl)-4-(prop-1-en-2-yl)isoquinolin-1(2H)-one;
6-(4-Ethyl-3-(hydroxymethyl)-5-oxo-4,5-dihydro-1H-1,2,4-triazol-1-yl)-7-fluoro-2-(3-fluoro-2-methylphenyl)-4-(prop-1-en-2-yl)isoquinolin-1(2H)-one;
2-(2,5-Dimethylphenyl)-6-(4-ethyl-3-(hydroxymethyl)-5-oxo-4,5-dihydro-1H-1,2,4-triazol-1-yl)-7-fluoro-4-(prop-1-en-2-yl)isoquinolin-1(2H)-one;
Racemic-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)isoquinolin-1(2H)-one;
6-(4-Ethyl-3-(hydroxymethyl)-5-oxo-4,5-dihydro-1H-1,2,4-triazol-1-yl)-2-(2-ethylphenyl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxypyridin-3-yl)isoquinolin-1(2H)-one;
6-(4-Ethyl-3-(hydroxymethyl)-5-oxo-4,5-dihydro-1H-1,2,4-triazol-1-yl)-7-fluoro-2-(5-fluoro-2-methoxypyridin-4-yl)-4-isopropylisoquinolin-1(2H)-one;
2-(2-Chloro-5-methylpyridin-3-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-(methyl-d₃)phenyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(4-methylpyrimidin-5-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxyphenyl)isoquinolin-1(2H)-one;
6-(4-Ethyl-3-(hydroxymethyl)-5-oxo-4,5-dihydro-1H-1,2,4-triazol-1-yl)-7-fluoro-2-(3-fluoro-6-methoxypyridin-2-yl)-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(3-methylpyrazin-2-yl)isoquinolin-1(2H)-one;
2-(2-Chloro-5-methylpyridin-3-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(4-Ethyl-3-(hydroxymethyl)-5-oxo-4,5-dihydro-1H-1,2,4-triazol-1-yl)-3-fluoro-6-(2-fluoro-5-methylphenyl)-8-isopropyl-1,6-naphthyridin-5(6H)-one;
6-(4-Ethyl-3-(hydroxymethyl)-5-oxo-4,5-dihydro-1H-1,2,4-triazol-1-yl)-7-fluoro 4-isopropyl-2-(4-methylpyridazin-3-yl)isoquinolin-1(2H)-one;
(S)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)isoquinolin-1(2H)-one;
(R)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-(trifluoromethyl)phenyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(5-methylpyrimidin-4-yl)isoquinolin-1(2H)-one;
2-(2-(Difluoromethyl)phenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(3-Chloro-2-methoxypyridin-4-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-Cyclohexyl-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(3-Chloro-6-methylpyridin-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-Cyclopentyl-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(3-Chloro-4-methoxypyridin-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1R,2S)-2-methylcyclohexyl)isoquinolin-1(2H)-one;
2-(1,3-Dimethoxypropan-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxy-5-methylpyridin-4-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1S,2R)-2-methylcyclohexyl)isoquinolin-1(2H)-one;
2-(Cyclopropylmethyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(1-methoxybutan-2-yl)isoquinolin-1(2H)-one;
2-(2-Chlorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(3-methylpyridin-2-yl)isoquinolin-1(2H)-one;
Racemic 6-(4-Ethyl-3-(hydroxymethyl)-5-oxo-4,5-dihydro-1H-1,2,4-triazol-1-yl)-7-fluoro-4-isopropyl-2-((cis)-3-methoxycyclopentyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1R*,2R*)-2-methylcyclohexyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1S*,2S*)-2-methylcyclohexyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(pentan-3-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1R*,2R*)-2-methylcyclopentyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1S*,2S*)-2-methylcyclopentyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1R*,2S*)-2-methylcyclopentyl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-((1S*,2R*)-2-methylcyclopentyl)isoquinolin-1(2H)-one;
Racemic 6-(4-Ethyl-3-(hydroxymethyl)-5-oxo-4,5-dihydro-1H-1,2,4-triazol-1-yl)-7-fluoro-4-isopropyl-2-((cis)-3-methoxycyclohexyl)isoquinolin-1(2H)-one;
2-(Bicyclo[2.2.1]heptan-1-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methoxy-3-methylpyridin-4-yl)isoquinolin-1(2H)-one;
2-(4-Ethyl-3-(hydroxymethyl)-5-oxo-4,5-dihydro-1H-1,2,4-triazol-1-yl)-3-fluoro-8-isopropyl-6-(2-methoxyphenyl)-1,6-naphthyridin-5(6H)-one;
6-(4-Ethyl-3-(hydroxymethyl)-5-oxo-4,5-dihydro-1H-1,2,4-triazol-1-yl)-7-fluoro-4-isopropyl-2-(3-methylisothiazol-4-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(5-methylisothiazol-4-yl)isoquinolin-1(2H)-one;
2-(4-Ethyl-3-(hydroxymethyl)-5-oxo-4,5-dihydro-1H-1,2,4-triazol-1-yl)-3-fluoro-8-isopropyl-6-(2-(trifluoromethyl)phenyl)-1,6-naphthyridin-5(6H)-one;
2-(3,6-Dimethylpyridin-2-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(2,5-Dimethylpyridin-4-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(4-methylpyridin-3-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(3-methylpyridin-4-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-methylpyridin-3-yl)isoquinolin-1(2H)-one;
6-(4-Ethyl-3-(hydroxymethyl)-5-oxo-4,5-dihydro-1H-1,2,4-triazol-1-yl)-7-fluoro-2-(2-hydroxy-5-methylpyridin-4-yl)-4-isopropylisoquinolin-1(2H)-one;
6-(2-(Difluoromethyl)phenyl)-2-(4-ethyl-3-(hydroxymethyl)-5-oxo-4,5-dihydro-1H-1,2,4-triazol-1-yl)-3-fluoro-8-isopropyl-1,6-naphthyridin-5(6H)-one;
6-(4-Ethyl-3-(hydroxymethyl)-5-oxo-4,5-dihydro-1H-1,2,4-triazol-1-yl)-7-fluoro-2-(2-hydroxy-3-methylpyridin-4-yl)-4-isopropylisoquinolin-1(2H)-one; and
2-(4-Ethyl-3-(hydroxymethyl)-5-oxo-4,5-dihydro-1H-1,2,4-triazol-1-yl)-3-fluoro-8-isopropyl-6-(o-D₃-tolyl)-1,6-naphthyridin-5(6H)-one;
   or a pharmaceutically acceptable salt, isotope, N-oxide, solvate, or stereoisomer thereof; or a compound selected from
2-(2-Chloro-6-fluorophenyl)-6-(4-ethyl-3-(hydroxymethyl)-5-oxo-4,5-dihydro-1H-1,2,4-triazol-1-yl)isoquinolin-1(2H)-one;
6-(4-Ethyl-3-(hydroxymethyl)-5-oxo-4,5-dihydro-1H-1,2,4-triazol-1-yl)-2-(2-fluoro-4-nitrophenyl)-4-iodoisoquinolin-1(2H)-one;
2-(2-Chloro-6-fluorophenyl)-7-(4-ethyl-3-(hydroxymethyl)-5-oxo-4,5-dihydro-1H-1,2,4-triazol-1-yl)-6-fluoro-4-(prop-1-en-2-yl)phthalazin-1(2H)-one;
2-(2-Chloro-6-fluorophenyl)-7-(4-ethyl-3-(hydroxymethyl)-5-oxo-4,5-dihydro-1H-1,2,4-triazol-1-yl)-6-methoxy-4-(prop-1-en-2-yl)phthalazin-1(2H)-one;
   or a pharmaceutically acceptable salt, N-oxide, solvate, or stereoisomer thereof.

In an embodiment of the invention, the DHODH inhibitor is a compound of Formula (Z) selected from the group consisting of:
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one;
2-(2-Chloro-4-methylpyridin-3-yl)-6-(4-ethyl-3-(hydroxymethyl)-5-oxo-4,5-dihydro-1H-1,2,4-triazol-1-yl)-7-fluoro-4-isopropylisoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-4-(1-methylcyclopropyl)isoquinolin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-(prop-1-en-2-yl)phthalazin-1(2H)-one;
2-(4-Ethyl-3-(hydroxymethyl)-5-oxo-4,5-dihydro-1H-1,2,4-triazol-1-yl)-3-fluoro-8-isopropyl-6-(o-tolyl)-1,6-naphthyridin-5(6H)-one;
6-(4-Ethyl-3-(hydroxymethyl)-5-oxo-4,5-dihydro-1H-1,2,4-triazol-1-yl)-7-fluoro-4-isopropyl-2-(o-tolyl)phthalazin-1(2H)-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropylphthalazin-1(2H)-one;
6-(4-Ethyl-3-(hydroxymethyl)-5-oxo-4,5-dihydro-1H-1,2,4-triazol-1-yl)-7-fluoro-4-isopropyl-2-(2-(methyl-d3)phenyl)isoquinolin-1(2H)-one;
(R)-6-(4-Ethyl-3-(hydroxymethyl)-5-oxo-4,5-dihydro-1H-1,2,4-triazol-1-yl)-7-fluoro-2-(o-tolyl)-4-(1,1,1-trifluoropropan-2-yl)isoquinolin-1(2H)-one;
6-(4-Ethyl-3-(hydroxymethyl)-5-oxo-4,5-dihydro-1H-1,2,4-triazol-1-yl)-7-fluoro-4-isopropyl-2-(2-(trifluoromethyl)phenyl)isoquinolin-1(2H)-one; and
2-(2-(Difluoromethyl)phenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
   or a pharmaceutically acceptable salt, isotope, N-oxide, solvate, or stereoisomer thereof.

In an embodiment of the invention, the DHODH inhibitor is 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one or a pharmaceutically acceptable salt, solvate, stereoisomer, isotopic variant, or N-oxide thereof.

In an embodiment of the invention, the DHODH inhibitor is a compound of Formula (Z) having the Formula (Za):

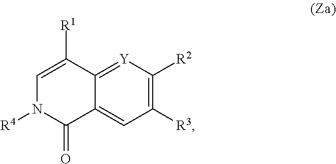

wherein
Y is CH or N;
R¹ is selected from the group consisting of: C₁₋₆alkyl; C₁₋₆alkyl substituted with OH, or OCH₃;
C₂₋₆alkenyl; C₁₋₆haloalkyl; C₁₋₆haloalkyl substituted with OH, or OCH₃; C₂₋₆haloalkenyl;
N(CH₃)₂; C₃₋₆cycloalkyl; C₃₋₆cycloalkyl substituted with C₁₋₆alkyl; and phenyl;
R² is

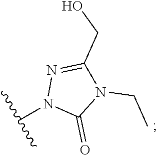 [SMILES: *n1nc(CO)n(CC)c1=O]

R³ is selected from the group consisting of: H, halo, CH₃ and OCH₃;
R⁴ is selected from the group consisting of:
C₁₋₆alkyl; C₁₋₆alkyl substituted with one or two OCH₃; C₃₋₆cycloalkyl; C₃₋₆cycloalkyl substituted with CH₃, or OCH₃; CH₂—C₃₋₆cycloalkyl; and

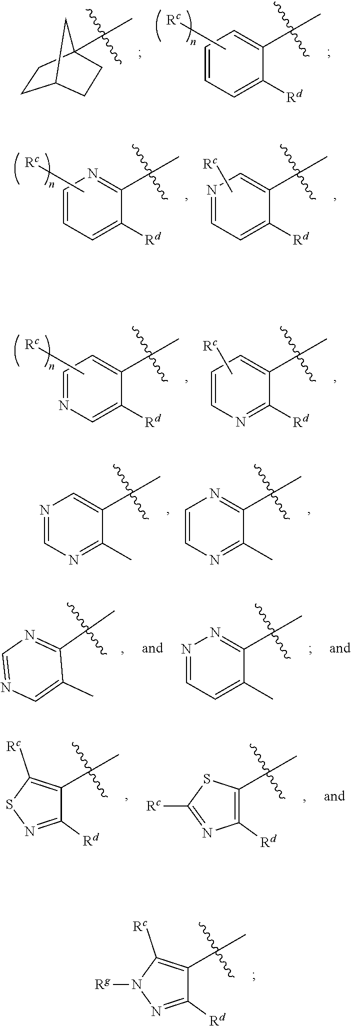

wherein
each R^{c} is independently selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; NO₂; OH; O—CH₂CH₂OH; and OC₁₋₆alkyl;
R^{d} is selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; CN; and OC₁₋₆alkyl;
R^{g} is selected from the group consisting of H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
or a pharmaceutically acceptable salt, solvate, stereoisomer, isotopic variant, or N-oxide thereof.

In an embodiment of the invention, the DHODH inhibitor is a compound of Formula (Z) having the Formula (Zb):

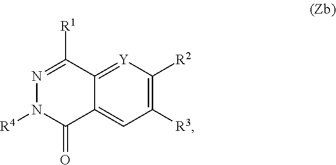

wherein
Y is CH or N:
R¹ is selected from the group consisting of: C₁₋₆alkyl, C₁₋₆haloalkyl and C₂₋₆alkenyl;
R² is

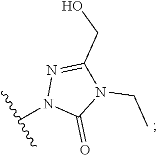 [SMILES: *n1nc(CO)n(CC)c1=O]

R³ is selected from the group consisting of: H, halo and OCH₃;
R⁴ is selected from the group consisting of:

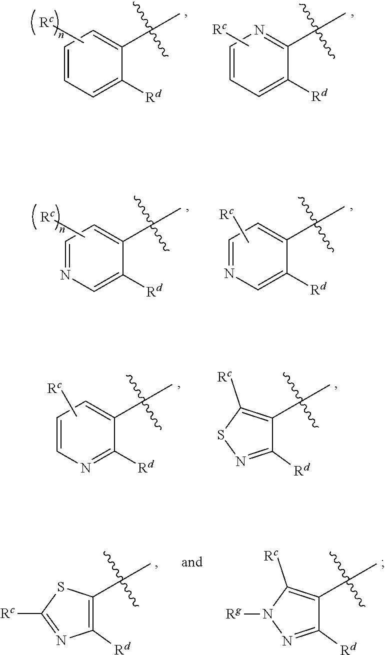

wherein
R^{c} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and NO₂;
R^{d} is selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; CN; and OC₁₋₆alkyl;
R^{g} is selected from the group consisting of H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1;
or a pharmaceutically acceptable salt, solvate, stereoisomer, isotopic variant, or N-oxide thereof.

Exemplary compounds of Formula (Z) useful in methods of the invention will now be described by reference to the illustrative synthetic schemes for their general preparation below and examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (Z). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

All abbreviations used in the general schemes and examples for Formula (Z) are as defined in Table 1A. Variables are as defined in the scope or as specifically defined in the general Schemes.

TABLE 1A
Abbreviations

| Abbreviation | Name |
|---|---|
| Å | angstrom |
| ACN or MeCN | acetonitrile |
| AcOH | glacial acetic acid |
| AcOK | Potassium acetate |
| AgBF₄ | Silver tetrafluoroborate |
| AlMe₃ | Trimethylaluminium |
| Ar | Argon |
| aq. | aqueous |
| AuCl₃ | Gold(III) chloride |
| BCl₃ | Boron trichloride |
| Bn or Bzl | benzyl |
| Boc | tert-butyloxycarbonyl |
| (Boc)₂O | Di-tert-butyl dicarbonate |
| Catacxium A Pd G2 | Chloro[(di(1-adamantyl)-N-butylphosphine)-2-(2- |
| | aminobiphenyl)]palladium(II) |
| CdCl₂ | Cadmium chloride |
| Celite ® | diatomaceous earth |
| conc. | concentrated |
| CO₂ | Carbon dioxide |
| (COCl)₂ | Oxalyl chloride |
| CuI | Copper(I) iodide |
| Cu(OAc)₂ | copper(II) acetate |
| Cy₂NMe | N,N-Dicyclohexylmethylamine |
| Cs₂CO₃ | Cesium carbonate |
| DCC | N,N′-dicyclohexyl-carbodiimide |
| DCE | dichloroethane |
| DCM | dichloromethane |
| DIPEA or DIEA | diisopropyl-ethyl amine |
| DEAD | Diethyl azodicarboxylate |
| DEA | Diethanolamine |
| DHP | 3,4-Dihydropyran |
| DMA | dimethylaniline |
| DMAP | 4-dimethylaminopyridine |
| DME | dimethoxyethane |
| DMF | N,N-dimethylformamide |
| DMP | Dess-Martin periodinane or 1,1,1-Tris(acetyloxy)-1,1-dihydro-1,2- |
| | benziodoxol-3-(1H)-one |
| DMSO | dimethylsulfoxide |
| Dppf or DPPF | 1,1′-Bis(diphenylphosphino)ferrocene |
| EDCI | 1-ethyl-3-(3-dimethylaminopropyl) carbodiimide |
| ES-API | electrospray-atmospheric pressure ionization |
| ESI | electrospray ionization |
| Et₃N•3HF | Triethylamine trihydrofluoride |
| EtOAc or EA | ethyl acetate |
| EtOH | ethanol |
| EtONa | sodium ethoxide |
| EtMgBr | Ethylmagnesium bromide |
| GCMS | gas chromatography-mass spectrometry |
| h or hr(s) | hour or hours |
| H₂ | Hydrogen gas |
| HCl | Hydrogen chloride |
| HPLC | high performance liquid chromatography |
| HPA | hypophosphorous acid |
| H₂SO₄ | Sulfuric acid |
| i-PrMgCl | Isopropylmagnesium chloride |
| IPA | Isopropylamine |
| iPrOH | Isopropyl alcohol or 2-propanol |
| isovaleraldehyde | 3-methylbutanal |
| KHMDS | Potassium bis(trimethylsilyl)amide |
| KI | Potassium iodide |
| K₂OsO₄•2H₂O | Potassium osmate (VI) dihydrate |
| K₂CO₃ | Potassium carbonate |
| K₃PO₄ | Potassium phosphate |
| LCMS or LC/MS | Liquid chromatography-mass spectrometry |
| LiHMDS | Lithium bis(trimethylsilyl)amide |
| MeOH | methanol |
| MeMgBr | methylmagnesium bromide |
| Mg(ClO₄)₂ | Magnesium perchlorate |
| MgSO₄ | Magnesium sulfate |
| MHz | megahertz |
| min | minute or minutes |
| MS | mass spectrometry |
| NaBH₄ | Sodium borohydride |
| NaHMDS | Sodium bis(trimethylsilyl)amide |
| NaOH | Sodium hydroxide |
| NaOEt | Sodium ethoxide |
| NaHCO₃ | Sodium bicarbonate |
| Na₂CO₃ | Sodium carbonate |
| NaIO₄ | Sodium periodate |
| NaNO₂ | Sodium nitrite |
| Na₂SO₄ | Sodium sulfate |
| N₂ | Nitrogen gas |
| NBS | N-Bromosuccinimide |
| NCS | N-chlorosuccinimide |
| NH₂NH₂•H₂O | Hydrazine monohydrate |
| NIS | N-iodosuccinimide |
| NH₄Cl | Ammonium chloride |
| NH₄HCO₃ | Ammonium bicarbonate |
| NH₃•H₂O or NH₄OH | Ammonium hydroxide |
| NMR | nuclear magnetic resonance |
| [Pd(allyl)Cl]₂ | Allylchloropalladium dimer or Bis(allyl)dichlorodipalladium or |
| | Bis(allyl)dichloropalladium or Bis(allylchloropalladium) or |
| | Diallyldichlorodipalladium |
| PdCl₂(PPh₃)₂ or | bis(triphenylphosphine)palladium(II) dichloride |
| Pd(PPh₃)₂Cl₂ | |
| Pd(PPh₃)₄ | tetrakis(triphenylphosphine)palladium |
| Pd(OAc)₂ | palladium (II) acetate |
| P(tBu₃)PdG₂ or | chloro[(tri-tert-butylphosphine)-2-(2-aminobiphenyl)] palladium(II) |
| tBu₃PPdG₂ | |
| PE | petroleum ether |
| PPh₃ | triphenylphosphine |
| ppm | parts per million |
| Pd(dppf)Cl₂•CH₂Cl₂ | 1,1′-bis(diphenylphosphino)ferrocene-palladium(II)dichloride |
| or Pd(dppf)Cl₂•DCM | dichloromethane complex |
| Pd/C | Palladium on carbon |
| PG | Protecting group |
| RP | reverse-phase |
| rt or RT | room temperature |
| Rₜ | retention time |
| RhCl(PPh₃)₃ or | Wilkinson's Catalyst or Chlorotris(triphenylphosphine)rhodium(I) |
| Rh(PPh₃)₃Cl | |
| Sec | second or seconds |
| SFC | supercritical fluid chromatography |
| SiO₂ | silica gel |
| SOCl₂ | Thionyl chloride |
| O₂ | Oxygen gas |
| TBAB | tetrabutylammonium bromide or tetra-n-butylammonium bromide |
| TBDPS | tert-Butyldiphenylchlorosilane |
| TBAF | tetrabutylammonium fluoride |
| TBHP | tert-butyl hydroperoxide |
| TBS | tert-Butyldimethylsilyl |
| TES | triethylsilane |
| TIPS | triisopropylsilane |
| TEA or Et₃N | triethylamine |
| TFA | trifluoroacetic acid |
| THF | tetrahydrofuran |
| TiCl₄ | Titanium tetrachloride |
| TLC | thin layer chromatography |
| TF₂NPh | N-phenylbis(trifluoromethanesufonimide) |
| triflate | trifluoromethanesulfonyl |
| Tf₂O | Triflic anhydride or Trifluoromethanesulfonic anhydride |
| TMSOiPr | Isopropoxytrimethylsilane |
| (PTSA or pTsOH) or | p-Toluenesulfonic acid |
| tosylic acid | |

### PREPARATIVE EXAMPLES

Exemplary compounds of Formula (Z) useful in methods of the invention will now be described by reference to the illustrative synthetic schemes for their general preparation below and the specific examples to follow.

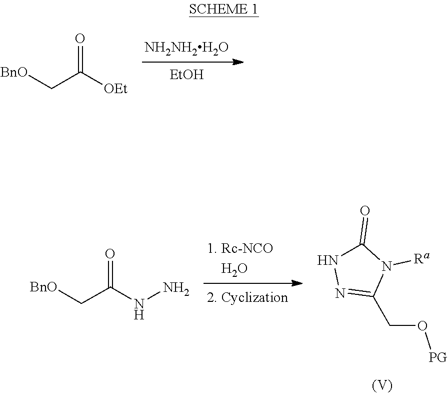

According to SCHEME 1, a 1,2,4-triazol-5(4H)-one compound of formula (II), where PG is Bn, is prepared from ethyl 2-(benzyloxy)acetate in three steps. In a first step 2-(benzyloxy)acetohydrazide is prepared by the reaction of ethyl 2-(benzyloxy)acetate with hydrazine hydrate, in a suitable solvent such as EtOH, and the like; at temperatures ranging from 70-85° C. Reaction of the hydrazide with an isocyanate of formula R^{a}—NCO, where R^{a} is C₁₋₆alkyl, in a suitable solvent such as water, and the like; provides the corresponding semicarbazide. Subsequent cyclization of the semicarbazide with a suitable base such as NaOH, in a suitable solvent such as water, provides a compound of formula (II), where PG is Bn.

A compound of formula (II), where R^{a} is C₁₋₆haloalkyl or C₃₋₆cycloalkyl; may be prepared as previously described employing a suitably substituted compound of formula R^{a}—NCO, where R^{a} is C₁₋₆haloalkyl or C₃₋₆cycloalkyl.

Protecting group exchange of a compound of formula (II), where PG is Bn to a compound of formula (II) where PG is TBDPS, is achieved in two steps employing established methodologies, such as those described in T. W. Greene and P. G. M. Wuts, “Protective Groups in Organic Synthesis,” 3 ed., John Wiley & Sons, 1999. In a first step, deprotection of benzyl group is achieved under hydrogenolytic conditions known to one skilled in the art provides the alcohol. For example, deprotection is achieved employing a palladium catalyst such Pd/C, and the like; under H₂; in a suitable solvent such as EtOH, MeOH, EtOAc, or a mixture thereof, preferably EtOH; with or without the presence HCl; for a period of 4 to 72 hrs. In a second step, protection of the corresponding alcohol as the silyl ether, is achieved with tert-butyldiphenylsilyl chloride, a suitable base such as imidazole, dimethylaminopyridine, pyridine, and the like; in a solvent such as DMF, DCM, and the like; at temperatures ranging from 0° C. to room temperature; affords a compound of formula (II) where PG is TBDPS.

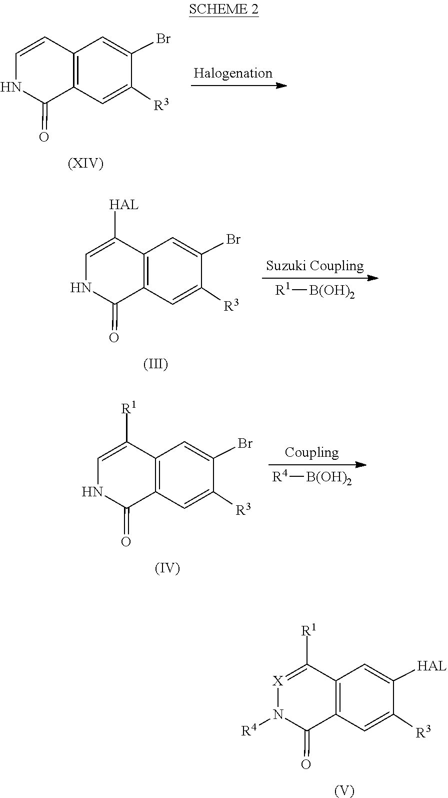

According to SCHEME 2, a compound of formula (XIV), where R³ is H is treated with a halogenating reagent such as N-iodosuccinimide (NIS), and the like; in an aprotic solvent such as acetonitrile, and the like; under heating conditions; to afford the halogenated compound of formula (III), where HAL is iodide. A compound of formula R¹—B(OH)₂; is reacted under Suzuki coupling conditions known to one skilled in the art with a compound of formula (III), to provide a compound of formula (IV). For example, a compound of formula (III), where HAL is iodide, is reacted a commercially available or synthetically accessible boronic acid (or boronic ester) such as R¹—B(OH)₂, where R¹ is an optionally substituted C₂₋₆alkenyl or aryl as defined herein with reference to Formula (Z); a palladium catalyst such as bis(triphenylphosphine)palladium(II) dichloride, tetrakis(triphenylphosphine)palladium, and the like; a suitable base such as potassium phosphate, Cs₂CO₃, and the like; in a suitable solvent such as dioxane, water, ethanol, or a mixture thereof; to provide a compound of formula compound (IV). A compound of formula (IV), where R³ is H, is reacted with a compound of formula R⁴—B(OH)₂; under copper (II) mediated Chan-Lam coupling conditions known to one skilled in the art, to provide a compound of formula (V), where HAL is bromide, X is CH and R³ is H. For example, a compound of formula (IV) is reacted with a compound of formula R⁴—B(OH)₂, where R⁴ is as defined herein with reference to Formula (Z); a catalyst such as copper(II) acetate, and the like; a base such as pyridine, NEt₃, and the like; in a suitable solvent such as DCM, ACN, dioxane, THF, and the like; to afford a compound of formula (V).

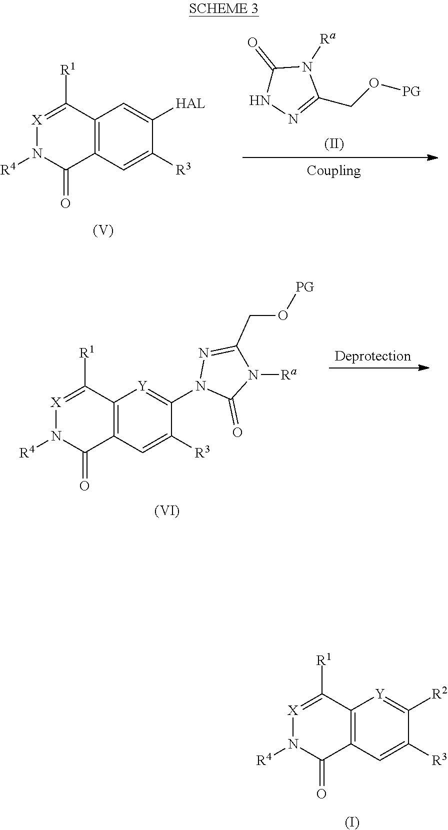

According to SCHEME 3, Ullmann-type aromatic amination reaction of compound of formula (V), where R¹ is optionally substituted C₂₋₆alkenyl, R³ is H, R⁴ is suitably substituted phenyl as described herein with reference to Formula (Z), and HAL is Br; with a commercially available or synthetically accessible nucleophilic compound of formula (II), where R^{a} is C₁₋₆alkyl; such as suitably protected triazolones, where PG is selected from: benzyl, 4-methoxy benzyl, or an alkyl or aryl silane such as TBDPS, TBS, TES, or TIPS; in the presence of catalytic CuI and a diamine such as trans-1,2-diaminocyclohexane, and a base such as K₃PO₄, K₂CO₃, Cs₂CO₃, NaHCO₃, triethylamine, and the like; in a suitable solvent such as 1,4-dioxane, DMSO, DMF, THF, ACN, and the like; provides a compound of formula (VI), where X is CH and Y is CH.

A compound of formula (VI), where PG is Bn and R¹ is C₂₋₆alkenyl, is reacted under Simmons-Smith cyclopropanation reaction conditions known to one skilled in the art to provide a compound of formula (VI) where R¹ is C₃₋₆cycloalkyl substituted with C₁₋₆alkyl. For example, a compound of formula (VI), where R¹ is

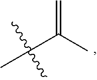 [SMILES: *C(=C)C]

is reacted with diiodomethane; diethylzinc; in a suitable solvent such as toluene, and the like; at temperatures ranging from 0° C. to room temperature; for a period of 3 to 26 h; to provide a compound of formula (VI), where R¹ is cyclopropyl substituted with CH₃.

Subsequent deprotection employing established methodologies, such as those described in T. W. Greene and P. G. M. Wuts, “Protective Groups in Organic Synthesis,” 3 ed., John Wiley & Sons, 1999), provides a compound of Formula (Z), where X and Y are CH. For example, compound of formula (VI), where R³ is H, and PG is TBDPS, is deprotected employing conditions known to one skilled in the art, preferably with TBAF in a suitable solvent such as THF, and the like. In a preferred method, PG is TBDPS, and R^{a} is C₁₋₆alkyl. Alternately, removal of a TBDPS protecting group is achieved employing triethylamine trihydrogen fluoride (Et₃N·3HF).

Removal of the Bn protecting group is achieved in the presence of hydrogen gas, in the presence of a catalyst such as Palladium on carbon (Pd/C). Removal of the protecting group Bn is also achieved employing TFA, at a temperature of about 80° C.

A compound of Formula (Z), where X is CH; Y is CH; R², R³, R⁴ is each defined as described herein with reference to Formula (Z); and R¹ is C₂₋₆alkenyl, is reduced employing hydrogenation conditions known to one skilled in the art, for example, reaction with Pd/C or Wilkinson's Catalyst [RhCl(PPh₃)₃] under H₂; in a suitable solvent such as MeOH, THF, EtOAc, and the like; provides a compound of Formula (Z) where R¹ is C₂₋₆alkyl.

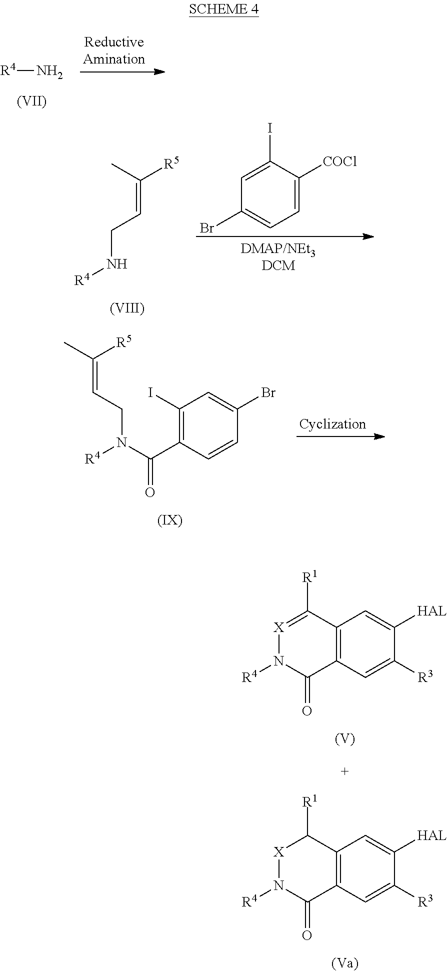

According to SCHEME 4, reductive amination of a compound of formula (VII), with a, (3-unsaturated aldehyde such as 3-methyl-2-butenal, 3-methylpent-2-enal, and the like; employing TiCl₄; and a base such as triethylamine; in an aprotic solvent such as dichloromethane (DCM), and the like; provides an enamine intermediate which is subsequently reduced employing a reducing agent such as NaBH₄, and the like; to afford a compound of formula (VIII) where R⁵ is C₁₋₄alkyl, R⁴ is as defined herein with reference to Formula (Z). A compound of formula (VIII) is coupled with commercially available or synthetically accessible 4-bromo-2-iodobenzoyl chloride employing a base such as triethylamine and 4-dimethylaminopyridine (DMAP); in an anhydrous aprotic solvent such as dichloromethane (DCM), and the like; to afford a compound of formula (IX). Treatment of a compound of formula (IX), with palladium (II) acetate, tetrabutylammonium bromide, and potassium acetate under heating Heck reaction conditions, affords the intramolecular cyclized compounds of formula (V), wherein R¹ is optionally substituted C₂₋₆alkyl, R³ is H, X is CH, and HAL is Br; and (Va) wherein R¹ is optionally substituted C₂₋₆alkenyl, R³ is H, X is CH₂, and HAL is Br.

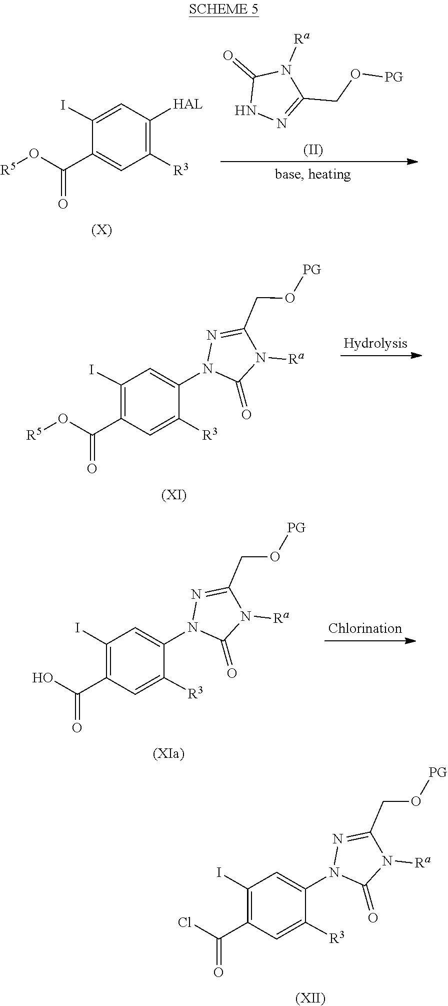

According to SCHEME 5, the reaction of a commercially available or synthetically accessible compound of formula (X), where HAL is F, R³ is F, and R⁵ is H or C₁₋₄alkyl; with a commercially available or synthetically accessible nucleophilic compound of formula (II), where R^{a} is C₁₋₆alkyl, such as suitably protected triazolones, where PG is selected from: benzyl, 4-methoxy benzyl, or an alkyl or aryl silane such as TBDPS, TBS, TES, or TIPS; in the presence of a base such as K₃PO₄, K₂CO₃, Cs₂CO₃, NaHCO₃, triethylamine, and the like; in a suitable solvent such as DMSO, DMF, THF, ACN, and the like; affords a compound of formula (XI). In a preferred method, PG is Bn, and R^{a} is C₁₋₆alkyl. The ester of formula (XI), when R⁵ is C₁₋₄alkyl, is hydrolyzed to its corresponding acid, under acidic or basic conditions. For example, the treatment of tert-butyl ester (R⁵ is tert-Bu) with TFA; or alternately, hydrolysis with a base like NaOH, in an aqueous solvent, affords a compound of formula (XIa), where R⁵ is H. A compound of formula (XIa) is chlorinated, employing conditions known to one skilled in the art, to provide the acyl chloride of formula (XII). For example, a compound of formula (XIa) is heated in SOCl₂; or treated with oxalyl chloride in DCM.

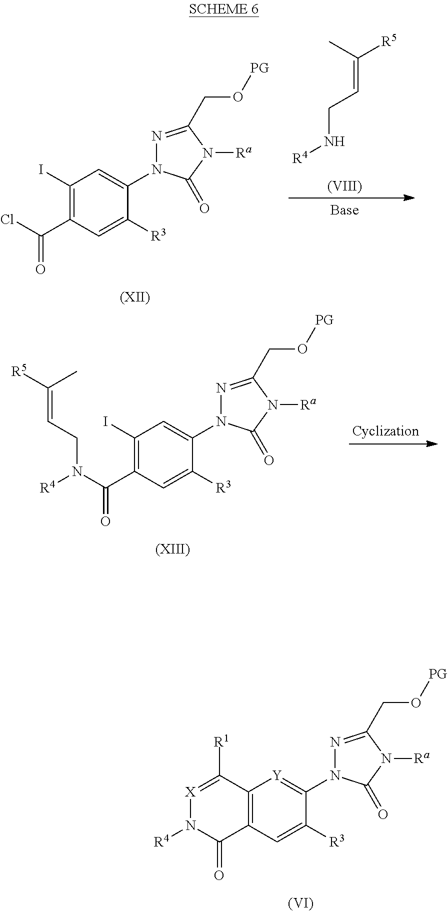

According to SCHEME 6, a compound of formula (XII), where R³ is H or F, PG is Bn, and R^{a} is C₁₋₆alkyl; is reacted with a compound of formula (VIII), where R⁵ is C₁₋₄alkyl, employing a base such as a mixture of triethylamine (TEA) and 4-dimethylaminopyridine (DMAP); in an anhydrous aprotic solvent such as dichloromethane (DCM), and the like; to afford a compound of formula (XIII). A compound of formula (VI), where X is CH and Y is CH, is obtained by treatment of a compound of formula (XIII), where R¹ is optionally substituted C₁₋₆alkyl as described herein with reference to Formula (Z); with palladium (II) acetate, tetrabutylammonium bromide, and potassium acetate under heating Heck reaction conditions, that affords a mixture of intramolecular cyclized compounds, which is then separated to isolate an intermediate compound where R¹ is C₂₋₆alkyl, and R³ is H or F.

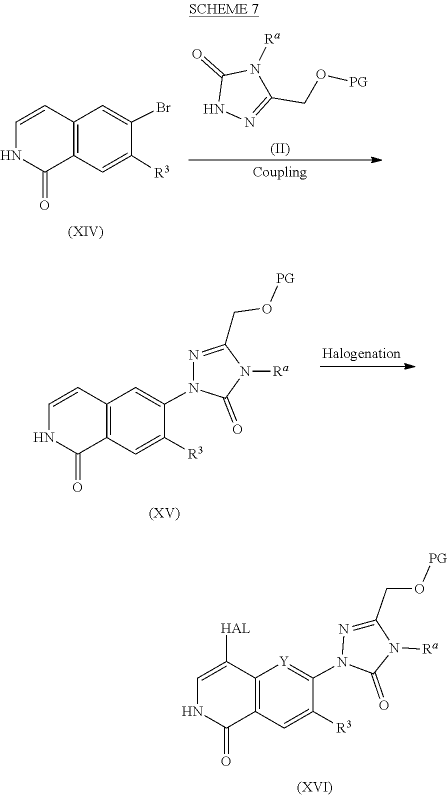

According to SCHEME 7, Ullmann-type aromatic amination reaction of a compound of formula (XIV), where R³ is H or F, with a compound of formula (II); such as suitably protected triazolones, where PG is selected from: benzyl, 4-methoxy benzyl, or an alkyl or aryl silane such as TBDPS, TBS, TES, or TIPS; according to methods previously described; affords a compound of formula (XV). In a preferred method, PG is Bn, and R^{a} is C₁₋₆alkyl. A compound of formula (XV) is treated with a halogenating reagent such as N-iodosuccinimide (NIS), and the like; in an aprotic solvent such as acetonitrile, and the like; under heating conditions; affords a halogenated compound of formula (XVI), where Y is CH and HAL is iodide.

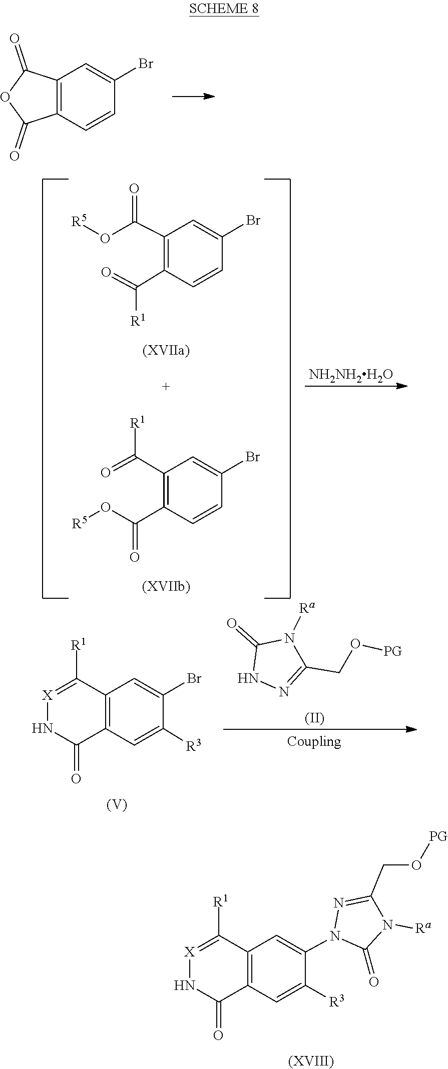

According to SCHEME 8, compounds of formula (XVIIa) and (XVIIb) are prepared from 5-bromoisobenzofuran-1,3-dione in two steps. 5-Bromoisobenzofuran-1,3-dione is reacted with a commercially available or synthetically accessible suitably substituted alkyl Grignard reagent such as i-PrMgCl, EtMgBr, and the like; in the presence of CdCl₂; in aprotic solvent like THF, and the like; followed by subsequent treatment with an alkylating agent of formula R⁵—I, where R⁵ is C₁₋₄alkyl (such as iodomethane or iodoethane); in the presence of base like K₂CO₃, Cs₂CO₃, and the like; in a aprotic solvent such as DMF, DMSO, and the like; affords a mixture of regio-isomeric esters of formula (XVIIa) and (XVIIb), where R¹ is an optionally substituted C₁₋₆alkyl. In a similar fashion, aryl Grignard reagents may be used to provide compounds of formula (XVIIa) and (VXIIb), where R¹ is a suitably substituted phenyl. The regio-isomers of formula (XVIIa) and (XVIIb) are not separated but are used directly and converted into the corresponding phthalazinone (mixture). For example, a mixture of formula (XVIIa) and formula (XVIIb) are treated with excess hydrazine; in a suitable solvent such as ethanol or methanol; at temperatures ranging from room temperature to 90° C.; for a period of 6 to 20 hours. The desired phthalazinone compound of formula (V) can be readily separated from the other regio-isomer by precipitation, crystallization, or purified by flash chromatography. Ullmann-type aromatic amination reaction of a compound of formula (V), with a suitably protected triazolone of formula (II), where R^{a} is C₁₋₆alkyl, and PG is selected from: benzyl, 4-methoxy benzyl, or an alkyl or aryl silane such as TBDPS, TBS, TES, or TIPS; in the presence of catalytic CuI and a diamine such as trans-1,2-diaminocyclohexane, and a base such as K₃PO₄, K₂CO₃, Cs₂CO₃, NaHCO₃, triethylamine, and the like; in a suitable solvent such as 1,4-dioxane, DMSO, DMF, THF, ACN, and the like; affords a compound of formula (XVIII), where X is N. A compound of formula (XVIII), where R¹ is C₂₋₆alkenyl, is reacted under Simmons-Smith cyclopropanation reaction conditions known to one skilled in the art, to provide a compound of formula (XVIII) where R¹ is C₃₋₆cycloalkyl substituted with C₁₋₆alkyl. For example, a compound of formula (XVIII), where R¹ is C₂₋₆alkenyl, is reacted with diiodomethane; diethylzinc; in a suitable solvent such as toluene, and the like; at temperatures ranging from 0° C. to room temperature; for a period of 24 to 26 h; to provide a compound of formula (XVIII), where R¹ is cyclopropyl substituted with CH₃.

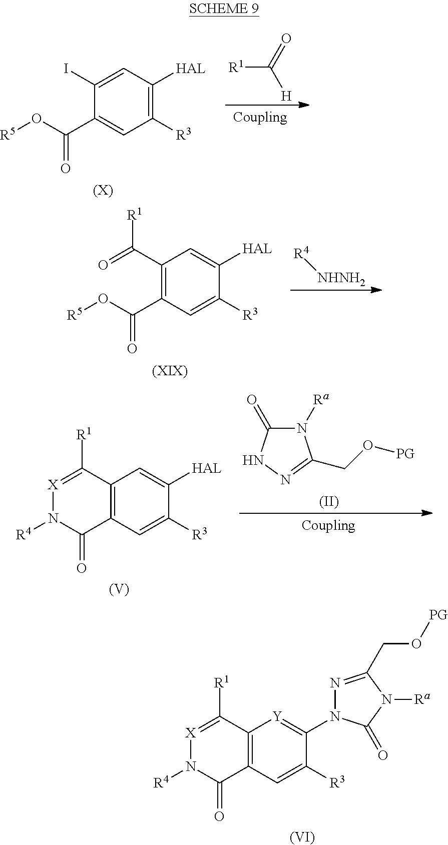

According to SCHEME 9, a compound of formula (X), where HAL is Br, R³ is H, and R⁵ is CH₃, is coupled in a palladium catalyzed carbonylation reaction with a commercially available or synthetically accessible aldehyde of formula R¹—CHO, where R¹ is C₁₋₆alkyl; to afford the corresponding ketone compound of formula (XIX), (similar transformation has been reported by Suchand et al, J. Org. Chem. 2016, 81, 6409-6423). For example, reaction of methyl 4-bromo-2-iodobenzoate with isobutylaldehye; in the presence of a palladium catalyst such as Pd(OAc)₂; Ag₂O; and an oxidizing agent such as aqueous solution of tert-butyl hydroperoxide (TBHP); at a temperature of about 120° C.; for a period of 10-14 h; provided methyl 4-bromo-2-isobutyrylbenzoate. A ketone compound of formula (XIX) is reacted with hydrazine R⁴—NHNH₂, where R⁴ is suitably substituted aryl such as 2-chloro-6-fluorophenylhydrazine; to afford a compound of formula (V), where X is N. Ullmann-type aromatic amination reaction of a compound of formula (V) with a suitably protected triazolone (II) as previously described, affords a compound of formula (VI), where Y is CH, and R¹ is selected from C₁₋₆alkyl. A compound of formula (VI), where Y is CH, and R¹ is phenyl and X is N, may be prepared in a similar fashion, employing methods previously describe by coupling methyl 4-bromo-2-iodobenzoate with a commercially available or synthetically accessible aldehyde of formula R¹—CHO, where R¹ is phenyl.

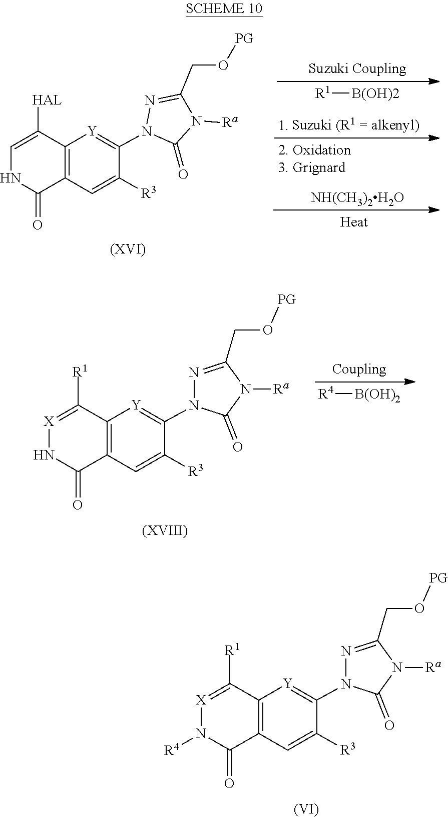

According to SCHEME 10, a compound of formula R¹—B(OH)₂; is reacted under Suzuki coupling conditions known to one skilled in the art, with a compound of formula (XVI), to provide a compound of formula (XVIII), where X is CH. For example, a compound of formula (XVI), where Y is CH and HAL is iodide, is reacted a commercially available or synthetically accessible boronic acid (or boronic ester) such as R¹—B(OH)₂, where R¹ is an optionally substituted C₂₋₆alkenyl, C₃₋₆cycloalkyl or aryl as defined herein with reference to Formula (Z); a palladium catalyst such as bis(triphenylphosphine)palladium(II) dichloride, and the like; a suitable base such a potassium phosphate, Cs₂CO₃, and the like; in a suitable solvent such as dioxane, water, ethanol, or a mixture thereof; to provide a compound of formula compound (XVIII), where X is CH. It has been noticed that during the coupling reaction as described above, loss of the iodide during the reaction conditions afforded a compound of formula compound (XVIII), where X is CH, and R¹ is H. A compound of formula (XVIII), where X is CH or N, is reacted with a compound of formula R⁴—B(OH)₂; under copper (II) mediated Chan-Lam coupling conditions known to one skilled in the art, or as previously described, to provide a compound of formula (VI), where X is CH or N, R¹ is optionally substituted C₂₋₆alkenyl, R³ is H or F, and R⁴ is a suitably substituted phenyl as described herein with reference to Formula (Z).

A compound of formula (XVIII), where R¹ is N(CH₃)₂ is prepared from a compound of formula (XVI), where HAL is Br and PG is Bn. Reaction of a compound of formula (XVI) with an amine bs such as NH(CH₃)₂ in water; at a temperature of about 110° C.; for a period of 96 hours h; affords a compound of formula (XVIII) where R¹ is N(CH₃)₂, and R^{a} is C₁₋₆alkyl. A compound of Formula (Z), where R¹ is N(CH₃)₂ is prepared according to methods described above.

A compound of formula (XVIII), where R¹ is C₁₋₆alkyl substituted with OH, is prepared from a compound of formula (XVIII), where R¹ is C₂₋₆alkenyl, and PG is Bn in two steps. In a first step, reaction of a compound of formula (XVIII), where R¹ is

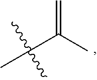 [SMILES: *C(=C)C]

under oxidizing conditions such as NaIO₄, and K₂OsO₄·2H₂O or OsO₄; in a suitable solvent such as THF/H₂O; at temperatures ranging from 0° C. to room temperature; for a period of 48 to 72 hours; affords a ketone intermediate compound. In a second step, reaction of the ketone intermediate compound with a Grignard reagent such as methylmagnesium bromide; in a suitable solvent such as diethyl ether; at temperatures ranging from 0° C. to room temperature; for a period of 3 to 30 hours; affords a compound of formula (XVIII), where R¹ is C₁₋₆alkyl substituted with OH.

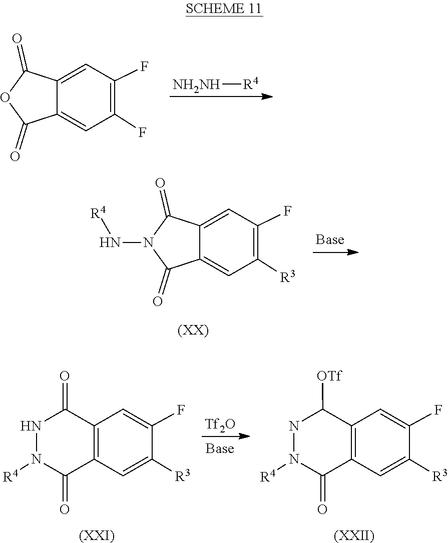

According to SCHEME 11, 4,5-difluorophthalic anhydride is reacted with a hydrazine compound of formula R⁴—NHNH₂, where R⁴ is a suitably substituted phenyl or heteroaryl such as (2-chloro-6-fluorophenyl)hydrazine hydrochloride; in acetic acid; at a temperature of about 125° C.; for a period of about 1.5 h to afford a compound of formula (XX), where R³ is F. Rearrangement of a compound of formula (XX) affords a ring expansion compound of formula (XXI), under basic conditions such as sodium ethoxide or sodium methoxide; in a protic solvent such as ethanol, methanol, and the like; at room temperature; for a period of about 1.5 h. Derivation of a compound of formula (XXI), with a sulfonate-based leaving group such as trifluoromethanesulfonyl (triflate), is achieved by is by reaction with a triflating agent such as trifluoromethanesulfonic anhydride (Tf₂O), a base such as triethylamine (TEA), pyridine, and the like, in a suitable solvent such as DCM and the like, to provide a compound of formula (XXII). Milder triflating agents such as N-phenylbis(trifluoromethanesufonimide) (TF₂NPh), a base such as TEA, DIEA, and the like, in a suitable solvent such as DCM, and the like; may be used.

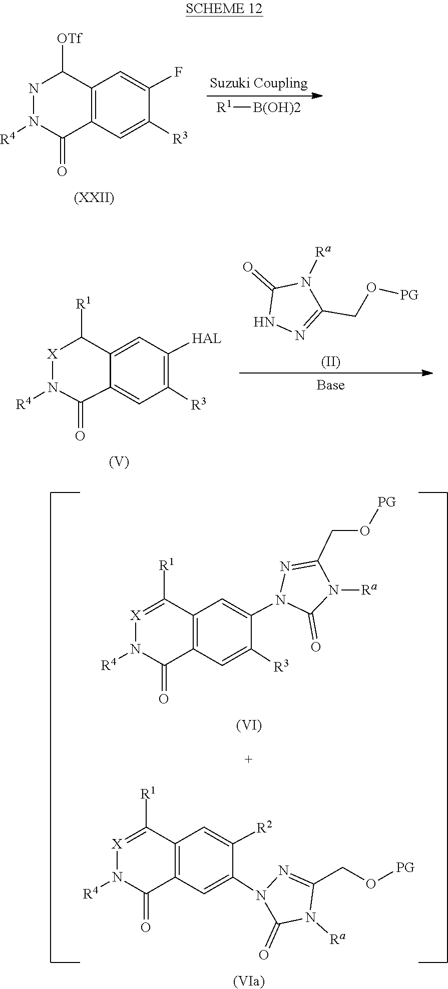

According to SCHEME 12, a compound of formula R¹—B(OH)₂; is reacted under Suzuki coupling conditions previously described, with a compound of formula (XXII), to provide a compound of formula (V), where X is N. For example, a compound of formula (XXII), is reacted a commercially available or synthetically accessible boronic acid (or boronic ester) such as R¹—B(OH)₂, where R¹ is C₂₋₆alkenyl or C₂₋₆haloalkenyl as defined herein with reference to Formula (Z); a palladium catalyst such as 1,1′-bis(diphenylphosphino)ferrocene-palladium(II)dichloride or bis(triphenylphosphine)palladium(II) dichloride, and the like; a suitable base such a potassium phosphate, Cs₂CO₃, K₂CO₃, and the like; in a suitable solvent such as dioxane, water, ethanol, or a mixture thereof; to provide a compound of formula compound (V). A compound of formula (V), where R¹ is C₂₋₆alkyl or C₂₋₆haloalkyl, is readily prepared by selective hydrogenation of a compound of formula (V), where R¹ is C₂₋₆alkenyl or C₂₋₆haloalkenyl. For example, reaction of a compound of formula (V), where R¹ is

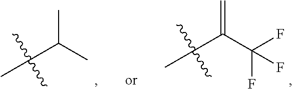

under hydrogenation conditions employing a catalyst such as Pd/C and the like, in a suitable solvent such as EtOAc, and the like; under an atmosphere of hydrogen gas (20-45 psi) at room temperature; for a period of 4 to 24 hours; affords a compound of formula (V), where R¹ is

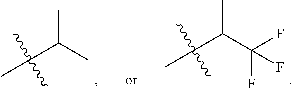

The reaction of a compound of formula (V), with a suitably protected triazolone of formula (II), employing conditions previously described, affords a mixture of compounds of formula (VI) and (VIa) which can be separated before or after deprotection of the protecting group.

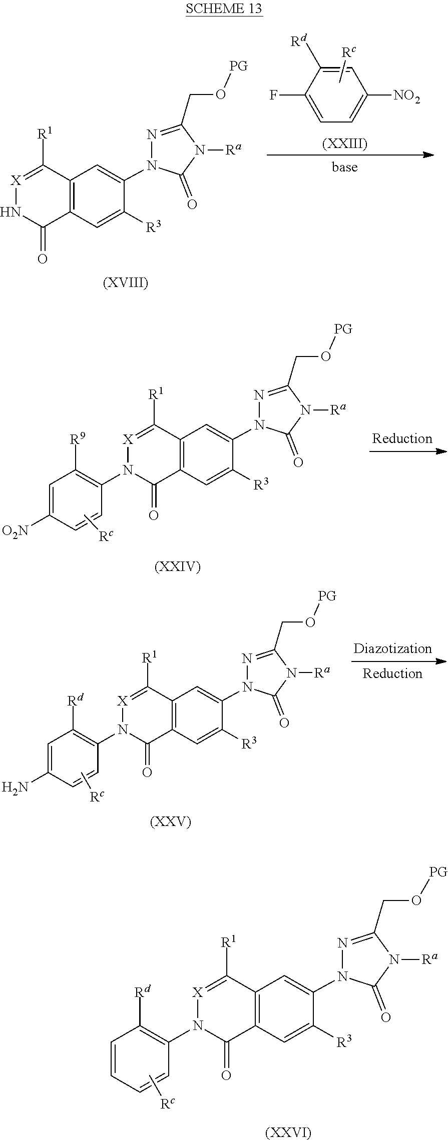

According to SCHEME 13, N-arylation of a compound of formula (XVIII) is achieved by reaction of suitably substituted commercially available or synthetically accessible fluoro compound of formula (XXIII), where R^{c} and R^{d} are as defined herein with reference to Formula (Z). A compound of formula (XVIII), where R¹ is H, C₂₋₆alkenyl, C₂₋₆haloalkenyl, C₃₋₆cycloalkyl, C₃₋₆cycloalkyl substituted with C₁₋₆alkyl, and X is CH or N, is reacted under nucleophilic displacement reaction conditions, with a commercially available or synthetically accessible fluoro compound of formula (XXIII); in the presence of a base like K₂CO₃, Cs₂CO₃, and the like; in aprotic solvent such as DMF, DMSO, and the like; at temperatures ranging from 65 to 100° C.; to afford a compound of formula (XXIV).

Reduction of compound of formula (XXIV) is achieved employing zinc or iron and NH₄Cl; in a mixed solvent of methanol and water; to provide an amino compound of formula (XXV).

Diazotization of a compound in formula (XXV) with NaNO₂; in an acidic aqueous solution or other nitrite reagents; in an organic solvent, such as EtOH, and the like; at a temperature of 0° C.; and subsequent the reduction of diazo group with zinc at temperatures ranging from 0 to 85° C.; or by treatment with H₃PO₂; affords a compound of formula (XXVI), where R^{c} and R^{d} are as defined as described herein with reference to Formula (Z).

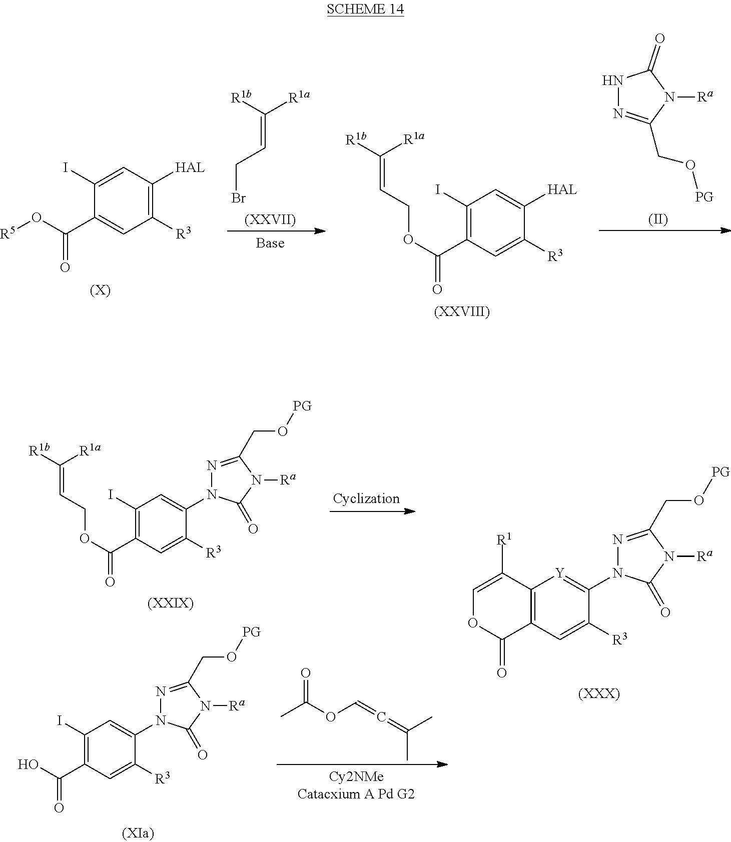

According to SCHEME 14, a compound of formula (X), where HAL is F, R⁵ is H and R³ is F, is reacted with a commercially available or synthetically accessible compound of formula (XXVII), where R^{1a} and R^{1b} are each independently H or C₁₋₄alkyl, such as 1-bromo-3-methyl-2-butene; in the presence of a base such as K₂CO₃, Cs₂CO₃, and the like; in a suitable solvent such as DMSO, DMF, THF, ACN, and the like; to afford an ester compound of formula (XXVIII), where R³ is F, and HAL is F. A compound of formula (XXVIII), where R^{1a} and R^{1b} are each independently selected from C₁₋₄haloalkyl or C₃₋₆cycloalkyl may be made in a similar fashion. The reaction of an ester of formula (XXVIII) with a suitably protected triazolone compound of formula (II); in the presence of a base such as K₃PO₄, K₂CO₃, Cs₂CO₃, NaHCO₃, triethylamine, and the like; in a suitable solvent such as 1,4-dioxane, DMSO, DMF, THF, ACN, and the like; affords a compound of formula (XXIX). In a preferred method, PG is Bn, and R^{a} is C₁₋₆alkyl. A compound of formula (XXIX), where R³ is H or F, undergoes intramolecular cyclization under Heck reaction conditions, such as employing at catalyst such as chloro[(tri-tert-butylphosphine)-2-(2-aminobiphenyl)]palladium(II) (P(tBu₃)PdG₂), N-cyclohexyl-N-methyl-cyclohexanamine, in a suitable solvent such as toluene, and the like; at a temperature of about 15 to 80° C.; for a period of about 18 to 36 hours; to provide an isocoumarin compound of formula (XXX), where Y is CH and R¹ is isopropyl, R³ is H or F, R^{a} and PG are defined as described above.

An isocoumarin compound of formula (XXX), where R¹ is

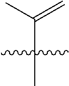 [SMILES: *C(=C)C]

is prepared from a compound of formula (XIa) and methylbuta-1,2-dien-1-yl acetate. Methylbuta-1,2-dien-1-yl acetate is commercially available or prepared in two steps from 2-methyl-3-butyn-2-ol. Acetic anhydride is reacted with 2-methyl-3-butyn-2-ol, in the presence of a catalyst such as Mg(ClO₄)₂; in a suitable solvent such as DCM, and the like; to afford 2-methylbut-3-yn-2-yl acetate. 2-Methylbut-3-yn-2-yl acetate is reacted with a catalytic amount of a Lewis acid such as AgBF₄, AgClO₄, PtCl₂, and the like; to provide 3-methylbuta-1,2-dien-1-yl acetate. 3-Methylbuta-1,2-dien-1-yl acetate is coupled with a compound of formula (XIa), where R⁵ is H, employing intermolecular cyclization under Heck reaction conditions as previously described, such as employing at catalyst such as Catacxium A Pd G2, and Cy₂NMe palladium (II) acetate, phase transfer reagent like tetrabutylammonium bromide, and a base like potassium acetate, in a suitable solvent such as DMF, and the like; at a temperature of 70 to 90° C.; for a period of 10 to 16 hours; to provide the isocoumarin compound of formula (XXX), where Y is CH and R¹ is

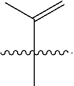 [SMILES: *C(=C)C]

A compound of formula (XXX), where Y is CH and R¹ is

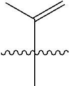 [SMILES: *C(=C)C]

is selectively reduced under hydrogenation conditions employing at catalyst such as Wilkinson's Catalyst [RhCl(PPh₃)₃] and the like, in a suitable solvent such as THF, and the like; at room temperature, provide an isocoumarin compound of formula (XXX), where R¹ is isopropyl.

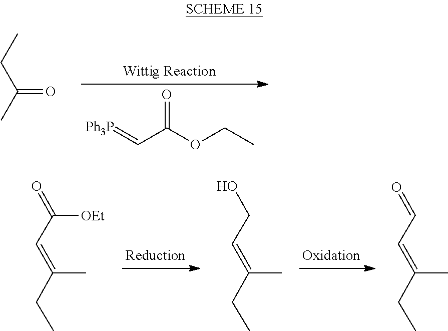

According to SCHEME 15, 2-butanone is converted to ethyl 3-methylpent-2-enoate employing Wittig reaction conditions known to one skilled in the art. For example, 2-butanone is reacted with a triphenyl phosphonium ylide such as (carbethoxymethylene) triphenylphosphorane, with or without an additive such as benzoic acid, LiCl, and sodium dodecyl sulfate (SDS), and the like, in a suitable solvent such as toluene, at temperatures ranging from rt to the reflux temperature of the solvent, for a period of 12-24 h. Ethyl 3-methylpent-2-enoate is reduced to 3-methylpent-2-en-1-ol employing a suitable reducing agent such as DIBAL-H, in a suitable solvent such as toluene, and the like, at temperatures ranging from −78° C. to room temperature. 3-Methylpent-2-en-1-ol is oxidized to 3-methylpent-2-enal employing oxidation conditions known to one skilled in the art, for example, DMP (Dess-Martin periodinane), SO₃-pyridine, Swern conditions [(COCl)₂, DMSO, Et₃N], PCC, and the like, in a solvent such as EtOAc, DMSO, DCM, and the like, at temperatures ranging from about −78° C. to room temperature (about 23° C.). In a preferred method, 3-methylpent-2-en-1-ol is oxidized to 3-methylpent-2-enal with Dess-Martin periodinane, in DCM, at 25° C. for a period of 1-4 h.

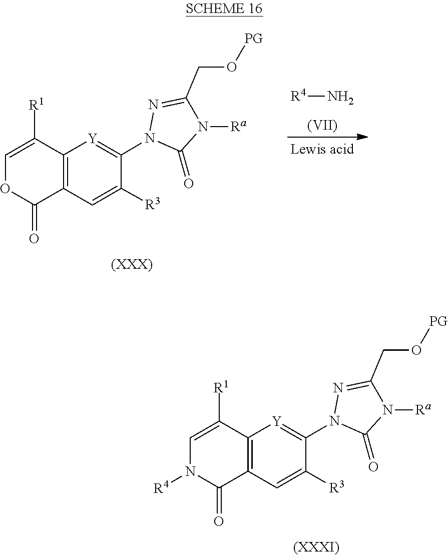

According to SCHEME 16, an isocoumarin of compound of formula (XXX), where Y is CH, is reacted with a commercially available or synthetically accessible amine compound of formula R⁴—NH₂, where R⁴ is as defined herein with reference to Formula (Z); a Lewis acid such as like AlMe₃, AlCl₃, and the like; in a suitable aprotic solvent such as DCM, toluene, and the like; to provide a compound of formula (XXXI), where Y is CH, and R¹, R³, R⁴ and R^{a} are defined as described herein with reference to Formula (Z).

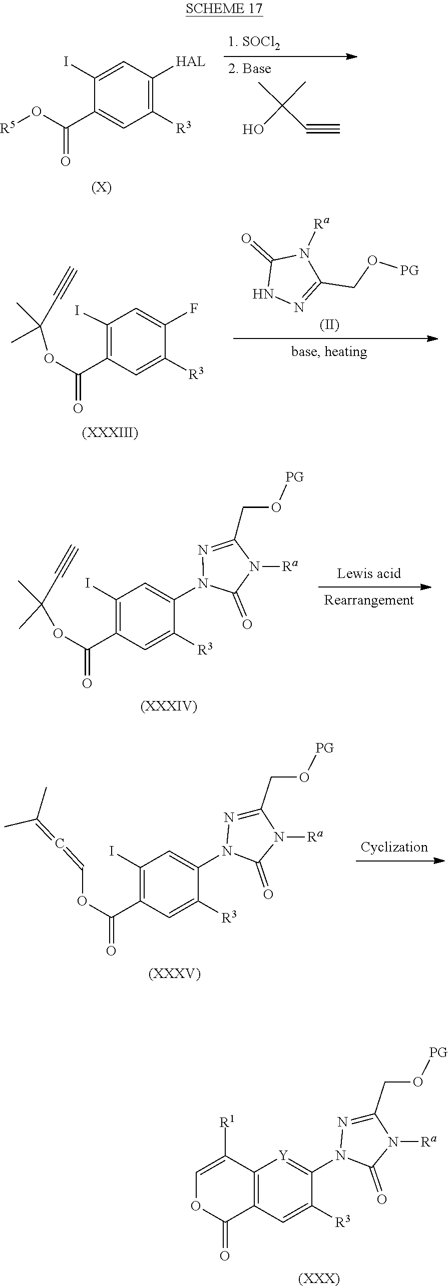

An isocoumarin compound of formula (XXX) may be prepared according to SCHEME 17. 4,5-Difluoro-2-iodobenzoyl chloride is prepared from a compound of formula (X), where HAL is F, R⁵ is H and R³ is F, employing conditions known to one skilled in the art such as oxalyl chloride or thionyl chloride, in the presence of a catalytic amount of DMF, in a suitable solvent such as an aprotic non-polar solvent such as dichloromethane (DCM), tetrahydrofuran (THF), acetonitrile (ACN), toluene, and the like, at a temperatures ranging from 0° C. to room temperature to form 4,5-difluoro-2-iodobenzoyl chloride. 4,5-Difluoro-2-iodobenzoyl chloride may be reacted with commercially available or synthetically accessible 2-methylbut-3-yn-2-ol, in the presence of a base such as triethylamine and DMAP; in a suitable solvent such as DCM, and the like; to afford an ester compound of formula (XXXIII). A compound of formula (XXXIII) may be reacted with a compound of formula (II), employing methods as previously described to afford a compound of formula (XXXIV). Treatment of a compound of formula (XXXIV) with a catalytic amount of a Lewis acid such as AgClO₄, PtCl₂, and the like; may afford the rearranged compound of formula (XXXV). A compound of formula (XXXV), where R³ is H or F, may undergo an intramolecular cyclization under Heck reaction conditions, such as employing a catalyst such as palladium (II) acetate, a phase transfer reagent like tetrabutylammonium bromide, and a base like potassium acetate, in a suitable solvent such as DMF, and the like; at a temperature of 70 to 90° C.; for a period of 1 to 3 hours; to provide an isocoumarin compound of formula (XXX), where Y is CH.

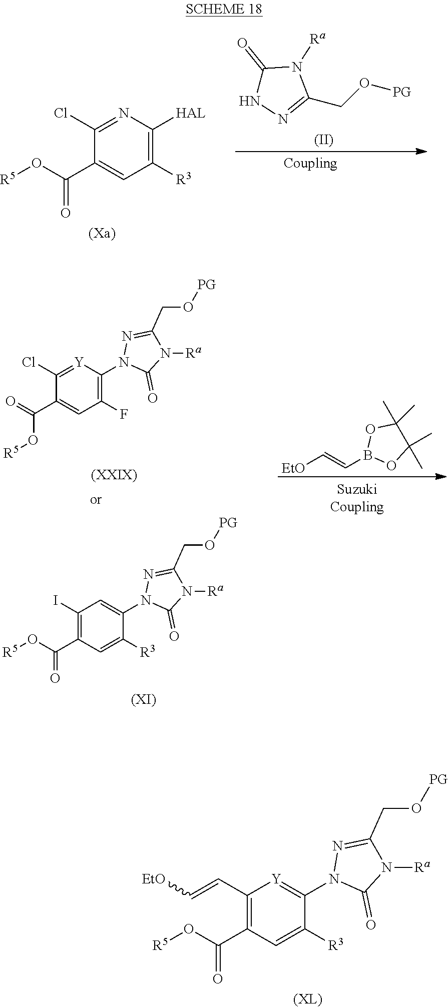

According to SCHEME 18, a compound of formula (Xa), where HAL is Cl, R⁵ is CH(CH₃)₂, and R³ is F, is commercially available or synthetically accessible according to methods as described in Chen, et al, US Patent Publication No. US2016-0176869. Reaction of a compound of formula (Xa) with a commercially available or synthetically accessible nucleophilic compound of formula (II), where PG is benzyl, and R^{c} is C₁₋₆alkyl; in the presence of a base such as K₂CO₃, Cs₂CO₃, NaHCO₃, triethylamine, and the like; in a suitable solvent such as dimethylsulfoxide (DMSO), DMF, THF, ACN, and the like; affords a compound of formula (XXXIX), where Y is N. A compound of formula (XXXIX) or formula (XI), where R³ is F, and R⁵ is C₁₋₄alkyl; is reacted a commercially available 1-ethoxyethene-2-boronic acid pinacol ester; a palladium catalyst such as bis(triphenylphosphine)palladium(II) dichloride, 1,1′-bis(diphenylphosphino)ferrocene-palladium(II)dichloride and the like; a suitable base such as Cs₂CO₃, and the like; in a suitable solvent such as dioxane, water, ethanol, or a mixture thereof; employing conventional or microwave heating; to provide a compound of formula (XL), where Y is N or CH.

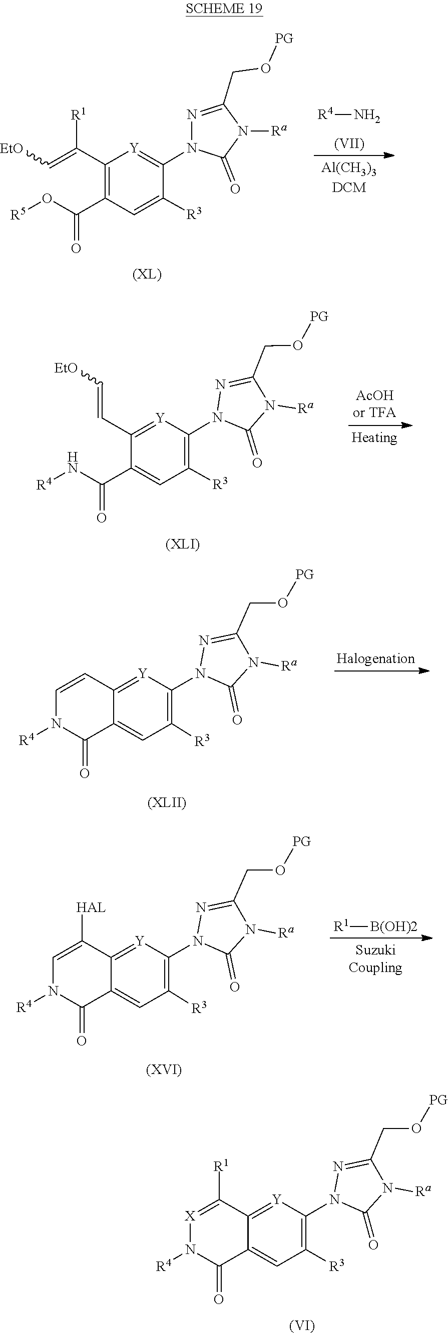

According to SCHEME 19, a compound of formula R⁴—NH₂, where R⁴ is as defined herein with reference to Formula (Z); is reacted with trimethyl aluminum; in a suitable solvent such as dichloromethane, toluene, or a mixture thereof; the resulting solution is combined with a compound of formula (XL), where Y is CH or N; to provide a compound of formula (XLI). A compound of formula (XLI), where Y is CH or N, is treated with acetic acid or trifluoroacetic acid under heating conditions between 50° C. to 90° C., to provide a compound of formula (XLII). A compound of formula (XLII) is halogenated employing N-bromosuccinimide in anhydrous dimethylformamide at room temperature, to provide a compound of formula (XVI), where HAL is Br. A compound of formula R¹—B(OH)₂; is reacted under Suzuki coupling conditions known to one skilled in the art, or as previously described with a compound of formula (XVI), to provide a compound of formula (VI), where R¹ is an optionally substituted C₂₋₆alkenyl, C₂₋₆haloalkenyl, or aryl as defined herein with reference to Formula (Z). A compound of formula (VI), where R¹ is an optionally substituted C₂₋₆alkenyl or C₂₋₆haloalkenyl is reacted under hydrogenation conditions using Wilkinson catalyst ((PPh₃)₃RhCl) to provide a compound of formula (VI), where R¹ is C₂₋₆alkyl or C₂₋₆haloalkyl.

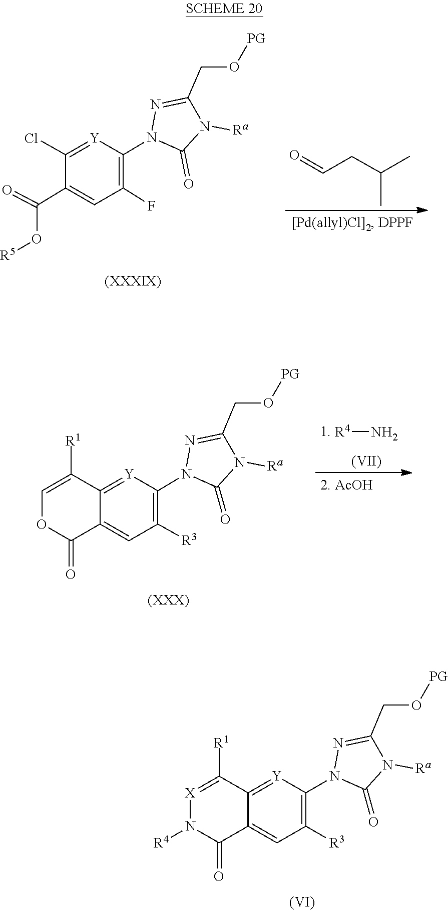

According to SCHEME 20, 3-methylbutanal is reacted with a compound of formula (XXXIX), where Y is N and R⁵ is CH(CH₃)₂, with a palladium catalyst such as allylpalladium(II) chloride dimer, and the like; a ligand such as 1,1′-bis(diphenylphosphino)ferrocene (dppf), and the like; a suitable base such as Cs₂CO₃, and the like; in the presence of water scavenger such as molecular sieve (4A); in a suitable solvent such as dioxane thereof; to provide a compound of formula compound (XXX), where R¹ is isopropyl. A compound of formula R⁴—NH₂, where R⁴ is as defined herein with reference to Formula (Z); is reacted with trimethyl aluminum; in a suitable solvent such as dichloromethane, toluene, or a mixture thereof; the resulting solution is combined with a compound of formula (XXX), followed by subsequent treatment with acetic acid under heating temperature of 80-100° C. for a period of time ranging from 5 to 24 hours; to provide a compound of formula (VI); where X is CH, Y is N, R¹ is isopropyl, R³ is F

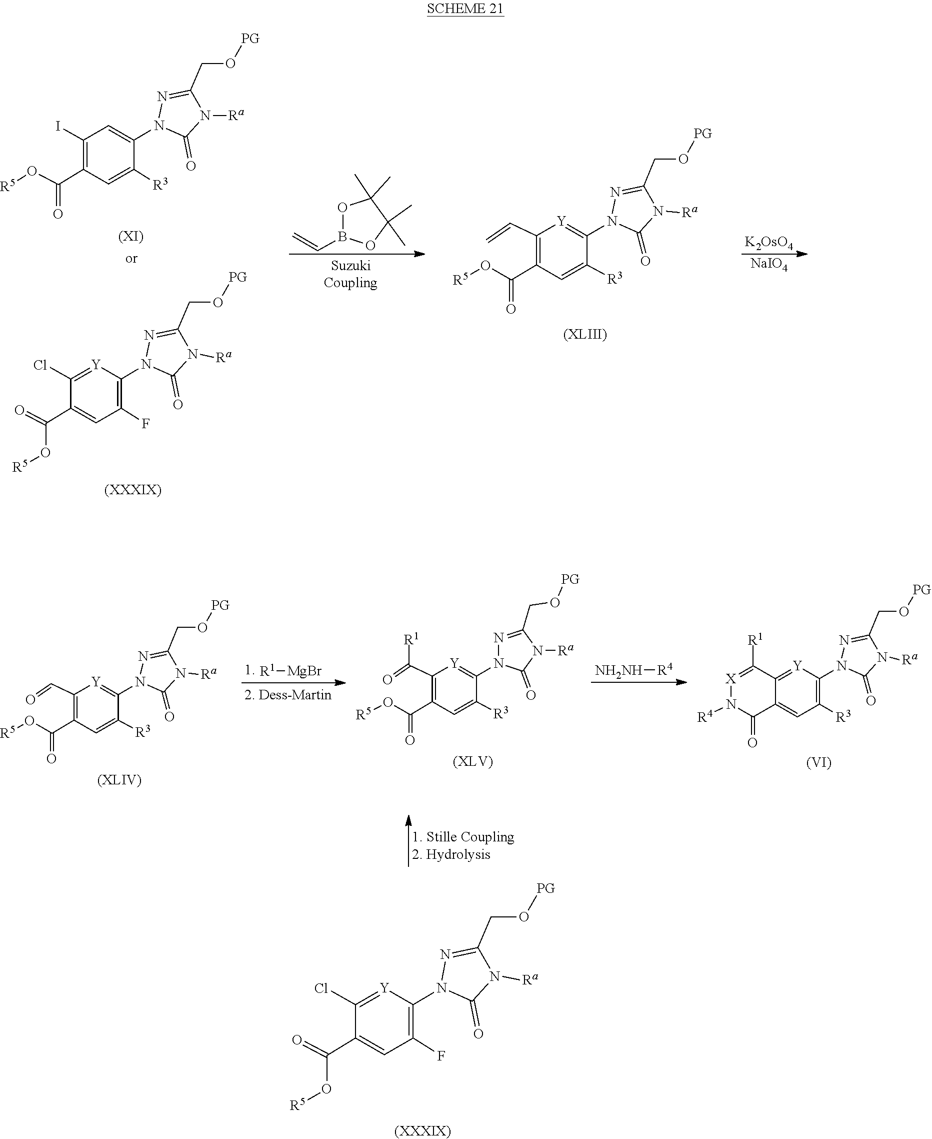

According to SCHEME 21, A compound of formula (XXXIX) or formula (XI), where R³ is F, and R⁵ is C₁₋₄alkyl; is reacted a commercially available vinylboronic acid pinacol ester; a palladium catalyst such as bis(triphenylphosphine)palladium(II) dichloride, or 1,1′-bis(diphenylphosphino)ferrocene-palladium(II)dichloride dichloromethane complex, and the like; a suitable base such as Cs₂CO₃, and the like; in a suitable solvent such as dioxane, water, ethanol, or a mixture thereof; to provide a compound of formula compound (XLIII), where Y is N or CH. The vinyl group in a compound of formula (XLIII) is selectively converted into an aldehyde group of formula (XLIV) employing potassium osmate (VI) dihydrate/sodium periodate, or ozonolysis, and the like. A compound of formula (XLIV) is reacted with a commercially available or synthetically accessible suitably substituted alkyl Grignard reagent such as i-PrMgCl, and the like; in aprotic solvent like THF, and the like; followed by subsequent treatment with an oxidizing reagent such as Dess-Martin reagent, or Swern oxidation conditions, and the like; to afford a ketone compound of formula (XLV).

A compound of formula (XLV) is prepared from a compound of formula (XXXIX) in two steps. A compound of formula (XXXIX), where R³ is F, and R⁵ is C₁₋₄alkyl; is reacted a commercially available tributyl(1-ethoxyvinyl)tin; a palladium catalyst such as bis(triphenylphosphine)palladium(II) dichloride, 1,1′-bis(diphenylphosphino)ferrocene-palladium(II)dichloride and the like; in a suitable solvent such as dioxane, water, ethanol, or a mixture thereof. Subsequent acidic hydrolysis employing conditions such as treatment with aqueous HCl solution at room temperature affords a compound of formula (XLV), where X is N, Y is N or CH, R¹ is methyl.

A commercially or synthetically available hydrazine R⁴—NHNH₂, where R⁴ is as defined herein with reference to Formula (Z), such as 2-chloro-6-fluorophenylhydrazine, o-tolylhydrazine; is condensed with a compound of formula (XLV); in the presence of a base such as potassium carbonate, and the like; under the heating conditions such as 70-120° C.; in a suitable solvent such as toluene, or a mixture thereof; afford a compound of formula (VI), where X is N, Y is CH or N, and R⁴ is as defined herein with reference to Formula (Z).

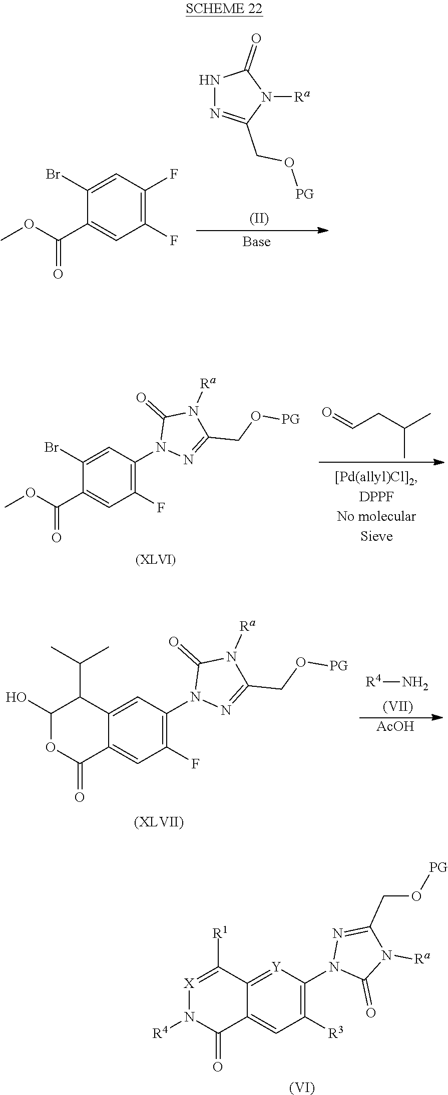

According to SCHEME 22, the reaction of methyl 2-bromo-4,5-difluorobenzoate with a suitably protected triazolone compound of formula (II); in the presence of a base such as K₃PO₄, K₂CO₃, Cs₂CO₃, NaHCO₃, triethylamine, and the like; in a suitable solvent such as 1,4-dioxane, DMSO, DMF, THF, ACN, and the like; affords a compound of formula (XLVI). In a preferred method, PG is Bn, and R^{a} is C₁₋₆alkyl (as previously described in Scheme 14). 3-Methylbutanal is reacted with a compound of formula (XLVI), with a palladium catalyst such as allylpalladium(II) chloride dimer, and the like; a ligand such as 1,1′-bis(diphenylphosphino)ferrocene (dppf), and the like; a suitable base such as Cs₂CO₃, and the like; in the absence of water scavenger such as molecular sieve (4A); in a suitable solvent such as dioxane thereof; to provide a compound of formula compound (XLVII). A compound of formula R⁴—NH₂, where R⁴ is as defined herein with reference to Formula (Z); is reacted with trimethyl aluminum; in a suitable solvent such as dichloromethane, dichloroethane, toluene, or a mixture thereof; the resulting solution is combined with a compound of formula (XLVII), followed by subsequent treatment with acetic acid under heating temperature of 80-100° C. for a period of time ranging from 5 to 24 hours; to provide a compound of formula (VI); where X is CH, Y is CH, R¹ is isopropyl, R³ is F. In certain cases, a compound of formula R⁴—NH₂ such as o-toluidine and the like; is directly condensed with a compound of formula compound (XLVII) in acetic acid under heating temperature of 80-100° C. for a period of time ranging from 10 to 24 hours; to provide a compound of formula (VI); where X, Y, R¹, R³ are defined above.

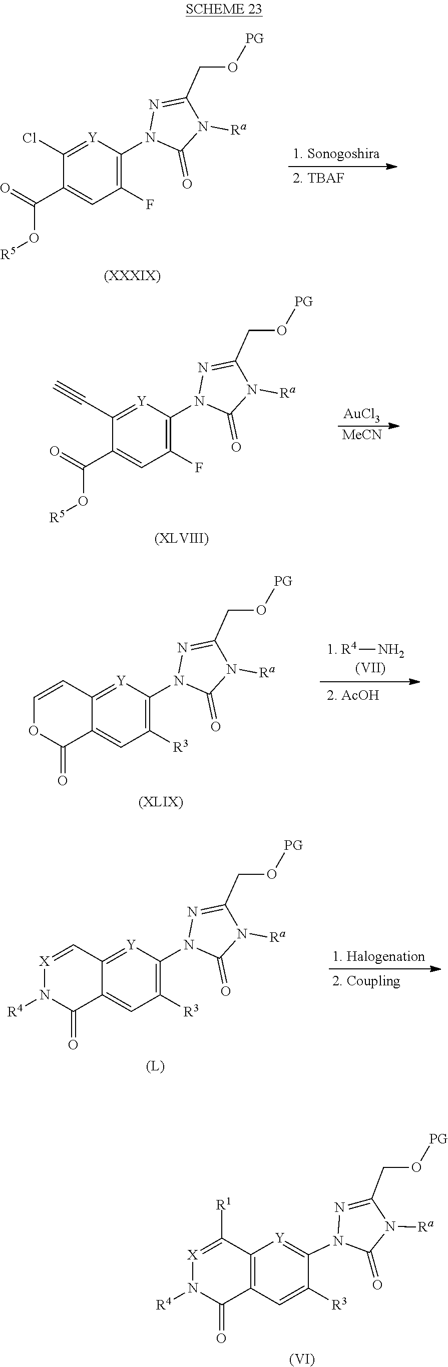

According to SCHEME 23, a compound of formula (XXXIX), where R⁵ is C₁₋₄alkyl, Y is N, and R³ is F, is subjected to a Sonogashira coupling reaction with a silyl protected alkyne, such as trimethylsilylacetylene, a palladium catalyst such as palladium(II)bis(triphenylphosphine) dichloride and the like; a copper catalyst such as copper iodide and the like; with a suitable base, such as triethylamine; in a suitable solvent such as ACN, toluene, and the like. Deprotection reaction employing TBAF in a suitable solvent such as THF, and the like; at room temperature affords a compound of formula (XLVIII). A compound of formula (XLIX) is obtained using a gold catalyst, preferably AuCl₃ in a suitable solvent mixture, such as MeCN. Similar transformation by AuCl₃-catalyzed cyclization has been described by Marchal, E. et al in Tetrahedron 2007, 63, 9979-9990. A compound of formula R⁴—NH₂, where R⁴ is as defined herein with reference to Formula (Z); is reacted with trimethylaluminum; in a suitable solvent such as dichloromethane, dichloroethane, toluene, or a mixture thereof; the resulting solution is combined with a compound of formula (XLIX), followed by subsequent treatment with acetic acid under heating temperature of 80-100° C. for a period of time ranging from 5 to 24 hours; to provide a compound of formula (L). Employing NBS in a suitable solvent, preferably DMF, at room temperature followed by cross coupling using conditions known to one skilled in the art, preferably a palladium catalyst such as palladium(II)bis(triphenylphosphine) dichloride, a base such as Cs₂CO₃, Na₂CO₃, and the like; in a solvent mixture composed of 1,4-dioxane and water; at a temperature of 100° C. provides a compound of formula (VI), where X is CH, Y is N, R³ is F, and R¹, R^{a} and PG are defined as previously described.

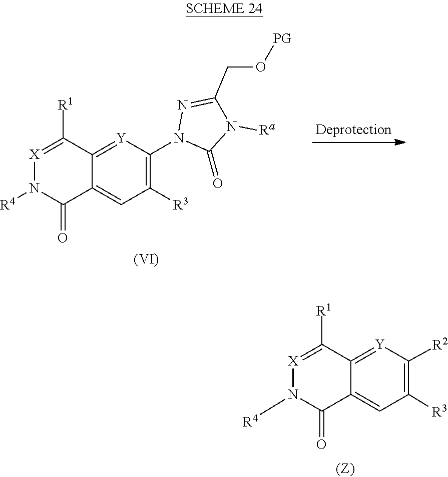

According to SCHEME 24, a compound of formula (VI), where PG is Bn, is deprotected employing conditions known to one skilled in the art, preferably in neat TFA in a sealed tube, at a temperature of about 60 to 90° C.; or employing BCl₃, at a temperature of about −78° C., in a suitable solvent such as in DCM; or treatment with hydrogen gas, in the presence of a catalyst such as Palladium on carbon (Pd/C), affords a compound of Formula (Z).

In a similar fashion, N-arylation and in-situ TBDPS deprotection of a compound of formula (XVIII), where R¹ is I and PG is TBDPS, and X is N; is achieved employing conditions known to one skilled in the art or as previously described, to afford a compound of Formula (Z).

A compound of Formula (Z), where R³ is F is reacted in a nucleophilic aromatic substitution reaction to provide a compound of Formula (Z), where R³ is OCH₃. For example, reaction of a compound of Formula (Z), where R³ is F, with a suitable base such as NaOH, and the like; in a suitable solvent such as MeOH, and the like; to provide a compound of Formula (Z) where Y is CH and R³ is OCH₃.

Compounds of Formula (Z) may be converted to their corresponding salts using methods known to one of ordinary skill in the art. For example, an amine of Formula (Z) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (Z) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate compounds of Formula (Z) and various preferred embodiments.

### EXAMPLES

In obtaining the compounds of Formula (Z) described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:
METHOD A. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (with 0.225% FA) over 10 min and then hold at 100% ACN for 2 min, at a flow rate of 25 mL/min; or
METHOD B. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.1% TFA) over 10 min and then hold at 100% ACN for 2 min, at a flow rate of 25 mL/min; or
METHOD C. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.05% HCl) over 10 min and then hold at 100% ACN for 2 min, at a flow rate of 25 mL/min; or
METHOD D. a Gilson GX-281 semi-prep-HPLC with Phenomenex Gemini C18 (10 μm, 150×25 mm), AD (10 μm, 250 mm×30 mm), or Waters XBridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min; or
METHOD E. a Gilson GX-281 semi-prep-HPLC with Phenomenex Gemini C18 (10 μm, 150×25 mm), or Waters XBridge C18 column (5 μm, 150×30 mm), mobile phase of 5-99% ACN in water (10 mM NH₄HCO₃) over 10 min and then hold at 100% ACN for 2 min, at a flow rate of 25 mL/min.

Preparative supercritical fluid high performance liquid chromatography (SFC) was performed either on a Thar 80 Prep-SFC system, or Waters 80Q Prep-SFC system from Waters. The ABPR was set to 100 bar to keep the CO2 in SF conditions, and the flow rate may verify according to the compound characteristics, with a flow rate ranging from 50 g/min to 70 g/min. The column temperature was ambient temperature.

Mass spectra (MS) were obtained on a SHIMADZU LCMS-2020 MSD or Agilent 1200\G6110A MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, dd=doublet of doublets, ddd=doublet of doublet of doublets, td=triplet of doublets, dt=doublet of triplets, spt=septet, quin=quintet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, MA) or ACD/Name Version 10.01 (Advanced Chemistry).

Compounds designated as R* or S* are enantiopure compounds where the absolute configuration was not determined.

### Intermediate 1: 3-((Benzyloxy)methyl)-4-ethyl-1H-1,2,4-triazol-5(4H)-one

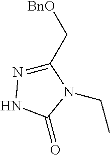 [SMILES: CCn1c(COCc2ccccc2)n[nH]c1=O]

### Step A. 2-(Benzyloxy)acetohydrazide

To a solution of ethyl 2-(benzyloxy)acetate (55 g, 283.17 mmol) in EtOH (500 mL) was added NH₂NH₂·H₂O (28.3 g, 566 mmol, 27.5 mL). The reaction mixture was heated at 78° C. for 6 h.

The reaction mixture was concentrated under reduced pressure to afford the title product (52 g, crude) as a colorless oil, which was used directly in the next step without further purification.

### Step B. 3-((Benzyloxy)methyl)-4-ethyl-1H-1,2,4-triazol-5(4H)-one

To a solution of 2-(benzyloxy)acetohydrazide (52 g, 288 mmol) in H₂O (500 mL) was added dropwise isocyanatoethane (25.1 g, 346 mmol, 27.9 mL) at 0° C. After the addition was complete, the mixture was stirred at 25° C. for 12 hr. To the mixture was added H₂O (20 mL), and an aqueous solution (120 mL) of NaOH (57.7 g, 1.44 mol). The mixture was stirred at 95° C. for 12 hr. The reaction mixture was cooled to rt, then quenched with HCl (12 M) at 0° C. and adjusted to “pH” 6. The solid was filtered and dried under reduced pressure to afford the title compound as a white solid (61 g, 91% yield). ¹H NMR (400 MHz, CDCl₃) δ −9.23-9.09 (m, 1H), −7.41-7.31 (m, 5H), −4.58-4.53 (m, 2H), −4.45-4.42 (m, 2H), −3.82-3.75 (m, 2H), −1.33-1.29 (m, 3H) ppm.

### Intermediate 2: 5-(((tert-Butyldiphenylsilyl)oxy)methyl)-4-ethyl-2,4-dihydro-3H-1,2,4-triazol-3-one

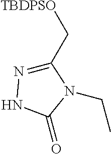

### Step A. 4-Ethyl-5-(hydroxymethyl)-2,4-dihydro-3H-1,2,4-triazol-3-one

To a solution of 5-[(benzyloxy)methyl]-4-methyl-2,4-dihydro-3H-1,2,4-triazol-3-one (8 g, 34.3 mmol, 1.0 eq.) in methanol (200 mL) was added Pd/C (2 g). The resulting mixture was maintained under hydrogen and stirred at rt for 6 h. Then the resulting mixture was filtered and the filtrate was concentrated to afford the crude product 4-ethyl-5-(hydroxymethyl)-2,4-dihydro-3H-1,2,4-triazol-3-one as a white solid (4.3 g, 88% yield). ¹H NMR (400 MHz, DMSO-d₆) δ 11.52 (s, 1H), 5.55 (t, J=5.50 Hz, 1H), 4.32 (d, J=5.50 Hz, 2H), 3.64 (q, J=6.97 Hz, 2H), 1.18 (t, J=6.97 Hz, 3H) ppm.

### Step B. 5-(((tert-Butyldiphenylsilyl)oxy)methyl)-4-ethyl-2,4-dihydro-3H-1,2,4-triazol-3-one

To a solution of 4-ethyl-5-(hydroxymethyl)-2,4-dihydro-3H-1,2,4-triazol-3-one (3 g, 21 mmol, 1.0 eq.) in DCM (30 mL) was added tert-butylchlorodiphenylsilane (6.5 mL, 25 mmol, 1.2 eq.) and pyridine (1.86 mL, 23 mmol, 1.1 eq.). The resulting mixture was stirred at rt overnight. The reaction mixture was quenched with water (100 mL). The resulting mixture was extracted with DCM (3×100 mL). The organic layers were combined, dried over anhydrous sodium sulfate, filtered and concentrated. The residue was purified by silica gel chromatography (SiO₂, 50-80% ethyl acetate/petroleum ether) to afford 5-(((tert-butyldiphenylsilyl)oxy)methyl)-4-ethyl-2,4-dihydro-3H-1,2,4-triazol-3-one as a white solid (4.9 g, 61% yield). LCMS (ES-API): mass calcd. for C₂₁H₂₇N₃O₂Si, 381.2; m/z found, 382.2 [M+H]⁺. ¹H NMR (400 MHz, CDCl₃) δ 9.98 (s, 1H), 7.61-7.72 (m, 4H), 7.32-7.54 (m, 6H), 4.54 (s, 2H), 3.84 (q, J=7.34 Hz, 2H), 1.33 (t, J=7.34 Hz, 3H), 1.07 (s, 9H) ppm.

### Intermediate 3: 5-((Benzyloxy)methyl)-4-ethyl-2-(7-fluoro-1-oxo-4-(prop-1-en-2-yl)-1H-isochromen-6-yl)-2,4-dihydro-3H-1,2,4-triazol-3-one

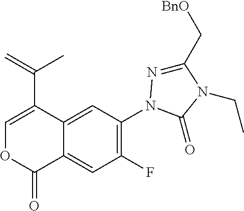 [SMILES: C=C(C)c1coc(=O)c2cc(F)c(-n3nc(COCc4ccccc4)n(CC)c3=O)cc12]

### Step A. tert-Butyl 4,5-difluoro-2-iodobenzoate

4,5-Difluoro-2-iodobenzoic acid (3 g, 11 mmol) was dissolved in THE (30 mL), then di-tert-butyl dicarbonate (4.6 g, 21 mmol) was added followed by DMAP (645 mg, 5.3 mmol). The reaction mixture was stirred under nitrogen at 50° C. overnight, then cooled down to room temperature. The solvent was evaporated under reduced pressure. The residue was diluted with EtOAc then washed with brine. The organic layer was separated, dried with Na₂SO₄, filtered, and concentrated. The residue was purified by silica column chromatography (gradient elution: 0-5% EtOAc in petroleum ether) to give the title compound as a yellow oil (2.9 g, yield: 79%). ¹H NMR (400 MHz, CDCl₃) δ 7.77 (dd, J=10.2, 7.9 Hz, 1H), 7.63 (dd, J=10.2, 7.9 Hz, 1H), 1.62 (s, 9H) ppm; ¹⁹F NMR (376 MHz, CDCl₃) δ −131.55-−131.13 (m, 1F), −136.97-−136.65 (m, 1F) ppm.

### Step B. tert-Butyl 4-(3-((benzyloxy)methyl)-4-ethyl-5-oxo-4,5-dihydro-1H-1,2,4-triazol-1-yl)-5-fluoro-2-iodobenzoate

A mixture of tert-butyl 4,5-difluoro-2-iodobenzoate (3.2 g, 9.4 mmol), 3-((benzyloxy)methyl)-4-ethyl-1H-1,2,4-triazol-5(4H)-one (Intermediate 1, 2.6 g, 11.2 mmol) and Cs₂CO₃ (6.1 g, 18.7 mmol) in anhydrous DMF (30 mL) was stirred under nitrogen at 75° C. for 1 h, then cooled to room temperature. The mixture was filtered through a pad of Celite®, and the pad was washed with EtOAc. The filtrate was combined, washed with brine, and concentrated. The residue was purified by silica column chromatography (gradient elution: 0-40% EtOAc in petroleum ether) to give the title compound as a colorless amorphous solid (5 g, yield: 96%). ESI-MS: mass calcd. for C₂₃H₂₅FIN₃O₄, 553.1; m/z found, 554.1 [M+H]⁺. ¹H NMR (400 MHz, CDCl₃) δ 8.16 (d, J=7.1 Hz, 1H), 7.62 (d, J=10.8 Hz, 1H), 7.29-7.45 (m, 5H), 4.61 (s, 2H), 4.50 (s, 2H), 3.84 (q, J=7.2 Hz, 2H), 1.63 (s, 9H), 1.35 (t, J=7.2 Hz, 3H) ppm; ¹⁹F NMR (376 MHz, CDCl₃) δ −119.09 (dd, J=10.6, 7.0 Hz, 1F) ppm.

### Step C. 4-(3-((Benzyloxy)methyl)-4-ethyl-5-oxo-4,5-dihydro-1H-1,2,4-triazol-1-yl)-5-fluoro-2-iodobenzoic acid

To a solution of tert-butyl 4-(3-((benzyloxy)methyl)-4-ethyl-5-oxo-4,5-dihydro-1H-1,2,4-triazol-1-yl)-5-fluoro-2-iodobenzoate (5 g, 9 mmol) in DCM (50 mL) was slowly added TFA (10 mL). The reaction mixture was stirred at room temperature overnight. The reaction mixture was concentrated under vacuum. The obtained residue was triturated with petroleum ether at room temperature for 30 min. The mixture was filtered and the solid was rinsed with petroleum ether. The precipitate was collected and dried in vacuo to give the title compound as a white solid (4.1 g, yield: 91%). ESI-MS: mass calcd. for C₁₉H₁₇FIN₃O₄, 497.0; m/z found, 498.0 [M+H]⁺. ¹H NMR (400 MHz, DMSO-d₆) δ 8.16 (d, J=7.3 Hz, 1H), 7.78 (d, J=11.0 Hz, 1H), 7.28-7.43 (m, 5H), 4.60 (s, 2H), 4.57 (s, 2H), 3.74 (q, J=7.2 Hz, 2H), 1.23 (t, J=7.2 Hz, 3H) ppm; ¹⁹F NMR (376 MHz, DMSO-d₆) δ −119.91 (s, 1F) ppm.

### Step D. 5-((Benzyloxy)methyl)-4-ethyl-2-(7-fluoro-1-oxo-4-(prop-1-en-2-yl)-1H-isochromen-6-yl)-2,4-dihydro-3H-1,2,4-triazol-3-one

To a mixture of 3-methylbuta-1,2-dien-1-yl acetate (Intermediate 12, 280 mg, 2.2 mmol), 4-(3-((benzyloxy)methyl)-4-ethyl-5-oxo-4,5-dihydro-1H-1,2,4-triazol-1-yl)-5-fluoro-2-iodobenzoic acid (1.1 g, 2.2 mmol) and Cy₂NMe (867 mg, 4.4 mmol) in DMF (7 mL) was added Catacxium A Pd G2 (74.2 mg, 0.11 mmol) under nitrogen. The reaction mixture was stirred under nitrogen at 90° C. for overnight. The mixture was then cooled to room temperature, diluted with EtOAc and washed with brine. The organic layer was separated and the aqueous layer was combined and extracted with EtOAc. The combined organic layer was dried over Na₂SO₄, filtered and concentrated. The residue was purified by silica column chromatography (gradient elution: 0-80% EtOAc in petroleum ether) to give the title compound as yellow solid (240 mg, yield: 25%). ESI-MS: mass calcd. for C₂₄H₂₂FN₃O₄, 435.2; m/z 436.2 [M+H]⁺. ¹H NMR (400 MHz, CDCl₃) δ 8.15 (d, J=10.5 Hz, 1H), 7.86 (d, J=6.8 Hz, 1H), 7.30-7.45 (m, 5H), 7.19 (s, 1H), 5.37-5.39 (m, 1H), 5.18 (s, 1H), 4.62 (s, 2H), 4.53 (s, 2H), 3.87 (q, J=7.1 Hz, 2H), 2.11 (s, 3H), 1.37 (t, J=7.2 Hz, 3H) ppm.

### Intermediate 4: 5-((Benzyloxy)methyl)-4-ethyl-2-(7-fluoro-4-isopropyl-1-oxo-1H-isochromen-6-yl)-2,4-dihydro-3H-1,2,4-triazol-3-one

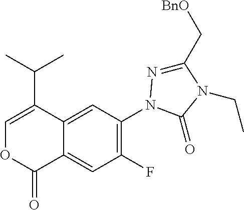 [SMILES: CCn1c(COCc2ccccc2)nn(-c2cc3c(C(C)C)coc(=O)c3cc2F)c1=O]

### Method I

### Step A. 3-Methylbut-2-en-1-yl 4,5-difluoro-2-iodobenzoate

To the mixture of 4,5-difluoro-2-iodobenzoic acid (1.4 g, 4.9 mmol) and Cs₂CO₃ (4.8 g, 14.8 mmol) in anhydrous DMF (20 mL) was added 1-bromo-3-methyl-2-butene (1.5 g, 9.9 mmol). The reaction mixture was stirred at room temperature for 18 h. The mixture was diluted with water, and the mixture was extracted with DCM and EtOAc. The combined organic extract was dried over Na₂SO₄, filtered and concentrated. The residue was purified by flash chromatography (SiO₂, gradient elution: 10-20% EtOAc in heptane) to give the desired product as a colorless oil (1.6 g, yield: 92%). ¹H NMR (400 MHz, CDCl₃) δ 7.80 (dd, J=7.58, 9.54 Hz, 1H), 7.73 (dd, J=7.83, 10.76 Hz, 1H), 5.42-5.52 (m, 1H), 4.82 (d, J=7.34 Hz, 2H), 1.80 (s, 3H), 1.78 (s, 3H) ppm.

### Step B. 3-Methylbut-2-en-1-yl 4-(3-((benzyloxy)methyl)-4-ethyl-5-oxo-4,5-dihydro-1H-1,2,4-triazol-1-yl)-5-fluoro-2-iodobenzoate

To a mixture of 3-methylbut-2-en-1-yl 4,5-difluoro-2-iodobenzoate (1.6 g, 4.5 mmol), 3-((benzyloxy)methyl)-4-ethyl-1H-1,2,4-triazol-5(4H)-one (Intermediate 1, 2.1 g, 9.1 mmol) in anhydrous DMF (25 mL) was added Cs₂CO₃ (2.9 g, 9.1 mmol). The reaction mixture was heated under nitrogen at 85° C. for 1 h, then cooled to room temperature. The mixture was diluted with water, and the mixture was extracted with DCM and EtOAc. The combined organic extract was dried over Na₂SO₄, filtered and concentrated. The residue was purified by flash chromatography (SiO₂, gradient elution: 20-50% EtOAc in heptane) to give the title compound as a white solid (2.4 g, yield: 93%). LCMS (ES-API): mass calcd. for C₂₄H₂₅FIN₃O₄, 565.1; m/z found, 566.2 [M+H]⁺. ¹H NMR (400 MHz, CDCl₃) δ 8.21 (d, J=6.85 Hz, 1H), 7.73 (d, J=11.25 Hz, 1H), 7.29-7.44 (m, 5H), 5.41-5.53 (m, 1H), 4.84 (d, J=7.34 Hz, 2H), 4.60 (s, 2H), 4.50 (s, 2H), 3.84 (q, J=7.22 Hz, 2H), 1.80 (s, 3H), 1.78 (d, J=0.98 Hz, 3H), 1.34 (t, J=7.22 Hz, 3H) ppm.

### Step C. 5-((Benzyloxy)methyl)-4-ethyl-2-(7-fluoro-4-isopropyl-1-oxo-1H-isochromen-6-yl)-2,4-dihydro-3H-1,2,4-triazol-3-one

To a mixture of 3-methylbut-2-en-1-yl 4-(3-((benzyloxy)methyl)-4-ethyl-5-oxo-4,5-dihydro-1H-1,2,4-triazol-1-yl)-5-fluoro-2-iodobenzoate (4 g, 6.86 mmol, 1 eq) in toluene (200 mL) was added (tBu₃P)PdG₂ (351 mg, 0.69 mmol, 0.1 eq), N-cyclohexyl-N-methyl-cyclohexanamine (1.60 mL, 7.54 mmol, 1.1 eq) respectively. The reaction mixture was degassed with nitrogen for three times, and then heated under nitrogen atmosphere at 80° C. for 18 h. LCMS analysis showed ~18% of starting material remained. The mixture was cooled to 15° C., and additional N-cyclohexyl-N-methyl-cyclohexanamine (0.72 mL, 3.43 mmol, 0.5 eq) and tBu₃PPdG₂ (176 mg, 0.34 mmol, 0.05 eq) were added. The reaction mixture was degassed with nitrogen, and then heated under nitrogen atmosphere at 80° C. for 16 h. The mixture was concentrated under reduced pressure, then diluted with H₂O (200 mL), and extracted with EtOAc (150 mL×3). The combined organic layers were washed with brine (100 mL×2), dried over anhydrous Na₂SO₄, filtered, and concentrated under reduced pressure. The residue was purified by flash chromatography (SiO₂, Petroleum ether/Ethyl acetate=5/1 to 3/1) to give the title compound as a yellow oil (1.1 g, yield: 35%). ESI-MS: mass calcd. for C₂₄H₂₄FN₃O₄, 437.2; m/z found, 438.5 [M+H]⁺. ¹H NMR (400 MHz, CDCl₃) δ 8.16 (d, J=6.6 Hz, 1H), 7.96 (d, J=6.6 Hz, 1H), 7.42-7.34 (m, 5H), 7.13 (s, 1H), 4.63 (s, 2H), 4.54 (s, 2H), 3.88 (dd, J=7.2, 14.4 Hz, 2H), 3.13-3.06 (m, 1H), 1.38 (t, J=7.2 Hz, 3H), 1.32 (d, J=6.8 Hz, 6H) ppm.

### Method II

To a mixture of 5-((benzyloxy)methyl)-4-ethyl-2-(7-fluoro-1-oxo-4-(prop-1-en-2-yl)-1H-isochromen-6-yl)-2,4-dihydro-3H-1,2,4-triazol-3-one (Intermediate 3, 5.9 g, 13.5 mmol) in THE (100 mL) at room temperature was added Wilkinson's Catalyst [RhCl(PPh₃)₃](3.8 g, 4.1 mmol). The mixture was degassed and purged with hydrogen gas. The reaction mixture was stirred under an atmosphere of hydrogen (15 Psi) at room temperature for 12 h. The mixture was concentrated. The residue was purified by silica column chromatography (elution: 0-25% EtOAc in petroleum ether) to give the title compound as a yellow solid (1.5 g, yield: 77%). ESI-MS: mass calcd. for C₂₄H₂₄FN₃O₄, 437.2; m/z found 438.2 [M+H]⁺. ¹H NMR (400 MHz, DMSO-d₆) δ 8.10 (d, J=10.5 Hz, 1H), 8.01 (d, J=7.0 Hz, 1H), 7.46 (s, 1H), 7.26-7.42 (m, 5H), 4.61 (s, 2H), 4.59 (s, 2H), 3.77 (q, J=7.3 Hz, 2H), 3.08 (dt, J=13.4, 6.8 Hz, 1H), 1.22-1.28 (m, 9H) ppm; ¹⁹F NMR (376 MHz, DMSO-d₆) δ −118.29 (br s, 1F) ppm.

### Intermediate 5: 4-(3-((Benzyloxy)methyl)-4-ethyl-5-oxo-4,5-dihydro-1H-1,2,4-triazol-1-yl)-5-fluoro-2-iodobenzoyl chloride

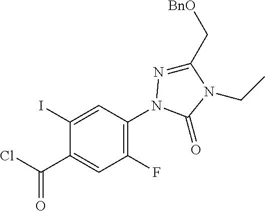 [SMILES: CCn1c(COCc2ccccc2)nn(-c2cc(I)c(C(=O)Cl)cc2F)c1=O]

### Step A. tert-Butyl 4,5-difluoro-2-iodobenzoate

4,5-Difluoro-2-iodobenzoic acid (3 g, 11 mmol) was dissolved in THE (30 mL), then di-tert-butyl dicarbonate (4.6 g, 21 mmol) was added followed by DMAP (645 mg, 5.3 mmol). The reaction mixture was stirred under nitrogen at 50° C. overnight, then cooled down to room temperature. The solvent was evaporated under reduced pressure. The residue was diluted with EtOAc then washed with brine. The organic layer was separated, dried with Na₂SO₄, filtered, and concentrated. The residue was purified by silica column chromatography (gradient elution: 0-5% EtOAc in petroleum ether) to give the title compound as a yellow oil (2.9 g, yield: 79%). ¹H NMR (400 MHz, CDCl₃) δ 7.77 (dd, J=10.2, 7.9 Hz, 1H), 7.63 (dd, J=10.2, 7.9 Hz, 1H), 1.62 (s, 9H) ppm; ¹⁹F NMR (376 MHz, CDCl₃) δ −131.55-−131.13 (m, 1F), −136.97-−136.65 (m, 1F) ppm.

### Step B. tert-Butyl 4-(3-((benzyloxy)methyl)-4-ethyl-5-oxo-4,5-dihydro-1H-1,2,4-triazol-1-yl)-5-fluoro-2-iodobenzoate

A mixture of tert-butyl 4,5-difluoro-2-iodobenzoate (3.2 g, 9.4 mmol), 3-((benzyloxy)methyl)-4-ethyl-1H-1,2,4-triazol-5(4H)-one (Intermediate 1, 2.6 g, 11.2 mmol) and Cs₂CO₃ (6.1 g, 18.7 mmol) in anhydrous DMF (30 mL) was stirred under nitrogen at 75° C. for 1 h, then cooled to room temperature. The mixture was filtered through a pad of Celite®, and the pad was washed with EtOAc. The filtrate was combined, washed with brine, and concentrated. The residue was purified by silica column chromatography (gradient elution: 0-40% EtOAc in petroleum ether) to give the title compound as a colorless amorphous solid (5 g, yield: 96%). ESI-MS: mass calcd. for C₂₃H₂₅FIN₃O₄, 553.1; m/z found, 554.1 [M+H]⁺. ¹H NMR (400 MHz, CDCl₃) δ 8.16 (d, J=7.1 Hz, 1H), 7.62 (d, J=10.8 Hz, 1H), 7.29-7.45 (m, 5H), 4.61 (s, 2H), 4.50 (s, 2H), 3.84 (q, J=7.2 Hz, 2H), 1.63 (s, 9H), 1.35 (t, J=7.2 Hz, 3H) ppm; ¹⁹F NMR (376 MHz, CDCl₃) δ −119.09 (dd, J=10.6, 7.0 Hz, 1F) ppm.

### Step C. 4-(3-((Benzyloxy)methyl)-4-ethyl-5-oxo-4,5-dihydro-1H-1,2,4-triazol-1-yl)-5-fluoro-2-iodobenzoic acid

To a solution of tert-butyl 4-(3-((benzyloxy)methyl)-4-ethyl-5-oxo-4,5-dihydro-1H-1,2,4-triazol-1-yl)-5-fluoro-2-iodobenzoate (5 g, 9 mmol) in DCM (50 mL) was slowly added TFA (10 mL). The reaction mixture was stirred at room temperature overnight. The reaction mixture was concentrated under vacuum. The obtained residue was triturated with petroleum ether at room temperature for 30 min. The mixture was filtered and the solid was rinsed with petroleum ether. The precipitate was collected and dried in vacuo to give the title compound as a white solid (4.1 g, yield: 91%). ESI-MS: mass calcd. for C₁₉H₁₇FIN₃O₄, 497.0; m/z found, 498.0 [M+H]⁺. ¹H NMR (400 MHz, DMSO-d₆) δ 8.16 (d, J=7.3 Hz, 1H), 7.78 (d, J=11.0 Hz, 1H), 7.28-7.43 (m, 5H), 4.60 (s, 2H), 4.57 (s, 2H), 3.74 (q, J=7.2 Hz, 2H), 1.23 (t, J=7.2 Hz, 3H) ppm; ¹⁹F NMR (376 MHz, DMSO-d₆) δ −119.91 (s, 1F) ppm.

### Step D. 4-(3-((Benzyloxy)methyl)-4-ethyl-5-oxo-4,5-dihydro-1H-1,2,4-triazol-1-yl)-5-fluoro-2-iodobenzoyl chloride

A solution of 4-(3-((benzyloxy)methyl)-4-ethyl-5-oxo-4,5-dihydro-1H-1,2,4-triazol-1-yl)-5-fluoro-2-iodobenzoic acid (3.5 g, 7 mmol) in SOCl₂ (14 mL) was heated at reflux for 15 min. The reaction mixture was cooled to room temperature and concentrated. To the residue was added anhydrous toluene, then the mixture was evaporated to give the crude product as a yellow gum (3.6 g), which was directly used for the next step without further purification.

### Intermediate 6: 2-Chloro-6-fluoro-N-(3-methylpent-2-en-1-yl)aniline

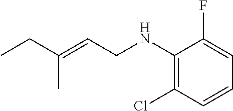

### Step A. Ethyl 3-methylpent-2-enoate

To a solution of 2-butanone (52 g, 717.6 mmol) and (carbethoxymethylene) triphenylphosphorane (50 g, 143.5 mmol) in toluene (65 mL) was added benzoic acid (3.5 g, 28.7 mmol). The reaction mixture was heated at reflux for 16 h. The mixture was diluted with petroleum ether and filtered through a short pad of silica gel. The silica gel was washed with hexane. The filtrate was concentrated under reduced pressure at 0-2° C. The residue was purified by silica column chromatography (elution: 0-10% EtOAc in petroleum ether) to give the title compound as a colorless liquid (23.3 g crude). ¹H NMR (400 MHz, CDCl₃) δ 5.58-5.69 (m, 1H), 4.13 (qd, J=7.1, 4.9 Hz, 2H), 2.62 (q, J=7.5 Hz, 1H), 2.09-2.20 (m, 3H), 1.86 (d, J=1.2 Hz, 1H), 1.24-1.28 (m, 3H), 1.01-1.09 (m, 3H) ppm.

### Step B. 3-Methylpent-2-en-1-ol

To a toluene solution (1 M) of DIBAL-H (118 mL, 118 mmol) at −78° C. was added a toluene solution (40 mL) of ethyl 3-methylpent-2-enoate (20 g crude) dropwise under nitrogen. The reaction mixture was stirred at −78° C. for 2 h. The mixture was warmed to room temperature and slowly poured into saturated aqueous potassium sodium tartrate solution at 0° C. The mixture was stirred for 2 h and filtered through a short pad of Celite©. The pad was washed with DCM/EtOAc (v/v, 3/1), and the filtrate was extracted with DCM. The organic extract was separated, dried over Na₂SO₄, filtered and concentrated. The residue was purified by silica column chromatography (elution: 0-100% DCM in petroleum ether, then 0-30% EtOAc in DCM) to give the title compound as a colorless liquid (7 g, yield of two steps: 57%). ¹H NMR (400 MHz, CDCl₃) δ 5.35-5.46 (m, 1H), 4.11-4.21 (m, 2H), 2.02-2.13 (m, 2H), 1.67-1.76 (m, 3H), 0.98-1.06 (m, 3H) ppm.

### Step C. 3-Methylpent-2-enal

To a solution of 3-methylpent-2-en-1-ol (2 g, 20.0 mmol) in DCM (20 mL) was added Dess-martin periodinane (10 g, 24.0 mmol). The reaction mixture was stirred at room temperature for 1 h. The mixture was filtered through a short pad of Celite©. The pad was washed with DCM. The combined filtrate was washed with saturated aqueous NaHCO₃ solution. The organic layer was separated, dried over Na₂SO₄, filtered and concentrated under reduced pressure at 0-2° C. The crude was purified by silica column chromatography (elution: DCM) to give the title compound as a colorless liquid (1.5 g, yield: 77%). ¹H NMR (400 MHz, CDCl₃) δ 9.91-10.04 (m, 1H), 5.78-5.90 (m, 1H), 2.58 (q, J=7.6 Hz, 1H), 2.23 (d, J=7.3 Hz, 1H), 2.16 (s, 2H), 1.96 (d, J=1.1 Hz, 1H), 1.16 (t, J=7.6 Hz, 1H), 1.09 (t, J=7.4 Hz, 2H) ppm.

### Step D. N-(2-Chloro-6-fluorophenyl)-3-methylpent-2-en-1-imine

To a mixture of 2-chloro-6-fluoroaniline (1.2 g, 8.2 mmol) and 3-methylpent-2-enal (0.97 g, 9.9 mmol) in DCM (18 mL) under nitrogen at 0° C. was added triethylamine (4.6 mL, 33 mmol), followed by the addition of a DCM solution (1 M) of TiCl₄ (5 mL, 5 mmol) dropwise. The resulting mixture was stirred at 0° C. for 1 h, then warmed to room temperature and stirred for 4 h. The mixture was poured into saturated aqueous NH₄Cl solution. The mixture became cloudy and filtered through a pad of Celite©. The pad was washed with EtOAc. The combined filtrate was diluted with DCM and water. The organic layer was separated, and aqueous layer was extracted with DCM. The combined organic layers were washed with brine, dried over Na₂SO₄, filtered and concentrated. The residue product was purified by silica gel column chromatography (gradient elution: 0-5% DCM in petroleum ether) to give the title compound as a pale yellow oil (1.3 g, yield: 70%).

### Step E. 2-Chloro-6-fluoro-N-(3-methylpent-2-en-1-yl)aniline

To a solution of N-(2-chloro-6-fluorophenyl)-3-methylpent-2-en-1-imine (1.3 g, 5.76 mmol) in MeOH (20 mL) was added NaBH₄ (218 mg, 5.8 mmol), and after 1 h, another batch of NaBH₄ (218 mg, 5.8 mmol) was added. A total of NaBH₄ (1.1 g, 29 mmol) was added. The reaction mixture was stirred at room temperature overnight. The mixture was concentrated, and then diluted with water and extracted with EtOAc. The organic layer was separated, washed with brine, dried over Na₂SO₄, filtered and concentrated. The residue was purified by combi flash column chromatography over silica gel (eluent: 0-5% DCM in petroleum ether) to give the title compound as a yellow oil (430 mg, yield: 33%). ¹H NMR (400 MHz, CDCl₃) δ 6.95-7.02 (m, 1H), 6.84 (ddd, J=12.2, 8.3, 1.3 Hz, 1H), 6.52-6.63 (m, 1H), 5.17-5.28 (m, 1H), 3.85 (d, J=5.6 Hz, 2H), 3.73 (s, 1H), 1.91-2.07 (m, 2H), 1.58-1.68 (m, 3H), 0.89-0.96 (m, 3H).

### Intermediate 7: 5-Chloro-3-methyl-1-(tetrahydro-2H-pyran-2-yl)-1H-pyrazol-4-amine

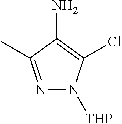

### Step A. 5-Chloro-3-methyl-4-nitro-1-(tetrahydro-2H-pyran-2-yl)-1H-pyrazole

To a solution of 3-methyl-4-nitropyrazole (2 g, 15.7 mmol) in EtOAc (20 mL) was added DHP (2 g, 23.6 mmol) and TsOH·H₂O (150 mg, 0.79 mmol) at room temperature. The mixture was stirred at room temperature for overnight. Et₃N (0.4 mL) was added and the mixture was washed with brine. Then the organic layer was separated, dried over anhydrous Na₂SO₄, filtered and concentrated. The residue was dissolved in THE (45 mL) and the temperature was lowered to −78° C. A THE (1 M) solution of LiHMDS (10.6 mL, 13.8 mmol) was added to the mixture under nitrogen. After 45 minutes at −78° C., the solution of hexachloroethane (8.9 g, 37.8 mmol) in THE (20 mL) was added dropwise. The reaction mixture was warmed to room temperature and stirred for overnight. The mixture was poured into saturated aqueous NH₄Cl solution and extracted with EtOAc. The organic phase was separated, washed with brine, dried over anhydrous Na₂SO₄, filtered and concentrated. The residue was purified by silica column chromatography (gradient elution: 0-40% EtOAc in petroleum ether) to give the title compound as white solid (1.8 g, yield: 58%). ¹H NMR (400 MHz, CDCl₃) δ 5.52 (dd, J=10.0, 2.7 Hz, 1H), 4.07-4.15 (m, 1H), 3.70 (td, J=11.3, 2.8 Hz, 1H), 2.57 (s, 3H), 2.37-2.47 (m, 1H), 2.11-2.19 (m, 1H), 1.86-1.90 (m, 1H), 1.72-1.75 (m, 1H), 1.64 (d, J=2.0 Hz, 1H), 1.53 (d, J=6.6 Hz, 1H) ppm.

### Step B. 5-Chloro-3-methyl-1-(tetrahydro-2H-pyran-2-yl)-1H-pyrazol-4-amine

To a mixture of 5-chloro-3-methyl-4-nitro-1-(tetrahydro-2H-pyran-2-yl)-1H-pyrazole (100 mg, 0.4 mmol) in MeOH/THF/H₂O (v/v/v, 1/1/1, 3 mL) was added iron powder (114 mg, 2.0 mmol) and NH₄Cl (109 mg, 2.0 mmol). The mixture was stirred at 70° C. for 1.5 h. The mixture was cooled to room temperature and filtered through a pad of Celite©. The pad was washed with EtOAc. The combined filtrate was washed with saturated aqueous NaHCO₃ solution. The organic layer was separated, and the aqueous layer was extracted with EtOAc. The combined organic extract was washed with brine, dried over anhydrous Na₂SO₄, filtered and concentrated. The residue was purified by silica column chromatography (gradient elution: 0-50% EtOAc in petroleum ether) to give the title compound as a yellow oil (70 mg, yield: 79%). ESI-MS: mass calcd. for C₉H₁₄ClN₃O, 215.1; m/z found, 216.1 [M+H]⁺.

### Intermediate 8: 3-(2-((tert-Butyldiphenylsilyl)oxy)ethoxy)-2-chloroaniline

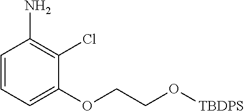

### Step A. tert-Butyl(2-(2-chloro-3-nitrophenoxy)ethoxy)diphenylsilane

To a mixture of 2-chloro-3-nitrophenol (200 mg, 1.2 mmol), 2-((tert-butyldiphenylsilyl)oxy)ethan-1-ol (554 mg, 1.8 mmol) and PPh₃ (453 mg, 1.7 mmol) in THE (10 mL) was added DEAD (281 mg, 161 mmol) at 0° C. under nitrogen. The mixture was warmed to room temperature and stirred at room temperature for 12 h. Saturated aqueous NH₄Cl solution was added, and the mixture was extracted with EtOAc. The organic was separated, washed with brine, dried over anhydrous Na₂SO₄, filtered and concentrated. The residue was purified by silica column chromatography (gradient elution: 0-10% EtOAc in petroleum ether) to give the title compound as a yellow oil (240 mg, yield: 46%). ¹H NMR (400 MHz, CDCl₃) δ 7.72 (dd, J=7.8, 1.5 Hz, 4H), 7.35-7.49 (m, 7H), 7.30 (t, J=8.2 Hz, 1H), 7.12 (dd, J=8.3, 1.2 Hz, 1H), 4.20-4.25 (m, 2H), 4.06 (t, J=4.9 Hz, 2H), 1.06 (s, 9H) ppm.

### Step B. 3-(2-((tert-Butyldiphenylsilyl)oxy)ethoxy)-2-chloroaniline

To a mixture of tert-butyl(2-(2-chloro-3-nitrophenoxy)ethoxy)diphenylsilane (220 mg, 0.5 mmol), NH₄Cl (258 mg, 4.8 mmol) in THE (3 mL), MeOH (3 mL) and H2O (3 mL) was added iron powder (269 mg, 4.8 mmol). The reaction mixture was stirred at 70° C. for 2 h. The mixture was cooled to room temperature, diluted with EtOAc, and filtered through a pad of Celite©. The Celite® was washed with EtOAc. The combined filtrate was washed with brine, dried over Na₂SO₄, filtered and concentrated. The residue was purified by silica column chromatography (gradient elution: 0-11% EtOAc in petroleum ether) to give the title compound as a yellow solid (192 mg, yield: 92%). ESI-MS: mass calcd. for C₂₄H₂₈ClNO₂Si, 425.2; m/z found, 426.1[M+H]⁺. ¹H NMR (400 MHz, CDCl₃) δ 7.75 (dd, J=7.9, 1.6 Hz, 4H), 7.35-7.48 (m, 6H), 6.97 (t, J=8.1 Hz, 1H), 6.42 (dd, J=8.2, 1.1 Hz, 1H), 6.33 (dd, J=8.2, 1.1 Hz, 1H), 4.13-4.17 (m, 2H), 4.07-4.13 (m, 2H), 4.01-4.06 (m, 2H), 1.06 (s, 9H) ppm.

### Intermediate 9: 5-((Benzyloxy)methyl)-4-ethyl-2-(7-methyl-1-oxo-4-(prop-1-en-2-yl)-1H-isochromen-6-yl)-2,4-dihydro-3H-1,2,4-triazol-3-one

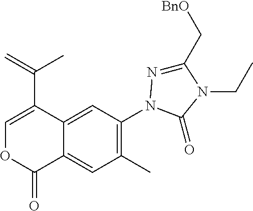 [SMILES: C=C(C)c1coc(=O)c2cc(C)c(-n3nc(COCc4ccccc4)n(CC)c3=O)cc12]

### Step A. tert-Butyl 2-bromo-4-fluoro-5-methylbenzoate

To a solution of 2-bromo-4-fluoro-5-methylbenzoic acid (1 g, 4.3 mmol) in THE (10 mL) was added (Boc)₂O (1.9 g, 8.6 mmol), followed by the addition of DMAP (262 mg, 2.1 mmol). The reaction mixture turned orange and was stirred under nitrogen at 50° C. for overnight. The mixture was cooled to room temperature, diluted with EtOAc, and then washed with brine. The organic layer was separated, dried over Na₂SO₄, filtered and concentrated. The residue was purified by silica column chromatography (elution: 0-3% EtOAc in petroleum ether) to give the title compound as colorless oil (900 mg, yield: 72%). ¹H NMR (400 MHz, CDCl₃) δ 7.57 (d, J=8.1 Hz, 1H), 7.24 (d, J=4.6 Hz, 1H), 2.22 (d, J=1.5 Hz, 3H), 1.58 (s, 9H) ppm.

### Step B. tert-Butyl 4-(3-((benzyloxy)methyl)-4-ethyl-5-oxo-4,5-dihydro-1H-1,2,4-triazol-1-yl)-2-bromo-5-methylbenzoate

A mixture of tert-butyl 2-bromo-4-fluoro-5-methylbenzoate. (750 mg, 2.6 mmol), 5-((benzyloxy)methyl)-4-ethyl-2,4-dihydro-3H-1,2,4-triazol-3-one (800 mg, 3.4 mmol) and Cs₂CO₃ (1.7 g, 5.2 mmol) in DMF (8 mL) was stirred at 90° C. for 16 h. The reaction was quenched by the addition of aqueous saturated NH₄Cl solution. The mixture was extracted with EtOAc. The organic layer was separated, washed with brine, dried over Na₂SO₄, filtered and concentrated. The residue was purified by combi-flash chromatography (SiO₂, eluent: 0-22% EtOAc in petroleum ether) to give the title compound as colorless gum (1 g, yield: 71%). ESI-MS: mass calcd. for C₂₄H₂₈BrN₃O₄, 501.1; m/z found, 502.1 [M+H]⁺. ¹H NMR (400 MHz, CDCl₃) δ 7.64 (d, J=6.1 Hz, 2H), 7.33-7.43 (m, 5H), 4.61 (s, 2H), 4.50 (s, 2H), 3.85 (q, J=7.3 Hz, 2H), 2.31 (s, 3H), 1.62 (s, 9H), 1.36 (t, J=7.2 Hz, 3H) ppm.

### Step C. 4-(3-((Benzyloxy)methyl)-4-ethyl-5-oxo-4,5-dihydro-1H-1,2,4-triazol-1-yl)-2-bromo-5-methylbenzoic acid

To a mixture of tert-butyl 4-(3-((benzyloxy)methyl)-4-ethyl-5-oxo-4,5-dihydro-1H-1,2,4-triazol-1-yl)-2-bromo-5-methylbenzoate (500 mg, 0.90 mmol) in DCM (5 mL) was added TFA (1 mL). The mixture was stirred at room temperature for 12 h. The mixture was concentrated. The residue was dissolved with DCM, and petroleum ether was added slowly. The mixture was stirred at room temperature for 30 min. The mixture was filtered, and the precipitate was rinsed with petroleum ether. The solid was collected and dried in vacuo to give the title compound as a white solid (360 mg, yield: 86%). ESI-MS: mass calcd. for C₂₀H₂₀BrN₃O₄, 445.1; m/z found, 446.0 [M+H]⁺. ¹H NMR (400 MHz, DMSO-d₆) δ 7.77 (s, 1H), 7.70 (s, 1H), 7.29-7.41 (m, 5H), 4.59 (s, 2H), 4.56 (s, 2H), 3.74 (q, J=7.0 Hz, 2H), 2.24 (s, 3H), 1.23 (t, J=7.2 Hz, 3H) ppm.

### Step D. 5-((Benzyloxy)methyl)-4-ethyl-2-(7-methyl-1-oxo-4-(prop-1-en-2-yl)-1H-isochromen-6-yl)-2,4-dihydro-3H-1,2,4-triazol-3-one

To a mixture of 4-(3-((benzyloxy)methyl)-4-ethyl-5-oxo-4,5-dihydro-1H-1,2,4-triazol-1-yl)-2-bromo-5-methylbenzoic acid (560 mg, 1.26 mmol), 3-methylbuta-1,2-dien-1-yl acetate (Intermediate 12, 1.58 g, 12.5 mmol), AcOK (369 mg, 3.76 mmol) and TBAB (809 mg, 2.51 mmol) in DMF (3.9 mL) under nitrogen was added Pd(OAc)₂ (141 mg, 0.63 mmol). The reaction mixture was stirred under nitrogen at 90° C. for overnight. The mixture was cooled to room temperature, diluted with EtOAc, and washed with brine. The organic layer was separated, and the aqueous layer was extracted with EtOAc. The combined organic layers were combined, dried over Na₂SO₄, filtered, and concentrated. The residue was purified by silica column chromatography (gradient elution: 0-70% EtOAc in petroleum ether) to give the title compound as a yellow solid (410 mg, yield: 73%). ESI-MS: mass calcd. for C₂₅H₂₅N₃O₄, 431.2; m/z found, 432.1 [M+H]⁺. ¹H NMR (400 MHz, CDCl₃) δ 8.28 (s, 1H), 7.59 (s, 1H), 7.31-7.46 (m, 5H), 7.17 (s, 1H), 5.30-5.37 (m, 1H), 5.15 (s, 1H), 4.63 (s, 2H), 4.52 (s, 2H), 3.87 (q, J=7.1 Hz, 2H), 2.46 (s, 3H), 2.10 (s, 3H), 1.38 (t, J=7.2 Hz, 3H) ppm.

### Intermediate 10: Isopropyl 6-(3-((benzyloxy)methyl)-4-ethyl-5-oxo-4,5-dihydro-1H-1,2,4-triazol-1-yl)-2-chloro-5-fluoronicotinate

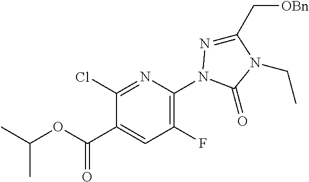 [SMILES: CCn1c(COCc2ccccc2)nn(-c2nc(Cl)c(C(=O)OC(C)C)cc2F)c1=O]

### Step A. 2,6-Dichloro-5-fluoronicotinoyl chloride

To a solution of 2,6-dichloro-5-fluoronicotinic acid (20 g, 95 mmol) in THE (200 mL) was added (COCl)₂ (12.7 g, 10.0 mmol) and DMF (69.6 mg, 0.952 mmol) at 0° C. dropwise. The mixture was stirred at 0° C. for 30 min, then warmed to 25° C., and stirred for 1 h. The reaction mixture was concentrated under reduced pressure to afford desired product (21.7 g, crude) as a colorless oil, which was used without further purification.

### Step B. Isopropyl 2,6-dichloro-5-fluoronicotinate

To a mixture of propan-2-ol (8.56 g, 142 mmol, 10.9 mL) and pyridine (9.02 g, 114 mmol) in THE (200 mL) was added a solution of 2,6-dichloro-5-fluoronicotinoyl chloride (21.7 g, 96.0 mmol) in THE (50 mL) at 0° C. The mixture was stirred at 25° C. for 1 h. The mixture was poured into water (300 mL). The aqueous phase was extracted with ethyl acetate (300 mL). The combined organic phase was dried with anhydrous Na₂SO₄, filtered and concentrated. The residue was purified by column chromatography (SiO₂, Petroleum ether/Ethyl acetate=1/1 to 10:1) to afford the title compound (21 g, 86.82% yield). MS (ESI): mass calcd. for C₉H₈Cl₂FNO₂, 250.1; m/z found, 252.0 [M+H]⁺. ¹H NMR (400 MHz, CDCl₃) δ 7.97-7.95 (d, J=7.2 Hz, 1H), 5.32-5.25 (m, 1H), 1.58-1.39 (m, 6H) ppm.

### Step C. Isopropyl 6-(3-((benzyloxy)methyl)-4-ethyl-5-oxo-4,5-dihydro-1H-1,2,4-triazol-1-yl)-2-chloro-5-fluoronicotinate

To a mixture of isopropyl 2,6-dichloro-5-fluoronicotinate (4 g, 15.87 mmol) in DMSO (40 mL) was added 3-((benzyloxy)methyl)-4-ethyl-1H-1,2,4-triazol-5(4H)-one (3.89 g, 16.66 mmol) and K₂CO₃ (3.29 g, 23.80 mmol). The mixture was stirred at 80° C. for 3 hr. LCMS showed the starting material was consumed and desired mass was detected. The mixture was diluted with H₂O (30 mL) and extracted with EtOAc (50 mL×3). The combined organic layers were washed with brine (100 mL), dried over Na₂SO₄, filtered and concentrated under reduced pressure. The residue was purified by column chromatography (SiO₂, Petroleum ether/Ethyl acetate=I/O to 1:1) to afford the title compound (5.7 g, 79.86% yield). MS (ESI): mass calcd. for C₂₁H₂₂ClFN₄O₄, 448.1; m/z found, 449.2 [M+H]⁺. ¹H NMR (400 MHz, CDCl₃) δ 8.10 (d, J=8.8 Hz, 1H), 7.43-7.31 (m, 5H), 5.30 (td, J=6.3, 12.5 Hz, 1H), 4.61 (s, 2H), 4.54 (s, 2H), 3.85 (q, J=7.2 Hz, 2H), 1.41 (d, J=6.2 Hz, 6H), 1.37-1.31 (m, 3H) ppm.

### Intermediate 11: 5-((Benzyloxy)methyl)-4-ethyl-2-(7-fluoro-3-hydroxy-4-isopropyl-1-oxoisochroman-6-yl)-2,4-dihydro-3H-1,2,4-triazol-3-one

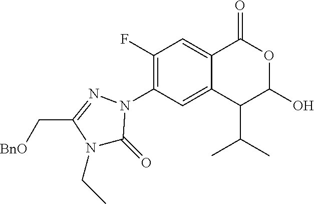 [SMILES: CCn1c(COCc2ccccc2)nn(-c2cc3c(cc2F)C(=O)OC(O)C3C(C)C)c1=O]

### Step A. Methyl 4-(3-((benzyloxy)methyl)-4-ethyl-5-oxo-4,5-dihydro-1H-1,2,4-triazol-1-yl)-2-bromo-5-fluorobenzoate

To a flask charged with methyl 2-bromo-4,5-difluorobenzoate (100.0 g, 398 mmol), 5-((benzyloxy)methyl)-4-ethyl-2,4-dihydro-3H-1,2,4-triazol-3-one (Intermediate 1, 113.5 g, 508 mmol) and K₂CO₃ (100.0 g, 724 mmol) was added anhydrous DMF (1000 mL). The reaction mixture was heated under nitrogen at 50° C. for 16 h, and then additional 5-((benzyloxy)methyl)-4-ethyl-2,4-dihydro-3H-1,2,4-triazol-3-one (11 g, 51 mmol) was added. The reaction mixture continued to be stirred at 50° C. The mixture was cooled to room temperature and stirred for 10 min. Water (1000 mL) was added dropwise, and the mixture was stirred at room temperature for 2 h. The precipitate was collected by filtration and dried to give the crude product (190 g). The product was stirred in DMF (500 mL) for 30 min, then water (500 mL) was added. The mixture was stirred for 2 h. The precipitate was collected by filtration and dried to give the title compound (180 g, yield: 90%). ¹H NMR (300 MHz, CDCl₃) δ 7.95 (d, J=6.7 Hz, 1H), 7.73 (d, J=10.7 Hz, 1H), 7.44-7.27 (m, 5H), 4.60 (s, 2H), 4.50 (s, 2H), 3.95 (s, 3H), 3.84 (q, J=7.2 Hz, 2H), 1.34 (t, J=7.2 Hz, 3H) ppm.

### Step B. 5-((Benzyloxy)methyl)-4-ethyl-2-(7-fluoro-3-hydroxy-4-isopropyl-1-oxoisochroman-6-yl)-2,4-dihydro-3H-1,2,4-triazol-3-one

To a mixture of methyl 4-(3-((benzyloxy)methyl)-4-ethyl-5-oxo-4,5-dihydro-1H-1,2,4-triazol-1-yl)-2-bromo-5-fluorobenzoate (30 g, 65.9 mmol), Xantphos (4.02 g, 6.6 mmol), [Pd(allyl)Cl]₂ (1.38 g, 3.77 mmol), Cs₂CO₃ (42.6 g, 130 mmol) in dimethylacetamide (300 mL) was added 3-methylbutanal (41.4 mL, 386 mmol) slowly. The reaction mixture was heated under nitrogen at 80° C. for 22 h. The mixture was filtered and quenched with aqueous NH₄Cl solution until “pH” turned 7-8. The mixture was extracted with ethyl acetate (2000 mL×2). The combined organic extract was concentrated. The residue was purified by column chromatography (SiO₂, gradient elution: 1-33% ethyl acetate in petroleum ether) to give the title compound as an oil (61.7 g, yield: 56%). ¹H NMR (300 MHz, CDCl₃) δ 7.93 (d, J=10.4 Hz, 1H), 7.55 (d, J=6.7 Hz, 1H), 7.44-7.28 (m, 5H), 5.92 (s, 1H), 4.61 (s, 2H), 4.51 (s, 2H), 4.37 (br s, 1H), 3.85 (q, J=7.2 Hz, 2H), 2.80 (d, J=7.1 Hz, 1H), 1.92 (spt, J=6.8 Hz, 1H), 1.35 (t, J=7.2 Hz, 3H), 1.05 (d, J=6.8 Hz, 3H), 0.93 (d, J=6.8 Hz, 3H) ppm.

### Intermediate 12: 3-Methylbuta-1,2-dien-1-yl acetate

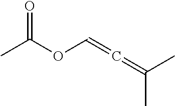

### Step A. 2-Methylbut-3-yn-2-yl acetate

To a mixture of Mg(ClO₄)₂ (796 mg, 3.6 mmol) in acetic anhydride (38 g, 371 mmol) at 0° C. was added 2-methyl-3-butyn-2-ol (30 g, 357 mmol) dropwise. The reaction mixture was stirred at 0° C. for 10 min, then warmed to room temperature and stirred for overnight. The reaction mixture was diluted with DCM, then washed with aqueous saturated NaHCO₃ solution and aqueous saturated Na₂CO₃ solution. The organic layer was separated, dried over Na₂SO₄, filtered and concentrated at 0° C. The residue was purified by silica column chromatography (elution: DCM) to give the title compound as pale yellow oil (35.8 g, yield: 80%). ¹H NMR (400 MHz, CDCl₃) δ 2.54 (s, 1H), 2.03 (s, 3H), 1.67 (s, 6H) ppm.

### Step B. 3-Methylbuta-1,2-dien-1-yl acetate

To a solution of 2-methylbut-3-yn-2-yl acetate (2.5 g, 20 mmol) in DCM (20 mL) was added AgBF₄ (117 mg, 0.6 mmol) under nitrogen. The resulting colorless solution was stirred under nitrogen at 35° C. for 2 h until the mixture turned into a black solution. The mixture was washed with aqueous ammonia (10%). The organic layer was separated, and the aqueous layer was extracted with DCM. The combined organic layer was dried over Na₂SO₄, filtered and concentrated. The residue was purified by silica column chromatography (gradient elution: 0-3% EtOAc in petroleum ether) to give the title compound as yellow oil (650 mg, yield: 26%). ¹H NMR (400 MHz, CDCl₃) δ 7.20 (dt, J=4.1, 2.0 Hz, 1H), 2.11 (s, 3H), 1.81 (d, J=2.0 Hz, 6H) ppm.

### Example 22 of PCT/IB2020/053601 (which Published as WO 2020/212897 on Oct. 22, 2020) Discloses 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one (Compound 22)

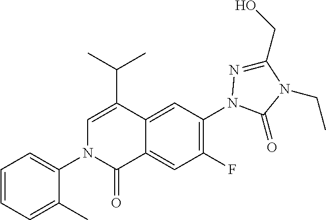 [SMILES: CCn1c(CO)nn(-c2cc3c(C(C)C)cn(-c4ccccc4C)c(=O)c3cc2F)c1=O]

### Step A. 6-(3-((Benzyloxy)methyl)-4-ethyl-5-oxo-4,5-dihydro-1H-1,2,4-triazol-1-yl)-7-fluoro-4-isopropyl-2-(o-tolyl)isoquinolin-1(2H)-one

To a mixture of 5-((benzyloxy)methyl)-4-ethyl-2-(7-fluoro-3-hydroxy-4-isopropyl-1-oxoisochroman-6-yl)-2,4-dihydro-3H-1,2,4-triazol-3-one (Intermediate 11, 56 g, 123 mmol) in AcOH (160 mL) was added o-toluidine (14.8 g, 138 mmol). The reaction mixture was heated at 80° C. for 16 h. The mixture was concentrated, and then the “pH” was adjusted to 7-8 with aqueous NaHCO₃ solution. The mixture was extracted with ethyl acetate (160 mL×2). The combined organic extract was concentrated. The residue was purified by flash chromatography (SiO₂, 0-20% Ethyl acetate in DCM) to give the title compound as an oil (32.5 g, yield: 50%). MS (ESI): mass calcd. for C₃₁H₃₁FN₄O₃, 526.2; m/z found, 527.4 [M+H]⁺. ¹H NMR (300 MHz, CDCl₃) δ 8.33 (d, J=10.9 Hz, 1H), 8.07 (d, J=6.8 Hz, 1H), 7.44-7.27 (m, 9H), 6.84 (s, 1H), 4.64 (s, 2H), 4.55 (s, 2H), 3.89 (q, J=7.2 Hz, 2H), 3.23 (spt, J=6.8 Hz, 1H), 2.16 (s, 3H), 1.39 (t, J=7.2 Hz, 3H), 1.31 (dd, J=6.8, 2.1 Hz, 6H) ppm.

### Step B. 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one

To a stirred solution of 6-(3-((benzyloxy)methyl)-4-ethyl-5-oxo-4,5-dihydro-1H-1,2,4-triazol-1-yl)-7-fluoro-4-isopropyl-2-(o-tolyl)isoquinolin-1(2H)-one (26.5 g, 50.3 mmol) in DCM (230 mL) at −78° C. was added a DCM solution (1 M) of BCl₃ (290 mL, 290 mL) under nitrogen. The reaction mixture was stirred at 15° C. for 0.5 h. The reaction was quenched by MeOH (100 mL) at −78° C. to −20° C. The mixture was partitioned between water and DCM. The organic layer was separated, and the aqueous layer was extracted with DCM (110 mL×2). The combined organic extract was washed with brine (30 mL×2), dried with anhydrous Na₂SO₄, filtered, and concentrated to give a crude product (27.5 g). The product was triturated with methyl ethyl ketone (82 mL) and heptane (290 mL) to give a pure product (17.5 g), which was re-crystallized in ethanol and water to give the title compound as a white solid (16 g, yield: 73%). MS (ESI): mass calcd. for C₂₄H₂₅FN₄O₃, 436.2; m/z found, 437.2 [M+H]⁺. ¹H NMR (400 MHz, CDCl₃) δ 8.33 (d, J=11.2, 1H), 8.08 (d, J=6.8, 1H), 7.39-7.33 (m, 3H), 7.28 (s, 1H), 6.85 (s, 1H), 4.69 (br s, 2H), 3.94 (q, J=7.11 Hz, 2H), 3.27 (td, J=13.66, 6.82 Hz, 1H), 2.32 (br s, 1H), 2.17 (s, 3H), 1.45 (t, J=7.11 Hz, 3H), 1.32 (dd, J=6.82, 1.83 Hz, 6H) ppm.

It is noted that compounds of Formula (Z) described herein are described in PCT/IB2020/053601 (which published as WO 2020/212897 on Oct. 22, 2020), which is incorporated by reference herein, in its entirety for all purposes.

In some embodiments, provided is a combination comprising a menin-MLL inhibitor of Formula (I), or a pharmaceutically acceptable salt or a solvate thereof, and at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor.

In some embodiments, provided is a combination comprising a menin-MLL inhibitor of Formula (I), or a pharmaceutically acceptable salt or a solvate thereof, and at least one other therapeutic agent, wherein at least one other therapeutic agent is a hypomethylating agent.

According to embodiments, the hypomethylating agent is azacitidine, decitabine, or pharmaceutically acceptable salts or solvates thereof.

According to particular embodiments, the hypomethylating agent is azacitidine, or a pharmaceutically acceptable salt or solvate thereof.

In some embodiments, provided is a combination comprising a menin-MLL inhibitor of In some embodiments, provided is a combination comprising a menin-MLL inhibitor of Formula (I), or a pharmaceutically acceptable salt or a solvate thereof, and at least one other therapeutic agent is azacitidine, or a pharmaceutically acceptable salt or solvate thereof.

In some embodiments, provided is a combination comprising Compound 51 or a pharmaceutically acceptable salt or solvate thereof, and azacitidine, or a pharmaceutically acceptable salt or solvate thereof.

In some embodiments, provided is a combination comprising Compound 51a, and azacitidine, or a pharmaceutically acceptable salt or solvate thereof.

In some embodiments, provided is a combination comprising a menin-MLL inhibitor of Formula (I), or a pharmaceutically acceptable salt or a solvate thereof, and at least one other therapeutic agent is decitabine, or a pharmaceutically acceptable salt or solvate thereof.

In some embodiments, provided is a combination comprising Compound 51 or a pharmaceutically acceptable salt or solvate thereof, and at least one other therapeutic agent is decitabine, or a pharmaceutically acceptable salt or solvate thereof.

In some embodiments, provided is a combination comprising Compound 51a, and at least one other therapeutic agent is decitabine, or a pharmaceutically acceptable salt or solvate thereof.

In some embodiments, provided is a combination comprising a menin-MLL inhibitor of Formula (I), or a pharmaceutically acceptable salt or a solvate thereof, and at least one other therapeutic agent is a DNA intercalating agent.

According to embodiments, the DNA intercalating agent is an anthracycline.

In some embodiments, provided is a combination comprising a menin-MLL inhibitor of Formula (I), or a pharmaceutically acceptable salt or a solvate thereof, and at least one other therapeutic agent is an anthracycline.

In some embodiments, provided is a combination comprising Compound 51 or a pharmaceutically acceptable salt or solvate thereof, and at least one other therapeutic agent is an anthracycline.

In some embodiments, provided is a combination comprising Compound 51a, and at least one other therapeutic agent is an anthracycline.

In some embodiments, provided is a combination comprising a menin-MLL inhibitor of Formula (I), or a pharmaceutically acceptable salt or a solvate thereof, and at least one other therapeutic agent, wherein at least one other therapeutic agent is a pyrimidine analog.

According to embodiments, the pyrimidine analog is cytarabine.

In some embodiments, provided is a combination comprising a menin-MLL inhibitor of Formula (I), or a pharmaceutically acceptable salt or a solvate thereof, and at least one other therapeutic agent is cytarabine.

In some embodiments, provided is a combination comprising Compound 51 or a pharmaceutically acceptable salt or solvate thereof, and at least one other therapeutic agent is cytarabine.

In some embodiments, provided is a combination comprising Compound 51a, and at least one other therapeutic agent is cytarabine.

In some embodiments, provided is a combination comprising a menin-MLL inhibitor of Formula (I), or a pharmaceutically acceptable salt or a solvate thereof, and at least one other therapeutic agent, wherein at least one other therapeutic agent is a purine analog.

According to embodiments, the purine analog is fludarabine.

In some embodiments, provided is a combination comprising a menin-MLL inhibitor of Formula (I), or a pharmaceutically acceptable salt or a solvate thereof, and at least one other therapeutic agent is fludarabine.

In some embodiments, provided is a combination comprising Compound 51 or a pharmaceutically acceptable salt or solvate thereof, and at least one other therapeutic agent is fludarabine.

In some embodiments, provided is a combination comprising Compound 51a, and at least one other therapeutic agent is fludarabine.

In some embodiments, provided is a combination comprising a menin-MLL inhibitor of Formula (I), or a pharmaceutically acceptable salt or a solvate thereof, and at least one other therapeutic agent, wherein at least one other therapeutic agent is an IDH inhibitor.

In some embodiments, the IDH inhibitor is an isocitrate dehydrogenase-1 inhibitor (e.g., ivosidenib).

According to embodiments, the IDH inhibitor is ivosidenib.

In some embodiments, provided is a combination comprising a menin-MLL inhibitor of Formula (I), or a pharmaceutically acceptable salt or a solvate thereof, and at least one other therapeutic agent is ivosidenib.

In some embodiments, provided is a combination comprising Compound 51 or a pharmaceutically acceptable salt or solvate thereof, and at least one other therapeutic agent is ivosidenib.

In some embodiments, provided is a combination comprising Compound 51a, and at least one other therapeutic agent is ivosidenib.

In some embodiments, the IDH is an isocitrate dehydrogenase-2 inhibitor (e.g., enasidenib).

According to embodiments, the IDH inhibitor is enasidenib.

In some embodiments, provided is a combination comprising a menin-MLL inhibitor of Formula (I), or a pharmaceutically acceptable salt or a solvate thereof, and at least one other therapeutic agent is enasidenib.

In some embodiments, provided is a combination comprising Compound 51 or a pharmaceutically acceptable salt or solvate thereof, and at least one other therapeutic agent is enasidenib.

In some embodiments, provided is a combination comprising Compound 51a, and at least one other therapeutic agent is enasidenib.

In some embodiments, provided is a combination comprising a menin-MLL inhibitor of Formula (I), or a pharmaceutically acceptable salt or a solvate thereof, and at least one other therapeutic agent, wherein at least one other therapeutic agent is an immunomodulatory agent.

In some embodiments, provided is a combination comprising a menin-MLL inhibitor of Formula (I), or a pharmaceutically acceptable salt or a solvate thereof, and at least one other therapeutic agent, wherein at least one other therapeutic agent is an immunomodulatory agent which is a PD-1 inhibitor.

According to embodiments, the immunomodulatory agent is nivolumab, atezolizumab, pembrolizumab, thalidomide, lenalidomide, pomalidomide, Bacillus Calmette-Guérin (BCG) or levamisole.

In some embodiments, provided is a combination comprising a menin-MLL inhibitor of Formula (I), or a pharmaceutically acceptable salt or a solvate thereof, and at least one other therapeutic agent is nivolumab.

In some embodiments, provided is a combination comprising Compound 51 or a pharmaceutically acceptable salt or solvate thereof, and at least one other therapeutic agent is nivolumab.

In some embodiments, provided is a combination comprising Compound 51a, and at least one other therapeutic agent is nivolumab.

In some embodiments, provided is a combination comprising a menin-MLL inhibitor of Formula (I), or a pharmaceutically acceptable salt or a solvate thereof, and at least one other therapeutic agent is atezolizumab.

In some embodiments, provided is a combination comprising Compound 51 or a pharmaceutically acceptable salt or solvate thereof, and at least one other therapeutic agent is atezolizumab.

In some embodiments, provided is a combination comprising Compound 51a, and at least one other therapeutic agent is atezolizumab.

In some embodiments, provided is a combination comprising a menin-MLL inhibitor of Formula (I), or a pharmaceutically acceptable salt or a solvate thereof, and at least one other therapeutic agent is pembrolizumab.

In some embodiments, provided is a combination comprising Compound 51 or a pharmaceutically acceptable salt or solvate thereof, and at least one other therapeutic agent is pembrolizumab.

In some embodiments, provided is a combination comprising Compound 51a, and at least one other therapeutic agent is pembrolizumab.

In some embodiments, provided is a combination comprising a menin-MLL inhibitor of Formula (I), or a pharmaceutically acceptable salt or a solvate thereof, and at least one other therapeutic agent is thalidomide.

In some embodiments, provided is a combination comprising Compound 51 or a pharmaceutically acceptable salt or solvate thereof, and at least one other therapeutic agent is thalidomide.

In some embodiments, provided is a combination comprising Compound 51a, and at least one other therapeutic agent is thalidomide.

In some embodiments, provided is a combination comprising a menin-MLL inhibitor of Formula (I), or a pharmaceutically acceptable salt or a solvate thereof, and at least one other therapeutic agent is lenalidomide.

In some embodiments, provided is a combination comprising Compound 51 or a pharmaceutically acceptable salt or solvate thereof, and at least one other therapeutic agent is lenalidomide.

In some embodiments, provided is a combination comprising Compound 51a, and at least one other therapeutic agent is lenalidomide.

In some embodiments, provided is a combination comprising a menin-MLL inhibitor of Formula (I), or a pharmaceutically acceptable salt or a solvate thereof, and at least one other therapeutic agent is pomalidomide.

In some embodiments, provided is a combination comprising Compound 51 or a pharmaceutically acceptable salt or solvate thereof, and at least one other therapeutic agent is pomalidomide.

In some embodiments, provided is a combination comprising Compound 51a, and at least one other therapeutic agent is pomalidomide.

In some embodiments, provided is a combination comprising a menin-MLL inhibitor of Formula (I), or a pharmaceutically acceptable salt or a solvate thereof, and at least one other therapeutic agent is BCG.

In some embodiments, provided is a combination comprising Compound 51 or a pharmaceutically acceptable salt or solvate thereof, and at least one other therapeutic agent is BCG.

In some embodiments, provided is a combination comprising Compound 51a, and at least one other therapeutic agent is BCG.

In some embodiments, provided is a combination comprising a menin-MLL inhibitor of Formula (I), or a pharmaceutically acceptable salt or a solvate thereof, and at least one other therapeutic agent is levamisole.

In some embodiments, provided is a combination comprising Compound 51 or a pharmaceutically acceptable salt or solvate thereof, and at least one other therapeutic agent is levamisole.

In some embodiments, provided is a combination comprising Compound 51a, and at least one other therapeutic agent is levamisole.

In some embodiments, provided is a combination comprising a menin-MLL inhibitor of Formula (I), or a pharmaceutically acceptable salt or a solvate thereof, and at least one other therapeutic agent, wherein at least one other therapeutic agent is a DHODH inhibitor.

In some embodiments, provided is a combination comprising a menin-MLL inhibitor of Formula (I), or a pharmaceutically acceptable salt or a solvate thereof, and at least one other therapeutic agent, wherein at least one other therapeutic agent is a DHODH inhibitor compound as described herein.

According to embodiments, the DHODH inhibitor is a compound of Formula (Z), or a pharmaceutically acceptable salt, isotope, N-oxide, solvate, or stereoisomer thereof.

In some embodiments, provided is a combination comprising a menin-MLL inhibitor of Formula (I), or a pharmaceutically acceptable salt or a solvate thereof, and at least one other therapeutic agent is a DHODH inhibitor of Formula (Z), or a pharmaceutically acceptable salt, isotope, N-oxide, solvate, or stereoisomer thereof.

In some embodiments, provided is a combination comprising Compound 51 or a pharmaceutically acceptable salt or solvate thereof, and at least one other therapeutic agent is a DHODH inhibitor of Formula (Z), or a pharmaceutically acceptable salt, isotope, N-oxide, solvate, or stereoisomer thereof.

In some embodiments, provided is a combination comprising Compound 51a, and at least one other therapeutic agent is a DHODH inhibitor of Formula (Z), or a pharmaceutically acceptable salt, isotope, N-oxide, solvate, or stereoisomer thereof.

In some embodiments, provided is a combination comprising a menin-MLL inhibitor of Formula (I), or a pharmaceutically acceptable salt or a solvate thereof, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination comprising Compound A or a pharmaceutically acceptable salt or solvate thereof, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination comprising Compound 51, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination comprising Compound 51a, and at least one other therapeutic agent is a DHODH inhibitor that is Compound 22 or a pharmaceutically acceptable salt, solvate, stereoisomer, isotopic variant, or N-oxide thereof (e.g., 6-(4-Ethyl-3-(hydroxymethyl)-5-oxo-4,5-dihydro-1H-1,2,4-triazol-1-yl)-7-fluoro-4-isopropyl-2-(o-tolyl)isoquinolin-1(2H)-one).

In some embodiments, provided is a combination comprising a menin-MLL inhibitor of Formula (I), or a pharmaceutically acceptable salt or a solvate thereof, and at least one other therapeutic agent, wherein at least one other therapeutic agent is a kinase inhibitor.

According to embodiments, the kinase inhibitor is a serine and/or tyrosine kinase inhibitor.

According to embodiments, the kinase inhibitor is an inhibitor of FLT3 and/or BTK.

In some embodiments, provided is a combination comprising a menin-MLL inhibitor of Formula (I), or a pharmaceutically acceptable salt or a solvate thereof, and at least one other therapeutic agent, wherein at least one other therapeutic agent is a FLT3 inhibitor.

According to embodiments, the FLT3 inhibitor is sorafenib, sunitinib, midostaurin (PKC412), lestaurtinib (CEP-701), tandutinib (MLN518), quizartinib (AC220), gilteritinib (ASP2215), or KW-2449.

In some embodiments, provided is a combination comprising a menin-MLL inhibitor of Formula (I), or a pharmaceutically acceptable salt or a solvate thereof, and at least one other therapeutic agent is sorafenib.

In some embodiments, provided is a combination comprising Compound 51 or a pharmaceutically acceptable salt or solvate thereof, and at least one other therapeutic agent is sorafenib.

In some embodiments, provided is a combination comprising Compound 51a, and at least one other therapeutic agent is sorafenib.

In some embodiments, provided is a combination comprising a menin-MLL inhibitor of Formula (I), or a pharmaceutically acceptable salt or a solvate thereof, and at least one other therapeutic agent is sunitinib.

In some embodiments, provided is a combination comprising Compound 51 or a pharmaceutically acceptable salt or solvate thereof, and at least one other therapeutic agent is sunitinib.

In some embodiments, provided is a combination comprising Compound 51a, and at least one other therapeutic agent is sunitinib.

In some embodiments, provided is a combination comprising a menin-MLL inhibitor of Formula (I), or a pharmaceutically acceptable salt or a solvate thereof, and at least one other therapeutic agent is midostaurin.

In some embodiments, provided is a combination comprising Compound 51 or a pharmaceutically acceptable salt or solvate thereof, and at least one other therapeutic agent is midostaurin.

In some embodiments, provided is a combination comprising Compound 51a, and at least one other therapeutic agent is midostaurin.

In some embodiments, provided is a combination comprising a menin-MLL inhibitor of Formula (I), or a pharmaceutically acceptable salt or a solvate thereof, and at least one other therapeutic agent is lestaurtinib.

In some embodiments, provided is a combination comprising Compound 51 or a pharmaceutically acceptable salt or solvate thereof, and at least one other therapeutic agent is lestaurtinib.

In some embodiments, provided is a combination comprising Compound 51a, and at least one other therapeutic agent is lestaurtinib.

In some embodiments, provided is a combination comprising a menin-MLL inhibitor of Formula (I), or a pharmaceutically acceptable salt or a solvate thereof, and at least one other therapeutic agent is tandutinib.

In some embodiments, provided is a combination comprising Compound 51 or a pharmaceutically acceptable salt or solvate thereof, and at least one other therapeutic agent is tandutinib.

In some embodiments, provided is a combination comprising Compound 51a, and at least one other therapeutic agent is tandutinib.

In some embodiments, provided is a combination comprising a menin-MLL inhibitor of Formula (I), or a pharmaceutically acceptable salt or a solvate thereof, and at least one other therapeutic agent is AC220.

In some embodiments, provided is a combination comprising Compound 51 or a pharmaceutically acceptable salt or solvate thereof, and at least one other therapeutic agent is AC220.

In some embodiments, provided is a combination comprising Compound 51a, and at least one other therapeutic agent is AC220.

In some embodiments, provided is a combination comprising a menin-MLL inhibitor of Formula (I), or a pharmaceutically acceptable salt or a solvate thereof, and at least one other therapeutic agent is ASP2215.

In some embodiments, provided is a combination comprising Compound 51 or a pharmaceutically acceptable salt or solvate thereof, and at least one other therapeutic agent is ASP2215.

In some embodiments, provided is a combination comprising Compound 51a, and at least one other therapeutic agent is ASP2215.

In some embodiments, provided is a combination comprising a menin-MLL inhibitor of Formula (I), or a pharmaceutically acceptable salt or a solvate thereof, and at least one other therapeutic agent is KW-2449.

In some embodiments, provided is a combination comprising Compound 51 or a pharmaceutically acceptable salt or solvate thereof, and at least one other therapeutic agent is KW-2449.

In some embodiments, provided is a combination comprising Compound 51a, and at least one other therapeutic agent is KW-2449.

In some embodiments, provided is a combination comprising a menin-MLL inhibitor of Formula (I), or a pharmaceutically acceptable salt or a solvate thereof, and at least one other therapeutic agent, wherein at least one other therapeutic agent is a BTK inhibitor.

In some embodiments, provided is a combination comprising Compound 51 or a pharmaceutically acceptable salt or solvate thereof, and at least one other therapeutic agent is a BTK inhibitor.

In some embodiments, provided is a combination comprising Compound 51a, and at least one other therapeutic agent is a BTK inhibitor.

According to embodiments, the BTK inhibitor is ibrutinib.

In some embodiments, provided is a combination comprising a menin-MLL inhibitor of Formula (I), or a pharmaceutically acceptable salt or a solvate thereof, and at least one other therapeutic agent is ibrutinib.

In some embodiments, provided is a combination comprising Compound 51 or a pharmaceutically acceptable salt or solvate thereof, and at least one other therapeutic agent is ibrutinib.

In some embodiments, provided is a combination comprising Compound 51a, and at least one other therapeutic agent is ibrutinib.

In some embodiments, provided is a combination comprising a menin-MLL inhibitor of Formula (I), or a pharmaceutically acceptable salt or a solvate thereof, and at least one other therapeutic agent, wherein at least one other therapeutic agent is a CD20 inhibitor.

According to embodiments, the CD20 inhibitor is an anti-CD20 antibody, in particular obinutuzumab (GA101).

In some embodiments, provided is a combination comprising Compound 51 or a pharmaceutically acceptable salt or solvate thereof, and at least one other therapeutic agent is a CD20 inhibitor.

In some embodiments, provided is a combination comprising Compound 51a, and at least one other therapeutic agent is a CD20 inhibitor.

In some embodiments, provided is a combination comprising a menin-MLL inhibitor of Formula (I), or a pharmaceutically acceptable salt or a solvate thereof, and at least one other therapeutic agent is GA101.

In some embodiments, provided is a combination comprising Compound 51 or a pharmaceutically acceptable salt or solvate thereof, and at least one other therapeutic agent is GA101.

In some embodiments, provided is a combination comprising Compound 51a, and at least one other therapeutic agent is GA101.

All possible combinations of the above indicated embodiments are considered to be embraced within the scope of the invention In some embodiments, provided are methods for treating a subject who has been diagnosed with a hematopoietic disorder. The present invention relates, for example, to novel methods that comprise administering to the subject a therapeutically effective amount of a menin-MLL inhibitor as described herein; and a therapeutically effective amount of at least one other therapeutic agent.

An additional embodiment of the invention relates to methods as described herein wherein the compound of Formula (I), or a pharmaceutically acceptable salt or a solvate thereof is administered orally to a subject.

One skilled in the art will recognize that a therapeutically effective amount of the compound of Formula (I), or a pharmaceutically acceptable salt or a solvate thereof, is the amount sufficient to have therapeutic activity and that this amount varies inter alias, depending on the type of disease, the concentration of the compound in the therapeutic formulation, and the condition of the patient. An effective therapeutic daily amount would be from about 0.005 mg/kg to 100 mg/kg. The amount of a compound according to the present invention, also referred to herein as the active ingredient, which is required to achieve a therapeutically effect may vary on case-by-case basis, for example with the particular compound, the route of administration, the age and condition of the recipient, and the particular disorder or disease being treated. A method of treatment may also include administering the active ingredient on a regimen of between one and four intakes per day. In these methods of treatment the compounds according to the invention are preferably formulated prior to administration.

An additional embodiment of the invention relates to methods as described herein wherein the at least one other therapeutic agent is administered orally to a subject.

An additional embodiment of the invention relates to methods as described herein wherein the at least one other therapeutic agent is administered orally in a dose of from about 1 mg to about 500 mg to the subject.

An additional embodiment of the invention relates to methods as described herein wherein at least one other therapeutic agent is administered to the subject intravenously or subcutaneously.

An additional embodiment of the invention relates to methods for treating a subject who has been diagnosed with cancer (e.g., wherein the cancer is a hematopoietic disorder, such as myelodysplastic syndrome (MDS), acute myeloid leukemia (AML), acute lymphoblastic leukemia (ALL), a small lymphocytic lymphoma (SLL) or chronic lymphocytic leukemia (CLL)), wherein the method comprises administering to the subject:
a therapeutically effective amount of a menin-MLL inhibitor of Formula (I), or a tautomer or a stereoisomeric form thereof, or a pharmaceutically acceptable salt or a solvate thereof; and
a therapeutically effective amount of a DHODH inhibitor of Formula (Z), or a pharmaceutically acceptable salt, isotope, N-oxide, solvate, or stereoisomer thereof.

Optimal dosages of any of the therapeutic compounds described herein to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect.

The therapeutic compounds described herein may be administered to the subject simultaneously or sequentially. When administered sequentially, the menin-MLL inhibitor of Formula (I) may be administered first. When administration is simultaneous, the combination may be administered either in the same or a different pharmaceutical composition. For instance, the menin-MLL inhibitor of Formula (I) may be administered prior to, simultaneous with, or after the administration of at least one other therapeutic agent. Likewise, at least one other therapeutic agent may be administered prior to, simultaneous with, or after the administration of another therapeutic agent. Adjunctive therapy, i.e., where one or two agent(s) are used as the primary treatment and the other agent is used to assist that primary treatment, is also an embodiment of the present invention.

An embodiment of the invention relates to a therapeutically effective amount of a menin-MLL inhibitor including the compounds of Formula (I) and the pharmaceutically acceptable salts, and the solvates thereof, or any subgroup thereof as mentioned in any of the embodiments, for use in combination with a therapeutically effective amount of at least one other therapeutic agent, wherein the at least one other therapeutic agent is a hypomethylating agent, cytidine deaminase inhibitor, a DNA intercalating agent, a pyrimidine analog, a purine analog, a kinase inhibitor, a CD20 inhibitor, an isocitrate dehydrogenase inhibitor, an immunomodulatory agent or a dihydroorotate dehydrogenase inhibitor. In certain embodiments, the therapeutic compounds described herein are administered in separate dosage forms for use in treating a subject who has been diagnosed with a hematopoietic disorder.

An embodiment of the invention relates to a pharmaceutical product including a menin-MLL inhibitor of Formula (I) and the pharmaceutically acceptable salts, and the solvates thereof, or any subgroup thereof as mentioned in any of the embodiments, and at least one other therapeutic agent is a hypomethylating agent, cytidine deaminase inhibitor, a DNA intercalating agent, a pyrimidine analog, a purine analog, a kinase inhibitor, a CD20 inhibitor, an isocitrate dehydrogenase inhibitor, an immunomodulatory agent or a dihydroorotate dehydrogenase inhibitor as a combined preparation for simultaneous, separate or sequential use in treating a subject who has been diagnosed with a hematopoietic disorder.

The following Examples are provided to illustrate some of the concepts described within this disclosure. While the Example is considered to provide an embodiment, it should not be considered to limit the more general embodiments described herein.

### EXAMPLES

### General Synthetic Schemes

### Compounds of Formula I

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups (PG) can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

### General Synthetic Schemes

All abbreviations used in the general schemes are as defined below or as in the Table in the part Examples. Variables are as defined in the scope or as specifically defined in the general Schemes. Where compounds/intermediates in the schemes below contain a double bond, the substituents may be in the E or the Z configuration or be mixtures thereof.

### Scheme 1

In general, compounds of Formula (I-aa), can be prepared according to the following reaction Scheme 1. In Scheme 1, PG represents a suitable protecting group, such as for example tert-butyloxycarbonyl, 9-fluorenylmethoxycarbonyl, or benzyl, and LG is a leaving group such as for example chloro, bromo, iodo or tosylate or mesylate or triflate; all other variables are defined according to the scope of the present invention.

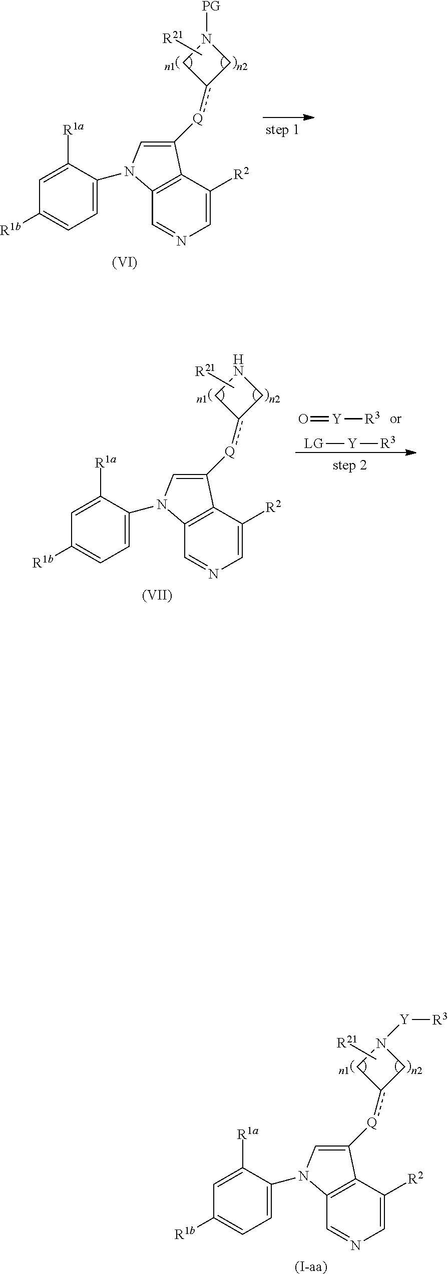

In Scheme 1, the following reaction conditions apply:
Step 1: when PG=Boc, at a suitable temperature in a range between 0° C. and 40° C., such as room temperature, in the presence of a suitable acid, for example a protic acid such as trifluoroacetic acid (TFA) or hydrochloric acid, in a suitable solvent such as dichloromethane (DCM) or 1,4-dioxane;
Alternatively, when PG=9-fluorenylmethoxycarbonyl, at a suitable temperature in a range between 0° C. and 40° C., such as room temperature, in the presence of a suitable base such as piperidine, in a suitable solvent such as dichloromethane (DCM);
Alternatively, when PG=benzyl, at a suitable temperature such as room temperature, in the presence of a suitable heterogenous catalyst such as palladium on charcoal (Pd/C), in a common solvent such as methanol, ethanol, THE or the like under hydrogen pressure such as for example from 1 to 3 bar, optionally in the presence of a base such as triethylamine;
Step 2:
In the case of a reductive amination reaction employing an aldehyde or a ketone: at a suitable temperature in a range between room temperature and 70° C., in the presence of a suitable reducing agent such as for example sodium triacetoxyborohydride or sodium cyanoborohydride, in a suitable solvent such as for example methanol, dichloromethane or 1,2-dichloroethane, optionally in the presence of zinc chloride or sodium acetate or acetic acid;
In the case of an alkylation reaction employing LG-Y—R³: at a suitable temperature such as for example room temperature, in the presence of a suitable deprotonating agent such as for example sodium hydride or potassium carbonate, or an amine base such as triethylamine in a suitable aprotic solvent such as for example dimethylformamide or dimethylsulfoxide or acetonitrile.

### Scheme 2

In general, compounds of Formula (I) wherein Q is limited to —O—, —NR^{q}—, can be prepared via intermediates of Formula (VIc). Intermediates of Formula (VIc) can be prepared according to the following reaction Scheme 2. In Scheme 2, PG represents a suitable protecting group, such as for example tert-butyloxycarbonyl; all other variables are defined according to the scope of the present invention.

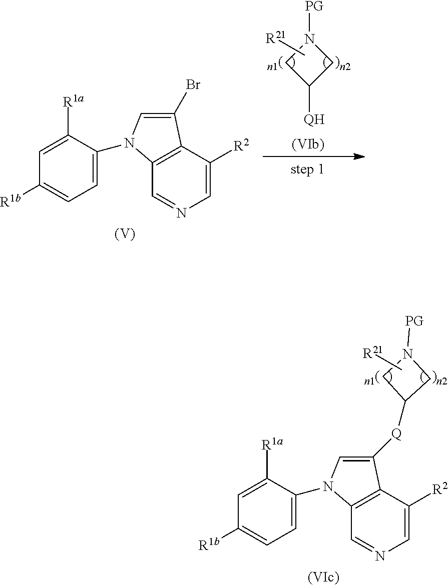

In Scheme 2, the following reaction conditions apply:
Step 1: at a suitable temperature in a range between 100° C. and 140° C., in the presence of a suitable base such as for example potassium tert-butoxide or potassium phosphate, in the presence of a suitable catalyst such as palladium acetate (Pd(OAc)₂) or tris(dibenzylideneacetone)dipalladium(0) (Pd₂dba₃) or [1,1′-bis(diphenylphosphino)ferrocene]dichloropalladium(II) (Pd(dppf)Cl₂), in the presence of a suitable ligand such as 9,9-dimethyl-4,5-bis(diphenylphosphino)xanthene (Xantphos), in a suitable solvent such as for example dioxane or dimethylformamide.

### Scheme 2B

In general, intermediates of Formula (V) can be prepared according to the following reaction Scheme 2B. In Scheme 2B, W¹ represents fluoro, chloro, bromo or iodo, BPin represents 4,4,5,5-tetramethyl-1,3,2-dioxaborolane, and all other variables are defined according to the scope of the present invention.

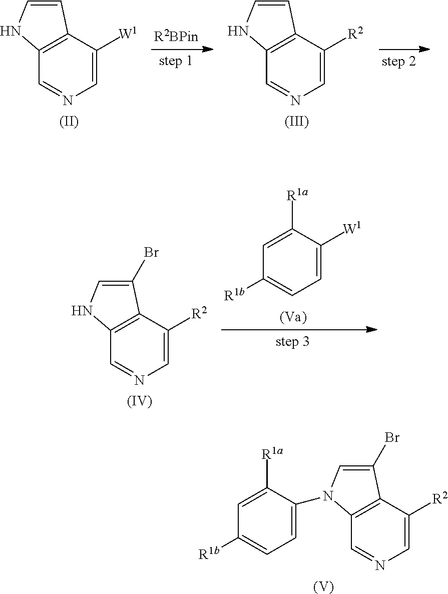

In Scheme 2B, the following reaction conditions apply:
Step 1: at a suitable temperature in a range between room temperature and 100° C., in the presence of a suitable base such as for example potassium carbonate, in the presence of a suitable catalyst such as [1,1′-Bis(diphenylphosphino)ferrocene]dichloropalladium(II) (Pd(dppf)Cl₂) in a suitable solvent such as for example dioxane or dimethylformamide and water; Alternatively, when R²=Me, a boron containing reagent such as trimethyl boroxine can be used in the presence of a suitable catalyst such as (Pd(dppf)Cl₂) in a suitable solvent such as for example dioxane or dimethylformamide and water in the presence of an inorganic base such as potassium carbonate at a reaction temperature between 80° C. and 120° C.;
Additional step to achieve the double bond reduction to obtain R² is C₃₋₆cycloalkyl, C₁₋₄alkyl, or C₁₋₄alkyl substituted with one, two or three halo substituents: at a suitable temperature such as room temperature, in the presence of a suitable catalyst such as palladium on charcoal (Pd/C), in a suitable solvent such as methanol, under H₂ pressure such as for example from 1 to 3 bar, optionally in the presence of a base such as triethylamine;
Step 2: at a suitable temperature such as for example between 0° C. and room temperature, in the presence of a suitable bromination reagent such as for example N-Bromosuccinimide or CuBr₂, in a suitable solvent such as for example dimethylformamide or acetonitrile;
Step 3: at a suitable temperature such as for example 80° C. and 130° C., in the presence of a suitable catalyst such as copper (Cu), in the presence of a base such as potassium carbonate, in a suitable solvent such as dimethylformamide; Alternatively a copper (I) source may be used, such as CuI in the presence of a suitable diamine ligand, such as trans-N,N′-dimethylcyclohexane-1,2-diamine in the presence of an inorganic base, such as potassium carbonate in an aprotic solvent such as dimethylformamide at a temperature between 80° C. and 150° C. In certain cases said conversion may also be effected by a nucleophilic aromatic substitution using an inorganic base such as potassium tert-butoxide or sodium hydride or the like, in an aprotic solvent such as dimethylformamide at a temperature between 0° C. and 80° C.;
Someone skilled in the art will appreciate that the steps 2 and 3 in Scheme 2B may also be reversed, i.e. first the cross coupling of intermediate (III) with the reagent (Va), followed by bromination of the aza indole moiety to provide the intermediate (V).

### Scheme 3

In general, intermediates of Formula (VIIa), can be prepared via intermediates of Formula (VIe). Intermediates of Formula (VIe) can also be prepared according to the following reaction Scheme 3. In Scheme 3, PG represents a suitable protecting group, such as for example tert-butyloxycarbonyl; all other variables are defined according to the scope of the present invention.

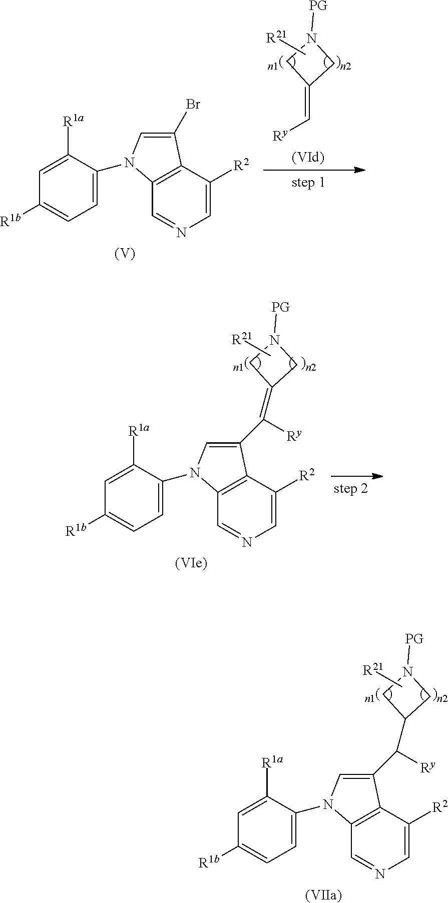

In Scheme 3, the following reaction conditions apply:
Step 1: at a suitable temperature in a range between 70° C. and 100° C., in the presence of a suitable base such as for example potassium phosphate, in presence of a suitable catalyst such as palladium acetate (Pd(OAc)₂), optionally in the presence of a suitable phosphine ligand such as 2-dicyclohexylphosphino-2′-(N,N-dimethylamino)biphenyl (Davephos), in a suitable solvent such as for example dioxane or dimethylformamide;
Step 2: at a suitable temperature such as room temperature, in the presence of a suitable heterogenous catalyst such as palladium on charcoal (Pd/C), in a common solvent such as methanol, ethanol, THE or the like under hydrogen pressure such as for example from 1 to 3 bar, optionally in the presence of a base such as triethylamine;

### Scheme 4

In general, intermediates of Formula (IXb) can be prepared according to the following reaction Scheme 4. In Scheme 4 PG represents a suitable protecting group, such as for example tert-butyloxycarbonyl; all other variables are defined according to the scope of the present invention.

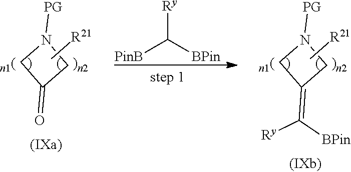

Step 1: at a suitable temperature such as for example −78° C., in the presence of a suitable deprotonating agent such as for example n-Butyllithium, in presence of a suitable reagent such as 2,2,6,6-Tetramethylpiperidine (HTMP), in a suitable solvent such as tetrahydrofuran;

### Scheme 5

In general, compounds of Formula (I-a), can be prepared according to the following reaction Scheme 5. In Scheme 5, PG represents a suitable protecting group, such as for example tert-butyloxycarbonyl and LG is a leaving group such as for example chloro, bromo, iodo or tosylate or mesylate or triflate; all other variables are defined according to the scope of the present invention.

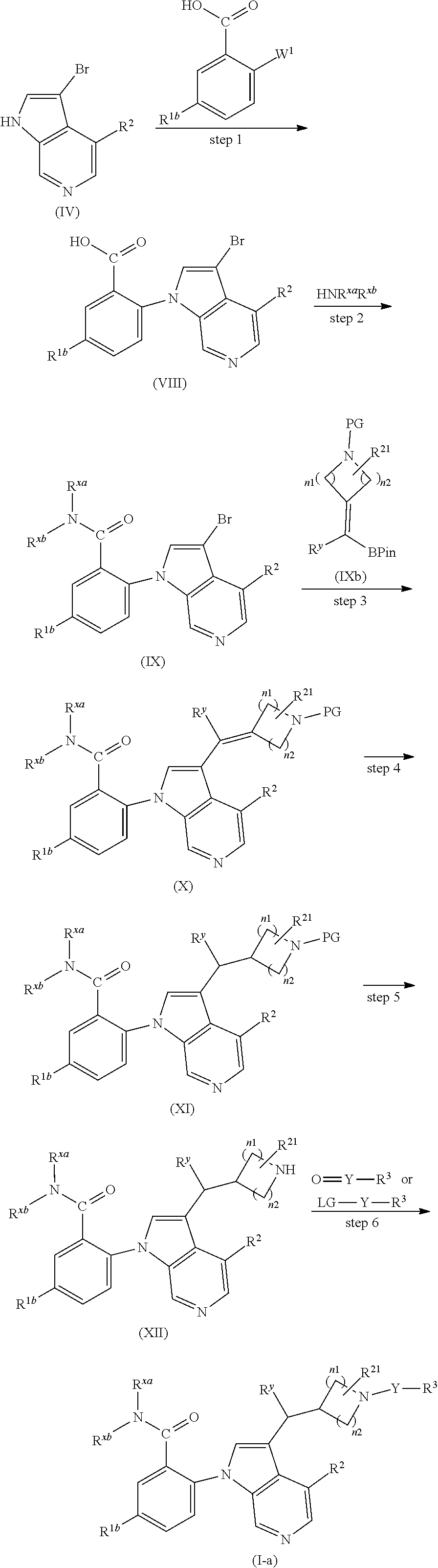

In Scheme 5, the following reaction conditions apply:
Step 1: at a suitable temperature such as for example 100° C., in the presence of a suitable catalyst such as copper (Cu), in the presence of a base such as potassium carbonate, in a suitable solvent such as dimethylformamide; Alternatively a copper (I) source may be used, such as CuI in the presence of a suitable diamine ligand, such as trans-N,N′-dimethylcyclohexane-1,2-diamine, in the presence of an inorganic base, such as potassium carbonate in an aprotic solvent such as dimethylformamide at a temperature between 80° C. and 150° C.;
Step 2: at a suitable temperature such as for example room temperature, in the presence of a suitable condensation reagent such as 2-(7-Azabenzotriazol-1-yl)-N,N,N′,N′-tetramethyluronium hexafluorophosphate (HATU), in the presence of a base such as N,N-Diisopropylethylamine (DIPEA), in a suitable solvent such as dimethylformamide;
Alternatively the acid chloride may be prepared by reacting intermediate VIII with thionyl chloride optionally in a halogenated solvent such as dichloromethane at a temperature in a range between 0° C. and room temperature. The intermediate acid chloride may then be reacted with the amine HNR^{xa}R^{xb} optionally in an aprotic solvent such as dimethylformamide and optionally in the presence of a tertiary amine such as N,N-diisopropylethylamine;
Step 3: at a suitable temperature in a range between 60° C. and 120° C., such as for example 100° C., in the presence of a suitable base such as for example potassium carbonate, in presence of a suitable catalyst such as [1,1′-Bis(diphenylphosphino)ferrocene]dichloropalladium(II) (Pd(dppf)Cl₂) in a suitable solvent such as for example dioxane or dimethylformamide and water;
Step 4: at a suitable temperature such as room temperature, in the presence of a suitable heterogenous catalyst such as palladium on charcoal (Pd/C), in a common solvent such as methanol, ethanol, THE or the like under hydrogen pressure such as for example from 1 to 3 bar, optionally in the presence of a base such as triethylamine;
Step 5: at a suitable temperature in a range between 0° C. and 40° C., such as room temperature, in the presence of a suitable acid, for example a protic acid such as trifluoroacetic acid (TFA) or hydrochloric acid, in a suitable solvent such as dichloromethane (DCM) or 1,4-dioxane;
Step 6:
In the case of a reductive amination reaction employing an aldehyde or a ketone: at a suitable temperature in a range between room temperature and 70° C., in the presence of a suitable reducing agent such as for example sodium triacetoxyborohydride or sodium cyanoborohydride, in a suitable solvent such as for example methanol, dichloromethane or 1,2-dichlorethane, optionally in the presence of zinc chloride or sodium acetate or acetic acid;
In the case of an alkylation reaction employing LG-Y—R³: at a suitable temperature such as for example room temperature, in the presence of a suitable deprotonating agent such as for example sodium hydride or potassium carbonate, or an amine base such as triethylamine in a suitable aprotic solvent such as for example dimethylformamide or dimethylsulfoxide or acetonitrile.

### Scheme 6

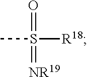

In general, compounds of Formula (I-b) wherein R^{1a} is limited to —S(═O)₂—R¹⁸, NR and Q represents —CHR^{y}—, can be prepared according to the following reaction Scheme 6. In Scheme 6, PG represents a suitable protecting group, such as for example tert-butyloxycarbonyl, 9-fluorenylmethoxycarbonyl, or benzyl; LG is a leaving group such as for example chloro, bromo, iodo or tosylate or mesylate; LG1 is a leaving group such as for example fluoro, chloro, bromo, iodo or tosylate or mesylate; all other variables are defined according to the scope of the present invention.

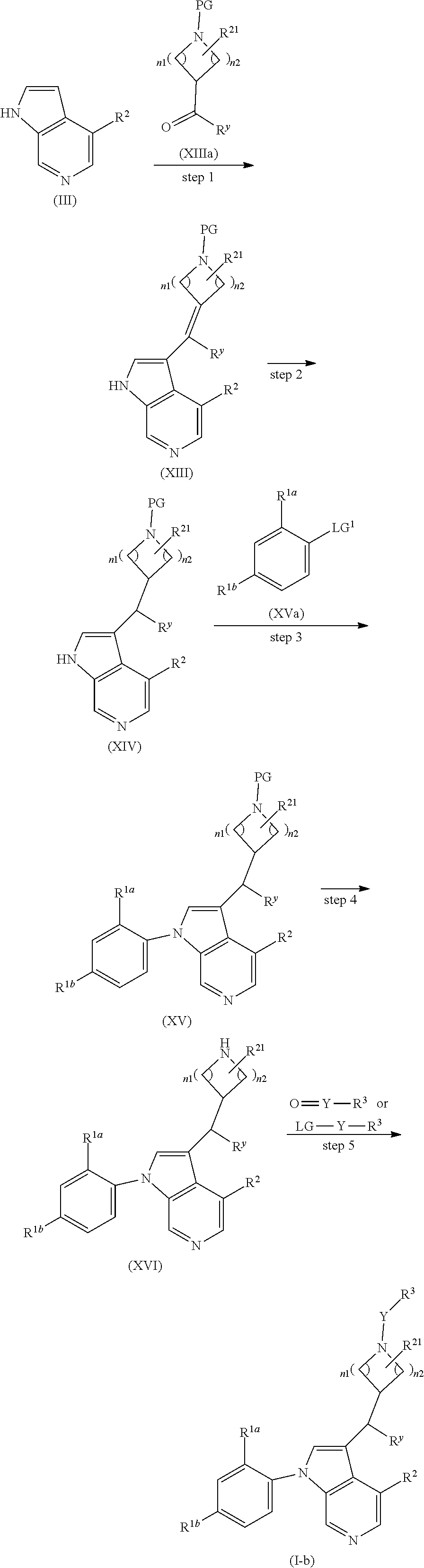

In Scheme 6, the following reaction conditions apply:
Step 1: at a suitable temperature in a range between 50° C. and 90° C., in the presence of a suitable base such as for example potassium hydroxide or sodium hydroxide, in a suitable solvent, preferably a protic solvent, such as methanol, ethanol or isopropanol.
Step 2: at a suitable temperature such as room temperature, in the presence of a suitable heterogenous catalyst such as palladium on charcoal (Pd/C), in a common solvent such as methanol, ethanol, THE or the like under hydrogen pressure such as for example from 1 to 3 bar, optionally in the presence of a base such as triethylamine;
Step 3: at a suitable temperature in a range between 50° C. and 100° C., in the presence of a suitable inorganic base such as for example potassium carbonate or potassium tert-butoxide, in a suitable aprotic solvent such as for example dioxane, dimethylformamide or acetonitrile or dimethylsulfoxide;
Step 4: when PG=Boc, at a suitable temperature in a range between 0° C. and 40° C., such as room temperature, in the presence of a suitable acid, for example a protic acid such as trifluoroacetic acid or hydrochloric acid, in a suitable solvent such as dichloromethane or 1,4-dioxane;
Alternatively, when PG=9-Fluorenylmethoxycarbonyl, at a suitable temperature in a range between 0° C. and 40° C., such as room temperature, in the presence of a suitable base such as piperidine, in a suitable solvent such as dichloromethane (DCM);
Alternatively, when PG=benzyl, at a suitable temperature such as room temperature, in the presence of a suitable heterogenous catalyst such as palladium on charcoal (Pd/C), in a common solvent such as methanol, ethanol, THE or the like under hydrogen pressure such as for example from 1 to 3 bar, optionally in the presence of a base such as triethylamine;
Step 5: In the case of a reductive amination reaction employing an aldehyde or a ketone: at a suitable temperature in a range between room temperature and 70° C., in the presence of a suitable reducing agent such as for example sodium triacetoxyborohydride or sodium cyanoborohydride, in a suitable solvent such as for example methanol, dichloromethane or 1,2-dichloroethane, optionally in the presence of zinc chloride or sodium acetate or acetic acid;
In the case of an alkylation reaction employing LG-Y—R³: at a suitable temperature such as for example room temperature, in the presence of a suitable deprotonating agent such as for example sodium hydride or potassium carbonate, or an amine base such as triethylamine in a suitable aprotic solvent such as for example dimethylformamide or dimethylsulfoxide or acetonitrile;

### Scheme 7

In general, compounds of Formula (I-c), can be prepared according to the following reaction Scheme 7. In Scheme 7, PG represents a suitable protecting group, such as for example tert-butyloxycarbonyl, 9-fluorenylmethoxycarbonyl, or benzyl, all other variables are defined according to the scope of the present invention.

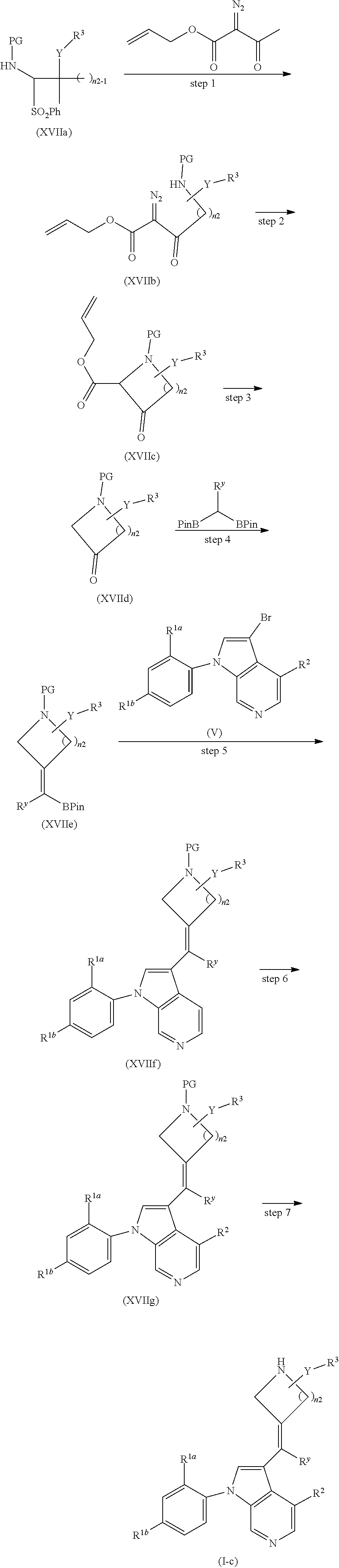

In Scheme 7, the following reaction conditions apply:
Step 1: at a suitable temperature such as for example −78° C., in the presence of a suitable deprotonating agent such as for example lithium bis(trimethylsilyl)amide (LiHMDS) and sodium hydride, in a suitable solvent such as for example tetrahydrofuran;
Step 2: at a suitable temperature in a range between room temperature and 100° C., in the presence of a suitable catalyst such as for example rhodium acetate dimer (Rh₂(OAc)₄), in a suitable solvent such as for example dichloromethane;
Step 3: at a suitable temperature in a range between room temperature and 100° C., in the presence of a suitable catalyst such as for example tetrakis(triphenylphosphine)palladium (Pd(PPh₃)₄), in the presence of a suitable base such as for example morpholine, in a suitable solvent such as for example tetrahydrofuran;
Step 4: at a suitable temperature such as for example −78° C., in the presence of a suitable deprotonating agent such as for example n-Butyllithium, in presence of a suitable reagent such as 2,2,6,6-Tetramethylpiperidine (HTMP), in a suitable solvent such as tetrahydrofuran;
Step 5: at a suitable temperature such as for example 100° C., in the presence of a suitable base such as for example potassium carbonate, in presence of a suitable catalyst such as [1,1′-Bis(diphenylphosphino)ferrocene]dichloropalladium(II) (Pd(dppf)Cl₂) in a suitable solvent such as for example dioxane or dimethylformamide and water;
Step 6: at a suitable temperature such as room temperature, in the presence of a suitable heterogenous catalyst such as palladium on charcoal (Pd/C), in a common solvent such as methanol, ethanol, THE or the like under hydrogen pressure such as for example from 1 to 3 bar, optionally in the presence of a base such as triethylamine;
Step 7: when PG=Boc, at a suitable temperature in a range between 0° C. and 40° C., such as room temperature, in the presence of a suitable acid, for example a protic acid such as trifluoroacetic acid or hydrochloric acid, in a suitable solvent such as dichloromethane or 1,4-dioxane;
Alternatively, when PG=9-fluorenylmethoxycarbonyl, at a suitable temperature in a range between 0° C. and 40° C., such as room temperature, in the presence of a suitable base such as piperidine, in a suitable solvent such as dichloromethane (DCM);
Alternatively, when PG=benzyl, at a suitable temperature such as room temperature, in the presence of a suitable heterogenous catalyst such as palladium on charcoal (Pd/C), in a common solvent such as methanol, ethanol, THE or the like under hydrogen pressure such as for example from 1 to 3 bar, optionally in the presence of a base such as triethylamine.

An example of steps 1 and 2 in Scheme 7 is the preparation of a 5-membered intermediate (XVIIcc), as shown in Scheme 7a which can be prepared according to the general procedures outlined in steps 1 and 2 in Scheme 7.

### Scheme 7a

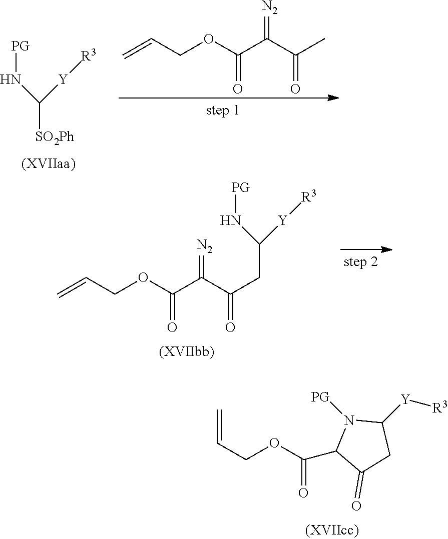

### Scheme 8

In general, intermediates of Formula (XVIIId), can be prepared according to the following reaction Scheme 8. In Scheme 8, W₂ represents chloro, bromo or iodo, all other variables are defined according to the scope of the present invention. A skilled person will realize that cyclobutyl in Scheme 8 can be C₃₋₆cycloalkyl in general, and that an intermediate of Formula (XVIIId) can be further functionalized into a compound of Formula (I) by analogous reaction protocols as described in the general schemes herein, combined with standard synthetic processes commonly used by those skilled in the art of organic chemistry.

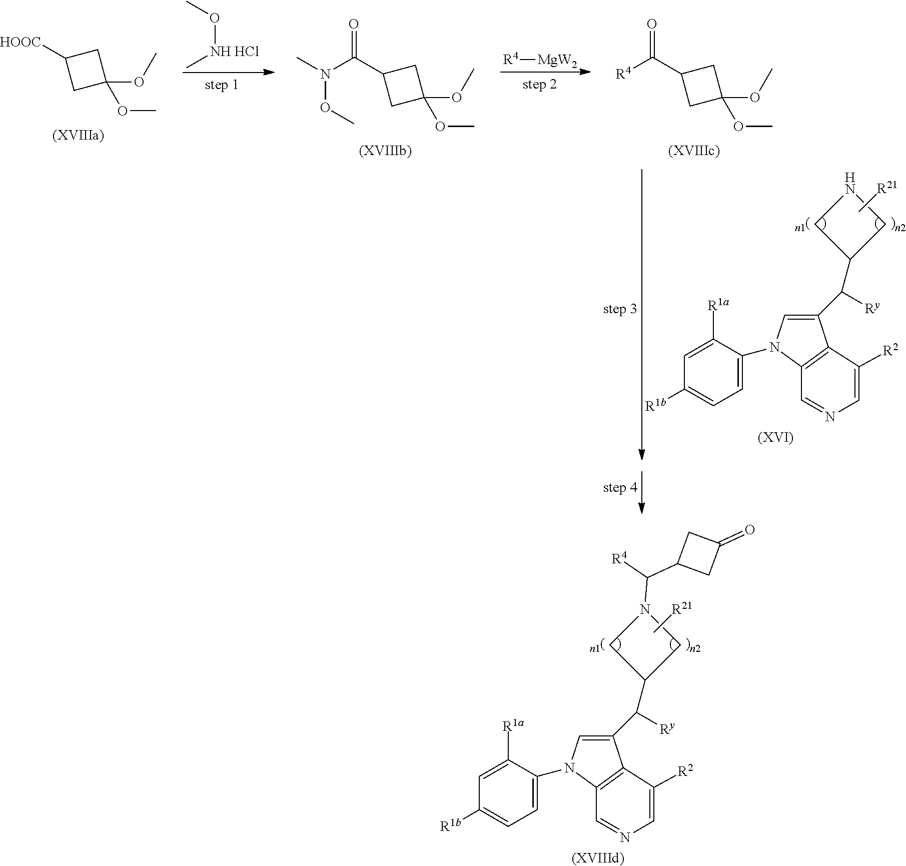

In Scheme 8, the following reaction conditions apply:
Step 1: at a suitable temperature such as for example 0° C., in the presence of a suitable condensation reagent such as propylphosphonic anhydride (T₃P), in the presence of a base such as N,N-Diisopropylethylamine (DIPEA), in a suitable solvent such as dimethylformamide or dichloromethane;
Step 2: at a suitable temperature such as for example 0° C., in a suitable solvent such as tetrahydrofuran;
Step 3: at a suitable temperature in a range between room temperature and 70° C., in the presence of a suitable reducing agent such as for example sodium triacetoxyborohydride or sodium cyanoborohydride, in a suitable solvent such as for example methanol, dichloromethane or 1,2-dichloroethane, optionally in the presence of zinc chloride or sodium acetate or acetic acid;
Step 4: at a suitable temperature in a range between 0° C. and 40° C., such as room temperature, in the presence of a suitable acid such as hydrochloric acid (HCl, 1N), in a suitable solvent such as acetonitrile.

### Scheme 9

In general, intermediates as described in Scheme 9, wherein Q represents —CHR^{y}—, can be prepared according to the following reaction Scheme. In Scheme 9, PG represents a suitable protecting group, such as for example tert-butyloxycarbonyl, all other variables are defined according to the scope of the present invention.

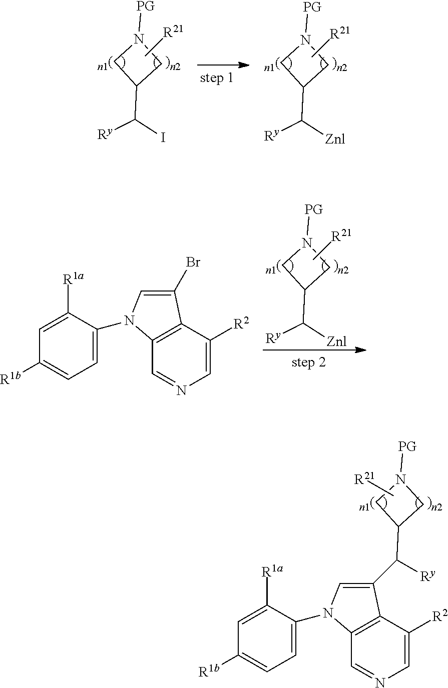

In Scheme 9, the following reaction conditions apply:
Step 1: at a suitable temperature, in a range between room temperature and 70° C., such as 60° C., in the presence of zinc, in the presence of suitable activating agents such as trimethylsilylchloride or 1-bromo, 2-chloroethane, in a suitable solvent such as tetrahydrofuran. Optionally, the procedure can also be performed with the use of a flow-apparatus;
Step 2: at a suitable temperature, in a range between room temperature and 70° C., such as 50° C., in the presence of a suitable catalyst such as 4^{th} generation RuPhos Pd precatalyst (RuPhos Pd G4), in a suitable solvent such as tetrahydrofuran.

### Scheme 10

In general, intermediates as described in Scheme 10, wherein Q represents —CHR^{y}—, can be prepared according to the following reaction Scheme. In Scheme 10, PG represents a suitable protecting group, such as for example tert-butyloxycarbonyl, all other variables are defined according to the scope of the present invention. BPin represents 4,4,5,5-tetramethyl-1,3,2-dioxaborolane. W¹ and W³ represent fluoro, chloro, bromo or iodo.

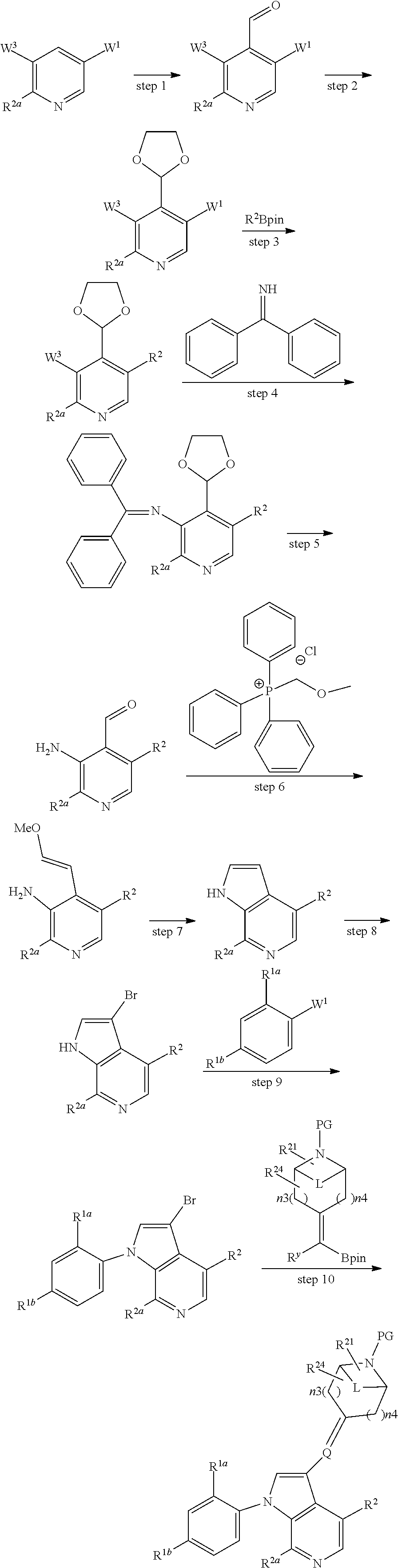

Step 1: at a suitable temperature, such as −78° C., in the presence of a suitable deprotonating agent such as n-Butyllithium, in a suitable solvent such as tetrahydrofuran, in the presence of suitable electrophile, such as DMF;
Step 2: at a suitable temperature in a range between 80° C. and 120° C., in the presence of a diol protection reagent such as for example glycol, in the presence of a Bronsted acid such as for example para-toluenesulfonic acid in a suitable solvent such as for example toluene;
Step 3: at a suitable temperature in a range between room temperature and 100° C., in the presence of a suitable base such as for example potassium carbonate, in the presence of a suitable catalyst such as [1,1′-Bis(diphenylphosphino)ferrocene]dichloropalladium(II) (Pd(dppf)Cl₂) in a suitable solvent such as for example dioxane or dimethylformamide and water; Alternatively, when R²=Me, a boron containing reagent such as trimethyl boroxine can be used in the presence of a suitable catalyst such as (Pd(dppf)Cl2) in a suitable solvent such as for example dioxane or dimethylformamide and water in the presence of an inorganic base such as potassium carbonate at a reaction temperature between 80° C. and 120° C.;
Step 4: in the presence of a suitable base, such as for example sodium t-butoxide, in the presence of a suitable palladium source such as palladium(II)acetate (Pd(OAc)₂), in the presence of a suitable ligand, such as 1,1′-(9,9-Dimethyl-9H-xanthene-4,5-diyl)bis[1,1-diphenylphosphine], Xantphos, in the presence of a suitable solvent, such as 1,4-dioxane, at a suitable temperature range between 50° C. and 120° C.;
Step 5: in the presence of a suitable Bronsted acid, such as hydrochloric acid, in the presence of a suitable solvent such as 1,4-dioxane or tetrahydrofuran, and water, at a suitable temperature range such a room temperature and 60° C.;
Step 6: in the presence of a suitable deprotonating agent, such as n-butyllithium, in a suitable solvent such as tetrahydrofuran, at a suitable temperature range such as −78° C. and room temperature.
Step 7: in the presence of a suitable Bronsted acid, such as hydrochloric acid, in the presence of a suitable solvent such as 1,4-dioxane or tetrahydrofuran, and water, at a suitable temperature range such a room temperature and 100° C.;
Step 8: at a suitable temperature such as for example between 0° C. and room temperature, in the presence of a suitable bromination reagent such as for example N-bromosuccinimide or CuBr₂, in a suitable solvent such as for example dimethylformamide or acetonitrile;
Step 9: at a suitable temperature such as for example 80° C. and 130° C., in the presence of a suitable catalyst such as copper (Cu), in the presence of a base such as potassium carbonate, in a suitable solvent such as dimethylformamide. Alternatively a copper (I) source may be used, such as CuI in the presence of a suitable diamine ligand, such as trans-N,N′-dimethylcyclohexane-1,2-diamine in the presence of an inorganic base, such as potassium carbonate in an aprotic solvent such as dimethylformamide at a temperature between 80° C. and 150° C. In certain cases said conversion may also be effected by a nucleophilic aromatic substitution using an inorganic base such as potassium tert-butoxide or sodium hydride or the like, in an aprotic solvent such as dimethylformamide at a temperature between 0° C. and 80° C.;
Step 10: at a suitable temperature such as for example 100° C., in the presence of a suitable base such as for example potassium carbonate, in presence of a suitable catalyst such as [1,1′-Bis(diphenylphosphino)ferrocene]dichloropalladium(II) (Pd(dppf)Cl₂) in a suitable solvent such as for example dioxane or dimethylformamide and water.

As can be appreciated by a person skilled in the art, the intermediate obtained in scheme 10, can be further elaborated to obtain compounds of Formula (A) by means of using the procedures outlined in the general schemes mentioned above, in particular in scheme 1 and scheme 3.

### Scheme 11

In general, intermediates as described in Scheme 11, can be prepared according to the following reaction Scheme. In Scheme 11, PG represents a suitable protecting group, such as for example tert-butyloxycarbonyl, all other variables are defined according to the scope of the present invention. BPin represents 4,4,5,5-tetramethyl-1,3,2-dioxaborolane.

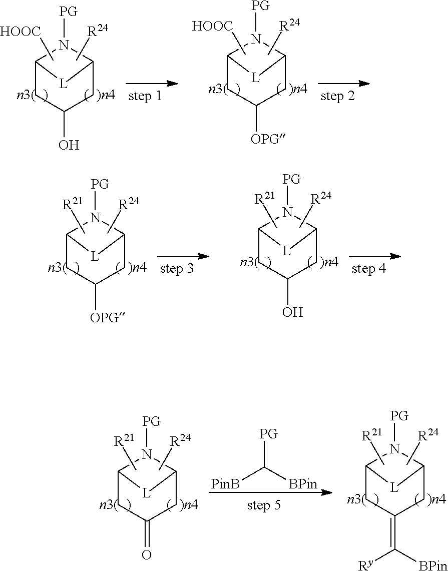

Step 1: when PG″=a silyl containing protecting group, such as tert-butyldimethylsilyl, at a suitable temperature in a range between room temperature and 80° C., such as room temperature, in the presence of a base, such as imidazole, in the presence of a suitable reagent, such as tert-butyldimethylsilylchloride, in a suitable solvent, such as DMF. When, PG″ is a different protecting group as defined herein, general protection conditions may be used, known to those skilled in the art.
Step 2: at a suitable temperature, between room temperature and 60° C., such as room temperature, for example in the presence of a suitable alkyl halide, in the presence of a suitable base, such as K₂CO₃, in the presence of a suitable photocatalyst, such as [4,4′-Bis(1,1-dimethylethyl)-2,2′-bipyridine-N¹,N^{1′}]bis[3,5-difluoro-2-[5-(trifluoromethyl)-2-pyridinyl-N]phenyl-C]Iridium(III) hexafluorophosphate, [Ir{dF(CF₃)ppy}₂(dtbpy)]PF₆, in the presence of suitable nickel salt, such as NiCl_{2′}glyme, in the presence of a suitable ligand, such as 4-4′-dimethoxy-2-2′-bipyridine, in a suitable solvent, such as acetonitrile and in the presence of water as an additive, employing blue LED irradiation (Johnston, C., Smith, R., Allmendinger, S. et al. Metallaphotoredox-catalysed sp³-sp³ cross-coupling of carboxylic acids with alkyl halides. Nature 536, 322-325 (2016)).
Step 3: at a suitable temperature, such as room temperature, in the presence of a suitable fluoride source, such a tetrabutylammonium fluoride, in a suitable solvent, such as tetrahydrofuran. When, PG is a different protecting group as defined herein, general protection conditions may be used, known to those skilled in the art.
Step 4: at a suitable temperature, such as between −78° C. and 40° C., in the presence of Dess-Martin periodinane, in a suitable solvent such as dichloromethane. Other oxidation methods, known to those skilled in the art may also be employed.
Step 5: at a suitable temperature such as for example −78° C., in the presence of a suitable deprotonating agent such as for example n-Butyllithium, in presence of a suitable reagent such as 2,2,6,6-Tetramethylpiperidine (HTMP), in a suitable solvent such as tetrahydrofuran.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, New Jersey, 2007.

Several methods for preparing the compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| CH₃COONH₄ | ammonium acetate |
| 1,2-DCE | 1,2-dichloroethane |
| sat. or Sat. | saturated |
| ° C. | degree Celsius |
| AcOH or CH₃COOH | acetic acid |
| Ac₂O | acetic anhydride |
| aq. | aqueous |
| atm | atmosphere |
| BH3•THF | Borane tetrahydrofuran complex |
| Boc or boc | tert-butyloxycarbonyl |
| BOC-anhydride | di-tert-butyl dicarbonate |
| BPin or PinB | 4,4,5,5-tetramethyl-1,3,2-dioxaborolane |
| Celite | diatomaceous earth |
| CO₂ | carbon dioxide |
| Cs₂CO₃ | cesium carbonate |
| CPME | cyclopentyl methylether |
| DCM or CH2Cl2 | dichloromethane |
| DEA | diethanolamine |
| DEE | diethyl ether |
| DIEA or DIPEA | N-ethyl-N-(propan-2-yl)propan-2-amine |
| DMA | N,N-dimethylacetamide |
| DME | 1,2-dimethoxyethane |
| DMF | N,N-dimethylformamide |
| DMSO | (methanesulfinyl)methane |
| DSC | differential scanning calorimetry |
| EDCI or EDCI•HCl | 3-{[(ethylimino)methylidene]amino}-N,N- |
| | dimethylpropan-1-amine |
| ee | enantiomeric excess |
| ESI | electrospray ionization |
| EtOAc or EA | ethyl acetate |
| EtOH | ethanol |
| FA | formic acid |
| FCC | flash column chromatography |
| h or hr | hour(s) |
| HATU | 1-[bis(dimethylamino)methylene]-1H-1,2,3- |
| | triazolo[4,5-b]pyridinium 3-oxide hexafluorophosphate |
| HCl | hydrochloric acid |
| Hex | hexane |
| HOBt | 1-hydroxybenzotriazole |
| IBX | 2-iodoxybenzoic acid |
| Insolute ® SCX-3 | ethylbenzene sulfonic acid cation exchange resin |
| i-PrNH₂ or iPrNH | isopropyl amine |
| i-PrOH, iPrOH or IPA | isopropyl alcohol |
| IPAC | isopropyl acetate |
| K₂CO₃ | potassium carbonate |
| KOAc | potassium acetate |
| LCMS | liquid chromatography-mass spectrometry |
| LiAlH₄ | lithium aluminium hydride |
| Li-HMDS or LiHMDS | lithium bis(trimethylsilyl)amide |
| Mor N | mol/L |
| MeCN or CH₃CN | acetonitrile |
| MeI | iodomethane |
| MeOH | methanol |
| mg | milligram |
| min | minute(s) |
| mL | milliliter |
| mmol | millimole |
| m-CPBA | meta-chloroperoxybenzoic acid |
| MS | mass spectrometry |
| N₂ | nitrogen |
| Na₂CO₃ | sodium carbonate |
| Na₂SO₄ | sodium sulfate |
| NaBH(OAc)₃ | sodium triacetoxyborohydride |
| NaBH₃CN | sodium cyanoborohydride |
| Na₄EDTA | Ethylenediaminetetraacetic acid tetrasodium salt |
| NaHCO₃ | sodium hydrogencarbonate |
| NaOAc | sodium acetate |
| NaOH | sodium hydroxide |
| NH₃ | ammonia |
| NH₃•H₂O or NH₄OH | ammonium hydroxide |
| NH₄HCO₃ | ammonium hydrogencarbonate |
| n-BuLi | n-butyllithium |
| NBS | N-Bromosuccinimide |
| NH₄Cl | ammonium chloride |
| NMP | 1-methyl-2-pyrrolidinone |
| NMR | nuclear magnetic resonance |
| Pd(dppf)Cl₂ | [1,1′-bis(diphenylphosphino)ferrocene]dichloropalladium(II) |
| Pd(PPh₃)₄ | tetrakis(triphenylphosphine)palladium(0) |
| Pd/C | palladium on carbon |
| PE | petroleum ether |
| PIDA | Diacetoxy iodobenze or iodobenzen diacetate |
| Prep. HPLC | preparative high-performance liquid chromatography |
| Prep. SFC | preparative supercritical fluid chromatography |
| Prep. TLC | preparative thin-layer chromatography |
| Rf | retention factor |
| Rh₂(OAc)₄ | rhodium acetate |
| RP | Reverse(d) phase |
| rt, r.t. or RT | room temperature |
| RuPhos Pd G4/4^{th} | Palladium, [[2′,6′-bis(1-methylethoxy)[1,1′-biphenyl]- |
| generation RuPhos Pd | 2-yl]dicyclohexylphosphine-κP](methanesulfonato- |
| precatalyst | κO)[2′-(methylamino-κN)[1,1′-biphenyl]-2-yl-κC]-, |
| | (SP-4-3)- (ACI) |
| | CAS 1599466-85-9 |
| sat. | saturated |
| SFC | supercritical fluid chromatography |
| SiliaBond ® propylsulfonic | Propylsulfonic acid bound to silica stationary phase |
| acid resin | support |
| SiliaMetS ® Diamine | N1-propylethane-1,2-diamine bound to silica stationary |
| | phase support |
| t-butyl | tert-butyl |
| t-BuOK | potassium tert-butoxide |
| T₃P | propylphosphonic anhydride |
| TEA or Et₃N | triethylamine |
| Temp | temperature |
| TFA | trifluo acetic acid |
| THF | tetrahydrofuran |
| TLC | thin-layer chromatography |
| Tₒₙₛₑₜ | Temperature at which melting onset occurs (measure |
| | by DSC) |
| Ts | tosyl |
| UV | ultraviolet |
| v/v | volume to volume |
| w/v | weight to volume |
| w/w | weight to weight |
| ZnCl₂ | zinc chloride |
| Xantphos | 4,5-bis(diphenylphosphino)-9,9-dimethylxanthene |

As understood by a person skilled in the art, compounds synthesized using the protocols as indicated may exist as a solvate e.g. hydrate, and/or contain residual solvent or minor impurities. Compounds or intermediates isolated as a salt form, may be integer stoichiometric i.e. mono- or di-salts, or of intermediate stoichiometry. When an intermediate or compound in the experimental part below is indicated as ‘HCl salt’ without indication of the number of equivalents of HCl, this means that the number of equivalents of HCl was not determined. The same principle will also apply to all other salt forms referred to in the experimental part, such as e.g. ‘oxalate salt’, ‘HCOOH salt’ (‘formate salt’), or

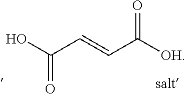

The stereochemical configuration for centers in some compounds may be designated “R” or “S” when the mixture(s) was separated and absolute stereochemistry was known, or when only one enantiomer was obtained and absolute stereochemistry was known; for some compounds, the stereochemical configuration at indicated centers has been designated as “*R” or “*S” when the absolute stereochemistry is undetermined (even if the bonds are drawn stereo specifically) although the compound itself has been isolated as a single stereoisomer and is enantiomerically pure. In case a compound designated as “*R” is converted into another compound, the “*R” indication of the resulting compound is derived from its starting material.

For example, it will be clear that Compound 135

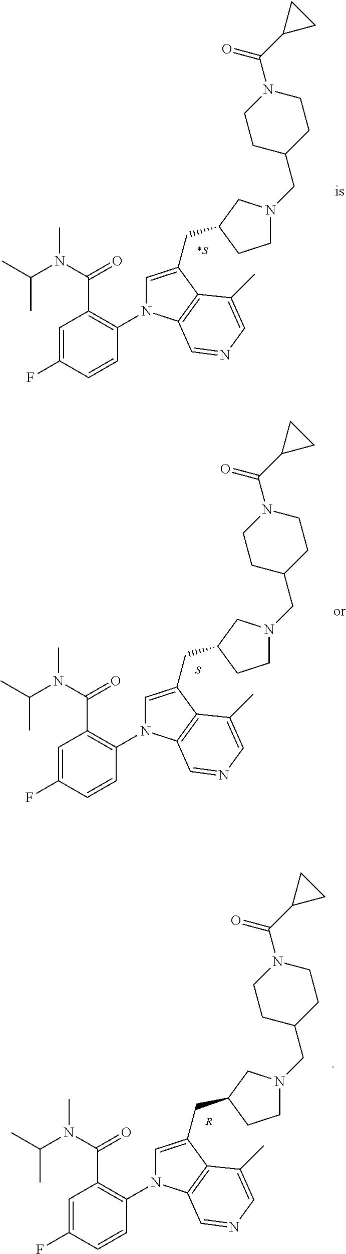

A skilled person will realize that the paragraphs above about stereochemical configurations, also apply to intermediates.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers or undetermined stereochemistry, unless otherwise is indicated or is clear from the context.

When a stereocenter is indicated with ‘RS’ this means that a racemic mixture was obtained at the indicated centre, unless otherwise indicated.

A double bond indicated with EZ means the compound/intermediate was obtained as a mixture of E and Z isomers.

### Preparation of Intermediates and Compounds

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

### Preparation of Intermediate 1:

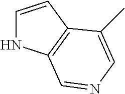

To a solution of 4-bromo-1H-pyrrolo[2,3-c]pyridine (2 g, 95% purity, 9.64 mmol) in 1,4-dioxane (30 mL) and water (4 mL) was added 2,4,6-trimethyl-1,3,5,2,4,6-trioxatriborinane (7.26 g, 50% in THF, 28.9 mmol) and potassium carbonate (4.0 g, 28.9 mmol). The suspension was degassed and exchanged with N₂ twice. [1,1′-bis(diphenylphosphino)ferrocene]dichloropalladium(II) (706 mg, 0.964 mmol) was added into the reaction mixture. The reaction mixture was heated up to 100° C. and stirred at this temperature overnight. After cooled down to r.t., the reaction mixture was filtered and the filtrate was concentrated. The resulting residue was purified by silica gel column chromatography eluting with ethyl acetate in petroleum ether from 0% to 80% to give intermediate 1 (1.01 g, 95% purity, 75.3% yield).

Alternatively, intermediate 1 can also be prepared with the following procedure:

Into a 20 L 4-necked round-bottom flask were added 4-bromo-1H-pyrrolo[2,3-c]pyridine (1330 g, 6750 mmol, 1.00 equiv), Pd(dppf)Cl₂ (493.9 g, 675 mmol, 0.10 equiv), K₂CO₃ (2798.69 g, 20250.21 mmol, 3.00 equiv), 1,4-dioxane (13 L), H₂O (2 L) and 2,4,6-trimethyl-1,3,5,2,4,6-trioxatriborinane (2542.01 g, 20250.21 mmol, 3.00 equiv) at room temperature. The resulting mixture was stirred for overnight at 100° C. The mixture was allowed to cool down to room temperature. The resulting mixture was concentrated under reduced pressure. The resulting mixture was diluted with water (15 L). The aqueous layer was extracted with EtOAc (3×10 L) and the organic layer was washed with water (2×5 L). The resulting liquid was dried with Na₂SO₄, filtered and concentrated under vacuum. The residue was purified by silica gel column chromatography, eluting with 10% methanol in dichloromethane to afford intermediate 1 (640 g, yield: 72%) as a grey solid.

### Preparation of Intermediate 2:

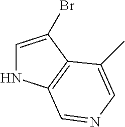

At 0° C., to a solution of intermediate 1 (918 mg, 95% purity, 6.6 mmol) in DMF (60 mL) was added a solution of N-bromosuccinimide (1.17 g, 6.6 mmol) in DMF (10 mL) dropwise. The reaction mixture was stirred at this temperature for 30 minutes. The reaction mixture was quenched with water and extracted with ethyl acetate (50 mL) twice. The organic layer was washed with brine (25 mL), dried over sodium sulfate, filtered and concentrated to afford the crude product, which was purified by silica gel column chromatography eluting with ethyl acetate in petroleum from 0% to 60% to give intermediate 2 (1.14 g, 97.1% purity, 79.5% yield) as a white solid.

Alternatively, intermediate 2 can also be prepared with the following procedure:

Into a 10 L 4-necked round-bottom flask were added intermediate 1 (640 g, 4842.39 mmol, 1.00 equiv) and DMF (5.00 L) at room temperature. To the above mixture was added NBS (861.87 g, 4842.40 mmol, 1.00 equiv) in portions over 1 h at room temperature. The resulting mixture was stirred for additional 30 min at room temperature. The reaction was quenched by the addition of aqueous solution of Na₂S₂O₃ (10 L, 10% (w/v)) at room temperature. The aqueous layer was extracted with EtOAc (3×5 L) and the organic layer was washed with brine (1×5 L). The resulting liquid was dried with Na₂SO₄ and concentrated. The residue was purified by silica gel column chromatography, eluting with 20% ethyl acetate in petroleum ether to afford intermediate 2 (800 g, yield: 78%) as a grey solid.

### Preparation of Intermediate 4:

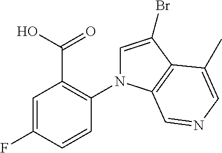 [SMILES: Cc1cncc2c1c(Br)cn2-c1ccc(F)cc1C(=O)O]

To a solution of intermediate 2 (1.14 g, 97.1% purity, 5.24 mmol) in DMF (80 mL) were added 5-fluoro-2-iodobenzoic acid (1.40 mg, 5.24 mmol), copper powder (333 mg, 5.24 mmol) and potassium carbonate (2.18 g, 15.7 mmol). The reaction mixture was heated up to 100° C. and stirred at this temperature overnight. After the mixture was cooled down to r.t., the reaction mixture was concentrated and the resulting residue was acidified with HCl (1 N) to pH=~3. The resulting mixture was filtered and the filter cake was washed with water twice. The filter cake was dried under vacuum to give crude intermediate 4 (1.8 g, 91% purity, 89.4% yield) as a yellow solid.

Alternatively, intermediate 4 can also be prepared with the following procedure:

Into a 10 L 4-necked round-bottom flask were added intermediate 2 (560 g, 2653.24 mmol, 1.00 equiv), Cu (252.91 g, 3979.87 mmol, 1.50 equiv), K₂CO₃ (1100.08 g, 7959.74 mmol, 3.00 equiv) and 5-fluoro-2-iodobenzoic acid (705.79 g, 2653.24 mmol, 1.00 equiv) in DMF (6.00 L) at room temperature. The resulting mixture was stirred for additional 2 h at 100° C. under nitrogen atmosphere. The resulting mixture was filtered, the filter cake was washed with DMF (1×5 L) and the filtrate was concentrated under reduced pressure. The resulting mixture was diluted with water (8 L). The mixture was acidified to pH 3 with aqueous HCl (conc.). The precipitated solids were collected by filtration and washed with water (3×3 L). The resulting solid was dried under vacuum to afford intermediate 4 (1300 g, crude) as a grey solid.

Intermediate 110 was synthesized by an analogous method as described for intermediate 4

| | | |
|---|---|---|
| Intermediate 110 | 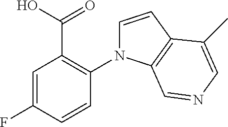 [SMILES: Cc1cncc2c1ccn2-c1ccc(F)cc1C(=O)O] | Intermediate 1 |

### Preparation of Intermediate 6:

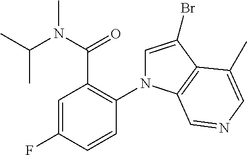 [SMILES: Cc1cncc2c1c(Br)cn2-c1ccc(F)cc1C(=O)N(C)C(C)C]

At 0° C., to a solution of intermediate 4 (1.8 g, 91% purity, 4.69 mmol) in DMF (50 mL) was added HATU (4.46 g, 11.7 mmol), N,N-diisopropylethylamine (3.03 g, 23.5 mmol) and N-methylpropan-2-amine (858 mg, 11.7 mmol). After addition, the mixture was stirred at room temperature overnight. The reaction mixture was concentrated and the resulting residue was purified by silica gel column chromatography eluted with methanol in dichloromethane from 0% to 5% to give intermediate 6 (2.0 g, 93% purity, 98.1% yield) as a yellow oil.

Alternatively, intermediate 6 can also be prepared with the following procedure:

Into a 20 L 4-necked round-bottom flask were added intermediate 4 (920 g, 2634.90 mmol, 1.00 equiv, same as 1300 g crude), DMF (7.5 L), HATU (1102.06 g, 2898.39 mmol, 1.10 equiv) and DIEA (1021.63 g, 7904.70 mmol, 3.00 equiv) at room temperature. The resulting mixture was stirred for additional 30 min at room temperature. To the above mixture was added N-methylpropan-2-amine (211.99 g, 2898.39 mmol, 1.10 equiv) dropwise over 10 min at 0° C. The resulting mixture was stirred overnight at room temperature. The reaction was quenched by the addition of water (20 L) at room temperature. The aqueous layer was extracted with EtOAc (3×7 L) and the organic layer was washed with water (3×5 L). The resulting liquid was dried with Na₂SO₄, filtered and concentrated under reduced pressure. The residue was purified by silica gel column chromatography, eluting with 50% ethyl acetate in petroleum ether (1:1) to afford intermediate 6 (700 g, yield: 66%) as a light yellow solid.

Intermediate 111 was synthesized by an analogous method as described for intermediate 6.

| | | |
|---|---|---|
| Intermediate 111 | 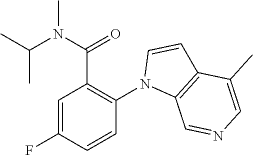 [SMILES: Cc1cncc2c1ccn2-c1ccc(F)cc1C(=O)N(C)C(C)C] | Intermediate 110 & N-methylpropan-2-amine |

### Alternative Approach for the Preparation of Intermediate 6

Intermediate 111 (1.3 g, 4.0 mmol) was dissolved in MeCN (40 mL). Next, CuBr₂ (2.7 g, 12 mmol) was added, and the mixture was stirred at room temperature for 5 h. Next, 7N NH₃/MeOH (20 mL) was added. The reaction mixture was stirred vigorously for ~30 min. Then, water (40 mL) and isopropyl acetate were added. The layers were separated, and the water layer was extracted twice with isopropyl acetate. The organic layers were combined, washed with brine, dried over Na₂SO₄, filtered and evaporated to dryness. The residue was purified by silica gel column chromatography eluting with methanol in dichloromethane from 0% to 3% to provide intermediate 6 (1.2 g, yield 72%) as an orange oil.

### Preparation of Intermediate 9:

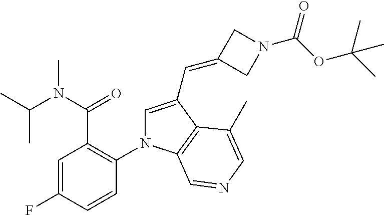 [SMILES: Cc1cncc2c1c(C=C1CN(C(=O)OC(C)(C)C)C1)cn2-c1ccc(F)cc1C(=O)N(C)C(C)C]

To a mixture of intermediate 6 (4 g, 4.312 mmol), tert-butyl 3-((4,4,5,5-tetramethyl-1,3,2-dioxaborolan-2-yl)methylene)azetidine-1-carboxylate (2.92 g, 9.9 mmol) and potassium carbonate (2.7 g, 19.7 mmol) in 1,4-dioxane (70 mL) and water (23 mL) was added Pd(dppf)Cl₂ (724 mg, 0.99 mmol). The mixture was degassed under nitrogen atmosphere three times and the reaction was stirred at 100° C. under nitrogen atmosphere for 16 h. After the mixture was cooled down to RT, the reaction mixture was diluted with H₂O and extracted with EtOAc. The combined organic phase was washed with brine, dried over Na₂SO₄, filtered and concentrated under vacuum. The residue was purified by silica gel column chromatography eluting with 90% ethyl acetate in petroleum ether to give intermediate 9 (1.8 g, 45.7% purity, 38.7% yield) as a yellow solid.

### Preparation of Intermediate 10:

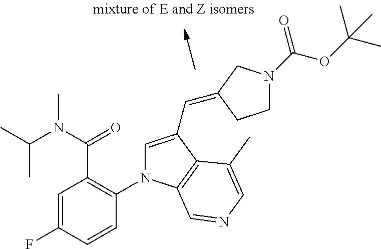 [SMILES: Cc1cncc2c1c(/C=C1\CCN(C(=O)OC(C)(C)C)C1)cn2-c1ccc(F)cc1C(=O)N(C)C(C)C]

A mixture intermediate 6 (12.0 g, 29.8 mmol), tert-butyl 3-((4,4,5,5-tetramethyl-1,3,2-dioxaborolan-2-yl)methylene)pyrrolidine-1-carboxylate (9.2 g, 29.8 mmol) and potassium carbonate (12.3 g, 89.1 mmol) in 1,4-dioxane (120 mL) and water (20 mL) was degassed and exchanged with N₂ twice. Pd(dppf)Cl₂ (2.16 g, 2.95 mmol) was added and the reaction mixture was heated up to 100° C. and stirred at this temperature overnight. After the reaction mixture was cooled down to r.t., the resulting mixture was concentrated and the residue was purified by silica gel column chromatography eluting with ethyl acetate in petroleum ether from 0% to 80% to give intermediate 10 (12.0 g, 79.4% yield) as a yellow oil.

### Preparation of Intermediate 15:

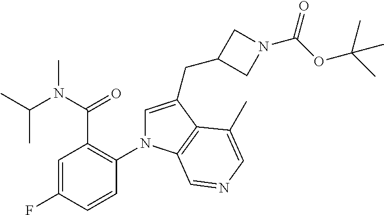 [SMILES: Cc1cncc2c1c(CC1CN(C(=O)OC(C)(C)C)C1)cn2-c1ccc(F)cc1C(=O)N(C)C(C)C]

A mixture of intermediate 9 (6.0 g, 12.2 mmol) in methanol (100 mL) was degassed under nitrogen atmosphere three times. 10 w/w % palladium on charcoal (3 g) was added and the mixture was degassed under hydrogen atmosphere three times. The mixture was stirred at r.t. under hydrogen atmosphere (balloon) for 16 h. The mixture was filtered and the filtrate was concentrated and purified by silica gel column chromatography eluting with 50% ethyl acetate in petroleum ether to give intermediate 15 (5.2 g, 97% purity, 83.7% yield) as a yellow solid.

### Preparation of Intermediate 16, 17 & 18:

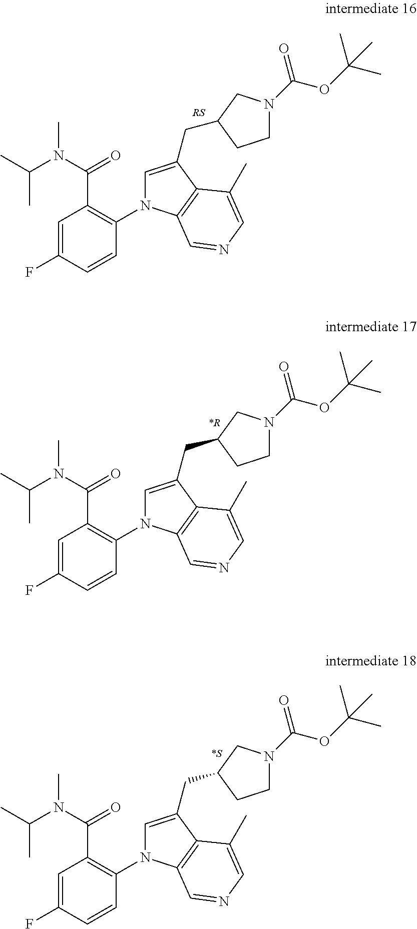

To a solution of intermediate 10 (2.5 g, 93% purity, 4.59 mmol) in methanol (40 mL) was added 10 w/w % palladium on charcoal (1 g) under N2. The suspension was degassed under vacuum and purged with H₂ several times. The reaction mixture was heated up to 30° C. and stirred at this temperature overnight. After the reaction was cooled down to r.t., the reaction mixture was filtered and the filtrate was concentrated and purified by silica gel column chromatography eluted with methanol in dichloromethane from 0% to 5% to give intermediate 16 (2.5 g, 93% purity, 99.6% yield) as a yellow oil.

Intermediate 16 (8 g, 95% purity, 14.9 mmol) was separated by chiral IG-SFC (separation condition: Column: IG; Mobile Phase: CO₂—IPA: 65:35, at 60 mL/min; Temp: 40° C.; Wavelength: 214 nm) to afford intermediate 17 (first fraction, 3.29 g, 98% purity, 42.4% yield) as a yellow oil and intermediate 18 (second fraction, 3.36 g, 98% purity, 43.3% yield) as a yellow solid.

Chiral SFC method 2 was employed to match the stereochemistry of intermediate 18 and intermediate 201, retention time=5.97-6.10 min.

### Preparation of Intermediate 25:

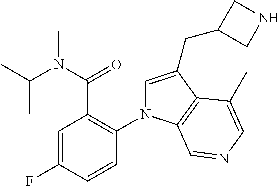 [SMILES: Cc1cncc2c1c(CC1CNC1)cn2-c1ccc(F)cc1C(=O)N(C)C(C)C]

To a cooled (ice bath) solution of intermediate 15 (1.1 g, 2.2 mmol) in dichloromethane (14 mL) was added dropwise TFA (7 mL). Then, the mixture was stirred at r.t. for 2 h. The solvent was removed by evaporation and the residue was dissolved in DCM, the pH was adjusted to 8-9 with saturated sodium carbonate aqueous solution, and extracted with DCM. The organic phase was dried over Na₂SO₄ and concentrated under vacuum to give intermediate 25 (680 mg, 72% yield) as a white solid.

The following intermediates and Compounds were synthesized by an analogous method as described for intermediate 25

| | | |
|---|---|---|
| Intermediate 27 | 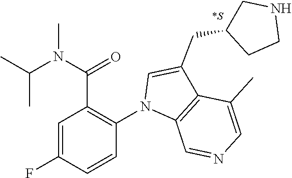 [SMILES: Cc1cncc2c1c(C[C@H]1CCNC1)cn2-c1ccc(F)cc1C(=O)N(C)C(C)C] | Intermediate 18 |

### Preparation of Intermediate 201—Method A:

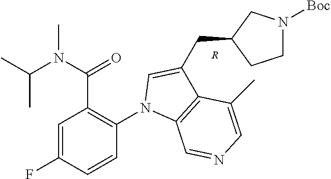

Into a 2 L 4-necked round-bottom flask were added THE (345 mL) and Zn (120.87 g, 1847.90 mmol, 5.00 equiv) at 30° C. under a nitrogen atmosphere. A solution of TMSCl (8.03 g, 73.91 mmol, 0.2 equiv) and 1-bromo-2-chloroethane (10.60 g, 73.91 mmol, 0.20 equiv) in THE (230 mL) were added into above round-bottom flask with a Lead Fluid-BT100F peristaltic pump (rate: 10 mL/min) under a nitrogen atmosphere. The resulting mixture was stirred for additional 40 min at 30° C. Next, a Lead Fluid-BT100F peristaltic pump was used to remove the solvent in above RBF quickly, and then fresh THE (575 mL) was re-charged under a nitrogen atmosphere. The mixture was heated to 60° C. Next, a solution of tert-butyl (3R)-3-(iodomethyl)pyrrolidine-1-carboxylate (115 g, 369.58 mmol, 1.00 equiv) in THE (575 mL) was added into above RBF with a Lead Fluid-BT100F peristaltic pump (rate: 15.0 mL/min) under a nitrogen atmosphere (temperature rises to 60-65° C.). The solution was stirred at 60° C. for an additional 1 h. The mixture was then cooled to 30° C. and allowed to stand for 1 h. The solution of {[(3R)-1-(tert-butoxycarbonyl)pyrrolidin-3-yl]methyl}(iodo)zinc was used directly in the next step. The concentration of the product was about 0.37 moL/L in THE.

Into a 2 L 4-necked round-bottom flask were added intermediate 6 (105 g, 259.71 mmol, 1.00 equiv) and THE (500 mL) at 30° C. under nitrogen atmosphere. To the stirred solution was added the 4th Generation RuPhos Pd precatalyst (5.65 g, 6.49 mmol, 0.025 equiv) under nitrogen atmosphere. Next, the solution of {[(3R)-1-(tert-butoxycarbonyl)pyrrolidin-3-yl]methyl}(iodo)zinc was added with a Lead Fluid-BT100F peristaltic pump into the 2 L 4-need RBF quickly under a nitrogen atmosphere (the excess zinc dust was not transferred). The resulting mixture was stirred for an additional 16 h at 50° C. The reaction was repeated 6 times in parallel. The reaction was quenched by the addition of aqueous sat. NH₄Cl solution (12 L). The aqueous layer was extracted with EtOAc (3×6 L), the organic layer was washed with water (2×3 L) and brine (1×3 L). The resulting mixture was dried with Na₂SO₄ and concentrated under reduced pressure. The crude product as a black oil (1100 g, crude) was used directly into the next step (preparation of intermediate 202)

### Alternatively, the Procedure Described Below can be Employed for the Preparation of Intermediate 201—Method B

A column (1.5 cm×15 cm) was stoppered with cotton wool and filled with granular zinc (20-30 mesh), 22 g. The column volume of the filled column was determined by measuring the time for THF to fill the column at 1 mL/min flow rate. Column volume=4.3 mL. The zinc was activated by flowing a strong activating solution through the column at 0.5 mL/min for 10 mins. The strong activating solution consists of 1 mL TMSCl (0.67 M) & 0.75 mL chlorobromoethane (0.71 M) in 10 mL THE. After activation, the column was washed with dry THE: 10 mL, 1 mL/min. tert-butyl (R)-3-(iodomethyl)pyrrolidine-1-carboxylate (10 g, 37 mmol) was dissolved in THF (60 mL). The iodide solution was flowed through the activated zinc column at 50° C., flow rate 0.45 mL/min. After reaction: titration with iodine shows a concentration of 0.30 M.

Intermediate 6 (1.2 g, 2.4 mmol) was added with RuPhos Pd G4 (0.051 g, 0.06 mmol) in a sealed vial with a stirring bar in a glove box. Then, a solution of freshly made R-((1-(tert-butoxycarbonyl)-3-yl)methyl)zinc(II) iodide (12 mL, 0.3 M, 3.6 mmol) which was prepared by the above procedure was added. Next, the solution was heated to 50° C. under nitrogen atmosphere during 16 h. The solution was concentrated in vacuo and the residue redissolved in DCM. Next, water was added, followed by aq. Na₄EDTA solution (pH>10). The layers were separated and the water layer was extracted once more with DCM. Organic layers were combined, dried over Na₂SO₄, filtered and evaporated to dryness. The residue was purified by silica gel column chromatography eluting with methanol in dichloromethane from 0% to 10% to give intermediate 201 (1.4 g, 1.5 mmol (55% purity), 63% yield).

The Following Intermediates were Synthesized by an Analogous Method (Method B) as Described for Intermediate 201

| | | |
|---|---|---|
| Intermediate 367 | 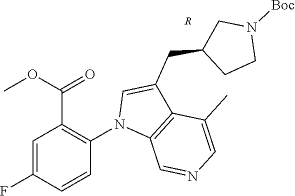 | Intermediate 361 & tert-butyl (R)-3-(iodomethyl)pyrrolidine-1- carboxylate |
| Intermediate 224 | 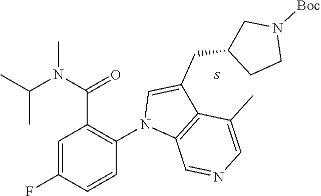 | Intermediate 6 & tert-butyl (S)- 3-(iodomethyl)pyrrolidine-1- carboxylate |

### Preparation of Intermediate 202:

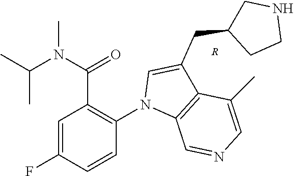

The mixture of intermediate 201 (17 g, 33.09 mmol) in dichloromethane (50 mL), was added the solution 24 mL of chlorine hydride (7 M in ethyl acetate). After stirring at r.t. for 5 h, the reaction mixture was concentrated, and the residue was diluted with DCM and basified with sodium hydroxide aqueous solution (1M) to pH~10. The layers were separated and the aqueous layer was extracted with DCM three times and the combined organic layer was washed with brine (30 mL), dried over sodium sulfate, filtered and concentrated to afford intermediate 202 (13 g, 31.1 mmol, 94.2% yield) as a yellow solid, which was used in the next step without purification.

Alternatively, intermediate 202 can also be prepared as a 0.2TFA salt by using the following procedure:

Intermediate 201 (5.2 g, 6.95 mmol, 68% pure) is dissolved in DCM (44.5 mL) and TFA (5.3 mL) was added and stirred for 4 h at rt. The solution was concentrated in vacuo and coevaporated with toluene. Next, the mixture was washed with 1M NaOH and extracted four times with 10 DCM and EtOAc and Me-THF to obtain the combined organics which were then dried with anhydrous Na₂SO₄, filtered and concentrated in vacuo. The residue was purified via by silica gel column chromatography eluting with methanol (containing 7N NH₃) in dichloromethane from 0% to 10% to give intermediate 202 as a 0.2TFA salt.

Alternatively, intermediate 202 can also be prepared with the following procedure:

Into a 10 L 4-necked round-bottom flask were added 4N HCl in 1,4-dioxane (1.8 L). Then, crude intermediate 201 in THE (3 L) was added dropwise (calculated by 735 g intermediate 201, 1.82 mol, 1.0 equiv) at 0° C. The resulting mixture was stirred for an additional 2 h at 0° C. The resulting mixture was diluted with ethyl acetate (3 L) and water (3 L). The aqueous layer was washed with DCM (10×1 L). The pH of the aqueous layer was adjusted to pH 8 with saturated aqueous Na₂CO₃ solution and extracted with CH₂Cl₂ (4×2 L). The organic layers were dried with Na₂SO₄ and concentrated under vacuum to afford intermediate 202 (389 g, yield 53% over 2 steps) as a light yellow solid.

The Following Intermediate were Synthesized by an Analogous Method as Described for Intermediate 202

| Int. No. | Structure | Starting Materials |
|---|---|---|
| Intermediate 225 | 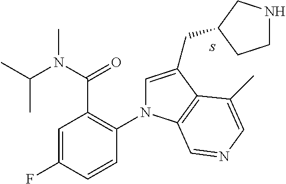 | Intermediate 224 |
| Intermediate 368 | 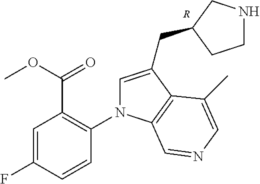 | Intermediate 367 |

### Preparation of Intermediate 361:

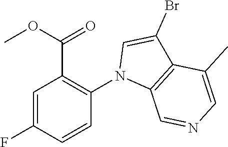 [SMILES: COC(=O)c1cc(F)ccc1-n1cc(Br)c2c(C)cncc21]

Intermediate 4 (3.3 g, 9.451 mmol) was dissolved in MeOH (38.2 mL) and cooled to 0° C. before thionyl chloride (13.7 mL, 189.0 mmol) was added dropwise. The solution was then heated to 70° C. for 2 hours. After cooling to ambient temperature, the solution was concentrated in vacuo and directly purified by silica gel column chromatography eluting with methanol (containing 7N NH₃) in dichloromethane from 0% to 10% to give intermediate 361 (3.7 g, 100% yield) as an oil.

### Preparation of Compound 43:

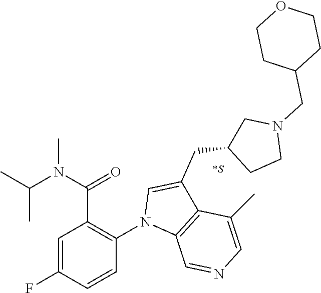

To a mixture of intermediate 27 (90 mg, 0.22 mmol) and tetrahydro-2H-pyran-4-carbaldehyde (74 mg, 0.65 mmol) in methanol (2 mL) was added sodium cyanoborohydride (40 mg, 0.65 mmol). The reaction mixture was stirred at 20° C. overnight. The mixture was concentrated and purified by Prep. HPLC (Column: GiLSON-2 Xbridge C18 (5 μm 19*150 mm), Mobile phase A: water (0.1% ammonium bicarbonate), Mobile phase B: acetonitrile, UV: 214 nm, Flow rate: 15 mL/min, Gradient: 20% B to 60% B). The collected fraction was lyophilized to give Compound 43 (48 mg, 95% purity, 41% yield) as a white solid.

The Following Compounds were Synthesized by an Analogous Method as Described for Compound 43

In case reactions were performed with a ketone starting material, a typical procedure makes use of either 2 eq. acetic acid or 2 eq. of zinc(II)chloride (ZnCl₂), in the presence of 2 eq. sodium cyanoborohydride (NaCNBH₃), in methanol at 50° C. or 70° C. overnight.

| Compound No. | Structure | Starting Materials |
|---|---|---|
| Compound 156 | 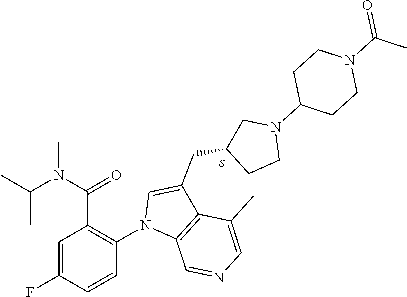 | Intermediate 225 & 1-acetylpiperidin-4- one |
| Compound 188a & | 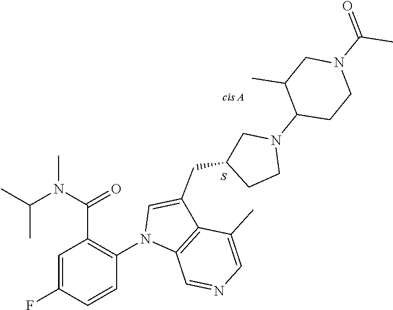 | Intermediate 225 & 1-acetyl-3- methylpiperidin-4-one The product was separated by Prep. HPLC (Column: SunFire C18 150*19 mm*5 um, Mobile Phase A: water (0.1% NH₄OAc), Mobile Phase B: acetonitrile, Flow rate: 15 mL/min, gradient condition from 10% B to 50%). The first fraction was collected as Compound 188a and the second fraction as Compound 188b. |
| Compound 188b | 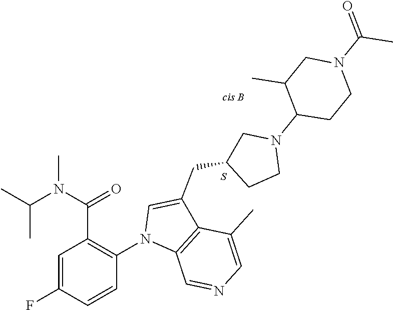 | |
| Compound 190a & | 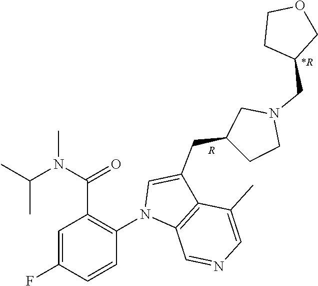 | Intermediate 202 & tetrahydrofuran-3- carbaldehyde The product was separated by Prep SFC (Stationary phase: Chiralpak Diacel AD 20 × 250 mm, Mobile phase: CO₂, EtOH-iPrOH (50-50) + 0.4% iPrNH₂). The first fraction was collected as Compound 190a and the second fraction as Compound 190b. |
| Compound 190b | 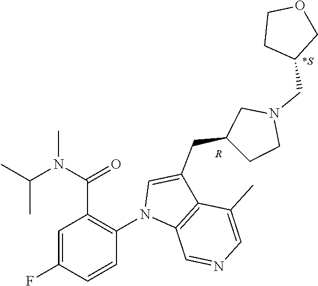 | |
| Compound 191a & | 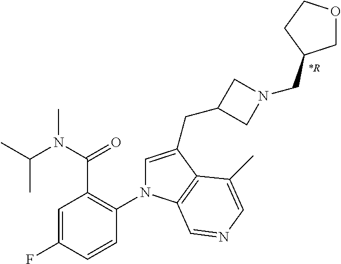 [SMILES: Cc1cncc2c1c(CC1CN(C[C@H]3CCOC3)C1)cn2-c1ccc(F)cc1C(=O)N(C)C(C)C] | Intermediate 25 & tetrahydrofuran-3- carbaldehyde The product was separated by P Prep SFC (Stationary phase: Chiralcel Diacel IH 20 × 250 mm, Mobile phase: CO₂, iPrOH + 0.4 iPrNH₂). The first fraction was collected as Compound 191a and the second fraction as Compound 191b. |
| Compound 191b | 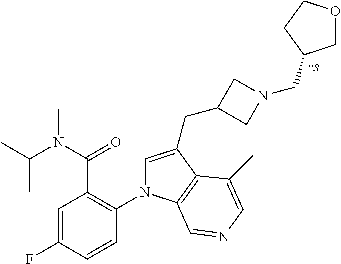 [SMILES: Cc1cncc2c1c(CC1CN(C[C@@H]3CCOC3)C1)cn2-c1ccc(F)cc1C(=O)N(C)C(C)C] | |
| Compound 196 | 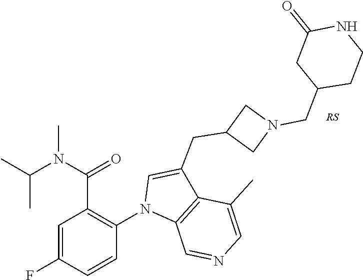 [SMILES: Cc1cncc2c1c(CC1CN(CC3CCNC(=O)C3)C1)cn2-c1ccc(F)cc1C(=O)N(C)C(C)C] | Intermediate 25 & 2-oxopiperidine-4- carbaldehyde |
| Compound 213a & | 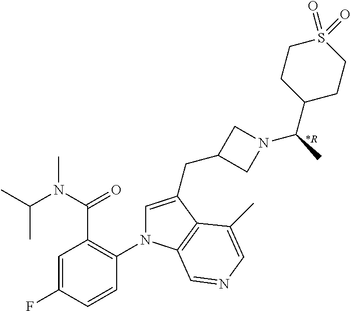 [SMILES: Cc1cncc2c1c(CC1CN([C@H](C)C3CCS(=O)(=O)CC3)C1)cn2-c1ccc(F)cc1C(=O)N(C)C(C)C] | Intermediate 25 & 1-(1,1- dioxidotetrahydro-2H-thiopyran-4- yl)ethan-1-one The product was separated by SFC (separation condition: DAICEL CHIRALPAK IG (250 mm*30 mm, 10 um); Mobile phase: A: Supercritical CO₂, B: 0.1%NH₃H₂O MeOH, A:B = 40:60 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 213a and the second fraction as Compound 213b. |
| Compound 213b | 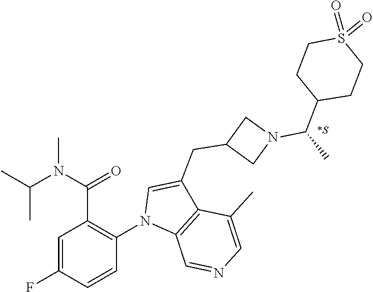 [SMILES: Cc1cncc2c1c(CC1CN([C@@H](C)C3CCS(=O)(=O)CC3)C1)cn2-c1ccc(F)cc1C(=O)N(C)C(C)C] | |
| Compound 194 | 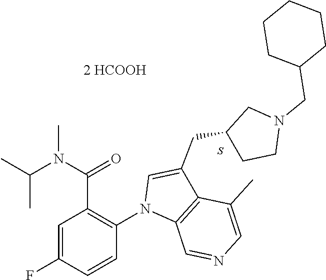 | Intermediate 225 & cyclohexanecarbaldehyde |
| Compound 169 | 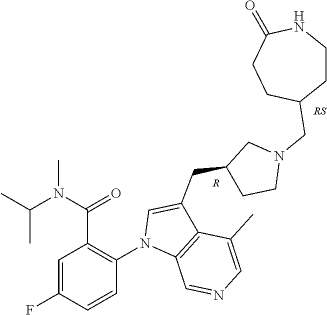 | Intermediate 202 & 7-oxoazepane-4- carbaldehyde |
| Compound 169a & | 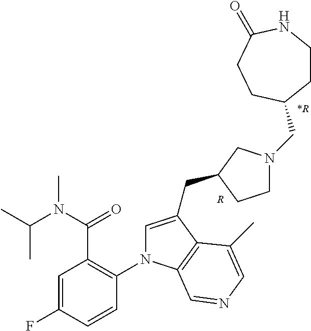 | Compound 169 was separated by Prep SFC (Stationary phase: Chiralcel Diacel IH 20 × 250 mm, Mobile phase: CO₂, EtOH + 0.4 iPrNH₂). The first fraction was collected as Compound 169a & the second fraction as Compound 169b. |
| Compound 169b | 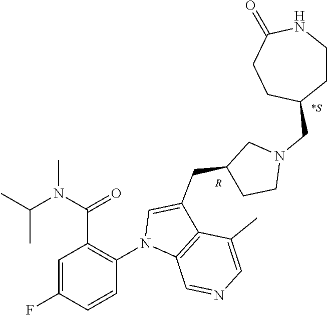 | |

### Preparation of Compound 50:

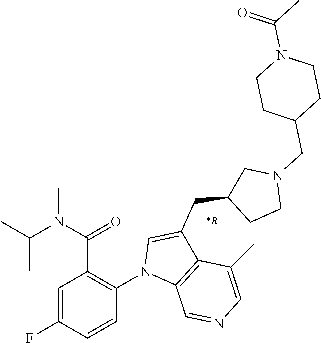

To a solution of Compound 381 (70 mg, 0.102 mmol) and DIEA (79 mg, 0.61 mmol) in DCM (4 mL) was added acetic anhydride (52 mg, 0.51 mmol). After stirring at r.t. for 4 hours, the reaction mixture was concentrated, and the residue was purified by Prep-HPLC: Waters Xbridge C18 5 μm 19*150 mm. Mobile phase A: 0.1% NH₄OH+10 mM NH₄HCO₃ in water. B: CH₃CN, gradient from 0% B to 100% B. The pure fraction was collected and lyophilized to afford Compound 50 (50 mg, 88% yield) as a white solid.

### Preparation of Compound 51:

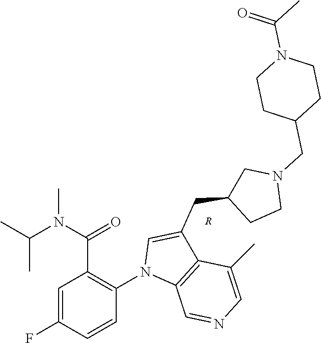

At 0° C., to a solution of Compound 485 (1.04 g, 95% purity, 1.95 mmol) in DCM (10 mL) was added acetyl chloride (160 mg, 2.05 mmol) and triethylamine (592 mg, 5.85 mmol). After stirring at room temperature for 2 hours, the resulting mixture was poured into water and extracted with dichloromethane (20 mL) twice. The combined organic layers were washed with brine (20 mL), dried over Na₂SO₄, filtered and concentrated to afford the crude product, which was purified by prep HPLC (Column: Xbridge C18 150*19 mm*5 um, Mobile Phase A: water (0.1% NH₄HCO₃), Mobile Phase B: acetonitrile, Flow rate: 15 mL/min, gradient condition from 15% B to 60% B). The collected fraction was lyophilized to give Compound 51 (1.25 g, 99.8% purity, 74.9% yield) as a white solid.

Alternatively, compound 51 can also be prepared with the following procedure:

Intermediate 202 (as 0.2TFA salt) (0.20 g, 0.49 mmol) and 1-acetylpiperidine-4-carbaldehyde (0.097 g, 0.62 mmol) were dissolved in MeOH (5.5 mL). After stirring at ambient temperature for ~5 min, solid NaCNBH₃ (0.039 g, 0.62 mmol) was added. The resulting mixture was stirred at ambient temperature for ~2 h, after which sat. aq. NaHCO₃ solution was added. Then, most of the MeOH was evaporated to dryness, and DCM was added. The pH of the water layer was adjusted to pH>10 with 1M aq. NaOH solution. The layers were separated and the water layer was extracted three times more with DCM. The organic layers were combined, dried over Na₂SO₄, filtered and evaporated. The residue was purified by silica gel column chromatography eluting with methanol (+1% 7N NH₃ in MeOH) in dichloromethane from 0% to 10% to give compound 51 (0.060 g, 0.11 mmol, 35% yield).

Compound 51 (originating from route via intermediate 202; 0.051 g, purity 99.7%, LC/MS method 32) was dissolved in 2-3 drops of isopropylacetate (IPAC), after which the resulting solution was stirred at 45° C. for ~5 h. Next, the mixture was allowed to stir at ambient temperature for 48 h, after which it was filtered to obtain a white solid material corresponding with Compound 51 in its crystalline free base Form. Melting point (via DSC): Tₒₙₛₑₜ=121.6° C.

Compound 51 ((originating from route via intermediate 202; ~1 g, 98.7% purity, LC/MS method 33) was dissolved in cyclopentylmethylether (CPME) (3 mL), after which heptane (2 mL) was slowly added, followed by the addition of ~10 mg of seeding crystals (obtained via previous procedure). Next, 1 mL of heptane was added and the mixture stirred for 20 h, after which the suspension was filtered to give solid material which was dried at 40° C. under vacuum to yield Compound 51 in its crystalline free base Form (96% yield).

Chiral SFC method 1 was employed to match the stereochemistry of compound 51 obtained through the route employing Compound 485 or intermediate 202; retention time=4.73-4.77 min.

### Preparation of Compound 51a:

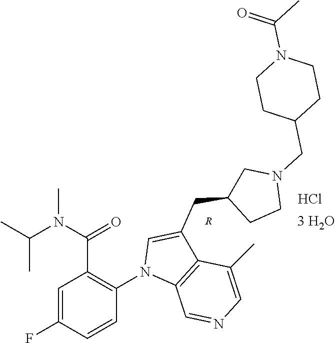

Compound 51 (0.50 g, 0.91 mmol, purity 95.2% (determined by LC/MS method 32)) was dissolved in acetone (0.50 mL) and stirred to give a clear solution. Next, a solution of 1M HCl in acetone was prepared as follows: 1 mL of concentrated aq. HCl solution was added to 11 mL of acetone. Then, a solution of 1M HCl in acetone (0.92 mL, 1 eq.) was added, keeping a solution. The solution was stirred at ambient temperature for ~30-60 min, after which heptane (5.0 mL) was added. Next, acetone was added (3.0 mL). Vigorous stirring was initiated, and the mixture was stirred overnight. Then, a fine white suspension was obtained, and the suspension was filtered. The solid was rinsed with heptane and dried to give Compound 51a as a mono HCl trihydrate salt (when determined via dynamic vapor sorption analysis around 3 equivalents water) as a white solid (0.48 g, yield 78%). Melting point (via DSC): Tₒₙₛₑₜ=139° C.

Compound 51a was obtained as a variable hydrate with equilibrated water content varying as function of humidity—mainly trihydrate at ambient % relative humidity.

The following compounds were synthesized by an analogous method as described for Compound 51

Alternatively, compounds can also be purified by the following method: prep. HPLC: (Column: Waters Sunfire C18 5 μm, 19*150 mm, Mobile Phase A: water (0.1% HCOOH), Mobile Phase B: acetonitrile, Flow rate: 17 mL/min, gradient condition from 0% B to 20% B).

| Compound No. | Structure | Starting Materials |
|---|---|---|
| Compound 121 | 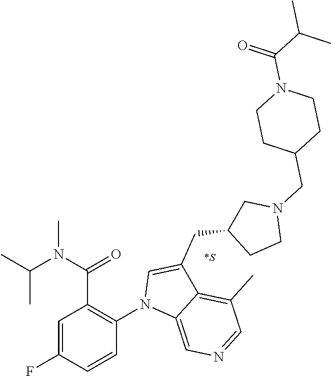 [SMILES: Cc1cncc2c1c(C[C@H]1CCN(CC3CCN(C(=O)C(C)C)CC3)C1)cn2-c1ccc(F)cc1C(=O)N(C)C(C)C] | Compound 485 & isobutyryl chloride |
| Compound 135 | 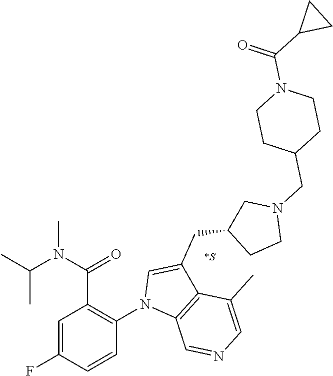 [SMILES: Cc1cncc2c1c(C[C@H]1CCN(CC3CCN(C(=O)C4CC4)CC3)C1)cn2-c1ccc(F)cc1C(=O)N(C)C(C)C] | Compound 485 & cyclopropanecarbonyl chloride |

### Preparation of Compound 59:

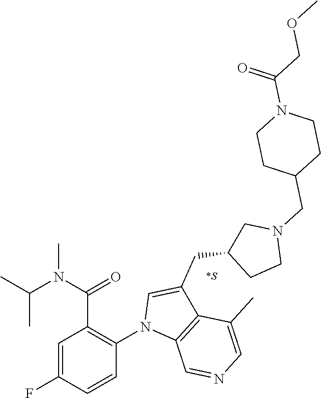

To a mixture of Compound 485 (70 mg, 0.138 mmol), methoxyacetic acid (18.7 mg, 0.208 mmol) and DIPEA (0.07 mL, 0.42 mmol) in DCM (4.2 mL) was added HATU (78.9 mg, 0.208 mmol). After stirring at rt for 16 hours, the reaction mixture was concentrated and the residue was purified by Prep. HPLC (Column: Waters Xbridge C18 5 μm, 19*150 mm, Mobile Phase A: water (0.1% NH₃H₂O+10 mM NH₄HCO₃), Mobile Phase B: acetonitrile, Flow rate: 17 mL/min, gradient condition from 30% B to 50% B). The pure fraction was collected and lyophilized to dryness to afford Compound 59 (65 mg, 79.6% yield).

### Preparation of Compound 60:

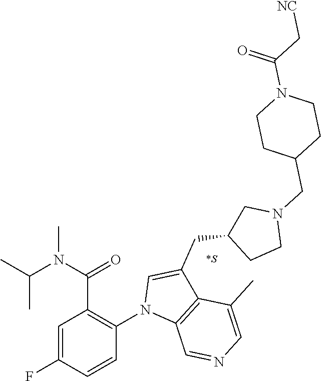

To a mixture of Compound 485, (70 mg, 0.138 mmol), cyanoacetic acid (17.7 mg, 0.208 mmol) and DIPEA (0.07 mL, 0.415 mmol) in DCM (5 mL) was added HATU (78.9 mg, 0.208 mmol). After stirring at RT for 16 hours, the reaction mixture was concentrated, and the residue was purified by Prep. HPLC (Column: Waters Xbridge C18 5 μm, 19*150 mm, Mobile Phase A: water (0.1% NH₃H₂O+10 mM NH₄HCO₃), Mobile Phase B: acetonitrile, Flow rate: 17 mL/min, gradient condition from 30% B to 50% B). The pure fraction was collected and lyophilized to dryness to afford Compound 60 (65 mg, 81% yield).

The Following Compounds were Synthesized by an Analogous Method as Described for Compound 60

Alternatively, purification can also be performed using the following method: Prep. HPLC (Column: Xbridge C18 150*19 mm*5 um, mobile phase A: water (0.1% HCOOH), mobile phase B: acetonitrile, flow rate: 15 mL/min, gradient condition from 5% B to 60% B)

| Compound No. | Structure | Starting Materials |
|---|---|---|
| Compound 82 | 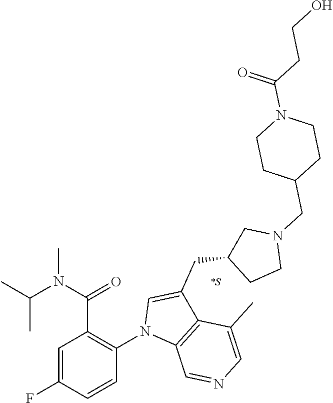 | Compound 485 & 3- hydroxypropanoic acid |
| Compound 85 | 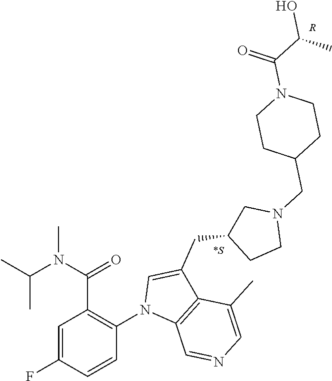 | Compound 485 & (R)-2- hydroxypropanoic acid |
| Compound 86 | 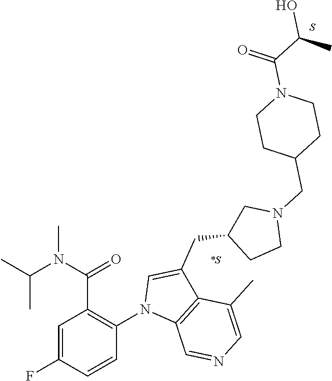 | Compound 485 & (S)-2- hydroxypropanoic acid |
| Compound 87 | 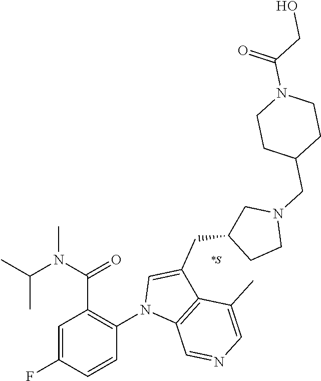 | Compound 485 & 2- hydroxyacetic acid |
| Compound 106 | 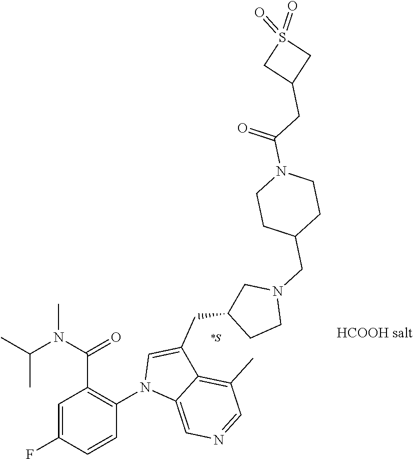 | Compound 485 & 2-(1,1- dioxidothietan-3-yl)acetic acid |
| Compound 108 | 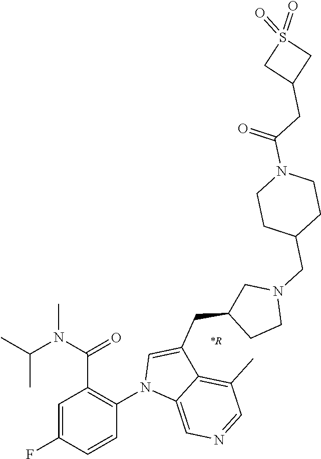 | Compound 381 & 2-(1,1- dioxidothietan-3-yl)acetic acid |
| Compound 125 | 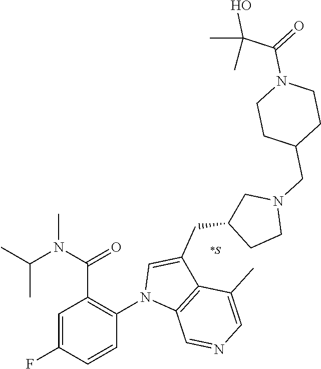 | Compound 485 & 2-hydroxy-2- methylpropanoic acid |
| Compound 126 | 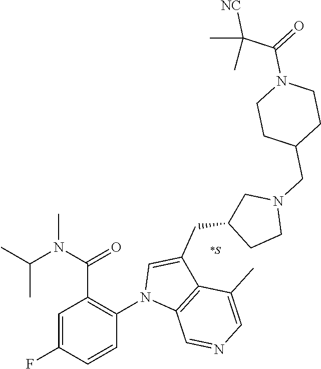 | Compound 485 & 2-cyano-2- methylpropanoic acid |
| Compound 117a | 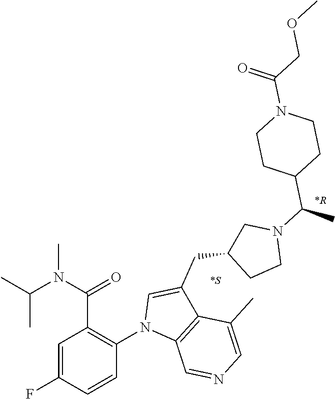 | Compound 442 & 2- methoxyacetic acid |

### Preparation of Compound 61

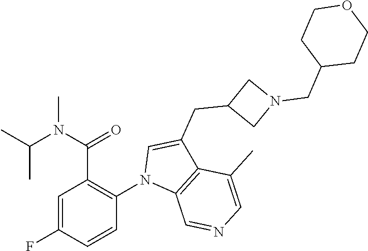 [SMILES: Cc1cncc2c1c(CC1CN(CC3CCOCC3)C1)cn2-c1ccc(F)cc1C(=O)N(C)C(C)C]

To a solution of intermediate 25 (0.082 g, 0.21 mmol) in 1,2-DCE (2.0 mL) was add tetrahydropyran-4-carbaldehyde (0.028 g, 0.25 mmol), and followed by NaBH(OAc)₃ (0.062 g, 0.29 mmol). After stirring at ambient temperature overnight, another portion of tetrahydropyran-4-carbaldehyde (0.028 g, 0.25 mmol) and NaBH(OAc)₃ (0.062 g, 0.29 mmol) was added. After stirring for another 1.5 h, 1M aq. NaOH solution was added, followed by DCM. The layers were separated, and the aqueous layer was extracted 4× with DCM. The organic layers were combined, dried over Na₂SO₄, filtered and evaporated. The residue was purified by RP-preparative HPLC (Stationary phase: RP Xbridge Prep C18 OBD—5 μm, 50×250 mm, Mobile phase: 0.5% NH₄HCO₃ solution in water, CH₃CN) to give Compound 61 (0.058 g, 57% yield), after lyophilization, as a white fluffy powder.

### Preparation of Compound 62:

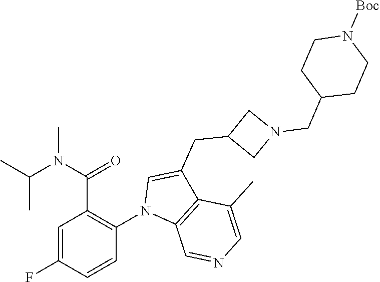 [SMILES: Cc1cncc2c1c(CC1CN(CC3CCN(C(=O)OC(C)(C)C)CC3)C1)cn2-c1ccc(F)cc1C(=O)N(C)C(C)C]

To a solution of intermediate 25 (0.18 g, 0.45 mmol) in 1,2-DCE (4.2 mL) was added N-Boc-piperidine-4-carboxaldehyde (0.11 g, 0.54 mmol), and followed by NaBH(OAc)₃ (0.13 g, 0.63 mmol). After stirring at ambient temperature overnight, DCM and 1M aq. NaOH were added. The layers were separated, and the aqueous layer was extracted 4× more with DCM. The organic layers were combined, dried over Na₂SO₄, filtered and evaporated. The residue was purified by silica gel column chromatography eluting with methanol (+1% 7N NH3 in MeOH) in dichloromethane from 0% to 10% to give Compound 62 (0.25 g, 94% yield) as a foam.

### Preparation of Compound 63:

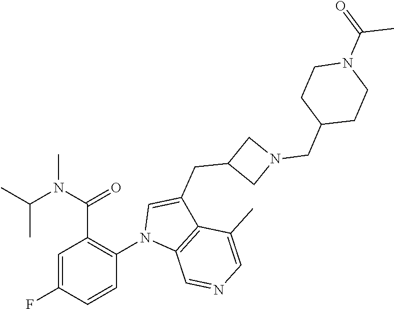 [SMILES: CC(=O)N1CCC(CN2CC(Cc3cn(-c4ccc(F)cc4C(=O)N(C)C(C)C)c4cncc(C)c34)C2)CC1]

To a solution of Compound 62 (0.25 g, 0.42 mmol) in DCM (5 mL) was added TFA (5 mL). After stirring at ambient temperature for 2 h, the reaction mixture was evaporated to dryness and the residue applied to SiliaBond® propylsulfonic acid resin as a solution in MeOH. The column was eluted with MeOH (8 fractions), followed by 3.5 N NH₃ in MeOH (8 fractions). Product containing fractions were pooled and evaporated to give an intermediate, which was dissolved in DCM (3.6 mL). The solution was cooled to 0° C. in an ice bath and DIPEA (0.13 mL, 0.76 mmol) was added, followed by Ac₂O (0.06 mL, 0.63 mmol). The resulting mixture was stirred at ambient temperature for 2 h, after which LC/MS showed full conversion of the starting material. Then, sat. aq. NaHCO₃ solution was added. The resulting mixture was partitioned between 1M aq. NaOH solution and DCM. The water layer was extracted 5× with DCM and the organic layers were combined, dried over Na₂SO₄, filtered, and evaporated to dryness. The residue was purified by RP-preparative HPLC (Stationary phase: RP Xbridge Prep C18 OBD—5 μm, 50×250 mm, Mobile phase: 0.5% NH₄HCO₃ solution in water, CH₃CN) to give Compound 63 (0.046 g, 68% yield) after lyophilization as a white fluffy powder.

The Following Compounds were Synthesized by an Analogous Method as Described for Compound 63

| Compound No. | Structure | Starting Materials |
|---|---|---|
| Compound 64 | 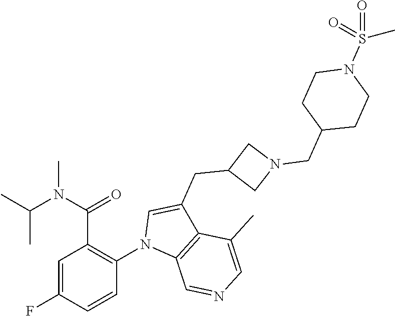 [SMILES: Cc1cncc2c1c(CC1CN(CC3CCN(S(C)(=O)=O)CC3)C1)cn2-c1ccc(F)cc1C(=O)N(C)C(C)C] | Compound 62 & methanesulfonyl chloride |
| Compound 65 | 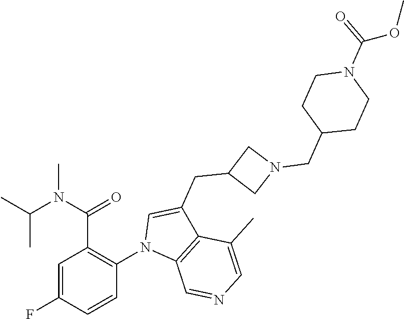 [SMILES: COC(=O)N1CCC(CN2CC(Cc3cn(-c4ccc(F)cc4C(=O)N(C)C(C)C)c4cncc(C)c34)C2)CC1] | Compound 62 & methyl carbonochloridate |

### Preparation of Compound 83:

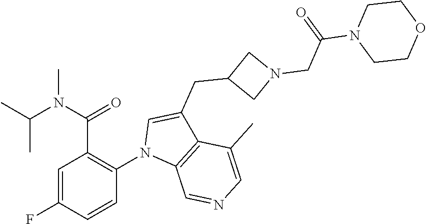 [SMILES: Cc1cncc2c1c(CC1CN(CC(=O)N3CCOCC3)C1)cn2-c1ccc(F)cc1C(=O)N(C)C(C)C]

Intermediate 25 (0.060 g, 0.152 mmol) was dissolved in MeCN (1.6 mL). Then, 4-(2-chloroacetyl)morpholine (0.027 g, 0.17 g) and triethylamine (0.13 mL, 0.91 mmol) was added and the resulting mixture stirred at ambient temperature for 2 h. Next, MeOH was added and the mixture was evaporated to dryness. The residue was purified by RP-preparative HPLC (Stationary phase: RP Xbridge Prep C18 OBD—5 μm, 50×250 mm, Mobile phase: 0.5% NH₄HCO₃ solution in water, CH₃CN) to give Compound 83 (32 mg, 0.059 mmol, 39% yield).

### The Following Compound was Synthesized by an Analogous Method as Described for Compound 83

| Compound No. | Structure | Starting Materials |
|---|---|---|
| Compound 98 | 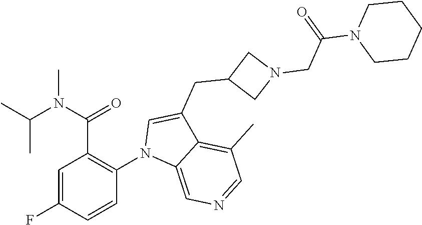 [SMILES: Cc1cncc2c1c(CC1CN(CC(=O)N3CCCCC3)C1)cn2-c1ccc(F)cc1C(=O)N(C)C(C)C] | Intermediate 25 & 2-chloro-1- piperidin-1-yl-ethanone |

### Preparation of Compound 91:

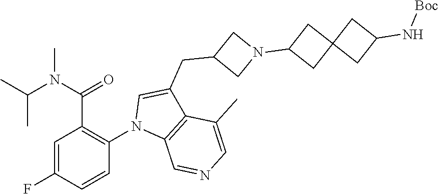 [SMILES: Cc1cncc2c1c(CC1CN(C3CC4(CC(NC(=O)OC(C)(C)C)C4)C3)C1)cn2-c1ccc(F)cc1C(=O)N(C)C(C)C]

A mixture of intermediate 25 (127 mg, 0.322 mmol), 2-(Boc-amino)-6-oxospiro[3.3]heptane (145 mg, 0.644 mmol) and AcOH (36.9 μL, 0.644 mmol) in MeOH (3.2 mL) was stirred for 30 min after which sodium cyanoborohydride (40.5 mg, 0.644 mmol) was added. The reaction mixture was stirred at 50° C. overnight. The reaction was cooled down to r.t., quenched with water, and evaporated to dryness. The residue was purified by silica gel column chromatography eluting with methanol (+1% NH3 in MeOH) in dichloromethane from 1% to 50%. The purest fractions were collected, evaporated to dryness to afford Compound 91 (64 mg, yield 32.6%) as a white solid.

The Following Compounds were Synthesized by an Analogous Method as Described for Compound 91

| Compound No. | Structure | Starting Materials |
|---|---|---|
| Compound 92 | 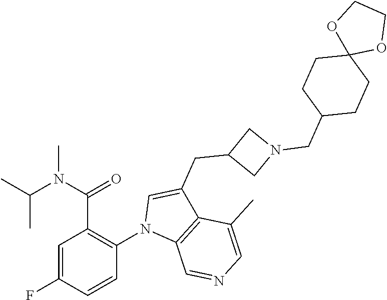 [SMILES: Cc1cncc2c1c(CC1CN(CC3CCC4(CC3)OCCO4)C1)cn2-c1ccc(F)cc1C(=O)N(C)C(C)C] | Intermediate 25 & 1,4- dioxaspiro[4.5]decane-8- carbaldehyde |
| Compound 104 | 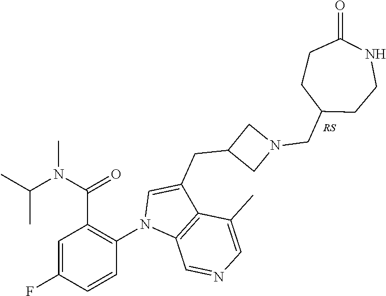 [SMILES: Cc1cncc2c1c(CC1CN(CC3CCNC(=O)CC3)C1)cn2-c1ccc(F)cc1C(=O)N(C)C(C)C] | Intermediate 25 & 7- oxoazepane-4-carbaldehyde |
| Compound 114 | 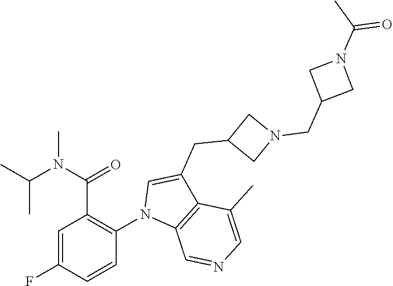 [SMILES: CC(=O)N1CC(CN2CC(Cc3cn(-c4ccc(F)cc4C(=O)N(C)C(C)C)c4cncc(C)c34)C2)C1] | Intermediate 25 & 1- acetylazetidine-3-carbaldehyde |
| Compound 120 | 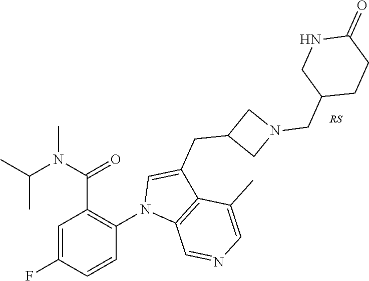 [SMILES: Cc1cncc2c1c(CC1CN(CC3CCC(=O)NC3)C1)cn2-c1ccc(F)cc1C(=O)N(C)C(C)C] | Intermediate 25 & 6- oxopiperidine-3-carbaldehyde |

### Preparation of Compound 101:

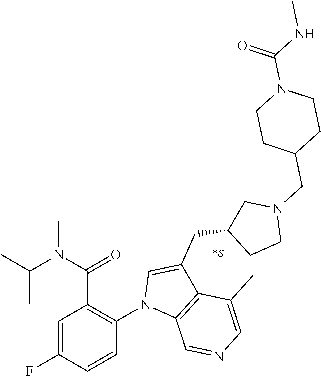 [SMILES: CNC(=O)N1CCC(CN2CC[C@H](Cc3cn(-c4ccc(F)cc4C(=O)N(C)C(C)C)c4cncc(C)c34)C2)CC1]

To a solution of Compound 485 (100 mg, 0.20 mmol), triethylamine (61 mg, 0.59 mmol) in dichloromethane (10 mL) was added a solution of methylaminoformyl chloride (23 mg, 0.22 mmol) in 2 mL of DCM. After stirring at 20° C. for 5 hr, the mixture was diluted with water (20 mL) and extracted with DCM (10 mL) for three times. The combined organic layers were washed with brine (30 mL), dried over Na₂SO₄ and filtered. The filtrate was concentrated under vacuum, which was purified by Prep-HPLC (Prep HPLC (Column: Xbridge C18 (5 μm 19*150 mm), Mobile Phase A: Water (0.1% NH₄HCO₃), Mobile Phase B: acetonitrile, UV: 214 nm, Flow rate: 15 mL/min, Gradient: 15% B to 55% B) to give Compound 101 (90 mg, 0.15 mmol, 76.8% yield) as a white solid.

### Preparation of Compound 207:

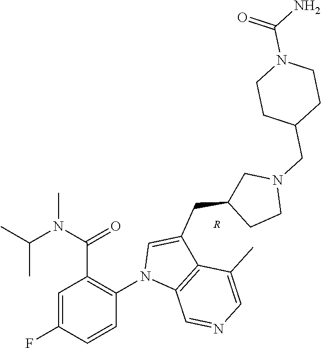

Compound 448 (120 mg, 0.237 mmol) and DIPEA (0.12 mL, 0.71 mmol) were added to DCM (5 mL). Isocyanatotrimethylsilane (32.8 mg, 0.28 mmol) was added and the mixture was stirred at r.t. for 16 hours. The solvent was removed and the residue was purified by preparative-HPLC (Column: Waters Xbridge C18 5 μm, 19*150 mm, Mobile Phase A: water (0.1% NH₄OH+10 mM NH₄HCO₃), Mobile Phase B: acetonitrile, Flow rate: 17 mL/min, gradient condition from 25% B to 35% B) to give Compound 207 (100 mg, 75% yield).

### Preparation of Compound 283:

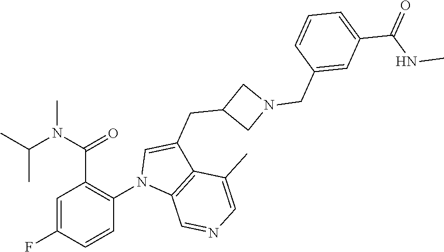 [SMILES: CNC(=O)c1cccc(CN2CC(Cc3cn(-c4ccc(F)cc4C(=O)N(C)C(C)C)c4cncc(C)c34)C2)c1]

The 3-formyl-N-methylbenzamide (0.4 mmol, 2 eq.) was pre-weighed into a 2-dram vial with a stirrer bar. Stock solutions of intermediate 25 (0.79 g, 7.5 mL, 0.27 M) and sodium cyanoborohydride (0.23 g, 7.5 mL, 0.48 M) were prepared in MeOH. 0.75 mL of intermediate 25 stock solution was added and the solutions stirred for 2 h. Next, sodium cyanoborohydride stock solution (0.75 mL) was then added. The reaction mixture was then stirred at room temperature overnight. After reaction completion, the solution was added to MeOH-washed ethylbenzenesulfonic acid resin cartridge (Isolute® SCX-3), and eluted with MeOH (3×2 mL) followed by 3.5 M NH₃ in MeOH (3×2 mL). The basic washes containing the product was evaporated and re-dissolved in 3 mL 1:1 MeCN/MeOH for purification. Purification was performed via Prep SFC (Stationary phase: Torus Diol 30×150 mm, Mobile phase: CO₂, MeOH+20 mM NH₄OH) to give Compound 283 (41 mg, 38% yield), after lyophilization.

The Following Compounds were Synthesized by an Analogous Method as Described for Compound 283

For reactions employing ketone building blocks, the following applies: acetic acid was added (23 μL, 2 eq.) into the reaction mixture before the addition of the sodium cyanoborohydride stock solution. The reaction mixture was stirred at 50° C. overnight (during the reductive amination step).

Alternative purification methods that can be employed for the purification of examples listed below are as follows:

Purifications can also be performed via Prep HPLC (Stationary phase: RP Xselect CSH Prep C18 OBD—10 μm, 30×150 mm, Mobile phase: 0.1% FA solution in water, CH₃CN or MeOH).

Purifications can also be performed via Prep HPLC: (Stationary phase: RP Xbridge Prep C18 OBD—10 μm, 30×150 mm, Mobile phase: 0.25% NH₄HCO₃ solution in water, CH₃CN or MeOH).

These purification methods can also be used in combination.

| Compound No. | Structure | Starting Materials |
|---|---|---|
| Compound 286 | 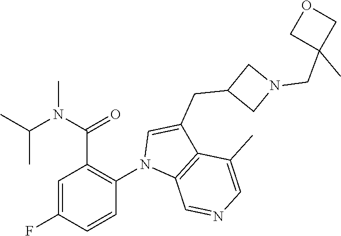 [SMILES: Cc1cncc2c1c(CC1CN(CC3(C)COC3)C1)cn2-c1ccc(F)cc1C(=O)N(C)C(C)C] | Intermediate 25 & 3- methyloxetane-3-carbaldehyde |
| Compound 287 | 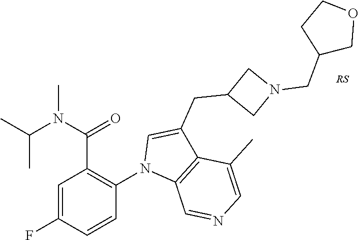 [SMILES: Cc1cncc2c1c(CC1CN(CC3CCOC3)C1)cn2-c1ccc(F)cc1C(=O)N(C)C(C)C] | Intermediate 25 & tetrahydrofuran-3- carboxaldehyde |
| Compound 288 | 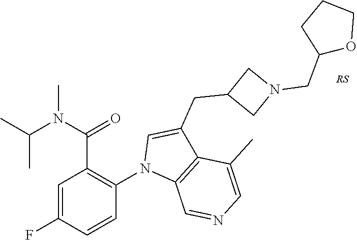 [SMILES: Cc1cncc2c1c(CC1CN(CC3CCCO3)C1)cn2-c1ccc(F)cc1C(=O)N(C)C(C)C] | Intermediate 25 & tetrahydro-2- furancarbaldehyde |
| Compound 289 | 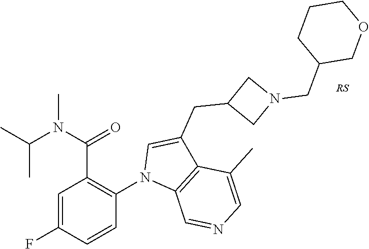 [SMILES: Cc1cncc2c1c(CC1CN(CC3CCCOC3)C1)cn2-c1ccc(F)cc1C(=O)N(C)C(C)C] | Intermediate 25 & tetrahydropyran-3-carbaldehyde |
| Compound 290 | 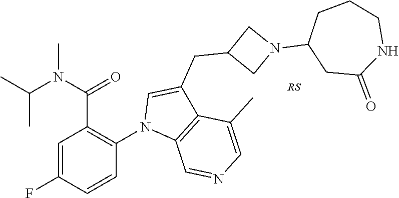 [SMILES: Cc1cncc2c1c(CC1CN(C3CCCNC(=O)C3)C1)cn2-c1ccc(F)cc1C(=O)N(C)C(C)C] | Intermediate 25 & azepane-2,4- dione |
| Compound 291 | 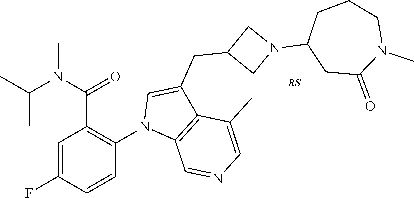 [SMILES: Cc1cncc2c1c(CC1CN(C3CCCN(C)C(=O)C3)C1)cn2-c1ccc(F)cc1C(=O)N(C)C(C)C] | Intermediate 25 & 1- methylazepane-2,4-dione |

### Preparation of Compound 292a:

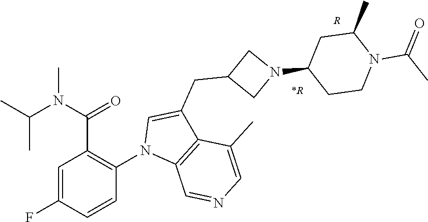

(R)-tert-Butyl 2-methyl-4-oxopiperidine-1-carboxylate (0.4 mmol, 2 eq.) was pre-weighed into a 2-dram vial with a stirrer bar. Stock solutions of intermediate 25 (0.63 g, 6.0 mL, 0.27 M) and sodium cyanoborohydride (0.18 g, 6.0 mL, 0.48 M) were prepared in MeOH. 0.75 mL of intermediate 25 stock solution was added to the reaction vial, together with acetic acid (23 μL, 2 eq.) and the solutions stirred for 1 h. The sodium cyanoborohydride stock solution (0.75 mL) was then added. The reaction mixtures were stirred at 50° C. overnight. After reaction completion, the solutions were added to MeOH-washed ethylbenzenesulfonic acid resin cartridge (Isolute® SCX-3), and eluted with MeOH (3×2 mL) followed by 3.5 M NH₃ in MeOH (3×2 mL). The basic washes containing the product were evaporated.

The crude products from the reductive amination were dissolved in DCM (1 mL) and TFA (2 mL), and stirred at 50° C. for 1 h. The solvents were evaporated and redissolved in MeCN (2 mL). Siliamet® Diamine resin was added and the mixture stirred for 0.5 h. The resin was removed via filtration on a 24-well filter plate, and the filtrate concentrated.

The Boc deprotected products were dissolved in 1 mL DCM, and DIPEA (0.55 mL, 3.2 mmol), and Ac₂O (0.25 mL, 2.6 mmol) were added. The reaction mixture was stirred for 2 h at room temperature, at which time they were quenched with MeOH (2 mL) and concentrated. The samples were re-dissolved in 3 mL 1:1 MeCN/MeOH for purification. Purification was performed via Prep SFC (Stationary phase: Torus Diol 30×150 mm, Mobile phase: CO₂, MeOH+20 mM NH₄OH) to give Compound 292a (22.9 mg, yield: 21%), after lyophilization.

The Following Compounds were Synthesized by an Analogous Method as Described for Compound 292a

For reactions employing ketone building blocks, the following applies: acetic acid was added (23 μL, 2 eq.) into the reaction mixture before the addition of the sodium cyanoborohydride stock solution. The reaction mixture was stirred at 50° C. overnight (during the reductive amination step).

Alternative purification methods that can be employed for the purification of examples listed below are as follows:

Purifications can also be performed via Prep HPLC (Stationary phase: RP Xselect CSH Prep C18 OBD—10 μm, 30×150 mm, Mobile phase: 0.1% FA solution in water, CH₃CN or MeOH).

Purifications can also be performed via Prep HPLC: (Stationary phase: RP Xbridge Prep C18 OBD—10 μm, 30×150 mm, Mobile phase: 0.25% NH₄HCO₃ solution in water, CH₃CN or MeOH).

These purification methods can also be used in combination.

| Compound No. | Structure | Starting Materials |
|---|---|---|
| Compound 292b | 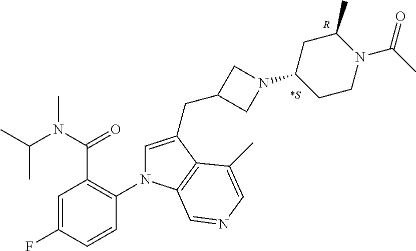 | Intermediate 25 & (R)-tert-Butyl 2-methyl-4-oxopiperidine-1- carboxylate |
| Compound 293a | 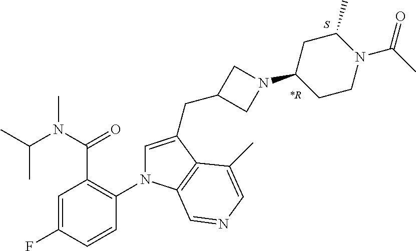 | Intermediate 25 & (S)-tert-Butyl 2-methyl-4-oxopiperidine-1- carboxylate |
| Compound 293b | 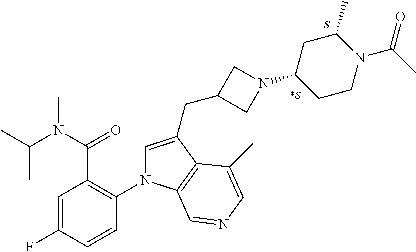 | Intermediate 25 & (S)-tert-Butyl 2-methyl-4-oxopiperidine-1- carboxylate |
| Compound 294a | 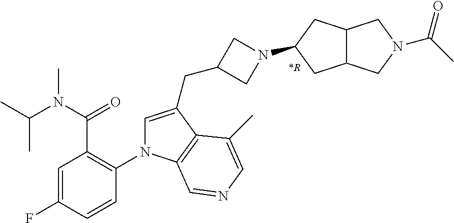 | Intermediate 25 & 2-Boc-5- oxohexahydrocyclopenta[c]pyrrole |
| Compound 294b | 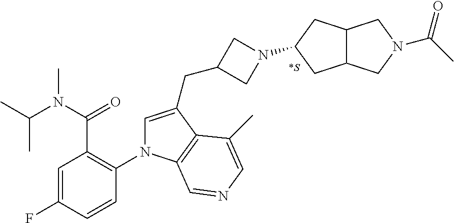 | Intermediate 25 & 2-Boc-5- oxohexahydrocyclopenta[c]pyrrole |
| Compound 295 | 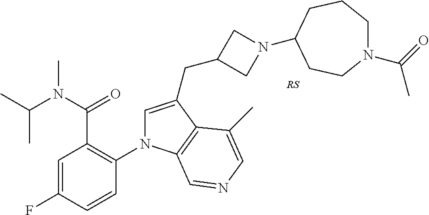 [SMILES: CC(=O)N1CCCC(N2CC(Cc3cn(-c4ccc(F)cc4C(=O)N(C)C(C)C)c4cncc(C)c34)C2)CC1] | Intermediate 25 & N-Boc- hexahydro-1H-azepin-4-one |
| Compound 296 | 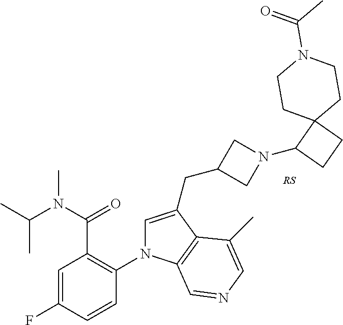 [SMILES: CC(=O)N1CCC2(CCC2N2CC(Cc3cn(-c4ccc(F)cc4C(=O)N(C)C(C)C)c4cncc(C)c34)C2)CC1] | Intermediate 25 & tert-butyl 1- oxo-7-azaspiro[3.5]nonane-7- carboxylate |

### Preparation of Compound 378:

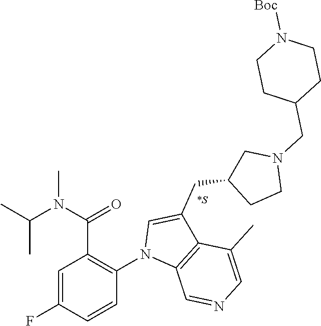 [SMILES: Cc1cncc2c1c(C[C@H]1CCN(CC3CCN(C(=O)OC(C)(C)C)CC3)C1)cn2-c1ccc(F)cc1C(=O)N(C)C(C)C]

To a solution of intermediate 27 (3.5 g, 95%, 8.14 mmol) in DCM (80 mL) was added tert-butyl 4-formylpiperidine-1-carboxylate (3.66 g, 16.3 mmol) and sodium triacetoxyborohydride (2.58 g, 12.2 mmol). After stirring at room temperature for 6 hours, the reaction mixture was poured into saturated aqueous sodium bicarbonate solution and extracted with dichloromethane (80 mL) twice. The combined organic layers were washed with brine (80 mL), dried over Na₂SO₄, filtered and concentrated under reduced pressure to afford the crude product, which was purified by silica gel column chromatography eluting with methanol in dichloromethane from 0% to 6% to give Compound 378 (4.66 g, 95% purity, 89.8% yield) as a white oil.

The Following Compounds were Synthesized by an Analogous Method as Described for Compound 378

In case reactions were performed with a ketone starting material, a typical procedure makes use of either 2 eq. acetic acid or 2 eq. of zinc(II)chloride (ZnCl₂), in the presence of 2 eq. sodium cyanoborohydride (NaCNBH₃), in methanol at 50° C. or 70° C. overnight.

| Co No. | Structure | Starting Materials |
|---|---|---|
| Compound 62 | 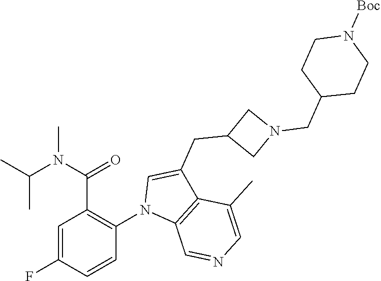 [SMILES: Cc1cncc2c1c(CC1CN(CC3CCN(C(=O)OC(C)(C)C)CC3)C1)cn2-c1ccc(F)cc1C(=O)N(C)C(C)C] | Intermediate 25 & tert-butyl 4- formylpiperidine-1-carboxylate |
| Compound 382 | 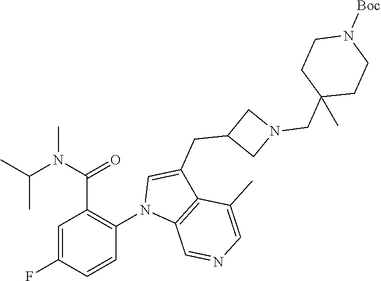 [SMILES: Cc1cncc2c1c(CC1CN(CC3(C)CCN(C(=O)OC(C)(C)C)CC3)C1)cn2-c1ccc(F)cc1C(=O)N(C)C(C)C] | Intermediate 25 & tert-butyl 4- formy1-4-methylpiperidine-1- carboxylate |
| Compound 383 | 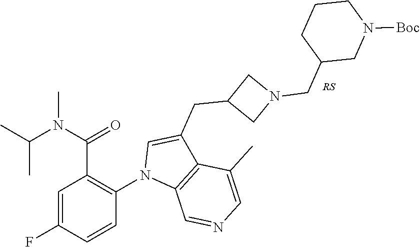 [SMILES: Cc1cncc2c1c(CC1CN(CC3CCCN(C(=O)OC(C)(C)C)C3)C1)cn2-c1ccc(F)cc1C(=O)N(C)C(C)C] | Intermediate 25 & tert-butyl 3- formylpiperidine-1-carboxylate |
| Compound 384 | 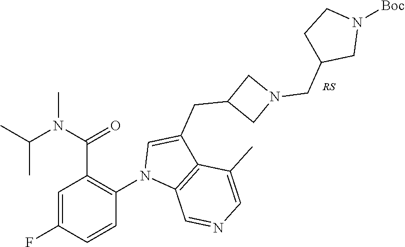 [SMILES: Cc1cncc2c1c(CC1CN(CC3CCN(C(=O)OC(C)(C)C)C3)C1)cn2-c1ccc(F)cc1C(=O)N(C)C(C)C] | Intermediate 25 & tert-butyl 3- formylpyrrolidine-1-carboxylate |
| Compound 531 | 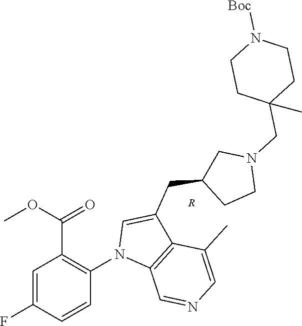 | Intermediate 368 & tert-butyl 4- formyl-4-methylpiperidine-1- carboxylate |
| Compound 397 | 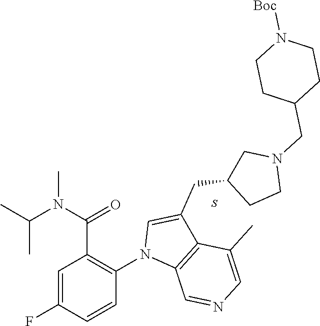 | Intermediate 225 & tert-butyl 4- formylpiperidine-1-carboxylate |
| Compound 408 | 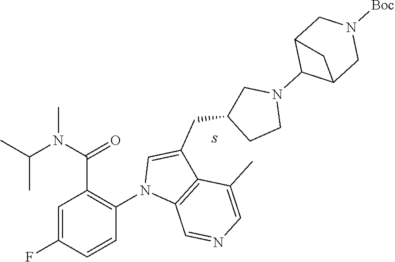 | Intermediate 225 & 3-Boc-6- oxo-3-aza-bicyclo[3.1.1]heptane |
| Compound 410 & | 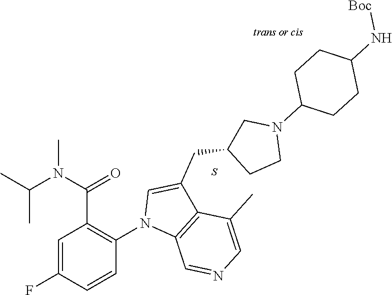 | Intermediate 225 & tert-butyl (4- oxocyclohexyl)carbamate |
| Compound 411 | 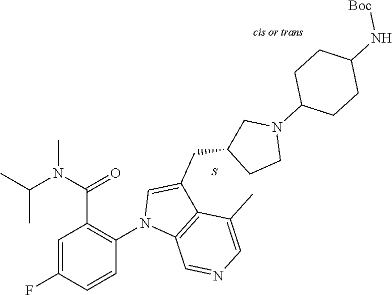 | |
| Compound 415 | 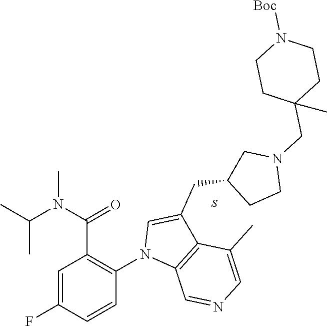 | Intermediate 225 & tert-butyl 4- formyl-4-methylpiperidine-1- carboxylate |
| Compound 416 | 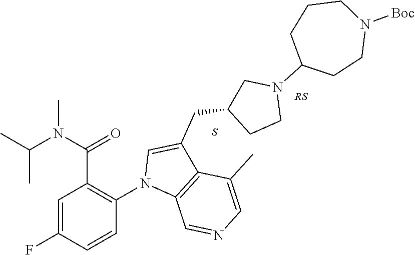 | Intermediate 225 & tert-butyl 4- oxoazepane-1-carboxylate |
| Compound 395 & | 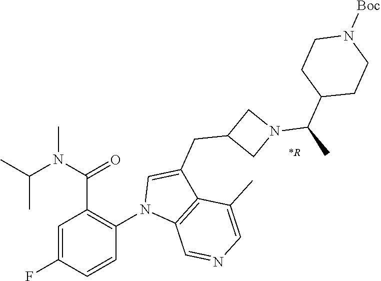 [SMILES: Cc1cncc2c1c(CC1CN([C@H](C)C3CCN(C(=O)OC(C)(C)C)CC3)C1)cn2-c1ccc(F)cc1C(=O)N(C)C(C)C] | Intermediate 25 & tert-butyl 4- acetylpiperidine-1-carboxylate The product was separated by SFC (separation condition: DAICEL CHIRALPAK AD (250 mm*30 mm, 10 um); Mobile phase: A: 0.1% NH₃H₂O, B: EtOH, A:B = 75:25 at 70 mL/min; Column Temp: 38; Nozzle Pressure: 100 Bar; Nozzle Temp: 60; Evaporator Temp: 20; Trimmer Temp: 25; Wavelength: 220 nm). The first fraction was collected as Compound 395 & the second fraction as Compound 396. |
| Compound 396 | 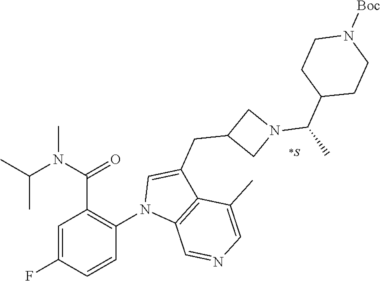 [SMILES: Cc1cncc2c1c(CC1CN([C@@H](C)C3CCN(C(=O)OC(C)(C)C)CC3)C1)cn2-c1ccc(F)cc1C(=O)N(C)C(C)C] | |
| Compound 526a & | 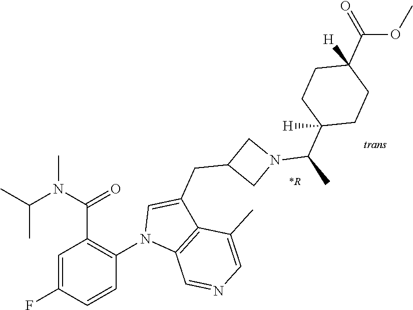 [SMILES: COC(=O)[C@H]1CC[C@H]([C@@H](C)N2CC(Cc3cn(-c4ccc(F)cc4C(=O)N(C)C(C)C)c4cncc(C)c34)C2)CC1] | Intermediate 25 & (trans)- methyl 4- acetylcyclohexanecarboxylate. The product was separated by SFC (separation condition: DAICEL CHIRALPAK IC (250 mm*30 mm, 10 um)); Mobile phase: A: Supercritical CO2, B: 0.1% NH₃H₂O MeOH, A:B = 50:50 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 526a & the second fraction as Compound 526b. |
| Compound 526b | 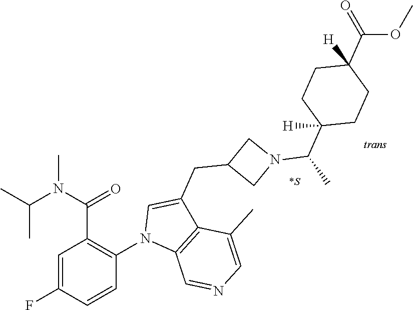 [SMILES: COC(=O)[C@H]1CC[C@H]([C@H](C)N2CC(Cc3cn(-c4ccc(F)cc4C(=O)N(C)C(C)C)c4cncc(C)c34)C2)CC1] | |
| Compound 401 | 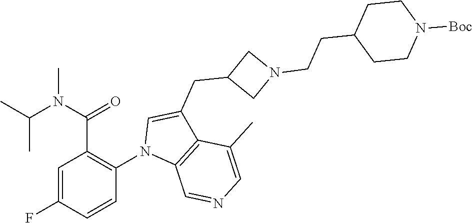 [SMILES: Cc1cncc2c1c(CC1CN(CCC3CCN(C(=O)OC(C)(C)C)CC3)C1)cn2-c1ccc(F)cc1C(=O)N(C)C(C)C] | Intermediate 25 & tert-butyl 4- (2-oxoethyl)piperidine-1- carboxylate |
| Compound 392 | 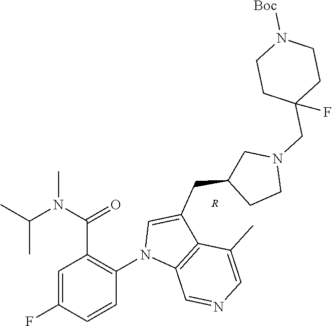 | Intermediate 202 & tert-butyl 4- fluoro-4-formylpiperidine-1- carboxylate |
| Compound 394 | 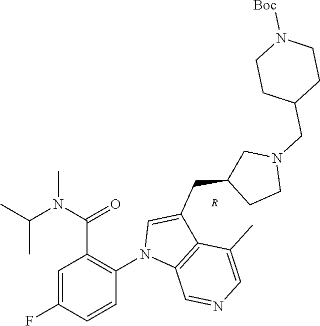 | Intermediate 202 & tert-butyl 4- formylpiperidine-1-carboxylate |
| Compound 387 & | 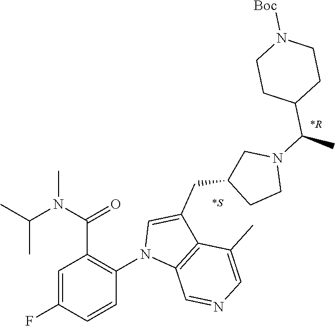 | Intermediate 27 & tert-butyl 4- acetylpiperidine-1-carboxylate The product was separated by supercritical fluid chromatography (separation condition: DAICEL CHIRALPAK AD (250 mm*30 mm, 10 um)); Mobile phase: A: Supercritical CO₂, B: 0.1% NH₃H₂O IPA, A:B = 75:25 at 70 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 387 and the second fraction as Compound 388 |
| Compound 388 | 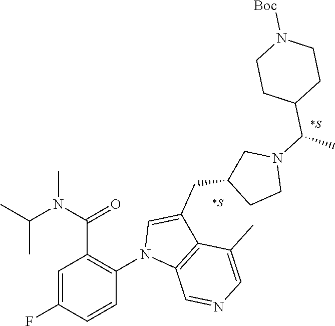 | |
| Compound 398 & | 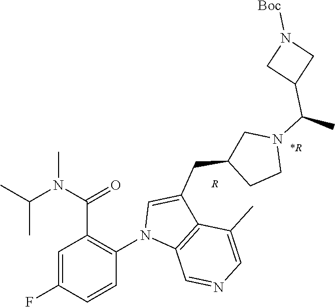 | Intermediate 202 & tert-butyl 3- acetylazetidine-1-carboxylate The product was separated by SFC (separation condition: DAICEL CHIRALPAK IC (250 mm*30 mm, 10 um)); Mobile phase: A: Supercritical CO₂, B: 0.1% NH₃H₂O MeOH, A:B = 50:50 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 398 & the second fraction as Compound 399. |
| Compound 399 | 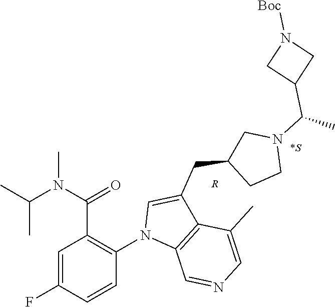 | |
| Compound 400 | 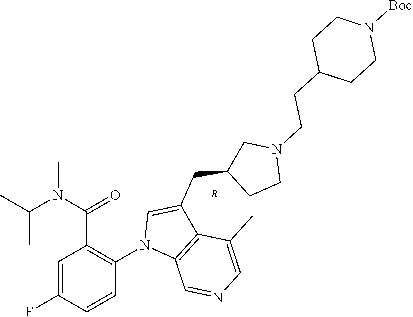 | Intermediate 202 & tert-butyl 4- (2-oxoethyl)piperidine-1- carboxylate |
| Compound 402 & | 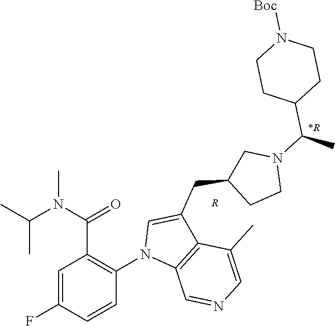 | Intermediate 202 & tert-butyl 4- acetylpiperidine-1-carboxylate The product was separated by SFC (separation condition: DAICEL CHIRALPAK AD (250 mm*50 mm, 10 um); Mobile phase: A: Supercritical CO₂, B: 0.1% NH₃H₂O IPA, A:B = 65:35 at 200 mL/min; Column Temp: 38; Nozzle Pressure: 100 Bar; Nozzle Temp: 60; Evaporator Temp: 20; Trimmer Temp: 25; Wavelength: 220 nm). The first fraction was collected as Compound 402 & the second as Compound 403. |
| Compound 403 | 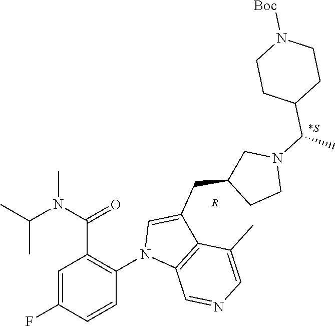 | |
| Compound 404 & | 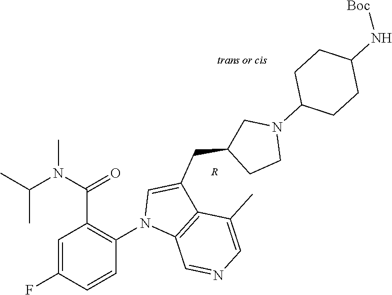 | Intermediate 202 & tert-butyl (4- oxocyclohexyl)carbamate |
| Compound 405 | 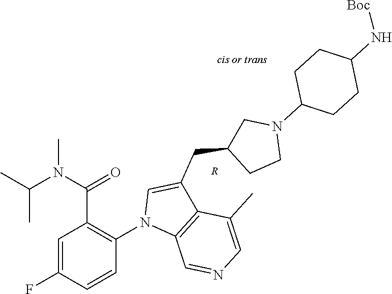 | |
| Compound 407 | 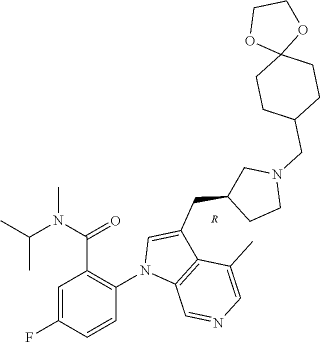 | Intermediate 202 & 1,4- dioxaspiro[4.5]decane-8- carbaldehyde |
| Compound 409 | 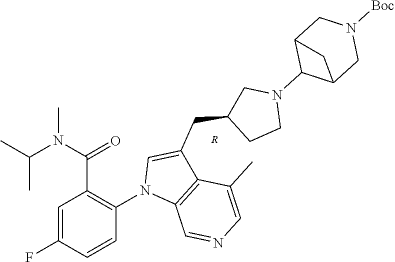 | Intermediate 202 & 3-Boc-6- oxo-3-aza-bicyclo[3.1.1]heptane |
| Compound 412 | 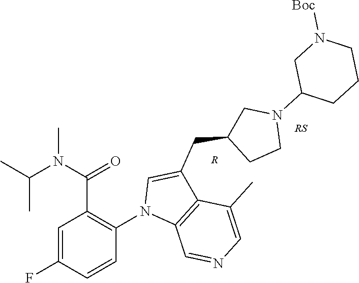 | Intermediate 202 & tert-butyl 3- oxopiperidine-1-carboxylate |
| Compound 413 | 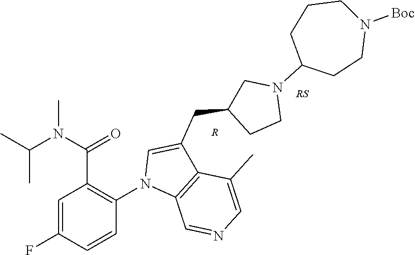 | Intermediate 202 & tert-butyl 4- oxoazepane-1-carboxylate |
| Compound 414 | 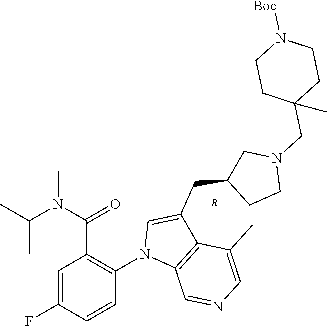 | Intermediate 202 & tert-butyl 4- formyl-4-methylpiperidine-1- carboxylate |
| Compound 417 | 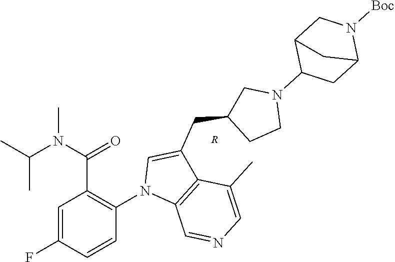 | Intermediate 202 & tert-butyl 5- oxo-2-azabicyclo[2.2.1]heptane- 2-carboxylate |
| Compound 418 | 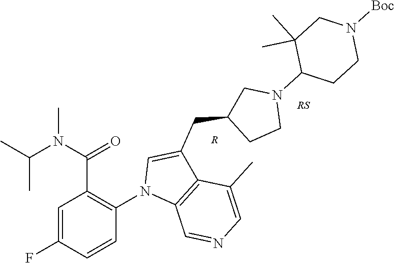 | Intermediate 202 & tert-butyl 3,3-dimethyl-4-oxopiperidine-1- carboxylate |

### Preparation of Compound 381:

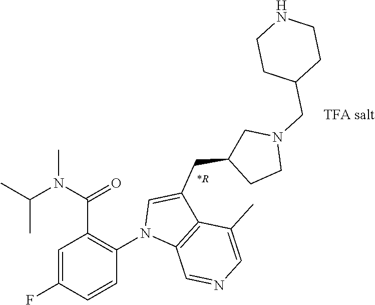 [SMILES: Cc1cncc2c1c(C[C@@H]1CCN(CC3CCNCC3)C1)cn2-c1ccc(F)cc1C(=O)N(C)C(C)C]

Compound 531 (70 mg, 0.104 mmol) was dissolved in DCM (3 mL, 46.837 mmol). TEA (1 mL, 13.067 mmol) was added. The mixture was stirred at RT for 2 hours. The solvent was removed to give Compound 381, which was used in the next step without further purification.

### The Following Compound was Synthesized by an Analogous Method as Described for Compound 381

| | | |
|---|---|---|
| Compound 442 | 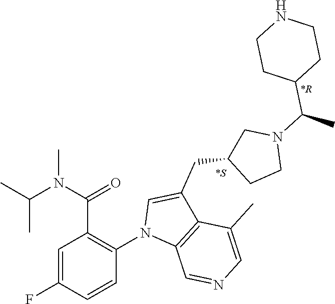 | Compound 387 |
| Compound 448 | 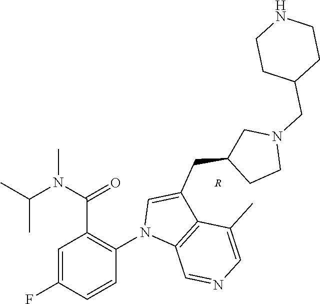 | Compound 394 |

### Preparation of Compound 485:

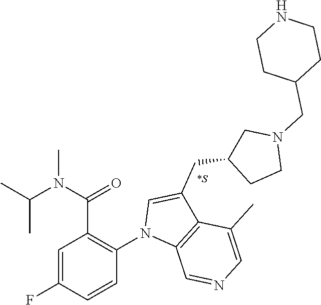 [SMILES: Cc1cncc2c1c(C[C@H]1CCN(CC3CCNCC3)C1)cn2-c1ccc(F)cc1C(=O)N(C)C(C)C]

At 0° C., to a solution of Compound 378 (650 mg, 95% purity, 1.02 mmol) in DCM (8 mL) was added hydrogen chloride in ethyl acetate (2.2 mL, 7 M). After stirring at room temperature for 2 hours, the reaction mixture was concentrated and the residue was basified with aqueous sodium hydroxide solution (1M) and extracted with DCM (20 mL) twice. The combined organic layers were washed with brine (20 mL), dried over Na₂SO₄, filtered and concentrated to afford Compound 485, which was used in the next step without purification.

### Preparation of Compound 498:

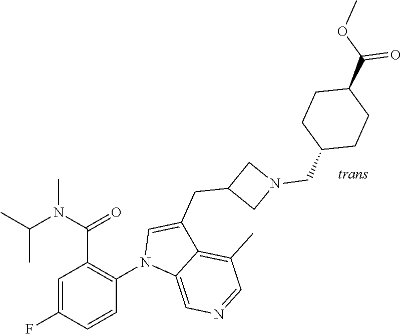 [SMILES: COC(=O)[C@H]1CC[C@H](CN2CC(Cc3cn(-c4ccc(F)cc4C(=O)N(C)C(C)C)c4cncc(C)c34)C2)CC1]

A stir bar, intermediate 25 (300 mg, 0.760 mmol), MeCN (3 mL), intermediate 207 (230 mg, 0.919 mmol), potassium carbonate (318 mg, 2.30 mmol) and potassium iodide (252 mg, 1.52 mmol) were added into a 8 mL glass. The reaction mixture was heated and stirred at 100° C. for 2 h under microwave irradiation. The reaction mixture was filtered through a pad of Celite®, the filter cake was washed with MeCN (5 mL×5). The combined filtrates were concentrated under reduced pressure to give the crude product which was purified by silica gel column chromatography eluting with methanol in dichloromethane from 0% to 9% to give Compound 498 (270 mg, 43.5% purity, 28.1% yield) as yellow solid.

### LCMS (Liquid Chromatography/Mass Spectrometry)

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc.,). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

TABLE 1a
LCMS Method codes (Flow expressed in mL/min; column temperature (T) in ° C.;
Run time in minutes).

| | | | | | Flow | |
|---|---|---|---|---|---|---|
| | | | | | (ml/min) | |
| | | | | | ---- | Run |
| Method | | | | | Column | time |
| code | Instrument | Column | Mobile phase | Gradient | T (° C.) | (min) |
| 1 | Agilent | Xbridge | A: 0.02% | 95% A for 0.50 min, to 5% | 1.5 | 6.5 |
| | Techno- | C18, 5 um | CH₃COONH₄; | A in 4.00 min, held for | ---- | |
| | logies 1200 | 4.6*50 mm | B: CH₃CN | 1.50min, back to 95% A in | 40 | |
| | Series, | | | 0.10 min, held for 0.40 min | | |
| | G6110A | | | | | |
| 4 | Agilent | Xbridge | A: 0.05% TFA; | 90% A for 0.50 min, to 70% | 1.5 | 6.5 |
| | Techno- | C18, 5 um | B: CH₃CN | A in 4.00 min, then to 5% A | ---- | |
| | logies 1200 | 4.6*50 mm | | in 0.50 min, held for 1.00 | 40 | |
| | Series, | | | min, back to 90% A in 0.10 | | |
| | G6110A | | | min, held for 0.40 min | | |
| 6 | Agilent | Xbridge | A: 0.05% TFA; | A in 4.00 min, held for 1.50 | 1.5 | 6.5 |
| | Techno- | C18, 5 um | B: CH₃CN | 95% A for | ---- | |
| | logies 1200 | 4.6*50 mm | | 0.50 min, to 5% | 40 | |
| | Series, | | | min, back to 95% A in 0.10 | | |
| | G6130A | | | min, held for 0.40 min | | |
| 8 | Agilent | ZORBAX | A: 0.05% TFA; | A in 8.00 min, held for 3.60 | 1.5 | 15 |
| | Techno- | SB. | B: CH₃CN | 90% A for | ---- | |
| | logies 1200 | C8, 3.5 um | | 3.00 min, to 5% | 40 | |
| | Series, | 4.6*150 mm | | min, back to 90% A in 0.10 | | |
| | G6110A | | | min, held for 0.30 min. | | |
| 12 | Agilent | Agilent | mobile phase | a gradient condition from | 1.5 | 3.0 |
| | Prime-6125B | Poroshell 120 | A: water(4 L) + | 95% A, 5% B to 20% A, | ---- | |
| | | EC-C18 | TFA(1.5 mL) | 80% B in 1.2 min, then to | 50 | |
| | | 1.9 um | | 5% A, 95% B in 1.3 min, and | | |
| | | 3.0*30 mm | | then to 95% A, 5% B in 0.01 | | |
| | | | | min and held for 0.49 min | | |
| 13 | Waters: H- | Waters: | A: 0.1% | Gradient start from 5% of B | 0.8 | 3.0 |
| | Class + | ACQUITY | NH₄OH in | increase to 95% within 1.5 | ---- | |
| | Zspray | UPLC BEH | water, B: | min and keep at 95% till 2.5 | 40 | |
| | | C18 (1.7 μm, | CH₃CN | min, then decrease to 5% | | |
| | | 2.1 × 30 mm) | | within 0.01 min and keep at | | |
| | | | | 5% till 3.0 min | | |
| 14 | Agilent: 1260 | Waters: | A: 0.1% FA | Gradient start from 5% of B | 1.2 | 3.5 |
| | Infinity and | Sunfire C18 | solution in | increase to 95% within 2.5 | ---- | |
| | 6120 | (2.5 μm, | water; B: | min and keep at 95% till 3.5 | 50 | |
| | Quadrupole | 3.0 × 30 mm) | CH₃CN | min | | |
| | LC/MS | | | | | |
| 15 | Waters: | Waters: BEH | A: 10 mM | From 100% A to 5% A in | 0.6 | 3.5 |
| | Acquity ® | (1.8 μm, | NH₄HCO₃ in | 2.10 min, to 0% A in 0.9 | ---- | |
| | UPLC ®- | 2.1*100 mm) | 95% H₂O + 5% | min, to 5% A in 0.5 min | 55 | |
| | DAD and | | CH₃CN; B: | | | |
| | SQD | | CH₃CN | | | |
| 16 | Waters: | Waters: BEH | A: 10 mM | From 100% A to 5% A in | 0.6 | 3.5 |
| | Acquity ® | (1.8 μm, | NH₄HCO₃ in | 2.10 min, to 0% A in 0.9 | ---- | |
| | UPLC ®- | 2.1*100 mm) | 95% H₂O + 5% | min, to 5% A in 0.5 min | 55 | |
| | DAD and | | CH₃CN; B: | | | |
| | SQD and | | CH₃CN | | | |
| | ELSD | | | | | |
| 17 | Waters: | Waters: BEH | A: 10 mM | From 100% A to 5% A in | 0.6 | 3.5 |
| | Acquity ® | (1.8 μm, | NH₄HCO₃ in | 2.10 min, to 0% A in 0.9 | ---- | |
| | UPLC ®- | 2.1*100 mm) | 95% H₂O + 5% | min, to 5% A in 0.5 min | 55 | |
| | DAD and | | CH₃CN; B: | | | |
| | SQD | | CH₃CN | | | |
| 30 | Waters: | Waters: BEH | CH₃COONH₄ | From 100% A to 5% A in | 0.6 | 3.5 |
| | Acquity ® | (1.7 μm, | 5% CH₃CN B: | 2.10 min, to 0% A in 0.9 | ---- | |
| | UPLC ®- | 2.1*100 mm) | A: 10 mM | min, to 5% A in 0.5 min | 55 | |
| | DAD and | | in 95% H₂O + | | | |
| | SQD | | CH₃CN | | | |
| 35 | Waters: | Waters: BEH | A: 0.1% | From 100% A to | 0.8 | 2.0 |
| | Acquity ® | (1.7 μm, | NH₄HCO₃ | 5% A in 1.3 min, | ---- | |
| | UPLC ®- | 2.1*50 mm) | in 95% H₂O + | hold 0.7 min | 55 | |
| | DAD and | | 5% CH₃CN | | | |
| | SQD | | B: CH₃CN | | | |
| 37 | SHIMADZU | MERCK, RP- | A: water(4 L)+ | a gradient condition from | 1.5 | 1.5 |
| | LC20- MS2010 | 18e 25-2 mm | TFA(1.5 mL) | 95% A, 5% B to 5% A, 95% | ---- | |
| | | | B: aceto-nitrile | B in 0.7 minutes, hold at | 50 | |
| | | | (4L) + TFA | these conditions for 0.4 | | |
| | | | (0.75 mL) | minutes, and then to 95% A, | | |
| | | | | 5% B in 0.01 min and held | | |
| | | | | for 0.49 min. | | |
| 39 | SHIMADZU | Xbridge | A: water(4 L)+ | a gradient condition from | 0.8 | 7.0 |
| | LC20- | Shield RP- | NH₃•H₂O | 90 % A to 20 % A, 80% B in | ---- | |
| | MS2020 | 18,5 um, 2.1* | (0.8 mL) | 6 minutes, and hold at these | 50 | |
| | | 50 mm | B: aceto-nitrile | conditions for 0.5 minutes, | | |
| | | | | then to 90% A and 10% B in | | |
| | | | | 0.1 min and held for 0.49 | | |
| | | | | min. | | |

“TFA” means trifluoroacetic acid;
“FA” means formic acid

TABLE 1b
LCMS and melting point data. Co. No. means compound
number; Rₜ means retention time in min.

| Co. No. | Rt | [M + 1]⁺ | LCMS method |
|---|---|---|---|
| 43 | 3.05 | 507.4 | 4 |
| 50 | 1.29 | 548.8 | 13 |
| 51 | 7.28 | 548.2 | 8 |
| 59 | 1.29 | 578.9 | 13 |
| 60 | 1.31 | 573.9 | 13 |
| 61 | 1.57 | 493.4 | 15 |
| 62 | 2.00 | 592.5 | 15 |
| 63 | 1.50 | 534.4 | 16 |
| 64 | 1.61 | 570.4 | 16 |
| 65 | 1.70 | 550.4 | 16 |
| 82 | 1.16 | 578.84 | 13 |
| 83 | 1.48 | 522.4 | 16 |
| 85 | 3.399 | 578.2 | 1 |
| 86 | 2.771 | 578.2 | 1 |
| 87 | 2.734 | 564.2 | 1 |
| 91 | 1.92 | 604.5 | 16 |
| 92 | 1.69 | 549.4 | 16 |
| 98 | 1.73 | 520.4 | 17 |
| 101 | 2.739 | 563.2 | 1 |
| 104 | 1.45 | 520.5 | 16 |
| 106 | 2.858 | 652.1 | 1 |
| 108 | 2.77 | 652.2 | 1 |
| 114 | 1.36 | 506.4 | 30 |
| 117a | 1.065 | 592.5 | 12 |
| 120 | 2.536 | 506.1 | 1 |
| 121 | 3.083 | 576.2 | 1 |
| 125 | 1.36 | 593.06 | 13 |
| 126 | 1.52 | 602.07 | 13 |
| 135 | 2.986 | 574.2 | 1 |
| 156 | 1.16 | 534.87 | 13 |
| 169 | 1.51 | 534.4 | 16 |
| 169a | 1.54 | 534.5 | 16 |
| 169b | 1.49 | 534.5 | 16 |
| 188a | 1.35 | 548.88 | 13 |
| 188b | 1.44 | 548.67 | 13 |
| 190a | 1.73 | 493.5 | 16 |
| 190b | 1.73 | 493.5 | 15 |
| 191a | 1.43 | 479.5 | 17 |
| 191b | 1.42 | 479.5 | 17 |
| 194 | 2.22 | 505.95 | 13 |
| 196 | 2.977 | 506.1 | 6 |
| 207 | 1.3 | 549.95 | 13 |
| 213a | 1.483 | 555.3 | 12 |
| 213b | 1.481 | 555.3 | 12 |
| 283 | 0.81 | 542 | 16 |
| 286 | 0.79 | 479 | 16 |
| 287 | 0.74 | 479 | 16 |
| 288 | 0.76 | 479 | 16 |
| 289 | 0.78 | 493 | 16 |
| 290 | 0.73 | 506 | 16 |
| 291 | 0.76 | 520 | 16 |
| 292a | 0.83 | 534 | 16 |
| 292b | 0.77 | 534 | 16 |
| 293a | 0.83 | 534 | 16 |
| 293b | 0.77 | 534 | 16 |
| 294a | 0.72 | 546 | 16 |
| 294b | 0.73 | 546 | 16 |
| 295 | 0.73 | 534 | 16 |
| 296 | 0.81 | 560 | 16 |
| 387 | 0.708 | 620.4 | 37 |
| 388 | 0.708 | 620.4 | 37 |
| 378 | 1.69 | 606.84 | 14 |
| 383 | 2.11 | 592.5 | 15 |
| 392 | 1.490 | 624.3 | 14 |
| 394 | 2.29 | 606.4 | 15 |
| 395 | 4.560 | 606.5 | 39 |
| 396 | 4.635 | 606.5 | 39 |
| 398 | 0.689 | 592.3 | 37 |
| 399 | 0.693 | 592.3 | 37 |
| 400 | 2.10 | 620.5 | 15 |
| 401 | 2.00 | 606.5 | 15 |
| 402 | 0.758 | 620.5 | 37 |
| 403 | 0.757 | 620.4 | 37 |
| 407 | 1.83 | 563.4 | 15 |
| 414 | 1.97 | 620.5 | 14 |
| 415 | 1.98 | 620.4 | 14 |
| 442 | 0.321 | 520.3 | 37 |
| 448 | 0.98 | 506.4 | 35 |
| 498 | 4.209 | 549.4 | 39 |
| 526a | 0.681 | 563.3 | 37 |
| 526b | 0.674 | 563.3 | 37 |

### Analytical SFC

### General Procedure for SFC Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 1c
Analytical SFC Methods (Flow expressed in mL/min; column temperature (T)
in ° C.; Run time in minutes, Backpressure (BPR) in bars or pound-
force per square inch (psi). “ACN” means acetonitrile; “MeOH” means
methanol; “EtOH” means ethanol; “iPrNH₂” means All other
abbreviations used in the table below are as defined before

| | | | | Flow - | Run time |
|---|---|---|---|---|---|
| | | | | (ml/mn) | (min) |
| | | | | — | — |
| Method | | | | Column T | BPR |
| code | Column | Mobile phase | Gradient | (° C.) | (bar) |
| 1 | Daicel | A: supercritical | 10%-50% B in 6 | 2.5 | 9.5 |
| | Chiralpak ® | CO₂ | min, hold 3.5 min | — | — |
| | AD3 | B: iPrOH + 0.2% | | 40 | 130 |
| | column (3.0 μm, | iPrNH₂ | | | |
| | 150 × 4.6 mm) | | | | |
| 2 | Daicel | A: supercritical | 10%-50% B in 6 | 2.5 | 9.5 |
| | Chiralpak ® | CO₂ | min, hold 3.5 min | — | — |
| | IG3 | B: EtOH + 0.2% | | 40 | 130 |
| | column (3.0 μm, | iPrNH₂ | | | |
| | 150 × 4.6 mm) | | | | |

### NMR:

### NMR-Methods

Some NMR experiments were carried out using a Bruker Avance I 400 spectrometer at ambient temperature (298.6 K), using internal deuterium lock and equipped with BBO 400 MHz S1 5 mm probe head with z gradients and operating at 400 MHz for the proton and 100 MHz for carbon. Chemical shifts (d) are reported in parts per million (ppm). J values are expressed in Hz.

Some NMR experiments were carried out using a Varian 400-MR spectrometer at ambient temperature (298.6 K), using internal deuterium lock and equipped with Varian 400 4NUC PFG probe head with z gradients and operating at 400 MHz for the proton and 100 MHz for carbon. Chemical shifts (d) are reported in parts per million (ppm). J values are expressed in Hz.

Some NMR experiments were carried out using a Varian 400-VNMRS spectrometer at ambient temperature (298.6 K), using internal deuterium lock and equipped with Varian 400 ASW PFG probe head with z gradients and operating at 400 MHz for the proton and 100 MHz for carbon. Chemical shifts (d) are reported in parts per million (ppm). J values are expressed in Hz.

| Compound | |
|---|---|
| number | NMR data |
| Compound 43 | ¹H NMR (400 MHz, DMSO-d6) d 8.43-8.34 (m, 1H), 7.94-7.92 (m, |
| | 1H), 7.65-7.62 (m, 1H), 7.50-7.42 (m, 2H), 7.28-7.22 (m, 1H), 4.41- |
| | 4.39 (m, 0.5H), 3.80 (dd, J = 7.2 Hz; 3.6 Hz, 2H), 3.51-3.48 (m, 1H), |
| | 3.31-3.23 (m, 2.5H), 2.93-2.86 (m, 2H), 2.68-2.66 (m, 1H), 2.61- |
| | 2.58 (m, 5H), 2.55-2.51 (m, 0.5H), 2.48-2.46 (m, 0.5H), 2.44-2.37 |
| | (m, 1.5H), 2.34 (s, 1.5H), 2.33-2.18 (m, 3H), 1.96-1.95 (m, 1H), 1.68- |
| | 1.60 (m, 3H), 1.49-1.45 (m, 1H), 1.16-1.06 (m, 2H), 0.96-0.94 (m, |
| | 2.5H), 0.48-0.47 (m, 1H), 0.20-0.17 (m, 1.5H). |
| Compound 51 | ¹H NMR (400 MHz, DMSO-d6): δ 8.43-8.34 (m, 1H), 7.94-7.92 (m, |
| | 1H), 7.67-7.62 (m, 1H), 7.50-7.42 (m, 2H), 7.28-7.22 (m, 1H), 4.42-4.39 |
| | (m, 0.43H), 4.34-4.31 (m, 1H), 3.78-3.74 (m, 1H), 3.51-3.46 (m, 0.53H), |
| | 3.00-2.84 (m, 3H), 2.71-2.55 (m, 7H), 2.48-2.34 (m, 4H), 2.28-2.14 (m, |
| | 3H), 2.00-1.90 (m, 4H), 1.75-1.65 (m, 3H), 1.50-1.44 (m, 1H), 1.06-0.84 |
| | (m, 5H), 0.48-0.17 (m, 3H). |
| Compound 51a | ¹H NMR (400 MHz, DMSO-d₆, 27° C.) δ ppm 0.18-0.53 (m, 3 H), 0.89- |
| | 1.29 (m, 5 H), 1.68-1.89 (m, 3 H), 1.98 (d, J = 1.3 Hz, 4 H), 2.07-2.29 |
| | (m, 1 H), 2.37 (s, 1 H), 2.57 (s, 2 H), 2.63 (s, 3 H), 2.94-3.15 (m, 6 H), |
| | 3.48-3.54 (m, 0.5 H), 3.56-3.75 (m, 2 H), 3.76-3.86 (m, 1 H), 4.35 |
| | (br d, J = 14.3 Hz, 2 H), 4.38-4.42 (m, 0.5 H), 7.32-7.42 (m, 1 H), 7.44- |
| | 7.54 (m, 2 H), 7.62-7.71 (m, 1 H), 7.97 (d, J = 5.5 Hz, 1 H), 8.34-8.47 |
| | (m, 1 H), 10.01-10.48 (m, 1 H). |
| Compound 59 | ¹H NMR (400 MHz, DMSO-d6) δ = 8.56-8.25 (m, 1H), 8.04-7.82 (m, |
| | 1H), 7.70-7.58 (m, 1H), 7.56-7.38 (m, 2H), 7.33-7.17 (m, 1H), 4.49- |
| | 4.35 (m, 0.5H), 4.35-4.24 (m, 1H), 4.10-3.93 (m, 2H), 3.82-3.66 (m, |
| | 1H), 3.55-3.37 (m, 0.5H), 3.27-3.21 (m, 3H), 2.99-2.79 (m, 3H), |
| | 2.73-2.55 (m, 7H), 2.43-2.30 (m, 3H), 2.30-2.09 (m, 4H), 2.04-1.84 |
| | (m, 1H), 1.81-1.59 (m, 3H), 1.54-1.36 (m, 1H), 1.12-0.81 (m, 5H), |
| | 0.63-0.10 (m, 3H). |
| Compound 60 | ¹H NMR (400 MHz, DMSO-d6) δ = 8.48-8.27 (m, 1H), 7.98-7.90 (m, |
| | 1H), 7.72-7.60 (m, 1H), 7.53-7.40 (m, 2H), 7.31-7.19 (m, 1H), 4.48- |
| | 4.33 (m, 0.5H), 4.33-4.23 (m, 1H), 4.08-3.92 (m, 2H), 3.66-3.57 (m, |
| | 1H), 3.55-3.42 (m, 0.5H), 3.08-2.79 (m, 3H), 2.74-2.55 (m, 7H), |
| | 2.43-2.11 (m, 6H), 2.06-1.82 (m, 1H), 1.80-1.61 (m, 3H), 1.56-1.37 |
| | (m, 1H), 1.16-0.82 (m, 5H), 0.62-0.09 (m, 3H). |
| Compound 117a | ¹H NMR (400 MHz, METHANOL-d4) 8.44-8.32 (m, 1H), 7.92 (d, J = |
| | 7.6 Hz, 1H), 7.67-7.59 (m, 1H), 7.47-7.39 (m, 1H), 7.38-7.30 (m, |
| | 2H), 4.52-4.46 (m, 0.5H), 4.22-4.07 (m, 2H), 3.92-3.84 (m, 1H), |
| | 3.63-3.54 (m, 0.5H), 3.39 (s, 3H), 3.11-2.80 (m, 4H), 2.79-2.48 (m, |
| | 9H), 2.46-1.85 (m, 6H), 1.84-1.74 (m, 1H), 1.72-1.51 (m, 2H), 1.30- |
| | 1.12 (m, 2H), 1.10-0.87 (m, 6H), 0.59-0.18 (m, 3H) |
| Compound 125 | ¹H NMR (400 MHz, DMSO-d6) δ ppm −0.04 (br d, J = 2.1 Hz, 1 H), 0.13- |
| | 0.22 (m, 2 H), 0.36-0.60 (m, 1 H), 0.85-0.88 (m, 1 H), 0.90-0.99 |
| | (m, 3 H), 1.24 (br s, 1 H), 1.29 (s, 6 H), 1.41-1.53 (m, 1 H), 1.64 (br d, |
| | J = 1.7 Hz, 1 H), 1.67-1.75 (m, 2 H), 1.87-2.02 (m, 1 H), 2.13-2.28 |
| | (m, 3 H), 2.32-2.42 (m, 3 H), 2.56-2.63 (m, 5 H), 2.64-2.74 (m, 1 H), |
| | 2.77-3.01 (m, 3 H), 3.14-3.27 (m, 1 H), 3.38-3.58 (m, 1 H), 4.33- |
| | 4.48 (m, 1 H), 5.30 (s, 1 H), 7.23 (d, J = 8.2 Hz, 1 H), 7.28 (s, 1 H), 7.42- |
| | 7.51 (m, 2 H), 7.61-7.68 (m, 1 H), 7.93 (d, J = 7.9 Hz, 1 H), 8.30-8.47 |
| | (m, 1 H), 8.34 (s, 1 H), 8.43 (d, J = 2.6 Hz, 1 H) |
| Compound 169a | ¹H NMR (400 MHz, DMSO-d6, 27° C.) d ppm 0.12-0.25 (m, 2 H), 0.36- |
| | 0.63 (m, 1 H), 0.81-1.12 (m, 5 H), 1.33-1.55 (m, 1 H), 1.59-2.06 |
| | (m, 4 H), 2.08-2.27 (m, 4 H), 2.34 (s, 5 H), 2.56-2.65 (m, 5 H), 2.79- |
| | 3.16 (m, 4 H), 3.42 (s, 1 H), 4.32-4.48 (m, 1 H), 7.18-7.31 (m, 1 H), |
| | 7.32-7.54 (m, 3 H), 7.57-7.72 (m, 1 H), 7.86-7.99 (m, 1 H), 8.27- |
| | 8.51 (m, 1 H) |

### DSC

For a number of compounds, melting points (MP) were determined with a TA Instrument (Discovery DSC 250 or a DSC 2500). Melting points were measured with a temperature gradient of 10° C./minute. Maximum temperature was 300° C. Values are melting peak onset values.

### XPRD

### Compound 51 as a Crystalline Free Base Form

Compound 51 as a crystalline free base Form may be characterized by an X-ray powder diffraction pattern.

X-ray powder diffraction (XRPD) analysis was carried out on a PANalytical Empyrean diffractometer. The compound was loaded onto a zero-background silicon wafer sample holder by gently pressing the powder sample onto the flat surface.

Samples were run on XRPD using the method below:
Radiation: Cu K-Alpha (λ=1.5418 Å)
Tube voltage/current: 45 kV/40 mA
Divergence slit: ⅛°
Geometry: Bragg-Brentano
Scan mode: Continuous Scan
Scan Range: 3-40° 2θ
Step size: 0.013° 2θ
Scan speed: 20.4 s/step
Rotation: On
Detector: PIXcel^{1D}

One skilled in the art will recognize that diffraction patterns and peak positions are typically substantially independent of the diffractometer used and whether a specific calibration method is utilized. Typically, the peak positions may differ by about ±0.2° 2θ, or less. The intensities (and relative intensities) of each specific diffraction peak may also vary as a function of various factors, including but not limited to particle size, orientation, sample purity, etc.

The X-ray powder diffraction pattern comprises peaks at 9.3, 12.6, 15.7, 21.9 and 22.5° 2θ±0.2° 2θ. The X-ray powder diffraction pattern may further comprise at least one peak selected from 8.1, 11.6, 13.2, 16.8, 18.5, 18.7, 19.2, 19.9, 20.5° 2θ±0.2° 2θ.

Compound 51 as a crystalline free base Form may further be characterized by an X-ray powder diffraction pattern having four, five, six, seven, eight, nine or more peaks selected from those peaks identified in Table 2a.

Compound 51 as a crystalline free base Form may further be characterized by an X-ray powder diffraction pattern comprising those peaks identified in Table 2a, wherein the relative intensity of the peaks is greater than about 2%, preferably greater than about 5%, more preferably greater than about 10%, more preferably greater than about 15%. However, a skilled person will realize that the relative intensity of the peaks may vary between different samples and different measurements on the same sample.

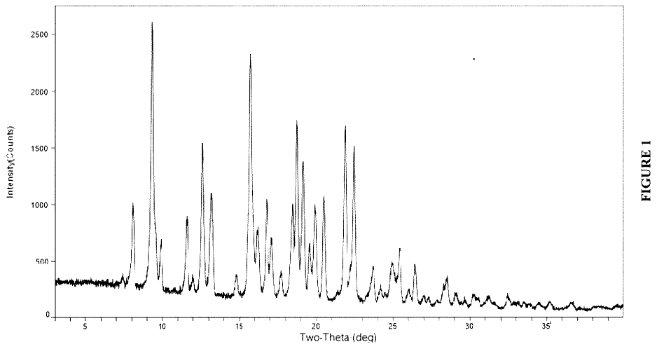

Compound 51 as a crystalline free base Form may further be characterized by an X-ray powder diffraction pattern substantially as depicted in FIG. 1.

Table 2a provides peak listing and relative intensity for the XPRD of Compound 51 as a crystalline free base Form:

| No. | Pos. (°2θ) | Rel. Int. (%) |
|---|---|---|
| 1 | 7.411 | 4.6 |
| 2 | 8.056 | 31.4 |
| 3 | 9.304 | 100 |
| 4 | 9.893 | 18.2 |
| 5 | 11.574 | 28.1 |
| 6 | 11.969 | 6.4 |
| 7 | 12.598 | 55.9 |
| 8 | 13.163 | 37.6 |
| 9 | 14.804 | 7.9 |
| 10 | 15.723 | 89.6 |
| 11 | 16.195 | 24.4 |
| 12 | 16.762 | 34.5 |
| 13 | 17.076 | 21.2 |
| 14 | 17.694 | 9.5 |
| 15 | 18.454 | 33.2 |
| 16 | 18.744 | 63.6 |
| 17 | 19.15 | 48.8 |
| 18 | 19.57 | 16.4 |
| 19 | 19.912 | 32.2 |
| 20 | 20.503 | 38.2 |
| 21 | 21.881 | 65.4 |
| 22 | 22.485 | 57.1 |
| 23 | 23.693 | 13 |
| 24 | 24.205 | 5.6 |
| 25 | 24.915 | 15.2 |
| 26 | 25.401 | 19.6 |
| 27 | 26.068 | 5.3 |
| 28 | 26.400 | 14.5 |
| 29 | 28.276 | 8 |
| 30 | 28.499 | 11 |

### Compound 51a Crystalline HCl Salt Form (Mono HCl Trihydrate Salt)

Compound 51a (Crystalline HCl salt Form—mono HCl trihydrate salt) may be characterized by an X-ray powder diffraction pattern.

X-ray powder diffraction (XRPD) analysis was carried out on a PANalytical Empyrean diffractometer. The compound was loaded onto a zero-background silicon wafer sample holder by gently pressing the powder sample onto the flat surface.

Samples were run on XRPD using the method below:
Radiation: Cu K-Alpha (λ=1.5418 Å)
Tube voltage/current: 45 kV/40 mA
Divergence slit: ⅛°
Geometry: Bragg-Brentano
Scan mode: Continuous Scan
Scan Range: 3-40° 2θ
Step size: 0.013° 2θ
Scan speed: 20.4 s/step
Rotation: On
Detector: PIXcel^{1D}

One skilled in the art will recognize that diffraction patterns and peak positions are typically substantially independent of the diffractometer used and whether a specific calibration method is utilized. Typically, the peak positions may differ by about ±0.2° 2θ, or less. The intensities (and relative intensities) of each specific diffraction peak may also vary as a function of various factors, including but not limited to particle size, orientation, sample purity, etc.

The X-ray powder diffraction pattern comprises peaks at 5.2, 13.2, 14.1, 18.8 and 20.3° 2θ±0.2° 2θ. The X-ray powder diffraction pattern may further comprise at least one peak selected from 9.7, 10.0, 15.4, 15.8, 18.3, 21.3, 24.3° 2θ±0.2° 2θ.

Compound 51a may further be characterized by an X-ray powder diffraction pattern having four, five, six, seven, eight, nine or more peaks selected from those peaks identified in Table 2b.

Compound 51a may further be characterized by an X-ray powder diffraction pattern comprising those peaks identified in Table 2b, wherein the relative intensity of the peaks is greater than about 2%, preferably greater than about 5%, more preferably greater than about 10%, more preferably greater than about 15%. However, a skilled person will realize that the relative intensity of the peaks may vary between different samples and different measurements on the same sample.

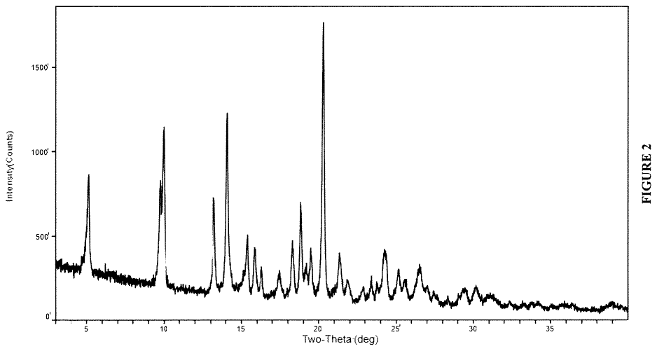

Compound 51a may further be characterized by an X-ray powder diffraction pattern substantially as depicted in FIG. 2.

Table 2b provides peak listing and relative intensity for the XPRD of Compound 51a.

| No. | Pos. (°2θ) | Rel. Int. (%) |
|---|---|---|
| 1 | 5.151 | 36 |
| 2 | 9.749 | 37.2 |
| 3 | 9.984 | 58.9 |
| 4 | 13.217 | 34 |
| 5 | 14.095 | 64.4 |
| 6 | 15.393 | 20.4 |
| 7 | 15.842 | 16.4 |
| 8 | 16.315 | 10.4 |
| 9 | 17.471 | 10.1 |
| 10 | 18.296 | 19.4 |
| 11 | 18.810 | 34.3 |
| 12 | 19.19 | 10.8 |
| 13 | 19.505 | 16.6 |
| 14 | 20.305 | 100 |
| 15 | 21.331 | 16.6 |
| 16 | 21.855 | 6.8 |
| 17 | 22.905 | 4.5 |
| 18 | 23.419 | 8.2 |
| 19 | 24.310 | 17.4 |
| 20 | 25.136 | 10.6 |
| 21 | 25.595 | 7.1 |
| 22 | 26.529 | 12.9 |
| 23 | 29.496 | 4.5 |
| 24 | 30.179 | 6.1 |

### Dynamic Vapor Sorption (DVS)

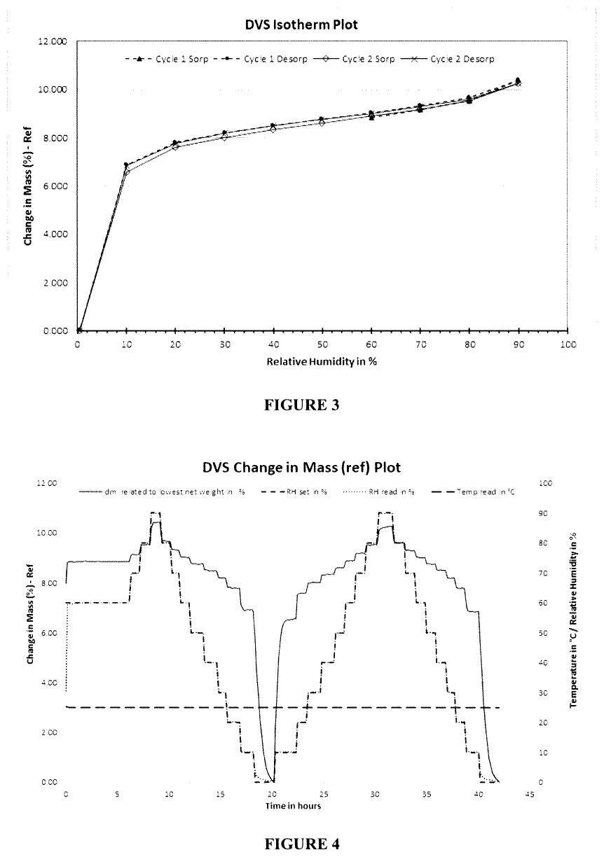

The moisture sorption analysis (DVS) was performed using a ProUmid GmbH & Co. KG Vsorp Enhanced dynamic vapor sorption apparatus. Results are shown in FIG. 3 and FIG. 4. The moisture profile was evaluated by monitoring vapor adsorption/desorption over the range of 0 to 90% relative humidity at 25° C. The sample weight equilibrium criteria were set at 0.01% change in 45 min with minimum and maximum time of acclimation at 50 min and 120 min, respectively. The moisture profile consisted of 2 cycles of vapor adsorption/desorption.

The DVS change in mass plot of crystalline HCl salt Form (Compound 51a) shows that the crystalline form is hygroscopic with the water content varying with relative humidity and dehydrates rapidly at below 10% RH (relative humidity) to complete dehydrated state at 0% RH. In the humidity range of 20-90% RH, the crystalline form adsorbs and desorbs moisture slowly and reversibly up to 2.5% by mass on average. Based on DVS, the crystalline HCl salt Form, at equilibrium, can contain around 3 equivalents of water (8.5-9.5% total moisture mass) at common ambient RH of 40% to 75%. The XRPD pattern of the fraction obtained after the DVS test was comparable to the starting material. No indication of a solid-state form change was observed.

### Pharmacological Part

### 1) Menin/MLL Homogenous Time-Resolved Fluorescence (HTRF) Assay

To an untreated, white 384-well microtiter plate was added 40 nL 200× test compound in DMSO and 4 μL 2× terbium chelate-labeled menin (vide infra for preparation) in assay buffer (40 mM Tris·HCl, pH 7.5, 50 mM NaCl, 1 mM DTT (dithiothreitol) and 0.05% Pluronic F-127). After incubation of test compound and terbium chelate-labeled menin for 30 min at ambient temperature, 4 μL 2×FITC-MBM1 peptide (FITC-O-alanine-SARWRFPARPGT-NH₂) (“FITC” means fluorescein isothiocyanate) in assay buffer was added, the microtiter plate centrifuged at 1000 rpm for 1 min and the assay mixtures incubated for 15 min at ambient temperature. The relative amount of menin-FITC-MBM1 complex present in an assay mixture is determined by measuring the homogenous time-resolved fluorescence (HTRF) of the terbium/FITC donor/acceptor fluorphore pair using an EnVision microplate reader (ex. 337 nm/terbium em. 490 nm/FITC em. 520 nm) at ambient temperature. The degree of fluorescence resonance energy transfer (the HTRF value) is expressed as the ratio of the fluorescence emission intensities of the FITC and terbium fluorophores (F^{em} 520 nm/F^{em} 490 nm). The final concentrations of reagents in the binding assay are 200 pM terbium chelate-labeled menin, 75 nM FITC-MBM1 peptide and 0.5% DMSO in assay buffer. Dose-response titrations of test compounds are conducted using an 11 point, four-fold serial dilution scheme, starting typically at 10 pM.

Compound potencies were determined by first calculating % inhibition at each compound concentration according to equation 1:

% inhibition=((HC−LC)−(HTRF^{compound}−LC))/(HC−LC))*100 (Eqn 1)

Where LC and HC are the HTRF values of the assay in the presence or absence of a saturating concentration of a compound that competes with FITC-MBM1 for binding to menin, and HTRF^{compound} is the measured HTRF value in the presence of the test compound. HC and LC HTRF values represent an average of at least 10 replicates per plate. For each test compound, % inhibition values were plotted vs. the logarithm of the test compound concentration, and the IC₅₀ value derived from fitting these data to equation 2:

% inhibition=Bottom+(Top−Bottom)/(1+10\hat{ }((log IC₅₀−log[cmpd])*h)) (Eqn 2)

Where Bottom and Top are the lower and upper asymptotes of the dose-response curve, respectively, IC₅₀ is the concentration of compound that yields 50% inhibition of signal and h is the Hill coefficient.

Preparation of Terbium cryptate labeling of Menin: Menin (a.a 1-610-6×his tag, 2.3 mg/mL in 20 mM Hepes (2-[4-(2-Hydroxyethyl)-1-piperazinyl]ethane sulfonic acid), 80 mM NaCl, 5 mM DTT (Dithiothreitol), pH 7.5) was labeled with terbium cryptate as follows. 200 μg of Menin was buffer exchanged into 1× Hepes buffer. 6.67 pM Menin was incubated with 8-fold molar excess NHS (N-hydroxysuccinimide)-terbium cryptate for 40 minutes at room temperature. Half of the labeled protein was purified away from free label by running the reaction over a NAP5 column with elution buffer (0.1M Hepes, pH 7+0.1% BSA (bovine serum albumin)). The other half was eluted with 0.1M phosphate buffered saline (PBS), pH7. 400 μl of eluent was collected for each, aliquoted and frozen at −80° C. The final concentration of terbium-labeled Menin protein was 115 μg/mL in Hepes buffer and 85 μg/mL in PBS buffer, respectively.

MENIN Protein Sequence (SEQ ID NO: 1):
MGLKAAQKTLFPLRSIDDVVRLFAAELGREEPDLVLLSLVLGFVEH
FLAVNRVIPTNVPELTFQPSPAPDPPGGLTYFPVADLSIIAALYA
RFTAQIRGAVDLSLYPREGGVSSRELVKKVSDVIWNSLSRSYFKD
RAHIQSLFSFITGTKLDSSGVAFAVVGACQALGLRDVHLALSEDH
AWVVFGPNGEQTAEVTWHGKGNEDRRGQTVNAGVAERSWLYLKGS
YMRCDRKMEVAFMVCAINPSIDLHTDSLELLQLQQKLLWLLYDLG
HLERYPMALGNLADLEELEPTPGRPDPLTLYHKGIASAKTYYRDE
HIYPYMYLAGYHCRNRNVREALQAWADTATVIQDYNYCREDEEIY
KEFFEVANDVIPNLLKEAASLLEAGEERPGEQSQGTQSQGSALQD
PECFAHLLRFYDGICKWEEGSPTPVLHVGWATFLVQSLGRFEGQV
RQKVRIVSREAEAAEAEEPWGEEAREGRRRGPRRESKPEEPPPPK
KPALDKGLGTGQGAVSGPPRKPPGTVAGTARGPEGGSTAQVPAPA
ASPPPEGPVLTFQSEKMKGMKELLVATKINSSAIKLQLTAQSQVQ
MKKQKVSTPSDYTLSFLKRQRKGLHHHHHH

### 2a) Proliferation Assay

The anti-proliferative effect of menin/MLL protein/protein interaction inhibitor test compounds was assessed in human leukemia cell lines. The cell line MOLM14 harbors a MLL translocation and expresses the MLL fusion protein MLL-AF9, respectively, as well as the wildtype protein from the second allele. OCI-AML3 cells that carry the NPM1c gene mutation were also tested. MLL rearranged cell lines (e.g. MOLM14) and NPM1c mutated cell lines exhibit stem cell-like HOXA/MEIS1 gene expression signatures. KO-52 was used as a control cell line containing two MLL (KMT2A) wildtype alleles in order to exclude compounds that display general cytotoxic effects.

MOLM14 cells were cultured in RPMI-1640 (Sigma Aldrich) supplemented with 10% heat-inactivated fetal bovine serum (HyClone), 2 mM L-glutamine (Sigma Aldrich) and 50 μg/ml gentamycin (Gibco). KO-52 and OCI-AML3 cell lines were propagated in alpha-MEM (Sigma Aldrich) supplemented with 20% heat-inactivated fetal bovine serum (HyClone), 2 mM L-glutamine (Sigma Aldrich) and 50 pg/ml gentamycin (Gibco). Cells were kept at 0.3-2.5 million cells per ml during culturing and passage numbers did not exceed 20.

In order to assess the anti-proliferative effects, 200 MOLM14 cells, 200° C. I-AML3 cells or 300 KO-52 cells were seeded in 200 μl media per well in 96-well round bottom, ultra-low attachment plates (Costar, catalogue number 7007). Cell seeding numbers were chosen based on growth curves to ensure linear growth throughout the experiment. Test compounds were added at different concentrations and the DMSO content was normalized to 0.3%. Cells were incubated for 8 days at 37° C. and 5% CO₂. Spheroid like growth was measured in real-time by live-cell imaging (IncuCyteZOOM, Essenbio, 4× objective) acquiring images at day 8. Confluence (%) as a measure of spheroid size was determined using an integrated analysis tool.

In order to determine the effect of the test compounds over time, the confluence in each well as a measure of spheroid size, was calculated. Confluence of the highest dose of a reference compound was used as baseline for the LC (Low control) and the confluence of DMSO treated cells was used as 0% cytotoxicity (High Control, HC).

Absolute IC50 values were calculated as percent change in confluence as follows:
LC=Low Control: cells treated with e.g. 1 pM of the cytotoxic agent staurosporin, or e.g. cells treated with a high concentration of an alternative reference compound
HC=High Control: Mean confluence (%) (DMSO treated cells)

$$\% Effect = 100 - ( 10 \; 0 * ( Sample - LC ) / ( HC - L C ) )$$

GraphPad Prism (version 7.00) was used to calculate the IC₅₀. Dose-response equation was used for the plot of % Effect vs Log 10 compound concentration with a variable slope and fixing the maximum to 100% and the minimum to 0%.

2b) MEIS1 mRNA Expression Assay

MEIS1 mRNA expression upon treatment of compound was examined by Quantigene Singleplex assay (Thermo Fisher Scientific). This technology allows for direct quantification of mRNA targets using probes hybridizing to defined target sequences of interest and the signal is detected using a Multimode plate reader Envision (PerkinElmer). The MOLM14 cell line was used for this experiment. Cells were plated in 96-well plates at 3,750 cells/well in the presence of increasing concentrations of compounds. After incubation of 48 hours with compounds, cells were lysed in lysis buffer and incubated for 45 minutes at 55° C. Cell lysates were mixed with human MEIS1 specific capture probe or human RPL28 (Ribosomal Protein L28) specific probe as a normalization control, as well as blocking probes. Cell lysates were then transferred to the custom assay hybridization plate (Thermo Fisher Scientific) and incubated for 18 to 22 hours at 55° C. Subsequently, plates were washed to remove unbound materials followed by sequential addition of preamplifiers, amplifiers, and label probe. Signals (=gene counts) were measured with a Multimode plate reader Envision. IC₅₀s were calculated by dose-response modelling using appropriate software. For all non-housekeeper genes response equal counts corrected for background and relative expression. For each sample, each test gene signal (background subtracted) was divided by the normalization gene signal (RPL28: background subtracted). Fold changes were calculated by dividing the normalized values for the treated samples by the normalized values for the DMSO treated sample. Fold changes of each target gene were used for the calculation of IC₅₀s.

TABLE 3
Biological data - HTRF assay, proliferation
assay, and MEIS1 mRNA expression assay

| | | | | spheroid | |
|---|---|---|---|---|---|
| | HTRF-30 min | | spheroid | assay_OCI- | spheroid |
| Co. | incubation | MEIS1 | assay_MOLM14 | AML3 | assay_KO-52 |
| No. | IC₅₀ (μM) | IC₅₀ (μM) | IC₅₀ (μM) | IC₅₀ (μM) | IC₅₀ (μM) |
| 43 | 0.000027 | 0.024 | 0.014 | 0.023 | 3.3 |
| 50 | 0.000079 | 0.089 | 0.039 | | 2.3 |
| 51 | 0.000042 | 0.011 | 0.008 | 0.024 | 1.9 |
| 51a | 0.000024 | 0.011 | 0.013 | 0.070 | 1.2 |
| 59 | 0.000054 | 0.009 | 0.011 | 0.012 | 2.1 |
| 60 | <0.0000095 | 0.007 | 0.013 | 0.013 | 2.7 |
| 61 | 0.000132 | 0.023 | 0.010 | | 7.9 |
| 62 | 0.000085 | 0.014 | | | |
| 63 | 0.000176 | 0.083 | 0.053 | 0.121 | 5.9 |
| 64 | 0.000284 | ~0.039 | | | |
| 65 | 0.000079 | 0.045 | | | |
| 82 | ~0.000035 | 0.011 | | | |
| 83 | 0.001300 | 2.220 | | | |
| 85 | 0.000113 | 0.022 | 0.012 | 0.022 | 2.2 |
| 86 | 0.000042 | 0.021 | 0.010 | 0.023 | 1.3 |
| 87 | 0.000042 | 0.027 | | | |
| 91 | 0.000155 | 0.094 | | | |
| 92 | 0.000219 | 0.052 | | | |
| 98 | 0.000747 | ~0.535 | | | |
| 101 | 0.000041 | 0.007 | 0.041 | 0.008 | 3.1 |
| 104 | 0.000147 | 0.254 | 0.275 | | >15 |
| 106 | 0.000039 | 0.104 | | | |
| 108 | 0.000161 | 0.153 | | | |
| 114 | 0.000173 | 0.487 | | | |
| 117a | 0.000088 | 0.007 | 0.005 | 0.007 | 1.6 |
| 120 | 0.000054 | 0.149 | 0.539 | | |
| 121 | 0.000049 | 0.013 | | | |
| 125 | 0.000027 | 0.016 | 0.011 | 0.012 | 7.0 |
| 126 | 0.000091 | 0.032 | 0.018 | | |
| 135 | ~0.000056 | 0.007 | | | |
| 156 | 0.000042 | 0.187 | 0.042 | | |
| 169 | 0.000061 | 0.071 | 0.016 | | 10.0 |
| 169a | 0.000061 | 0.015 | 0.017 | | |
| 169b | 0.000195 | ~0.22 | 0.087 | | |
| 190a | 0.000101 | 0.113 | 0.040 | | 10.0 |
| 190b | 0.000063 | 0.065 | 0.046 | | 4.9 |
| 191a | 0.000056 | 0.039 | 0.007 | | 4.6 |
| 191b | 0.000071 | 0.041 | 0.033 | | 7.0 |
| 194 | 0.000072 | 0.066 | | | |
| 196 | 0.000190 | 0.438 | | | |
| 207 | 0.000016 | 0.029 | 0.016 | | 14.3 |
| 213a | 0.000567 | 1.149 | | | |
| 213b | 0.001629 | ~2.30 | | | |
| 283 | 0.000610 | 0.440 | | | |
| 286 | 0.000046 | 0.059 | | | |
| 287 | 0.000034 | 0.026 | | | |
| 288 | 0.000059 | 0.043 | | | |
| 289 | 0.000055 | 0.031 | | | |
| 290 | 0.000393 | 0.170 | | | |
| 291 | 0.000594 | 0.468 | | | |
| 292a | 0.000132 | | | | |
| 292b | 0.000562 | ~0.586 | | | |
| 293a | 0.000459 | 0.148 | | | |
| 293b | 0.000152 | 0.259 | | | |
| 294a | 0.000237 | 0.365 | | | |
| 294b | 0.000077 | 0.153 | | | |
| 295 | 0.000171 | 0.338 | | | |
| 296 | 0.000339 | 0.549 | | | |

3) In Vitro Proliferation—Menin-MLL Inhibitor in Combination with FLT3 Inhibitor (Gilteritinib or Midostaurin), DNA Intercalating Agent (Idarubicin) or Hypomethylating Agent (Decitabine)

The effect of Compound 51 in combination with gilteritinib, midostaurin, idarubicin or decitabine was determined in proliferation assays.

### Cell Lines

AML cell lines MOLM-13, OCI-AML3 and MV4-11 were purchased from DSMZ. MOLM-13 were grown in RPMI medium supplemented with 10% Fetal Bovine Serum (FBS) and 1% penicillin/streptomycin. MV4-11 cells were grown in IMDM medium supplemented with 10% FBS and 1% penicillin/streptomycin. OCI-AML3 cells were cultured in 80-90% alpha-MEM (with or without ribo- and deoxyribonucleosides)+10-20% FBS. All cell lines were cultured at 37° C. in 5% CO₂ atmosphere.

### Cell Titer Glo Assays

AML cell lines (10-20×10³ cells/well) were seeded in 96-well plates and grown for 6 days in serum (10%)-containing medium in the presence or absence of inhibitors at the indicated concentrations. Proliferation was analyzed by means of a Cell Titer Glo assay using the CellTiter 96 Aqueous One Solution Cell Proliferation Assay (Promega, Madison, WI, USA), according to the manufacturer's instructions. Data are mean with standard deviation from two to four independent experiments in technical triplicates.

### Synergy Calculations

R-based Biochemically Intuitive Generalized Loewe (BIGL) model implemented with a highest single agent (HSA) null model. Specifically, the BIGL methodology was applied to calculate drug-drug interactions (Van der Borght, K., Tourny, A., Bagdziunas, R. et al. BIGL: Biochemically Intuitive Generalized Loewe null model for prediction of the expected combined effect compatible with partial agonism and antagonism. Sci Rep 7, 17935 (2017); Thas, O., Tourny, A., Verbist, B., Hawinkel, S., Nazarov, M., Mutambanengwe, K., & Bijnens, L. Statistical detection of synergy: New methods and a comparative study. Pharmaceutical Statistics (2021)).

Synergy matrix results of the BIGL analysis of the cell line data with HSA as mean model calculated on the basis of the cellular metabolic activity using Cell Titer-Glo assay. Bootstrap confidence intervals are indicated. Effect sizes and their confidence intervals are shown. Notably, each data point based on the p-value and sign of the respective maxR statistic with the size of the dots reflecting the degree of synergy or antagonism corresponding to graded scale. No significant average effect if zero is included in the interval.

### Results

(A) Menin-MLL Inhibitor in Combination with FLT3 Inhibitor (Gilteritinib or Midostaurin)

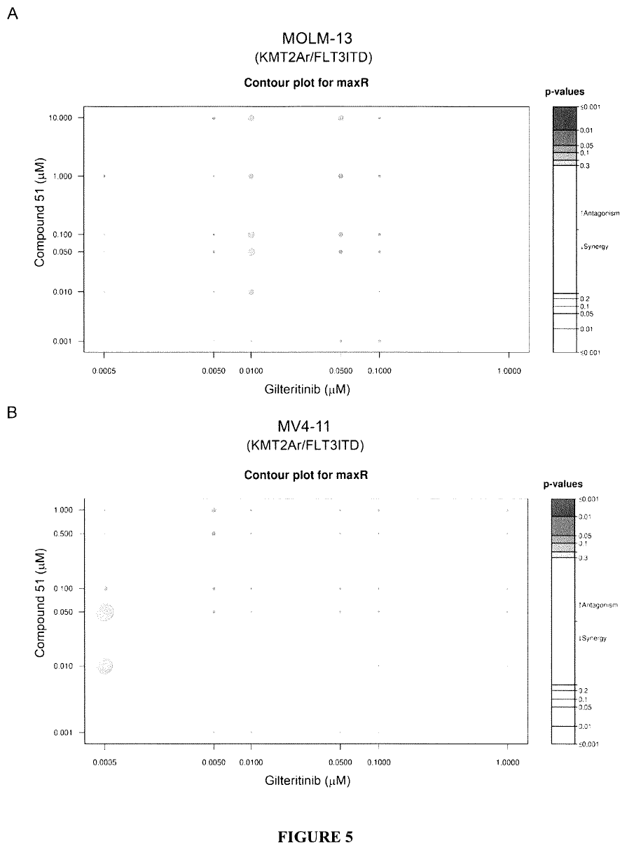

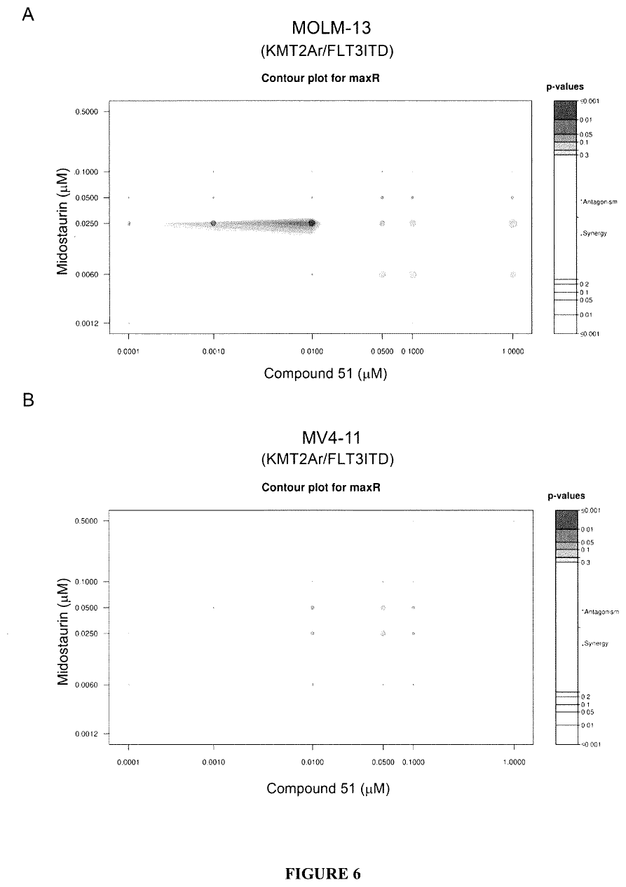

A pairwise matrix combination of gilteritinib or midostaurin with Compound 51 was evaluated in MOLM-13 and MV4-11 (KMT2A-rearranged; FLT3-ITD) AML cell lines using a 6-day CellTiter-Glo assay format. Notably, the combination of Compound 51 with either gilteritinib or midostaurin is not antagonistically cytotoxic in either of these cell lines (as reflected in FIGS. 5A and 5B (FIGs means Figures) plus detailed below in Tables 4A and 4B for gilteritinib; and in FIGS. 6A and 6B plus detailed below in Tables 5A and 5B for midostaurin).

Low doses of the more selective FLT3 inhibitor gilteritinib (i.e., 500 pM or 10 nM) in combination with Compound 51 were found to have a synergistic effect in MOLM-13 and MV4-11 cell lines, respectively, as indicated by a greater decrease in cell proliferation compared to that which would be expected from an additive response (see FIGS. 5A and 5B as well as Tables 4A and 4B).

TABLE 4A
Effect of Compound 51 in Combination with Gilteritinib on MOLM-13 Cell
Proliferation.

| | MOLM-13 | | | | | | |
|---|---|---|---|---|---|---|---|
| Compound 51 | 10 | −0.05 | −0.17 | −0.26** | −0.27** | −0.14 | −0.01 |
| (μm) | | (−0.2, 0.09) | (−0.31, −0.03) | (−0.39, −0.12) | (−0.4, −0.14) | (−0.26, −0.03) | (−0.13, 0.11) |
| | 1 | 0.03 | −0.1 | −0.22 | −0.25 | −0.14 | −0.01 |
| | | (−0.11, 0.17) | (−0.24, 0.03) | (−0.35, −0.1) | (−0.38, −0.12) | (−0.26, −0.02) | (−0.13, 0.11) |
| | 0.1 | −0.03 | −0.13 | −0.3** | −0.24 | −0.14 | −0.01 |
| | | (−0.15, 0.13) | (−0.27, 0) | (−0.43, −0.17) | (−0.37, −0.11) | (−0.26, −0.02) | (−0.13, 0.11) |
| | 0.05 | −0.03 | −0.14 | −0.32** | −0.22 | −0.14 | −0.01 |
| | | (−0.17, 0.12) | (−0.29, 0) | (−0.46, −0.19) | (−0.35, −0.09) | (−0.26, −0.02) | (−0.13, 0.11) |
| | 0.01 | 0.01 | −0.08 | −0.25** | −0.09 | 0.1 | −0.01 |
| | | (−0.15, 0.18) | (−0.23, 0.08) | (−0.39, −0.1) | (−0.23, 0.05) | (−0.22, 0.02) | (−0.13, 0.11) |
| | 0.001 | −0.01 | −0.06 | −0.1 | −0.02 | 0.02 | −0.01 |
| | | (−0.17, 0.14) | (−0.21, 0.09) | (−0.26, 0.05) | (−0.16, 0.12) | (−0.11, 0.14) | (−0.13, 0.11) |

| | | | | | | |
|---|---|---|---|---|---|---|
| Gilteritinib (μM) | 0.0005 | 0.005 | 0.01 | 0.05 | 0.1 | 1 |

Note
that absence of any asterisk indicates an additive effect, antagonism is indicated by “*” and synergy is indicated by “**”.
Antagonistic and synergistic effects are derived from the significant calls based on the maxR test and numbers associated with same interpreted as antagonistic or synergistic, respectively.

TABLE 4B
Effect of Compound 51 in Combination with Gilteritinib on MV4-11 Cell
Proliferation.

| | MV4-11 | | | | | | |
|---|---|---|---|---|---|---|---|
| Compound 51 | 1 | −0.02 | −0.02 | −0.02 | −0.02 | −0.02 | −0.02 |
| (μm) | | (−0.06, 0.02) | (−0.05, 0) | (−0.04, 0) | (−0.04, 0) | (−0.04, 0) | (−0.04, 0) |
| | 0.5 | −0.02 | −0.03 | −0.02 | −0.02 | −0.02 | −0.02 |
| | | (−0.06, 0.02) | (−0.05, 0) | (−0.04, 0) | (−0.04, 0) | (−0.04, 0) | (−0.04, 0) |
| | 0.1 | −0.07 | −0.03 | −0.02 | −0.02 | −0.02 | −0.02 |
| | | (−0.11, −0.03) | (−0.07, 0.02) | (−0.04, 0) | (−0.04, 0) | (−0.04, 0.01) | (−0.04, 0.01) |
| | 0.05 | −0.18** | −0.03 | −0.02 | −0.02 | −0.02 | −0.02 |
| | | (−0.25, −0.12) | (−0.07, 0.02) | (−0.04, 0) | (−0.04, 0.01) | (−0.04, 0) | (−0.04, 0.01) |
| | 0.01 | −0.36** | −0.02 | −0.02 | −0.02 | −0.02 | −0.02 |
| | | (−0.5, −0.21) | (−0.07, 0.02) | (−0.05, 0.01) | (−0.04, 0.01) | (−0.04, 0.01) | (−0.04, 0.01) |
| | 0.001 | −0.03 | −0.01 | −0.03 | −0.01 | −0.01 | −0.01 |
| | | (−0.23, 0.17) | (−0.06, 0.04) | (−0.04, 0.03) | (−0.04, 0.03) | (−0.04, 0.03) | (−0.04, 0.03) |

| | | | | | | |
|---|---|---|---|---|---|---|
| Gilteritinib (μM) | 0.0005 | 0.005 | 0.01 | 0.05 | 0.3 | 1 |

Note
that absence of any asterisk indicates an additive effect, antagonism is indicated by “*” and synergy is indicated by “**”.
Antagonistic and synergistic effects are derived from the significant calls based on the maxR test and numbers associated with same interpreted as antagonistic or synergistic, respectively.

Low doses of the FLT3 inhibitor Midostaurin (i.e. 6 to 25 nM) synergized with Compound 51 in MOLM-13 cells (see FIG. 6A (FIG. means Figure) as well as Table 5 Å).

TABLE 5A
Effect of Compound 51 in Combination with Midostaurin on MOLM-13 Cell
Proliferation.

| | MOLM-13 | | | | | | |
|---|---|---|---|---|---|---|---|
| Compound 51 | 1 | −0.03 | −0.18** | −0.19** | −0.08 | −0.04 | −0.02 |
| (μm) | | (−0.12, 0.06) | (−0.26, −0.1) | (−0.26,−0.13) | (−0.14, −0.03) | (−0.09, 0.01) | (−0.07, 0.03) |
| | 0.1 | −0.08 | −0.23** | −0.17** | −0.08 | −0.04 | −0.02 |
| | | (−0.16, 0.01) | (−0.3, −0.15) | (−0.24, 0.11) | (−0.13, −0.03) | (−0.09, 0.01) | (−0.07, 0.04) |
| | 0.05 | −0.03 | −0.21** | −0.14 | 0.08 | −0.04 | −0.02 |
| | | (−0.12, 0.06) | (−0.29, −0.13) | (−0.21, −0.08) | (−0.13, −0.03) | (−0.09, 0.01) | (−0.07, 0.03) |
| | 0.01 | 0.01 | −0.06 | 0.1 | −0.06 | −0.04 | −0.02 |
| | | (−0.09, 0.11) | (−0.16, 0.04) | (0.02, 0.38) | (−0.12, 0.01) | (−0.09, 0.01) | (−0.07, 0.03) |
| | 0.001 | −0.03 | −0.02 | 0.07 | −0.06 | −0.04 | −0.02 |
| | | (−0.13, 0.07) | (−0.12, 0.09) | (−0.01, 0.15) | {−0.12, −0.01) | (−0.09, 0.01) | (−0.07, 0.03) |
| | 0.0001 | −0.07 | −0.02 | 0.05 | −0.06 | −0.04 | −0.02 |
| | | (−0.16, 0.03) | (−0.12, 0.08) | (−0.03, 0.3.3) | (−0.12, −0.01) | (−0.09, 0.01) | (−0.07, 0.04) |

| | | | | | | |
|---|---|---|---|---|---|---|
| Midostaurin (μM) | 0.0012 | 0.006 | 0.025 | 0.05 | 0.1 | 0.5 |

Note
that absence of any asterisk indicates an additive effect, antagonism is indicated by “*” and synergy is indicated by “**”.
Antagonistic and synergistic effects are derived from the significant calls based on the maxR test and numbers associated with same interpreted as antagonistic or synergistic, respectively.

TABLE 5B
Effect of Compound 51 in Combination with Midostaurin on MV4-11 Cell
Proliferation.

| | MV4-11 | | | | | | |
|---|---|---|---|---|---|---|---|
| Compound | 1 | 0.02 | 0.01 | 0 | 0 | 0 | 0.03 |
| 51 | | (−0.16, 0.2) | (−0.17, 0.19) | (−0.17, 0.18) | (−0.17, 0.18) | (−0.16, 0.17) | (−0.11, 0.18) |
| (μm) | 0.1 | −0.03 | −0.06 | −0.08 | −0.09 | −0.04 | 0.03 |
| | | (−0.2, 0.14) | (−0.22, 0.1) | (−0.23, 0.06) | (−0.23, 0.06) | (−0.18, 0.09) | (−0.12, 0.19) |
| | 0.05 | −0.02 | −0.07 | −0.14 | −0.13 | −0.04 | 0.03 |
| | | (−0.22, 0.18) | (−0.26, 0.11) | (−0.3, 0.01) | (−0.27, 0) | (−0.18, 0.1) | (−0.13, 0.2) |
| | 0.01 | −0.06 | −0.13 | −0.14 | −0.11 | −0.04 | 0.03 |
| | | (−0.34, 0.22) | (−0.39, 0.14) | (−0.36, 0.07) | (−0.27, 0.04) | (−0.18, 0.11) | (−0.13, 0.2) |
| | 0.001 | −0.02 | −0.01 | 0.05 | 0.07 | −0.03 | 0.03 |
| | | (−0.39, 0.36) | (−0.37, 0.35) | (−0.29, 0.19) | (−0.24, 0.1) | (−0.18, 0.12) | (−0.13, 0.2) |
| | 0.0001 | −0.03 | 0.13 | 0.02 | −0.05 | −0.02 | 0.03 |
| | | (−0.39, 0.34) | (−0.27, 0.49) | (−0.24, 0.28) | (−0.23, 0.12) | (−0.17, 0.13) | (−0.13, 0.2) |

| | | | | | | |
|---|---|---|---|---|---|---|
| Midostaurin (μM) | 0.0012 | 0.006 | 0.025 | 0.05 | 0.1 | 0.5 |

Note
that absence of any asterisk indicates an additive effect, antagonism is indicated by “*” and synergy is indicated by “**”.
Antagonistic and synergistic effects are derived from the significant calls based on the maxR test and numbers associated with same interpreted as antagonistic or synergistic, respectively.

(b) Menin-MLL Inhibitor in Combination with Idarubicin

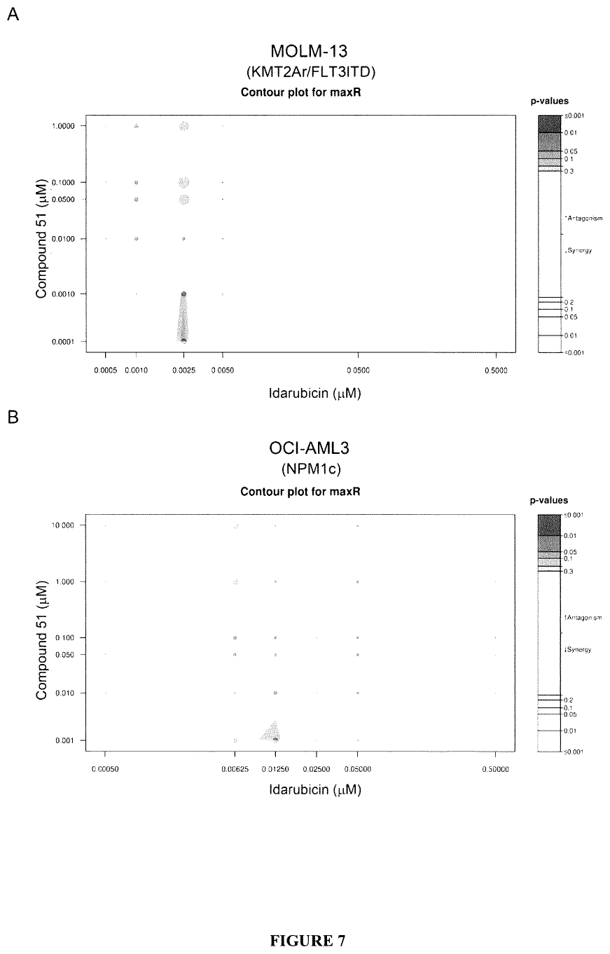

A pairwise matrix combination of idarubicin and Compound 51 was evaluated in MOLM-13 (KMT2A-rearranged; FLT3-ITD) and OCI-AML3 (NPM1c) cells using a 6-day Cell Titer-Glo assay format. Notably, the combination of idarubicin and Compound 51 is synergistically cytotoxic in MOLM-13 (KMT2A-rearranged; FLT3-ITD) cells as reflected in the contour plot (FIG. 7A) and detailed below in Table 6 Å.

TABLE 6A
Effect of Compound 51 in Combination with Idarubicin on MOLM-13 Cell
Proliferation.

| | MOLM-13 | | | | | | |
|---|---|---|---|---|---|---|---|
| Compound | 1 | −0.08 | −0.38** | −0.24 | −0.11 | 0.02 | 0.02 |
| 51 | | (−0.34, 0.18) | (−0.57, −0.19) | (−0.47, 0) | (−0.26, 0.05) | (−0.12, 0.17} | (−0.12, 0.17) |
| (μm) | 0.1 | −0.13 | −0.42** | −0.23 | −0.11 | 0.02 | 0.02 |
| | | (−0.37, 0.11) | (−0.6, −0.24) | (−0.43, 0.01) | (−0.26, 0.05) | (−0.12, 0.17) | (−0.12, 0.17) |
| | 0.05 | −0.1 | −0.41** | −0.23 | −0.11 | 0.02 | 0.02 |
| | | (−0.37, 0.17) | (−0.61, 0.21) | (−0.48, 0.02) | (−0.26, 0.05) | (−0.12, 0.17) | (−0.12, 0.17) |
| | 0.01 | −0.35 | −0.73 | −0.26 | −0.11 | 0.02 | 0.02 |
| | | (−0.47, 0.17) | (−0.51, 0.1) | (−0.5, −0.03) | (−0.26, 0.05) | (−0.12, 0.17) | (−0.12, 0.17) |
| | 0.001 | −0.03 | −0.07 | 0.04 | −0.1 | 0.02 | 0.02 |
| | | (−0.36, 0.3) | (−0.41, 0.26) | (−0.25, 0.33) | (−0.26, 0.05) | (−0.12, 0.17) | (−0.12, 0.17) |
| | 0.0001 | −0.05 | −0.02 | 0.07 | −0.1 | 0.03 | 0.02 |
| | | (−0.37, 0.27) | (−0.37, 0.32) | {−0.23, 0.36) | (−0.25, 0.06) | (−0.12, 0.17) | (−0.12, 0.17) |

| | | | | | | |
|---|---|---|---|---|---|---|
| Idarubicin (μM) | 0.0005 | 0.001 | 0.0025 | 0.005 | 0.05 | 0.5 |

Note
that absence of any asterisk indicates an additive effect, antagonism is indicated by “*” and synergy is indicated by “**”.
Antagonistic and synergistic effects are derived from the significant calls based on the maxR test and numbers associated with same interpreted as antagonistic or synergistic, respectively.

The combination of idarubicin and Compound 51 is not antagonistically cytotoxic in OCI-AML3 (NPM1c) cells as reflected in the contour plot (FIG. 7B) and detailed below in Table 6B.

TABLE 6B
Effect of Compound 51 in Combination with Idarubicin on OCI-AML3 Cell
Proliferation.

| | OCI-AML3 | | | | | | |
|---|---|---|---|---|---|---|---|
| Compound | 10 | 0.04 | −0.06 | 0 | −0.01 | −0.01 | −0.01 |
| 51 | | (−0.11, 0.2) | (−0.14, 0.02) | (−0.05, 0.04) | (−0.05, 0.03) | (−0.05, 0.03) | (−0.05, 0.03) |
| (μm) | 1 | −0.03 | −0.06 | 0 | −0.01 | −0.01 | −0.01 |
| | | (−0.17, 0.12) | ( 0.14, 0.03) | (−0.05, 0.04) | 1−0.05, 0.03) | (−0.05, 0.03) | (−0.05, 0.03) |
| | 0.1 | 0.01 | −0.05 | 0 | 0 | −0.01 | −0.01 |
| | | (−0.34, 0.16) | (−0.13, 0.04) | (−0.05, 0.05) | (−0.04, 0.04) | (−0.05, 0.03) | (−0.05, 0.03) |
| | 0.05 | 0.04 | −0.04 | 0 | −0.01 | −0.01 | −0.01 |
| | | (−0.13, 0.22) | (−0.13, 0.04) | (−0.05, 0.05) | (−0.05, 0.03) | (−0.05, 0.03) | (−0.05, 0.03) |
| | 0.01 | 0.05 | −0.02 | 0.01 | 0 | −0.01 | −0.01 |
| | | (−0.15, 0.26) | (−0.1, 0.07) | (−0.04, 0.06) | (−0.04, 0.04) | (−0.05, 0.03) | (−0.05, 0.03) |
| | 0.001 | −0.02 | 0 | 0.02 | 0 | −0.01 | −0.01 |
| | | (−0.22, 0.18) | (−0.09, 0.1) | (−0.04, 0.07) | (−0.04, 0.04) | (−0.05, 0.03) | (−0.05, 0.03) |

| | | | | | | |
|---|---|---|---|---|---|---|
| Idarubicin (μM) | 0.0005 | 0.00625 | 0.0125 | 0.025 | 0.05 | 0.5 |

Note
that absence of any asterisk indicates an additive effect, antagonism is indicated by “*” and synergy is indicated by “**”.
Antagonistic and synergistic effects are derived from the significant calls based on the maxR test and numbers associated with same interpreted as antagonistic or synergistic, respectively.

(C) Menin-MLL Inhibitor in Combination with Decitabine

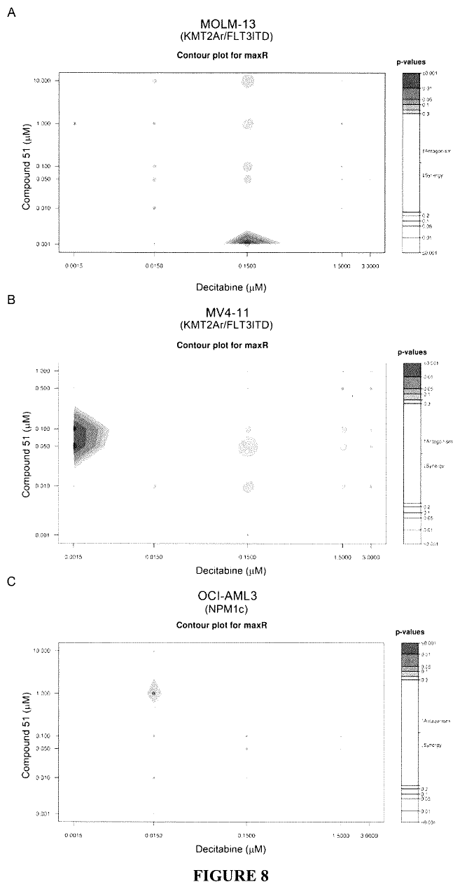

A pairwise matrix combination of decitabine and Compound 51 was evaluated in MOLM-13, MV4-11 (KMT2A-rearranged; FLT3-ITD) and OCI-AML3 (NPM1c) cells using a 6-day CellTiter-Glo assay format. Notably, the combination of decitabine and Compound 51 was found to be synergistically cytotoxic in MOLM-13 (KMT2A-rearranged; FLT3-ITD) cells as reflected in the contour plot (FIG. 8A) and detailed below in Table 7 Å.

TABLE 7A
Effect of Compound 51 in Combination with Decitabine on MOLM-13 Cell
Proliferation.

| | | MOLM-13 | | | | |
|---|---|---|---|---|---|---|
| Compound | 10 | −0.06 | −0.16** | −0.38** | −0.05 | −0.03 |
| 51 | | (−0.12, 0.01) | (−0.22, −0.1) | (−0.44, −0.33) | (−0.1, −0.01) | (−0.07, 0.02) |
| (μm) | 1 | 0.03 | −0.1 | −0.38** | −0.05 | −0.03 |
| | | (−0.03, 0.1) | (−0.16, −0.04) | (−0.43, −0.32) | (−0.1, −0.01) | (−0.07, 0.02) |
| | 0.1 | −0.01 | −0.33** | −0.35** | −0.05 | −0.03 |
| | | (−0.08, 0.05) | (−0.19, −0.06) | (−0.41, −0.29) | (−0.1, −0.01) | (−0.07, 0.02) |
| | 0.05 | −0.03 | −0.13** | −0.32** | −0.05 | −0.03 |
| | | (−0.1, 0.03) | (−0.2, −0.07) | (−0.39, −0.26) | (−0.1, −0.01) | (−0.07, 0.02) |
| | 0.01 | 0 | −0.09 | −0.16 | −0.05 | −0.02 |
| | | (−0.08, 0.07) | (−0.17, −0.02) | (−0.22, −0.09) | (−0.09, 0) | (−0.07, 0.02) |
| | 0.001 | −0.03 | −0.07 | −0.03 | −0.03 | −0.02 |
| | | (−0.11, 0.04) | (−0.15, 0) | (−0.1, 0.04) | (−0.08, 0.02) | (−0.06, 0.03) |

| | | | | | |
|---|---|---|---|---|---|
| Decitabine (μM) | 0.0015 | 0.015 | 0.15 | 1.5 | 3 |

Note that absence of any asterisk indicates an additive effect, antagonism is indicated by “*” and synergy is indicated by “**”.
Antagonistic and synergistic effects are derived from the significant calls based on the maxR test and numbers associated with same interpreted as antagonistic or synergistic, respectively.

Likewise, the combination of decitabine and Compound 51 is synergistically cytotoxic in MV4-11 (KMT2A-AF4; FLT3-ITD) cells as reflected in the contour plot (FIG. 8B) and detailed below in Table 7B.

TABLE 7B
Effect of Compound 51 in Combination with Decitabine on MV4-11 Cell
Proliferation.

| | | MV4-11 | | | | |
|---|---|---|---|---|---|---|
| Compound | 1 | 0 | −0.01 | −0.02 | −0.03 | −0.03 |
| 51 | | (−0.03, 0.02) | (−0.04, 0.01) | (−0.05, 0.01) | (−0.06, 0) | (−0.06, 0) |
| (μm) | 0.5 | 0 | −0.01 | −0.02 | −0.03 | −0.03 |
| | | (−0.03, 0.03) | (−0.04, 0.02) | (−0.05, 0) | (−0.06, 0) | (−0.06, −0.01) |
| | 0.1 | 0.01 | −0.04 | −0.09** | −0.11** | −0.06 |
| | | (−0.03, 0.04) | (−0.07, −0.01) | (−0.12, −0.06) | (−0.14, −0.08) | (−0.09, −0.03) |
| | 0.05 | 0.02 | −0.07 | −0.23** | −0.11** | −0.06 |
| | | (−0.04, 0.07) | (−0.12, −0.02) | (−0.27, −0.19) | (−0.15, −0.08) | (−0.09, −0.03) |
| | 0.01 | −0.1** | −0.11** | −0.23** | 0.01 | −0.05 |
| | | (−0.13, −0.07) | (−0.18, −0.04) | (−0.29, −0.18) | (−0.06, 0.09) | (−0.08, −0.02) |
| | 0.001 | −0.01 | −0.03 | 0 | −0.05 | −0.04 |
| | | (−0.07, 0.06) | (−0.1, 0.04) | (−0.07, 0.06) | (−0.09, −0.01) | (−0.07, −0.01) |

| | | | | | |
|---|---|---|---|---|---|
| Decitabine (μM) | 0.0015 | 0.035 | 0.15 | 1.5 | 3 |

Note that absence of any asterisk indicates an additive effect, antagonism is indicated by “*” and synergy is indicated by “**”.
Antagonistic and synergistic effects are derived from the significant calls based on the maxR test and numbers associated with same interpreted as antagonistic or synergistic, respectively.

In OCI-AML3 (NPM1c) cells, Compound 51 is not antagonistically cytotoxic with Decitabine as reflected in the contour plot (FIG. 8C) and detailed below in Table 7C.

TABLE 7C
Effect of Compound 51 in Combination with Decitabine on OCI-AML3 Cell
Proliferation.

| | | OCI-AML3 | | | | |
|---|---|---|---|---|---|---|
| Compound | 10 | 0.01 | 0.1 | −0.02 | −0.01 | 0.01 |
| 51 | | (−0.16, 0.17) | (−0.07, 0.27) | (−0.18, 0.14) | (−0.12, 0.1) | (−0.09, 0.11) |
| (μm) | 1 | 0.02 | 0.13 | 0.01 | −0.02 | 0 |
| | | (−0.15, 0.18) | (−0.05, 0.3) | (−0.16, 0.17) | (−0.13, 0.09) | (−0.1, 0.1) |
| | 0.1 | 0.01 | 0.08 | −0.07 | −0.02 | 0 |
| | | (−0.17, 0.2) | (−0.11, 0.27) | (−0.25, 0.1) | (−0.33, 0.09) | (−0.1, 0.1) |
| | 0.05 | 0 | 0 | −0.1 | −0.03 | 0 |
| | | (−0.19, 0.2) | (−0.2, 0.19) | (−0.29, 0.09) | (−0.13, 0.08) | (−0.1, 0.1) |
| | 0.01 | −0.01 | −0.1 | −0.07 | −0.02 | 0 |
| | | (−0.22, 0.19) | (−0.29, 0.09) | (−0.26, 0.12) | (−0.13, 0.09) | (−0.1, 0.1) |
| | 0.001 | −0.02 | −0.06 | −0.04 | −0.02 | 0.01 |
| | | (−0.2, 0.17) | (−0.25, 0.14) | (−0.24, 0.15) | (−0.13, 0.09) | (−0.09, 0.11) |

| | | | | | |
|---|---|---|---|---|---|
| Decitabine (μM) | 0.0015 | 0.015 | 0.35 | 3.5 | 3 |

4) Efficacy of Menin-MLL Inhibitor in Combination with Azacitidine in MOLM-13 (KMT2A-Rearranged) Model in NSG Mice

### Test Agents and Controls

Compound 51 was formulated in purified pyrogen-free deionized water, pH 4.0 and prepared to reach a total volume of 8 mL/kg. Doses were adjusted by individual body weight each day. Working stocks of Compound 51 were prepared once per week for each study and stored at 25° C.

### Animals

Female NSG (NOD.Cg-Prkdc^{scid} Il2rg^{tm1Wjl}/SzJ) mice were used when they were approximately 6 to 8 weeks of age and weighed approximately 25 g. All animals could acclimate and recover from any shipping-related stress for a minimum of 7 days prior to experimental use. Autoclaved water and irradiated food were provided ad libitum, and the animals were maintained on a 12 hour light and dark cycle. Cages, bedding, and water bottles were autoclaved before use and changed weekly. The tissue culture and cell injection reagents are summarized below in Table 8.

TABLE 8
Tissue Culture and Cell Injection Reagents

DPBS (Dulbecco's phosphate-buffered saline)
Heat-inactivated fetal bovine serum
RPMI 1640 medium
L-glutamine
Gentamycin
T175 Culture Flask
Roller Bottle

### Tumor Model and Cell Culture Method

Human AML MOLM-13 cells were cultured at 37° C., 5% CO₂ in the indicated complete culture media (RPMI 1640+10% HI-FBS+2 mM L-glutamine+50 μg/ml Gentamycin). Cells were harvested while in logarithmic growth and resuspended in cold (4° C.) Roswell Park Memorial Institute (RPMI) 1640 in serum-free medium.

For the disseminated MOLM-13 model, each mouse received 1×10⁵ cells via IV injection in a total volume of 0.2 mL using a 26-gauge needle.

### Study Designs

Day 0 is the day of tumor cell implantation and study initiation.

Mice bearing IV MOLM-13 xenograft tumors were randomly assigned to treatment groups 5 days post-tumor cell engraftment.

### Animal Monitoring

Animals were monitored daily for clinical signs related to either compound toxicity or tumor burden (i.e., hind limb paralysis, lethargy, etc.).

### Calculations

For survival assessment, results were plotted as the percentage survival against days post tumor implant. Negative clinical signs and/or ≥20% body weight loss was used as a surrogate endpoint for death. Median survival was determined utilizing Kaplan-Meier survival analysis. The percent increased life span (ILS) was calculated as: ((median survival day of treated group−median survival day of control group)/median survival day of control group)×100. Animals failing to reach the surrogate endpoint due to adverse clinical signs or death unrelated to treatment were censored for the survival assessment. As defined by NCI criteria, ≥25% ILS is considered biologically significant. (Johnson J I et al. Br J Cancer. 2001. 84(10), 1424-1431).

### Data Analysis

Survival and body weight data were graphically represented utilizing Prism (Version 9). Statistical significance for body weights was evaluated as described above. Statistical significance was evaluated for Kaplan-Meier survival plots comparing therapeutic treatment group vs. appropriate vehicle-treated control using log-rank (Mantel-Cox) test in R software version 3.4.2. Differences between groups were considered significant when the p value was ≤0.05.

### Survival

The disseminated MOLM-13 model described above was used to examine the efficacy of menin-KMT2A-inhibitor (formulated as described above) in combination with azacitidine (formulated in 0.9% saline). Treatment with compound(s) was initiated 5 days after cell injection. Compound 51 was given in the morning, and azacitidine was given in the afternoon, approximately 6-8 hr apart. The treatment of each group using compound(s) in this efficacy study is summarized in Table 9.

TABLE 9
Treatment of MOLM-13 tumors in mice (‘ip’ means
intraperitoneal; ‘po’ means oral)

| | TREATMENT | | | | |
|---|---|---|---|---|---|
| GROUP | (n = 10) | DOSE | | ROUTE | SCHEDULE |
| 1 | Vehicles | 8 | mL/kg | ip/po | QD × 7/QD × 28 |
| 2 | Azacitidine | 2 | mg/kg | ip | QD × 7 |
| 3 | Compound 51 | 2 | mg/kg | po | QD × 28 |
| 4 | Azacitidine + | 2 | mg/kg | ip/po | QD × 7/QD × 28 |
| | Compound 51 | 2 | mg/kg | | |

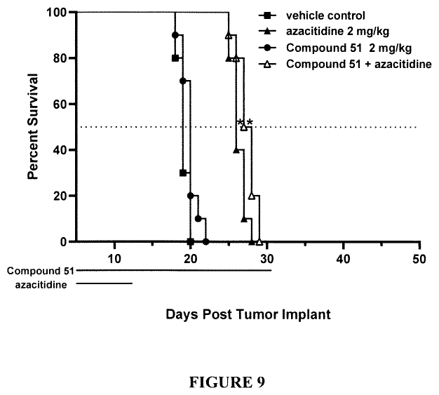

The Kaplan-Meier survival curve is shown in FIG. 9. The impact on life span of treating mice bearing established MOLM-13 tumors for 4 weeks is summarized in Table 10.

TABLE 10
Impact on Life Span following Treatment
of OCI-AML3-tumors in mice

| TREATMENT | % ILS |
|---|---|
| Azacitidine | *37 |
| Compound 51 | *5 |
| Azacitidine + Compound 51 | *†45 |

*p < 0.05 as compared with vehicle control and †azacitidine treated mice.

As reflected in Table 10, treatment of mice with Compound 51 in combination with azacitidine resulted in statistically significant increased lifespan (ILS) of MOLM-13 tumor-bearing mice as compared to that of mice treated with azacitidine alone.

1. A combination comprising:
a therapeutically effective amount of a menin-mixed-lineage leukemia 1 (MLL) inhibitor of Formula (I), or a tautomer or a stereoisomeric form thereof, or a pharmaceutically acceptable salt or a solvate thereof; and
a therapeutically effective amount of at least one other therapeutic agent, wherein the at least one other therapeutic agent is a hypomethylating agent, cytidine deaminase inhibitor, a DNA intercalating agent, a pyrimidine analog, a purine analog, a kinase inhibitor, a CD20 inhibitor, an isocitrate dehydrogenase inhibitor, an immunomodulatory agent or a dihydroorotate dehydrogenase inhibitor;
wherein the menin-MLL inhibitor of Formula (I) has the structure: 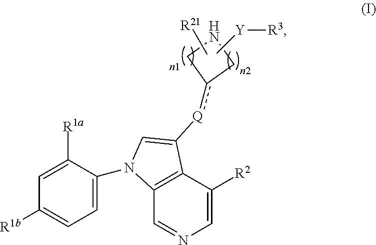
wherein
Q represents —CHRy-, —O—, —C(═O)—, —NRq-, or —CRy=; the dotted line is an optional additional bond to form a double bond in case Q represents —CRy=;
R1a represents hydrogen; cyano; halo; Het; —C(═O)—NRxaRxb; —S(═O)₂—R18;
—C(═O)—O—C₁₋₄alkyl-NR22aR22b; —C(═O)—O—C₁₋₄alkyl; 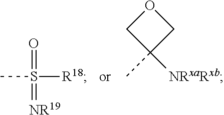
R18 represents C1-6alkyl or C3-6cycloalkyl;
R19 represents hydrogen or C1-6alkyl;
or R18 and R19 are taken together to form —(CH2)3-, —(CH2)4- or —(CH2)5-;
Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C1-4alkyl, C3-6cycloalkyl, or cyano;
Rxa and Rxb are each independently selected from the group consisting of hydrogen;
Het3; C3-6cycloalkyl; and C1-6alkyl; wherein optionally said C3-6cycloalkyl and C1-6alkyl are substituted with 1, 2 or 3 substituents each independently selected from the group consisting of —OH, —OC1-4alkyl, —C1-4alkyl-OH, halo, CF3, C3-6cycloalkyl, Het3, and NR11cR11d;
or Rxa and Rxb are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C1-4alkyl, halo, —OH, —O—C1-4alkyl, cyano, and C1-4alkyl substituted with one, two or three substituents selected from the group consisting of halo and OR23;
or Rxa and Rxb are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C1-4alkyl, halo, —OH, —O—C1-4alkyl, cyano, and C1-4alkyl substituted with one, two or three substituents each independently selected from the group consisting of halo and OR23;
R23 represents hydrogen or C1-4alkyl optionally substituted with one, two or three halo;
R1b represents hydrogen, F, Cl, or —O—C1-4alkyl;
R2 represents halo, C3-6cycloalkyl, C1-4alkyl, —O—C1-4alkyl, cyano, or C1-4alkyl substituted with one, two or three halo substituents;
R21 represents hydrogen or —Ya-R3a; provided that when R21 represents —Ya-R3a, one of —Ya-R3a and —Y—R3 is attached to the nitrogen atom of the ring;
Y and Ya each independently represent a covalent bond or 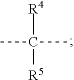
n1 is selected from 1 and 2;
n2 is selected from 1, 2, 3 and 4;
Ry represents hydrogen, —OH, C1-4alkyl, —C1-4alkyl-OH, or —C1-4alkyl-O—C1-4alkyl;
Rq represents hydrogen or C1-4alkyl;
R5 represents hydrogen, C1-4alkyl, or C3-6cycloalkyl;
R3, R3a, and R4 are each independently selected from the group consisting of Heti; Het2; Cy2;
C1-8alkyl; and C1-8alkyl substituted with one, two, three or four substituents each independently selected from the group consisting of —C(═O)—NR10aR10b, —C(═O)—Het6a, —C(═O)—Het6b, —NR10c-C(═O)—C1-4alkyl, —S(═O)₂-C1-4alkyl, —NRxcRxd, —NR8aR8b, —CF3, cyano, halo, —OH, —O—C1-4alkyl, Het1, Het2, Ar1, and Cy2;
Rxc represents Cy1; Het5; —C1-6alkyl-Cy1; —C1-6alkyl-Het3; —C1-6alkyl-Het4;
or —C1-6alkyl-phenyl;
Rxd represents hydrogen; C1-4alkyl; or C1-4alkyl substituted with one, two or three substituents selected from the group consisting of halo, —OH, —O—C1-4alkyl, and cyano;
or Rxc and Rxd are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo,
—OH, —O—C1-4alkyl, —(C═O)—C1-4alkyl, —S(═O)₂-C1-4alkyl, and cyano;
or Rxc and Rxd are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C1-4alkyl, —(C═O)—C1-4alkyl-S(═O)₂-C1-4alkyl, and cyano;
R8a and R8b are each independently selected from the group consisting of hydrogen;
C1-6alkyl; —(C═O)—C1-4alkyl; and C1-6alkyl substituted with one, two or three substituents each independently selected from the group consisting of —OH, cyano, halo, —S(═O)₂-C1-4alkyl, —O—C1-4alkyl, —C(═O)—NR10aR10b, and —NR10c-C(═O)—C1-4alkyl;
Ar represents phenyl optionally substituted with one, two or three substituents each independently selected from the group consisting of C1-4alkyl, halo, —O—C1-4alkyl, —CF3, —OH, —S(═O)₂-C1-4alkyl, and —C(═O)—NR10aR10b;
Het1 represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R6, —C(═O)—Cy1, and —C(═O)—R8; and wherein said heterocyclyl is optionally substituted on one or two carbon atoms with in total one, two, three or four substituents each independently selected from the group consisting of halo, R6, Het6a, Het6b, C1-4alkyl, oxo, —NR9aR9b and —OH;
Het2 represents C-linked pyrazolyl, 1,2,4-oxadiazolyl, pyridazinyl or triazolyl; which may be optionally substituted on one nitrogen atom with R6a;
R6 and R6a are each independently selected from the group consisting of
Het3; Het4; —C(═O)—NH—Cy1; —C(═O)—NH—R8; —C(═O)—Het6a; —C(═O)—NR10dR10e;
—C(═O)—O—C1-4alkyl; —S(═O)₂-C1-4alkyl;
C1-6alkyl optionally substituted with one or two substituents each independently selected from the group consisting of Het3, Het4, Het6a, Het6b, Cy1, —CN, —OH, —O—C1-4alkyl, —C(═O)—NH—C1-4alkyl, —C(═O)—N(C1-4alkyl)2, —C(═O)—NH—C1-4alkyl-C3-6cycloalkyl, —C(═O)—OH, —NR11aR11b, and —NH—S(═O)₂-C1-4alkyl; and
C3-6cycloalkyl optionally substituted by one or two substituents each independently selected from the group consisting of —CN, —OH, —O—C1-4alkyl, —C(═O)—NH—C1-4alkyl, —C(═O)—N(C1-4alkyl)2, —NH—S(═O)₂-C1-4alkyl, and C1-4alkyl optionally substituted with one substituent selected from the group consisting of OH, —O—C1-4alkyl, —C(═O)—NH—C1-4alkyl and —NH—S(═O)₂-C1-4alkyl;
R8 represents hydrogen, —O—C1-6alkyl, C1-6alkyl; or C1-6alkyl substituted with one, two or three substituents each independently selected from —OH, —O—C1-4alkyl, halo, cyano, —NR11aR11b,
—S(═O)₂-C1-4alkyl, Het3a, and Het6a;
Het3, Het3a, Het5 and Het5a each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C1-4alkyl, halo, —OH, —NR11aR11b, or oxo; and wherein said heterocyclyl is optionally substituted on one nitrogen atom with C1-4alkyl or —(C═O)—C1-4alkyl;
Het4 and Het7 each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C1-4alkyl or —(C═O)—O—C1-4alkyl; and
wherein said aromatic ring is optionally substituted on one or two carbon atoms with in total one or two substituents each independently selected from the group consisting of —OH, halo, C1-4alkyl, —O—C1-4alkyl, —NR11aR11b, C1-4alkyl-NR11aR11b, —NH—C(═O)—C1-4alkyl, cyano, —COOH, —NH—C(═O)—O—C1-4alkyl, —NH—C(═O)—Cy3, —NH—C(═O)—NR10aR10b, —(C═O)—O—C1-4alkyl, —NH—S(═O)₂-C1-4alkyl, Het8a, —C1-4alkyl-Het8a, Het8b, Het9, and —C(═O)—NR10aR10b;
Het6a, Het8 and Het8a each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C1-4alkyl, —NH—C(═O)—Cy3, —(C═O)—NR10aR10b, —O—C3-6cycloalkyl, —S(═O)₂-C1-4alkyl, cyano, C1-4alkyl, —C1-4alkyl-OH, —O—C1-4alkyl, —O—(C═O)—NR10aR10b, and
—O—(C═O)—C1-4alkyl; and wherein said heterocyclyl is optionally substituted on one nitrogen with a substituent selected from the group consisting of —C(═O)—C1-4alkyl, —S(═O)₂-C1-4alkyl, and —(C═O)—NR10aR10b;
Het6b and Het8b each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C1-4alkyl, —OH, oxo, —(C═O)—NR10aR10b, —NH—C(═O)—C1-4alkyl, —NH—C(═O)—Cy3, and —O—C1-4alkyl; and wherein said heterocyclyl is optionally substituted on one nitrogen with a substituent selected from the group consisting of —C(═O)—C1-4alkyl, —C(═O)—Cy3, —(C═O)—C1-4alkyl-OH, —C(═O)—C1-4alkyl-O—C1-4alkyl, —C(═O)—C1-4alkyl-NR11aR11b, and C1-4alkyl;
Het9 represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C1-4alkyl; and wherein said aromatic ring is optionally substituted on one or two carbon atoms with in total one or two substituents each independently selected from the group consisting of —OH, halo, and C1-4alkyl;
Cy1 represents C3-6cycloalkyl optionally substituted with one, two or three substituents selected from the group consisting of —OH, —NH—C(═O)—C1-4alkyl, C1-4alkyl,
—NH—S(═O)₂-C1-4alkyl, —S(═O)₂-C1-4alkyl, and —O—C1-4alkyl;
Cy2 represents C3-7cycloalkyl or a 5- to 12-membered saturated carbobicyclic system;
wherein said C3-7cycloalkyl or said carbobicyclic system is optionally substituted with one, two, three or four substituents each independently selected from the group consisting of halo, R6, —C(═O)—Het6a, Het6a, Het6b, —NR9aR9b, —OH, C1-4alkyl, —O—C1-4alkyl, cyano, 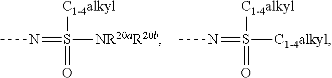
and
C1-4alkyl substituted with one or two substituents each independently selected from the group consisting of Het3a, Het6a, Het6b, and —NR9aR9b;
Cy3 represents C3-7cycloalkyl; wherein said C3-7cycloalkyl is optionally substituted with one, two or three halo substituents;
R9a and R9b are each independently selected from the group consisting of hydrogen;
C1-4alkyl; C3-6cycloalkyl; —C(═O)—C1-4alkyl; —C(═O)—C3-6cycloalkyl; —S(═O)₂-C1-4alkyl;
Het5; Het7; —C1-4alkyl-R16; —C(═O)—C1-4alkyl-Het3a; —C(═O)—R14;
C3-6cycloalkyl substituted with one, two or three substituents selected from the group consisting of halo, —OH, —O—C1-4alkyl, —NR11aR11b, and cyano; and
C1-4alkyl substituted with one, two or three substituents selected from the group consisting of halo, —OH, —O—C1-4alkyl, —NR11aR11b, and cyano;
R11a, R11b, R13a, R13b, R15a, R15b, R17a, R17b, R20a, R20b, R22a, and R22b are each independently selected from the group consisting of hydrogen and C1-4alkyl;
R11c and R11d are each independently selected from the group consisting of hydrogen, C1-6alkyl, and —C(═O)—C1-4alkyl;
R10a, R10b and R10c are each independently selected from the group consisting of hydrogen, C1-4alkyl, and C3-6cycloalkyl;
R10d and R10e are each independently selected from the group consisting of C1-4alkyl, —O—C1-4alkyl and C3-6cycloalkyl;
R14 represents Het5a; Het7; Het8a; —O—C1-4alkyl; —C(═O)NR15aR15b; C3-6cycloalkyl substituted with one, two or three substituents selected from the group consisting of —O—C1-4alkyl and halo; or C1-4alkyl substituted with one, two or three substituents selected from the group consisting of —O—C1-4alkyl, —NR13aR13b, halo, cyano, —OH, Het8a, and Cy1;
R16 represents —C(═O)—NR17aR17b, —S(═O)₂-C1-4alkyl, Het5, Het7, or Het8.

2. The combination according to claim 1, wherein the at least one other therapeutic agent is a hypomethylating agent.

3. The combination according to claim 2, wherein the hypomethylating agent is azacitidine or a pharmaceutically acceptable salt or solvate thereof.

4. A pharmaceutical composition comprising a combination as claimed in claim 1 and a pharmaceutically acceptable carrier.

5. (canceled)

6. (canceled)

7. (canceled)

8. (canceled)

9. A method for treating a subject who has been diagnosed with cancer comprising administering to the subject:
a therapeutically effective amount of a menin-mixed lineage leukemia 1 (MLL) inhibitor of Formula (I), or a tautomer or a stereoisomeric form thereof, or a pharmaceutically acceptable salt or a solvate thereof; and
a therapeutically effective amount of at least one other therapeutic agent, wherein the at least one other therapeutic agent is a hypomethylating agent, a cytidine deaminase inhibitor, a DNA intercalating agent, a pyrimidine analog, a purine analog, a kinase inhibitor, a CD20 inhibitor, an IDH inhibitor, an immunomodulatory agent or a DHODH inhibitor;
wherein the menin-MLL inhibitor of Formula (I) has the structure: 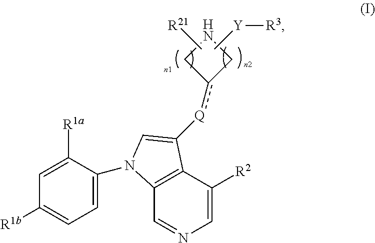
wherein
Q represents —CHRy-, —O—, —C(═O)—, —NRq-, or —CRy=; the dotted line is an optional additional bond to form a double bond in case Q represents —CRy=;
R1a represents hydrogen; cyano; halo; Het; —C(═O)—NRxaRxb; —S(═O)₂-R18;
—C(═O)—O—C1-4alkyl-NR22aR22b; —C(═O)—O—C1-4alkyl; 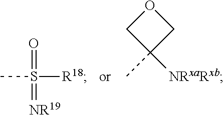
R18 represents C1-6alkyl or C3-6cycloalkyl;
R19 represents hydrogen or C1-6alkyl;
or R18 and R19 are taken together to form —(CH2)3-, —(CH2)4- or —(CH2)5-;
Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C1-4alkyl, C3-6cycloalkyl, or cyano;
Rxa and Rxb are each independently selected from the group consisting of hydrogen;
Het3; C3-6cycloalkyl; and C1-6alkyl; wherein optionally said C3-6cycloalkyl and C1-6alkyl are substituted with 1, 2 or 3 substituents each independently selected from the group consisting of —OH, —OC1-4alkyl, —C1-4alkyl-OH, halo, CF3, C3-6cycloalkyl, Het3, and NR11cR11d;
or Rxa and Rxb are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C1-4alkyl, halo, —OH, —O—C1-4alkyl, cyano, and C1-4alkyl substituted with one, two or three substituents selected from the group consisting of halo and OR23;
or Rxa and Rxb are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C1-4alkyl, halo, —OH, —O—C1-4alkyl, cyano, and C1-4alkyl substituted with one, two or three substituents each independently selected from the group consisting of halo and OR23;
R23 represents hydrogen or C1-4alkyl optionally substituted with one, two or three halo;
R1b represents hydrogen, F, Cl, or —O—C1-4alkyl;
R2 represents halo, C3-6cycloalkyl, C1-4alkyl, —O—C1-4alkyl, cyano, or C1-4alkyl substituted with one, two or three halo substituents;
R21 represents hydrogen or —Ya-R3a; provided that when R21 represents —Ya-R3a, one of —Ya-R3a and —Y—R3 is attached to the nitrogen atom of the ring;
Y and Ya each independently represent a covalent bond or 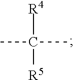
n1 is selected from 1 and 2;
n2 is selected from 1, 2, 3 and 4;
Ry represents hydrogen, —OH, C1-4alkyl, —C1-4alkyl-OH, or —C1-4alkyl-O—C1-4alkyl;
Rq represents hydrogen or C1-4alkyl;
R5 represents hydrogen, C1-4alkyl, or C3-6cycloalkyl;
R3, R3a, and R4 are each independently selected from the group consisting of Heti; Het2; Cy2;
C1-8alkyl; and C1-8alkyl substituted with one, two, three or four substituents each independently selected from the group consisting of —C(═O)—NR10aR10b, —C(═O)—Het6a, —C(═O)—Het6b, —NR10c-C(═O)—C1-4alkyl, —S(═O)₂-C1-4alkyl, —NRxcRxd, —NR8aR8b, —CF3, cyano, halo, —OH, —O—C1-4alkyl, Het¹, Het2, Ar1, and Cy2;
Rxc represents Cy1; Het5; —C1-6alkyl-Cy1; —C1-6alkyl-Het3; —C1-6alkyl-Het4;
or —C1-6alkyl-phenyl;
Rxd represents hydrogen; C1-4alkyl; or C1-4alkyl substituted with one, two or three substituents selected from the group consisting of halo, —OH, —O—C1-4alkyl, and cyano;
or Rxc and Rxd are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo,
—OH, —O—C1-4alkyl, —(C═O)—C1-4alkyl, —S(═O)₂-C1-4alkyl, and cyano;
or Rxc and Rxd are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C1-4alkyl, —(C═O)—C1-4alkyl-S(═O)₂-C1-4alkyl, and cyano;
R8a and R8b are each independently selected from the group consisting of hydrogen;
C1-6alkyl; —(C═O)—C1-4alkyl; and C1-6alkyl substituted with one, two or three substituents each independently selected from the group consisting of —OH, cyano, halo, —S(═O)₂-C1-4alkyl, —O—C1-4alkyl, —C(═O)—NR10aR10b, and —NR10c-C(═O)—C1-4alkyl;
Ar represents phenyl optionally substituted with one, two or three substituents each independently selected from the group consisting of C1-4alkyl, halo, —O—C1-4alkyl, —CF3, —OH, —S(═O)₂-C1-4alkyl, and —C(═O)—NR10aR10b;
Het1 represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R6, —C(═O)—Cy1, and —C(═O)—R8; and wherein said heterocyclyl is optionally substituted on one or two carbon atoms with in total one, two, three or four substituents each independently selected from the group consisting of halo, R6, Het6a, Het6b, C1-4alkyl, oxo, —NR9aR9b and —OH;
Het2 represents C-linked pyrazolyl, 1,2,4-oxadiazolyl, pyridazinyl or triazolyl; which may be optionally substituted on one nitrogen atom with R6a;
R6 and R6a are each independently selected from the group consisting of
Het3; Het4; —C(═O)—NH—Cy1; —C(═O)—NH—R8; —C(═O)—Het6a; —C(═O)—NR10dR10e;
—C(═O)—O—C1-4alkyl; —S(═O)₂-C1-4alkyl;
C1-6alkyl optionally substituted with one or two substituents each independently selected from the group consisting of Het3, Het4, Het6a, Het6b, Cy1, —CN, —OH, —O—C1-4alkyl, —C(═O)—NH—C1-4alkyl, —C(═O)—N(C1-4alkyl)2, —C(═O)—NH—C1-4alkyl-C3-6cycloalkyl, —C(═O)—OH, —NR11aR11b, and —NH—S(═O)₂-C1-4alkyl; and
C3-6cycloalkyl optionally substituted by one or two substituents each independently selected from the group consisting of —CN, —OH, —O—C1-4alkyl, —C(═O)—NH—C1-4alkyl, —C(═O)—N(C1-4alkyl)2, —NH—S(═O)₂-C1-4alkyl, and C1-4alkyl optionally substituted with one substituent selected from the group consisting of OH, —O—C1-4alkyl, —C(═O)—NH—C1-4alkyl and —NH—S(═O)₂-C1-4alkyl;
R8 represents hydrogen, —O—C1-6alkyl, C1-6alkyl; or C1-6alkyl substituted with one, two or three substituents each independently selected from —OH, —O—C1-4alkyl, halo, cyano, —NR11aR11b,
—S(═O)₂-C1-4alkyl, Het3a, and Het6a;
Het3, Het3a, Het5 and Het5a each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C1-4alkyl, halo, —OH, —NR11aR11b, or oxo; and wherein said heterocyclyl is optionally substituted on one nitrogen atom with C1-4alkyl or —(C═O)—C1-4alkyl;
Het4 and Het7 each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C1-4alkyl or —(C═O)—O—C1-4alkyl; and
wherein said aromatic ring is optionally substituted on one or two carbon atoms with in total one or two substituents each independently selected from the group consisting of —OH, halo, C1-4alkyl, —O—C1-4alkyl, —NR11aR11b, C1-4alkyl-NR11aR11b, —NH—C(═O)—C1-4alkyl, cyano, —COOH, —NH—C(═O)—O—C1-4alkyl, —NH—C(═O)—Cy3, —NH—C(═O)—NR10aR10b, —(C═O)—O—C1-4alkyl, —NH—S(═O)₂-C1-4alkyl, Het8a, —C1-4alkyl-Het8a, Het8b, Het9, and —C(═O)—NR10aR10b;
Het6a, Het8 and Het8a each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C1-4alkyl, —NH—C(═O)—Cy3, —(C═O)—NR10aR10b, —O—C3-6cycloalkyl, —S(═O)₂-C1-4alkyl, cyano, C1-4alkyl, —C1-4alkyl-OH, —O—C1-4alkyl, —O—(C═O)—NR10aR10b, and
—O—(C═O)—C1-4alkyl; and wherein said heterocyclyl is optionally substituted on one nitrogen with a substituent selected from the group consisting of —C(═O)—C1-4alkyl, —S(═O)₂-C1-4alkyl, and —(C═O)—NR10aR10b;
Het6b and Het8b each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C1-4alkyl, —OH, oxo, —(C═O)—NR10aR10b, —NH—C(═O)—C1-4alkyl, —NH—C(═O)—Cy3, and —O—C1-4alkyl; and wherein said heterocyclyl is optionally substituted on one nitrogen with a substituent selected from the group consisting of —C(═O)—C1-4alkyl, —C(═O)—Cy3, —(C═O)—C1-4alkyl-OH, —C(═O)—C1-4alkyl-O—C1-4alkyl, —C(═O)—C1-4alkyl-NR11aR11b, and C1-4alkyl;
Het9 represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C1-4alkyl; and wherein said aromatic ring is optionally substituted on one or two carbon atoms with in total one or two substituents each independently selected from the group consisting of —OH, halo, and C1-4alkyl;
Cy1 represents C3-6cycloalkyl optionally substituted with one, two or three substituents selected from the group consisting of —OH, —NH—C(═O)—C1-4alkyl, C1-4alkyl,
—NH—S(═O)₂-C1-4alkyl, —S(═O)₂-C1-4alkyl, and —O—C1-4alkyl;
Cy2 represents C3-7cycloalkyl or a 5- to 12-membered saturated carbobicyclic system;
wherein said C3-7cycloalkyl or said carbobicyclic system is optionally substituted with one, two, three or four substituents each independently selected from the group consisting of halo, R6, —C(═O)—Het6a, Het6a, Het6b, —NR9aR9b, —OH, C1-4alkyl, —O—C1-4alkyl, cyano, 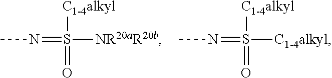
and
C1-4alkyl substituted with one or two substituents each independently selected from the group consisting of Het3a, Het6a, Het6b, and —NR9aR9b;
Cy3 represents C3-7cycloalkyl; wherein said C3-7cycloalkyl is optionally substituted with one, two or three halo substituents;
R9a and R9b are each independently selected from the group consisting of hydrogen;
C1-4alkyl; C3-6cycloalkyl; —C(═O)—C1-4alkyl; —C(═O)—C3-6cycloalkyl; —S(═O)₂-C1-4alkyl;
Het5; Het7; —C1-4alkyl-R16; —C(═O)—C1-4alkyl-Het3a; —C(═O)—R14;
C3-6cycloalkyl substituted with one, two or three substituents selected from the group consisting of halo, —OH, —O—C1-4alkyl, —NR11aR11b, and cyano; and
C1-4alkyl substituted with one, two or three substituents selected from the group consisting of halo, —OH, —O—C1-4alkyl, —NR11aR11b, and cyano;
R11a, R11b, R13a, R13b, R15a, R15b, R17a, R17b, R20a, R20b, R22a, and R22b are each independently selected from the group consisting of hydrogen and C1-4alkyl;
R11c and R11d are each independently selected from the group consisting of hydrogen, C1-6alkyl, and —C(═O)—C1-4alkyl;
R10a, R10b and R10c are each independently selected from the group consisting of hydrogen, C1-4alkyl, and C3-6cycloalkyl;
R10d and R10e are each independently selected from the group consisting of C1-4alkyl, —O—C1-4alkyl and C3-6cycloalkyl;
R14 represents Het5a; Het7; Het8a; —O—C1-4alkyl; —C(═O)NR15aR15b; C3-6cycloalkyl substituted with one, two or three substituents selected from the group consisting of —O—C1-4alkyl and halo; or C1-4alkyl substituted with one, two or three substituents selected from the group consisting of —O—C1-4alkyl, —NR13aR13b, halo, cyano, —OH, Het8a, and Cy1;
R16 represents —C(═O)—NR17aR17b, —S(═O)₂-C1-4alkyl, Het5, Het7, or Het8.

10. The method according to claim 9, wherein the at least one other therapeutic agent is a hypomethylating agent.

11. The method according to claim 10, wherein the hypomethylating agent is azacitidine or a pharmaceutically acceptable salt or solvate thereof.

12. The method according to claim 9, wherein the cancer is a hematopoietic disorder.

13. The method according to claim 12, wherein the hematopoietic disorder is a nucleophosmin 1 (NPM1)-mutated leukemia or MLL-rearranged leukemia.

14. The method according to claim 12, wherein the hematopoietic disorder is acute myeloid leukemia (AML) or acute lymphoblastic leukemia (ALL).


SEQUENCE LISTING (1 sequence)

SEQ ID NO: 1 - protein, 616 aa, synthetic construct
MGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGLHHHHHH